(19) United States
(12) Patent Application Publication
(10) Pub. No.: US 2025/0304683 A1
(43) Pub. Date: Oct. 2, 2025

(54) ENGINEERED BISPECIFIC MOLECULES AND METHODS OF USE

(71) Applicant: Cantai Therapeutics Inc., Philadelphia, PA (US)

(72) Inventors: Geeta VEMURI, Bryn Mawr, PA (US); Mai TANAKA-WAKEFIELD, Boston, MA (US)

(21) Appl. No.: 19/238,328

(22) Filed: Jun. 13, 2025

Related U.S. Application Data
(63) Continuation of application No. PCT/US24/41785, filed on Aug. 9, 2024.
(60) Provisional application No. 63/518,463, filed on Aug. 9, 2023.

(51) Int. Cl. C07K 16/28; A61K 39/00; A61P 29/00; A61P 37/06; C07K 16/24

(52) U.S. Cl. CPC C07K 16/2803; A61P 29/00; A61P 37/06; C07K 16/244; A61K 2039/542; C07K 2317/31; C07K 2317/52; C07K 2317/56; C07K 2317/71; C07K 2317/72; C07K 2317/76

(57) ABSTRACT

Provided herein are engineered proteins and methods of treating using the engineered proteins, wherein the engineered proteins comprise a first region and a second region, wherein the first region binds TREM1, a variant thereof or a functional fragment thereof, and wherein the second region binds an interleukin, wherein the interleukin comprises a protein selected from any one of IL-1 family, IL-6 family, IL-12 family and IL-23 family, a variant thereof or a functional fragment thereof.

This application is a continuation of International Application No. PCT/US2024/041785, filed Aug. 9, 2024, which claims the benefit of priority to U.S. Provisional Application No. 63/518,463, filed on Aug. 9, 2023 the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 220710-702601_PCT_SL.xml, which was created on Aug. 8, 2024, and is 908,921 bytes in size, is hereby incorporated by reference in its entirety.

### FIELD

The disclosure generally relates to engineered protein molecules that bind TREM1 and an interleukin.

### BACKGROUND OF THE DISCLOSURE

Bispecific antibodies (BsAbs) are antibodies with two binding sites each independently directed at two different antigens, or alternatively, two different epitopes on the same antigen. The therapeutic utility of BsAbs has shown to result in the potential for enhanced activity in comparison to that of mono-specific antibodies. BsAbs are understood to have broader applications for immunotherapy in treatment of various diseases.

### SUMMARY OF THE DISCLOSURE

Provided are engineered protein construct and other exemplary compositions that bind both TREM1 and an interleukin. For example, in some embodiments, an engineered protein construct, such as a bispecific molecule, comprises a first region and a second region. In some embodiments, the first region binds TREM1, a variant thereof or a functional fragment thereof. In some embodiments, the second region binds an interleukin, wherein the interleukin comprises a protein selected from any one of IL-1 family, IL-6 family, IL-12 family and IL-23 family, a variant thereof or a functional fragment thereof. In some embodiments, the engineered protein construct is an antibody, a variant thereof, or a functional fragment thereof. In some embodiments, the engineered protein construct is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the engineered protein construct comprises a heterodimeric antibody or a functional fragment thereof. In some embodiments, the engineered protein construct comprises a constant region. In some embodiments, the first region comprises a TREM1-binding heavy chain variable domain. In some embodiments, the first region comprises a TREM1-binding light chain variable domain. In some embodiments, the second region comprises an interleukin binding heavy chain variable domain. In some embodiments, the second region comprises an interleukin binding light chain variable domain. In some embodiments, a binding affinity of the first region for TREM1 is lower than a binding affinity of the second region for the interleukin. In some embodiments, the binding affinity of second binding region for the interleukin is at least two times the binding affinity of the first region for TREM1. In some embodiments, a binding affinity of the first region for TREM1 is higher than a binding affinity of the second region for the interleukin. In some embodiments, the binding affinity of the first region for TREM1 is at least two times the binding affinity of second binding region for the interleukin. In some embodiments, at least one of the first region and the second region comprises a light chain constant domain and/or heavy chain constant domain. In some embodiments, the engineered protein construct comprises at least one of a Fc region and/or a Fab region. In some embodiments, the Fc region comprises an amino acid sequence that is at least 60%, at least 65%, at least 70%, at least 75%, at least 80%, at least 85%, at least 90%, at least 95% or 100% identical to any one of amino acid sequences of SEQ ID NO: 453-455. In some embodiments, the heavy chain constant domain of the first region comprises the Fc region having S354C mutation and T366W mutation, per EU numbering, and the heavy chain constant domain of the second region comprises the Fc region having Y349C mutation, T366S mutation and Y407V mutation, per EU numbering. In some embodiments, the heavy chain constant domain of the second region comprises the Fc region having S354C mutation and T366W mutation, per EU numbering, and the heavy chain constant domain of the first region comprises the Fc region having Y349C mutation, T366S mutation and Y407V mutation, per EU numbering. In some embodiments, the Fc region comprises a human IgG1 heavy chain constant chain having at least one substitution is selected from positions N297, C226, C229, E233, L234, L235, G236, G237, P238, F243, M252, 5254, T256, D265, 5267, H268, D270, P271, R292, Y300, K322, A327, L328, P329, A330, P331, and P396, per EU numbering. In some embodiments, the Fc region comprises a human IgG2 heavy chain constant chain having at least one substitution is selected from positions C232, C233, V234, G237, P238, M252, S254, T256, H268, N297, V309, A330, and P331, per EU numbering. In some embodiments, the Fc region comprises a human IgG4 heavy chain constant chain having at least one substitution is selected from positions 5228, E233, F234, L235, L236, G237, S241, L248, M252, S254, T256, N297, E318, and T394, per EU numbering. In some embodiments, the engineered protein constructs described herein exhibit a pH-dependent target binding activity for a target peptide, wherein the target peptide is selected from TREM1, an interleukin, a variant thereof and a functional fragment thereof, and wherein the interleukin is selected from IL-1 family of proteins, IL-6 family of proteins, IL-12 family of proteins, and IL-23 family of proteins. In some embodiments, the engineered protein construct comprises anti-inflammatory activity that is at least 10%, at least 20%, at least 30%, at least 40%, at least 50%, at least 60%, at least 70%, at least 80%, at least 90%, at least 100%, or more relative to a combined anti-inflammatory activity of a monospecific antibody that binds TREM1 and a monospecific antibody that binds interleukin.

Also described herein are compositions comprising engineered protein constructs described herein.

Also described herein are engineered protein constructs for use in the treatment of an inflammatory disease or condition, wherein the engineered protein constructs are any one of the engineered protein constructs described herein. In some embodiments, the inflammatory disease or condition is selected from the group consisting of: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis.

Also described herein are pharmaceutical compositions, wherein the pharmaceutical compositions comprise any one of the engineered protein constructs (e.g., bispecific) described herein, and a pharmaceutically acceptable carrier.

Also described herein are pharmaceutical compositions for use in treating an inflammatory disease or condition, wherein the pharmaceutical composition comprises: a TREM1 binding moiety, an interleukin binding moiety and a pharmaceutically acceptable carrier, wherein the interleukin binding moiety comprises a protein selected from an IL-1 binding moiety, an IL-6 binding moiety, an IL-12 binding moiety, and an IL-23 binding moiety, and wherein administration of an effective amount of the composition to a subject in need thereof results in the treatment of inflammatory disease or condition. In some embodiments, the inflammatory disease or condition is selected from the group consisting of: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis.

Also described herein are methods of treating an inflammatory disease or condition in a subject. In some embodiments, the methods comprise administering to the subject an effective amount of the engineered protein construct, the composition or the pharmaceutical composition described herein, thereby treating the inflammatory disease or condition. In some embodiments, the inflammatory disease or condition is associated with increased activity and/or expression of TREM1, the interleukin, one or more downstream inflammatory signaling proteins thereof, or combinations thereof relative to a subject not having the inflammatory disease or condition. In some embodiments, the method reduces occurrence of candida infection in the subject relative to the subject being treated with a monospecific antibody that reduces interleukin activity.

Also described herein are compositions. In some embodiments, the compositions comprise an engineered protein construct comprising a first region and a second region, wherein the first region binds TREM1, a variant thereof or a functional fragment thereof, wherein the second region binds an interleukin, wherein the interleukin comprises a protein selected from any one of IL-1 family, IL-6 family, IL-12 family and IL-23 family, a variant thereof or a functional fragment thereof, and wherein administration of an effective amount of the composition to a subject in need thereof results in treatment of a disease or condition. In some embodiments, the inflammatory disease or condition is selected from the group consisting of: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis. In some embodiments, the inflammatory disease or condition is rheumatoid arthritis, juvenile arthritis, psoriatic arthritis, axial spondyloarthritis or ankylosing spondylitis. In some embodiments, the inflammatory disease or condition is psoriasis or hidradenitis suppurativa. In some embodiments, the inflammatory disease or condition is ulcerative colitis, Crohn's disease, necrotizing enterocolitis, sepsis, or multiple sclerosis. In some embodiments, the inflammatory disease or condition is sepsis. In some embodiments, the inflammatory disease or condition is multiple sclerosis.

Also described herein are nucleic acids. In some embodiments, the nucleic acids encode at least a portion of any one of the engineered protein constructs (e.g., multispecifics, (e.g., bispecific)) described herein or the engineered protein constructs of the composition described herein.

Also described herein are methods of reducing an IL-1 associated inflammatory condition in a subject, the methods comprise administering to the subject an effective amount of a pharmaceutical composition comprising an IL-1 binding moiety, a TREM1 binding moiety, and a pharmaceutically acceptable carrier, thereby reducing IL-1 associated inflammatory condition in the subject relative to the IL-1 associated inflammatory condition in the subject prior to administration of the pharmaceutical composition. In some embodiments, the method increases expression of at least one of nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), and Interferon lambda receptor 1 (IFNLR1). In some embodiments, the method restores pentose phosphate pathway (PPP). In some embodiments, the pharmaceutical composition comprises any one of the pharmaceutical compositions described herein.

Also described herein are methods of reducing an IL-6 associated inflammatory condition in a subject, the methods comprise administering to the subject an effective amount of a pharmaceutical composition comprising an IL-6 binding moiety, a TREM1 binding moiety and a pharmaceutically acceptable carrier, thereby reducing IL-6 associated inflammatory condition in the subject relative to the IL-6 associated inflammatory condition in the subject prior to administration of the pharmaceutical composition. In some embodiments, the method increases expression of at least one of nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), and Interferon lambda receptor 1 (IFNLR1). In some embodiments, the method restores pentose phosphate pathway (PPP). In some embodiments, the pharmaceutical composition comprises any one of the pharmaceutical compositions described herein.

Also described herein are methods of reducing an IL-12 associated inflammatory condition in a subject, the methods comprise administering to the subject an effective amount of a pharmaceutical composition comprising an IL-12 binding moiety, a TREM1 binding moiety, and a pharmaceutically acceptable carrier, thereby reducing IL-12 associated inflammatory condition in the subject relative to the IL-12 associated inflammatory condition in the subject prior to administration of the pharmaceutical composition. In some embodiments, the method increases expression of at least one of nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), and Interferon lambda receptor 1 (IFNLR1). In some embodiments, the method restores pentose phosphate pathway (PPP). In some embodiments, the pharmaceutical composition comprises any one of the pharmaceutical compositions described herein.

Also described herein are methods of reducing an IL-23 associated inflammatory condition in a subject, the methods comprise administering to the subject an effective amount of a pharmaceutical composition comprising an IL-1 binding moiety, a TREM1 binding moiety, and a pharmaceutically acceptable carrier, thereby reducing IL-23 associated inflammatory condition in the subject relative to the IL-23 associated inflammatory condition in the subject prior to administration of the pharmaceutical composition. In some embodiments, the method increases expression of at least one of nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), and Interferon lambda receptor 1 (IFNLR1). In some embodiments, the method restores pentose phosphate pathway (PPP). In some embodiments, the pharmaceutical composition comprises any one of the pharmaceutical compositions described herein.

Also described herein are methods of reducing a TREM1 associated inflammatory condition in a subject, the methods comprise administering to the subject an effective amount of a pharmaceutical composition comprising a TREM1 binding moiety, an interleukin binding moiety, and a pharmaceutically acceptable carrier, thereby reducing TREM1 associated inflammatory condition in the subject relative to the TREM1 associated inflammatory condition in the subject prior to administration of the pharmaceutical composition. In some embodiments, the method increases expression of at least one of nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), and Interferon lambda receptor 1 (IFNLR1). In some embodiments, the method restores pentose phosphate pathway (PPP). In some embodiments, the pharmaceutical composition comprises any one of the pharmaceutical compositions described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a bispecific antibody comprising a TREM1 binding region and an interleukin binding region.

FIG. 2 show effects of contacting human peripheral blood mononuclear cells (PBMC) with a combination of antibodies, wherein the combination of a TREM1 binding antibody and an interleukin (e.g., IL-6 and IL-23) binding antibody. Briefly, FIG. 2 shows amount of tumor necrosis factor α (TNFα), IL-1β, IL-17, IL-23 and Macrophage Inflammatory Protein-3 Alpha (MIP-3α) present in supernatant of the human PBMC following contacting with the combination of the TREM1 binding antibody and the interleukin binding antibody (IL-6 or IL-23).

### DETAILED DESCRIPTION OF EMBODIMENTS

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features can also be provided separately or in any suitable combination. Conversely, although the present disclosure may be described herein in the context of separate embodiments for clarity, the present disclosure can also be implemented in a single embodiment.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms, “disease”, “disorder”, and “condition,” which are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

As used herein, the term, “in need thereof,” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the term, “administering,” refers to the placement of a compound (e.g., an antibody or antigen binding fragment thereof as disclosed herein) into a subject by a method or route that results in at least partial delivery of the agent at a desired site. Pharmaceutical compositions comprising an antibody or antigen binding fragment thereof, disclosed herein can be administered by any appropriate route which results in an effective treatment in the subject, including but not limited to intravenous, intraarterial, subcutaneous injection or infusion directly into a tissue parenchyma, etc. Where necessary or desired, administration can include, for example, intracerebroventricular (“icv”) administration, intranasal administration, intracranial administration, intracelial administration, intracerebellar administration, subcutaneous administration, or intrathecal administration.

As used herein, the term, “subject”, “patient”, “individual” and like terms, which are used interchangeably, refer to a vertebrate, a mammal, a primate, or a human. Mammals include, without limitation, humans, primates, rodents, wild or domesticated animals, including feral animals, farm animals, sport animals, and pets. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. A subject can be male or female. In some embodiments, the subject is a mammal. The mammal can be a human, non-human primate, mouse, rat, dog, cat, horse, or cow, but is not limited to these examples. Mammals other than humans can be advantageously used as subjects that represent animal models of conditions or disorders. Non-limiting examples include murine models. In addition, the compositions and methods described herein can be used to treat domesticated animals and/or pets. A subject can be one who is diagnosed and currently being treated for, or seeking treatment, monitoring, adjustment or modification of an existing therapeutic treatment, or is at a risk of developing a given disorder.

As used herein, the terms, “protein”, “peptide” and “polypeptide,” which are used interchangeably, refer to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. A polypeptide can have the amino acid sequence of naturally occurring polypeptide from any mammal. Such native sequence polypeptide can be isolated from nature or can be produced by recombinant or synthetic means. In some embodiments, the polypeptide is a “variant”. “Variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide. A “derivative” of a polypeptide is a polypeptide (e.g., an antibody) that has been chemically modified, e.g., via conjugation to another chemical moiety (such as, for example, polyethylene glycol or albumin, e.g., human serum albumin), phosphorylation, and glycosylation.

As used herein, the term, “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

As used herein, the terms, “increased”,“increase”, and “enhance,” refer to an increase by a statistically significant amount; for the avoidance of doubt, the terms “increased”, “increase”, or “enhance”, mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “antibody” refers to an immunoglobulin molecule that specifically binds to, or is immunologically reactive toward, a specific antigen which in the current instance can be, for example, TREM1, IL-1 family, IL-6 family, IL-12 family or IL-23 family. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, Fab′, F(ab′)2, Fab, Fv, rlgG, scFv, hcAbs (heavy chain antibodies), a single domain antibody, V_{HH}, V_{NAR}, sdAbs, or nanobody. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody may comprise four polypeptides: two identical copies of a heavy (H) chain polypeptide and two identical copies of a light (L) chain polypeptide. Each of the heavy chains may contain one N-terminal variable (VH) region and three C-terminal constant (CH1, CH2 and CH3) regions, and each light chain may contain one N-terminal variable (VL) region and one C-terminal constant (CL) region. The variable regions of each pair of light and heavy chains may form an antigen binding site of an antibody. In exemplary embodiments of bispecific antibodies, multiple distinct antigen binding sites may be present. The VH and VL regions may have a similar general structure, with each region comprising four framework regions, whose sequences are relatively conserved. In some embodiments, the framework regions may be connected by three complementarity determining regions (CDRs). In some embodiments, the three CDRs, known as CDR1, CDR2, and CDR3, form the “hypervariable region” of an antibody, which is responsible for antigen binding.

As used herein, the term, “chimeric antibody,” refers to an antibody in which a portion of the heavy and/or light chain is derived from a particular source or species, while the remainder of the heavy and/or light chain is derived from a different source or species.

As used herein, the term, “human antibody,” refers to an antibody comprising an amino acid sequence corresponding to that of an antibody produced by a human or a human cell, or derived from a non-human source that utilizes a human antibody repertoire or human antibody-encoding sequences (e.g., obtained from human sources or designed de novo).

As used herein, the term, “humanized antibody,” refers to an amino acid sequence that differs from the amino acid sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In some embodiments, certain amino acids in the framework and constant domains of the heavy and/or light chains of the non-human species antibody are mutated to produce the humanized antibody. In some embodiments, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

As used herein, the term, “epitope,” means a portion of an antigen that specifically binds to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TREM1, a variant thereof or a fragment thereof, any one of IL-1 family, IL-6 family, IL-12 family and IL-23 family, a variant thereof or a fragment thereof, or a combination thereof.

As used herein, the term, “Complementarity Determining Regions” (CDRs, i.e., CDR1, CDR2, and CDR3), refers to the amino acid residues of an antibody variable domain the presence of which are necessary for antigen binding. Each variable domain typically has three CDR regions identified as CDR1, CDR2 and CDR3. The CDRs of variable heavy chain can be CDR-H1, CDR-H2 and CDR-H3. The CDRs of variable light chain can be CDR-L1, CDR-L2 and CDR-L3. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th ed. (1991)). Thus, the HVs may be comprised within the corresponding CDRs and references herein to the “hypervariable loops” of VH and VL domains should be interpreted as also encompassing the corresponding CDRs, and vice versa, unless otherwise indicated. The more highly conserved regions of variable domains are called the framework region (FR), as defined below. The variable domains of native heavy and light chains each comprise four FRs (FR1, FR2, FR3 and FR4, respectively), largely adopting a [beta]-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215: 175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations. The antibodies or antigen-binding fragment thereof of the present disclosure can comprise a CDR3 region that is a length of at least about 10, 11, 12, 13, 14, 15, 16, 17, 18, 19, 20, 21, 22, 23, 24, 25, 26, 27, 28, 29, or 30 amino acids in length. The antibodies or antigen-binding fragment thereof of the present disclosure can comprise a CDR3 region that is at least about 18 amino acids in length.

As used herein, the term, “variable region,” when used in reference to an antibody, refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Allazikani et al (1997) J. Molec. Biol. 273:927-948)). A CDR may refer to CDRs defined by either approach or by a combination of both approaches. Six hypervariable loops (three loops each from the Heavy and Light chain) contribute the amino acid residues for antigen-binding and confer antigen-binding specificity to the antibody. However, even a single variable domain (or half of an Fv comprising only three CDRs specific for an antigen) has the ability to recognize and bind antigen, although at a lower affinity than the entire binding site.

As used herein, the term, “constant region,” when used in reference to an antibody, refers to the constant region of the antibody light chain (i.e., a light chain constant region) or the constant region of the antibody heavy chain (i.e., a heavy chain constant region) either alone or in combination. The constant region does not vary with respect to antigen specificity.

As used herein, the terms, “heavy chain region,” includes amino acid sequences derived from the constant domains of an immunoglobulin heavy chain. A polypeptide comprising a heavy chain region comprises at least one of: a CH1 domain, a hinge (e.g., upper, middle, and/or lower hinge region) domain, a CH2 domain, a CH3 domain, or a variant or fragment thereof. In an embodiment, an antibody or an antigen-binding fragment thereof may comprise the Fc region of an immunoglobulin heavy chain (e.g., a hinge portion, a CH2 domain, and a CH3 domain). In another embodiment, an antibody or an antigen-binding fragment thereof lacks at least a region of a constant domain (e.g., all or part of a CH2 domain). In some embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, CH2 and CH3 domain sequences from a human immunoglobulin). In some embodiments, the constituent constant domains of the heavy chain region are from different immunoglobulin molecules.

As used herein, the term, “hinge region,” includes the region of a heavy chain molecule that joins the CH1 domain to the CH2 domain. The hinge region can comprise approximately 25 residues and is flexible, thus allowing the two N-terminal antigen binding regions to move independently. Hinge regions can be subdivided into three distinct domains: upper, middle, and lower hinge domains (Roux et al. J. Immunol. 1998 161:4083).

As used herein, the term “Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

As used herein, the term, “heavy chain variable region” or “VH,” when used in reference to an antibody, refers to the fragment of the heavy chain that contains three CDRs interposed between flanking stretches known as framework regions, these framework regions are generally more highly conserved than the CDRs and form a scaffold to support the CDRs.

As used herein, the term, “light chain variable region” or “VL,” when used in reference to an antibody, refers to the fragment of the light heavy chain that contains three CDRs interposed between flanking stretches known as framework regions, these framework regions are generally more highly conserved than the CDRs and form a scaffold to support the CDRs.

As used herein, the term, “framework residues” or “FR,” are those variable domain amino acid residues other than the hypervariable region amino acid residues.

As used herein, the term, “antibody heavy chain,” refers to the larger of the two types of polypeptide chains present in antibody molecules in their naturally occurring conformations, and which normally determines the class to which the antibody belongs.

As used herein, the term, “antibody light chain,” refers to the smaller of the two types of polypeptide chains present in antibody molecules in their naturally occurring conformations. Kappa (“x”) and lambda (“X”) light chains refer to the two major antibody light chain isotypes.

As used herein, the phrase, “specifically binds” or “preferentially binds,” refers to an antibody or antigen-binding fragment thereof that binds to a target with greater affinity and/or avidity than it binds to epitopes on unrelated polypeptides. The specificity of an antibody or antigen-binding fragment or portion thereof can be determined based on affinity and/or avidity. Methods to determine such specific binding are also well known in the art.

As used herein, the term, “multispecific antibody,” is an antibody that comprises two or more different antigen-binding domains that collectively specifically bind two or more different epitopes. The two or more different epitopes may be epitopes on the same cell or on different cells. In some embodiments, a multi-specific antibody binds two different epitopes (i.e., a “bispecific antibody”). In some embodiments, a multi-specific antibody binds three different epitopes (i.e., a “trispecific antibody”).

As used herein a “recombinant antibody” is an antibody that comprises an amino acid sequence derived from two different species, or two different sources, and includes synthetic and/or non-naturally-occurring molecules. By way of non-limiting example, a recombinant antibody may comprise a non-human CDR and a human variable region framework or constant or Fc region, an antibody with binding domains from two different monoclonal antibodies, or an antibody comprising a mutation of one or more amino acid residues to increase or decrease biological activity or binding of a part of the antibody. In certain embodiments, recombinant antibodies are produced from a recombinant DNA molecule or synthesized. In certain embodiments, the antibodies described herein are a polypeptide(s) encoded by one or more polynucleotides.

As used herein, “recognize” or “bind” or “selective for” refers to the association or binding between an antigen binding domain and an antigen. As used herein, an “antigen” refers to an antigenic substance that can trigger an immune response in a host. An antigenic substance can be a molecule, such as a costimulatory molecule that can trigger an immune response in a host.

As used herein, an “antibody construct” refers to a construct that may contain an antigen binding domain and an Fc domain.

As used herein, a “binding domain” refers to an antibody or non-antibody domain.

As used herein, an “antigen binding domain” refers to a binding domain from an antibody or from a non-antibody that can bind to an antigen. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can target the same antigen or different antigens.

As used herein, an “antibody antigen binding domain” refers to a binding domain from an antibody that can bind to an antigen.

As used herein, an “Fc domain” refers to an Fc domain from an antibody or from a non-antibody that can bind to an Fc receptor. As used herein, an “Fc domain” and an “Fe comprising domain” can be used interchangeably.

As used herein, a “target binding domain” refers to a construct that contains an antigen binding domain from an antibody or from a non-antibody that can bind to an antigen.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, He); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid.

The phrase “pharmaceutically acceptable” is employed herein to refer to those compounds, materials, compositions, and/or dosage forms which are, within the scope of sound medical judgment, suitable for use in contact with the tissues of subjects for instance, human beings and animals, without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An antigen can elicit an immune response. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

“Antigen recognition moiety” or “antibody recognition domain” refers to a molecule or portion of a molecule that specifically binds to an antigen. In one embodiment, the antigen recognition moiety is an antibody, antibody like molecule or fragment thereof and the antigen is an exogenous antigen or an infectious disease antigen.

The terms “fragment of an antibody,” “antibody fragment,” “functional fragment of an antibody,” “antigen binding domain” or their grammatical equivalents are used interchangeably herein to mean one or more fragments or portions of an antibody that retain the ability to specifically bind to an antigen (see, generally, Holliger et al., Nat. Biotech., 23(9):1126-1129 (2005)). The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment that may comprise VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (v) a diabody, which is a dimer of polypeptide chains, wherein each polypeptide chain may comprise a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Pat. No. 8,603,950. Other antibody fragments can include variable fragments of heavy chain antibodies (VHH).

As used herein, the term, “Fab,” refers to a region of an antibody composed of one constant and one variable domain of each of the heavy and the light chains (monovalent antigen-binding fragment), but wherein the heavy chain is truncated such that it lacks the CH2 and CH3 domain (i.e., VH, CH1, VL, and CL), and may also lack some or all of the hinge region. It can be produced by digestion of a whole antibody with the enzyme papain. Fab may refer to this region in isolation, or this region in the context of a full-length antibody, immunoglobulin construct or Fab fusion protein. Fab can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner.

As used herein, the term, “scFv,” refers to an antibody fragment comprising the VH and VL domains of an antibody, wherein these domains are present in a single polypeptide chain. See, for example, U.S. Pat. Nos. 4,946,778, 5,260,203, 5,455,030, and 5,856,456. Generally, the Fv polypeptide further comprises a polypeptide linker between the VH and VL domains that enables the scFv to form the desired structure for antigen-binding. For a review of scFv see Pluckthun (1994) The Pharmacology of Monoclonal Antibodies vol 113 ed. Rosenburg and Moore (Springer-Verlag, New York) pp 269-315. The VH and VL domain complex of Fv fragments may also be stabilized by a disulfide bond (U.S. Pat. No. 5,747,654).

The term, “conservative amino acid substitution” or “conservative mutation,” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids may be defined where amino acids within a group exchange preferentially with each other, and therefore resemble each other most in their impact on the overall protein structure. Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained. Alternatively or additionally, the therapeutic agents can comprise the amino acid sequence of the reference protein with at least one non-conservative amino acid substitution.

The terms “non-conservative mutation” or “non-conservative amino acid substitution” involve amino acid substitutions between different groups, for example, lysine for tryptophan, or phenylalanine for serine, etc. In this case, it is preferable for the non-conservative amino acid substitution to not interfere with, or inhibit the biological activity of the therapeutic agent. The non-conservative amino acid substitution may enhance the biological activity of the therapeutic agent, such that the biological activity of the therapeutic agent is increased as compared to the wild type therapeutic agent.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody is bivalent because it has two binding arms but is monospecific because it binds to one epitope. Multispecific, multivalent antibodies are constructs that have more than one binding region of different specificity. For example, the bispecific antibody constructs disclosed herein have a first antigen binding region and a second antigen binding region, wherein the first and second antigen binding regions are distinct.

A “bispecific antibody” is an antibody that can bind simultaneously to two targets which are of different structure. Bispecific antibodies (BsAbs) and bispecific antibody fragments (bsFab) may have at least one arm (or binding domain) that specifically binds to, for example, a first antigen, and at least one other arm (or binding domain) that specifically binds to a second antigen. At least one of the first and the second antigens may be an antigen produced by or associated with a diseased cell, tissue, organ or pathogen. A variety of bispecific antibodies can be produced using molecular engineering.

As used herein, the term, “vector,” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the terms, “host cell,” “host cell line” and “host cell culture,” are interchangeable and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

A bispecific antibody construct, or a composition described herein, is said to be administered in a “therapeutically effective amount” if the amount administered is physiologically significant. An agent is physiologically significant if its presence results in a detectable change in the physiology of a recipient subject. In particular embodiments, a bispecific antibody construct disclosed herein is physiologically significant if its presence invokes a response or mitigates the signs and symptoms of an infectious or autoimmune disease state. A physiologically significant effect could also be the evocation of a humoral and/or cellular immune response in the recipient subject.

The term “linker” is used to denote polypeptides comprising two or more amino acid residues joined by peptide bonds and are used to link one or more antigen binding portions or variable domains. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). In some embodiments, the linker peptide comprises an amino acid sequence that is at least 90%, 91%, 92%, 93%, 94%, 95%, 96%, 97%, 98%, 99%, or 100% identical to the amino acid sequence of SEQ ID NO: 44. In some embodiments, the linker peptide comprises an amino acid sequence that is at least 90%, 91%, 92%, 93%, 94%, 95%, 96%, 97%, 98%, 99%, or 100% identical to the amino acid sequence of SEQ ID NO: 45.

An “Fv” or “Fv fragment” may consist of only the light chain variable domain (VL) and heavy chain variable domain (VH) of a “single arm” of an immunoglobulin. Thus an “Fv” is the minimum antibody fragment which contains a complete antigen-recognition and binding site. A “two-chain” Fv fragment consists of a dimer of one heavy- and one light-chain variable domain in tight, non-covalent association. A single-chain Fv species (scFv) may include a VH and a VL domain of an immunoglobulin, with these domains being present in a single polypeptide chain in which they are covalently linked to each other by a linker peptide. Typically, in a scFv fragment the variable domains of the light and heavy chain associate in a dimeric structure analogous to that in a two-chain Fv species. In single chain Fv fragments, it is possible to either have the variable domain of the light chain arranged at the N-terminus of the single polypeptide chain, followed by the linker and the variable domain of the heavy chain arranged at the C-terminus of the polypeptide chain or vice versa, having the variable domain of the heavy chain arranged on the N-terminus and the variable domain of the light chain at the C-terminus with the linker peptide arranged in between. The linker peptide can be any flexible linker known in the art, for example, made from glycine and serine residues. It is also possible to additionally stabilize the domain association between the VH and the VL domain by introducing disulfide bonds into conserved framework regions (see Reiter et al. Stabilization of the Fv fragments in recombinant immunotoxins by disulfide bonds engineered into conserved framework regions, Biochemistry 1994, 33, 6551-5459). Such scFv fragments are also known as disulfide-stabilized scFv fragments (ds-scFv).

As used herein, the term, “treating” (and variations thereof such as “treat” or “treatment”), refers to clinical intervention in an attempt to alter the natural course of a disease or condition in a subject in need thereof. Treatment can be performed during the course of clinical pathology. Desirable effects of treatment include cure (if applicable), delay the onset of, reduce the severity of, alleviate, ameliorate one or more symptoms of the disease, improve the disease, reduce or improve any associated symptoms of the disease or the predisposition toward the development of the disease.

As used herein, the term, “sufficient amount,” means an amount sufficient to produce a desired effect, e.g., an amount sufficient to modulate an immune response in a subject.

As used herein, the terms, “modulate” and “modulation,” refer to reducing or inhibiting or, alternatively, activating or increasing, a recited variable.

### Functional Antibody Fragments

Functional antibody fragments bind to one or more target proteins. In some embodiments, functional antibody fragments promote degradation of one or more target proteins. In some embodiments, functional antibody fragments induce degradation of one or more target proteins. In some embodiments, functional antibody fragments induce/promote cleavage of one or more target proteins. In some embodiments, functional antibody fragments induce/promote internalization of one or more target proteins. In some embodiments, functional antibody fragments induce/promote shedding of one or more target proteins. In some embodiments, functional antibody fragments induce/promote downregulation of expression of one or more target proteins. In some embodiments, functional antibody fragments prevent one or more target proteins mediated activities of one or more downstream signaling proteins. In some embodiments, functional antibody fragments prevent one or more target protein mediated expression of one or more downstream signaling proteins. In some embodiments, a target protein is directly and/or indirectly associated with inflammation. Accordingly, in some embodiments, administration of functional antibody fragments in a subject can result in reduced inflammation relative to a subject that is not administered with the functional antibody fragments. In some embodiments, a target protein comprises TREM1, any one of IL-1 family of proteins, any one of IL-6 family of proteins, any one of IL-12 family of proteins and any one of IL-23 family of proteins, a variant thereof, a functional fragment thereof, or a combination thereof.

Function antibody fragments, which recognize specific epitopes, can be generated by known techniques. Functional antibody fragments are antigen binding portions of an antibody, such as, for example, F(ab′)2, Fab′, F(ab)2, Fab, Fv, scFv and the like. F(ab′)2 fragments can be produced by pepsin digestion of an antibody molecule and Fab′ fragments can be generated by reducing disulfide bridges of the F(ab′)2 fragments. Alternatively, Fab′ expression libraries can be constructed (Huse et al., 1989, Science, 246:1274-1281) to allow rapid and easy identification of monoclonal Fab′ fragments with a desired specificity. F(ab)2 fragments may be generated by papain digestion of an antibody.

A single chain Fv molecule (scFv) may comprise a VL domain and a VH domain. The VL and VH domains associate to form a target binding site. These two domains may be further covalently linked by a peptide linker (L). Methods for making scFv molecules and designing suitable peptide linkers are described in U.S. Pat. Nos. 4,704,692; 4,946,778; Raag and Whitlow, FASEB 9:73-80 (1995) and Bird and Walker, TIBTECH, 9: 132-137 (1991).

Techniques for producing single domain antibodies (DABs or VHH) are also known in the art, as disclosed for example in Cossins et al. (2006, Prot Express Purif 51:253-259), incorporated herein by reference. Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; Maass et al., J Immunol Methods 324:13-25, 2007). The VHH may have potent antigen-binding capacity and can interact with novel epitopes that are inaccessible to conventional VH-VL pairs. (Muyldermans et al., 2001). Alpaca serum IgG contains about 50% camelid heavy chain only IgG antibodies (HCAbs) (Maass et al., 2007). Alpacas may be immunized with known antigens, such as TNF-α, and VHHs can be isolated that bind to and neutralize the target antigen (Maass et al., 2007). PCR primers that amplify virtually all alpaca VHH coding sequences have been identified and may be used to construct alpaca VHH phage display libraries, which can be used for antibody fragment isolation by standard biopanning techniques well known in the art (Maass et al., 2007). In certain embodiments, VHH antibody fragments may be utilized in the claimed compositions and methods.

An antibody fragment can be prepared by proteolytic hydrolysis of the full-length antibody or by expression in E. coli or another host of the DNA coding for the fragment. An antibody fragment can be obtained by pepsin or papain digestion of full-length antibodies by conventional methods. These methods are described, for example, by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

### Engineered Protein Constructs

Disclosed herein are engineered protein constructs. In some embodiments, an engineered protein constructs described herein comprise a TREM1 binding moiety, an IL-1 binding moiety, an IL-6 binding moiety, an IL-12 binding moiety, an IL-23 binding moiety, or a combination thereof. In some embodiments, an engineered protein construct described herein comprises multispecific molecules described herein. Multispecific molecules are antibodies that are capable of binding at least two different targets. In some embodiments, a multispecific molecule targets at least two epitopes selected from TREM1, a protein selected from IL-1 family, a protein selected from IL-6 family, a protein selected from IL-12 family, a protein selected from IL-23 family, a variant thereof, or a functional fragment thereof. In some embodiments, the at least two different targets comprise two different epitopes. In some embodiments, the two different epitopes are: (a) TREM1, a variant thereof or a functional fragment thereof, and (b) a protein selected from any one of IL-1 family, IL-6 family, IL-12 family and IL-23 family, a variant thereof or a functional fragment thereof. Accordingly, in some embodiments, an engineered protein construct described herein is capable of binding at least two different epitopes, wherein the at least two different epitopes are: (a) TREM1, a variant thereof or a functional fragment thereof, and (b) a protein selected from any one of IL-1 family, IL-6 family, IL-12 family and IL-23 family, a variant thereof or a functional fragment thereof.

Methods for making multispecific antibodies are known in the art. Traditionally, recombinant production of multispecific antibodies is based on co-expression of two immunoglobulin heavy-chain/light-chain pairs, where the two heavy chains have different specificities (Milstein and Cuello, Nature, 305:537-539 (1983)). Purification of correct molecule is usually accomplished by affinity chromatography steps. Similar procedures are disclosed in WO 93/08829, and in Traunecker et al., EMBO J., 10:3655-3659 (1991).

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. Accordingly, multispecific antibodies described herein comprise kappa constant region, lambda constant region, alpha constant region, gamma constant region, delta constant region, epsilon constant region, mu constant region, a functional fragment thereof, or a combination thereof.

A class of antibody or immunoglobulin refers to the type of constant domain or constant region possessed by its heavy chain. In some embodiment, a heavy chain is an IgA. In some embodiment, a heavy chain is an IgD. In some embodiment, a heavy chain is an IgE. In some embodiment, a heavy chain is an IgG. In some embodiment, a heavy chain is an IgM. In some embodiment, a heavy chain is an IgG1. In some embodiment, a heavy chain is an IgG2. In some embodiment, a heavy chain is an IgG3. In some embodiment, a heavy chain is an IgG4. In some embodiment, a heavy chain is an IgA1. In some embodiment, a heavy chain is an IgA2. In some embodiments, an antibody is an IgG1 antibody.

In some embodiments, an antibody is an IgG3 antibody. In some embodiments, an antibody is an IgG2 antibody. In some embodiments, an antibody is an IgG4 antibody.

In some embodiments, multispecific antibodies described herein comprise light chains. In some embodiments, a light chain comprises a kappa light chain or a lambda light chain. Multispecific antibodies, such as kappa or lambda antibodies, can be made using any of a variety of art-recognized techniques, including those disclosed in WO 2012/023053, the contents of which are hereby incorporated by reference in their entirety.

In some embodiments, antibody variable domains with desired binding specificities (antibody-antigen combining sites) can be linked to immunoglobulin constant domain sequences to form multispecific antibodies. In some embodiments, a fusion comprising antibody variable domains is preferably linked with an immunoglobulin heavy-chain constant domain, wherein the immunoglobulin heavy-chain constant domain comprises at least part of a hinge, CH2, and CH3 regions. In some embodiments, it is preferred to have a first heavy-chain constant region (CH1) containing a site necessary for light-chain binding present in at least one of fusions. DNAs encoding immunoglobulin heavy-chain fusions and, if desired, the immunoglobulin light chain may be inserted into separate expression vectors and may be co-transfected into a suitable host organism. For further details of generating bispecific antibodies see, for example, Suresh et al., Methods in Enzymology, 121:210 (1986), the contents of which are incorporated by reference.

In some embodiments, an interface between a pair of antibody molecules described herein is engineered to maximize percentage of heterodimers that can be recovered from recombinant cell culture. In this method, one or more small amino acid side chains from the interface of a first antibody molecule is replaced with larger side chains to form a protuberance or knob (e.g., tyrosine or tryptophan). Compensatory cavities or holes of identical or similar size to the large side chain(s) are created on the interface of a second antibody molecule by replacing large amino acid side chains with smaller ones (e.g., alanine or threonine). This provides a mechanism for increasing yield of the heterodimer over other unwanted end-products such as homodimers.

Techniques for generating bispecific antibodies from functional antibody fragments have been described in the literature. For example, bispecific antibodies can be prepared using chemical linkage. The bispecific antibodies can be used as agents for the selective immobilization of enzymes.

Various techniques for making and isolating functional bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making functional bispecific antibody fragments. The functional fragments comprise a heavy-chain variable domain (VH) connected to a light-chain variable domain (VL) by a linker which is too short to allow pairing between the two domains on the same chain. Accordingly, VH and VL domains of one functional fragment are forced to pair with a complementary VL and VH domains of another functional fragment, thereby forming two antigen-binding sites. Another strategy for making functional bispecific antibody fragments by the use of single-chain Fv (sFv) dimers has also been reported. See, Gruber et al., J. Immunol. 152:5368 (1994).

Antibodies with more than two valences are contemplated. For example, trispecific antibodies can be prepared. Tutt et al., J. Immunol. 147:60 (1991). Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies may possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Several strategies have been used to generate multispecific molecules described herein (e.g., bispecific molecules, trispecific molecules), such as chemical cross-linking of functional antibody fragments, forced heterodimerization, quadroma technology, fusion of functional antibody fragments via polypeptide linkers, and use of single domain antibodies. Availability of recombinant DNA technologies has led to the generation of a multitude of bispecific antibody formats (see e.g., Ridgway J B et al. (1996) Protein Eng 9: 617-621). Linkers and mutations have frequently been introduced into different regions of the antibody to force heterodimer formation or to connect different binding moieties into a single molecule.

### IL-1 Family

The IL-1 family, as described herein, include Interleukin-1α cytokine (IL-1α), Interleukin-1β cytokine (IL-1β), Interleukin-1 receptor antagonist (IL1RN), Interleukin-18 (IL-18), Interleukin-36α (IL-36α), Interleukin-36β (IL-36β), Interleukin-36γ (IL-36 γ), Interleukin-36 receptor antagonist (IL36RN), Interleukin-37 (IL-37), Interleukin-38 (IL-38), a fragment thereof, a variant thereof, a multimeric form thereof or a combination thereof. In some embodiments, IL-1 family proteins interact with Interleukin-1 receptor-like 1 (IL1RL1), Interleukin-1 receptor-like 2 (IL1RL2), Interleukin-1 receptor accessory protein (IL1RAP) or combinations thereof. Amino acid sequences of IL-1α, IL-1β, IL-18, IL-36α, IL-36β, IL-36 γ, IL36RN, IL-37, IL-38, IL1RL1, IL1RL2 and IL1RAP are recited in TABLE 1.

TABLE 1
Amino Acid Sequences for Proteins from IL-1 Family

| | SEQ ID | |
|---|---|---|
| Name | NO: | Amino Acid Sequences |
| IL-1α | 1 | MAKVPDMFEDLKNCYSENEEDSSSIDHLSLNQKSFYHVSYGPLHEGCMDQS |
| | | VSLSISETSKTSKLTFKESMVVVATNGKVLKKRRLSLSQSITDDDLEAIAN |
| | | DSEEEIIKPRSAPFSFLSNVKYNFMRIIKYEFILNDALNQSIIRANDQYLT |
| | | AAALHNLDEAVKFDMGAYKSSKDDAKITVILRISKTQLYVTAQDEDQPVLL |
| | | KEMPEIPKTITGSETNLLFFWETHGTKNYFTSVAHPNLFIATKQDYWVCLA |
| | | GGPPSITDFQILENQA |
| IL-1β | 2 | MAEVPELASEMMAYYSGNEDDLFFEADGPKQMKCSFQDLDLCPLDGGIQLR |
| | | ISDHHYSKGFRQAASVVVAMDKLRKMLVPCPQTFQENDLSTFFPFIFEEEP |
| | | IFFDTWDNEAYVHDAPVRSLNCTLRDSQQKSLVMSGPYELKALHLQGQDME |
| | | QQVVFSMSFVQGEESNDKIPVALGLKEKNLYLSCVLKDDKPTLQLESVDPK |
| | | NYPKKKMEKRFVFNKIEINNKLEFESAQFPNWYISTSQAENMPVFLGGTKG |
| | | GQDITDFTMQFVSS |
| IL1RL1 | 3 | MGFWILAILTILMYSTAAKFSKQSWGLENEALIVRCPRQGKPSYTVDWYYS |
| | | QTNKSIPTQERNRVFASGQLLKFLPAAVADSGIYTCIVRSPTFNRTGYANV |
| | | TIYKKQSDCNVPDYLMYSTVSGSEKNSKIYCPTIDLYNWTAPLEWFKNCQA |
| | | LQGSRYRAHKSFLVIDNVMTEDAGDYTCKFIHNENGANYSVTATRSFTVKD |
| | | EQGFSLFPVIGAPAQNEIKEVEIGKNANLTCSACFGKGTQFLAAVLWQLNG |
| | | TKITDFGEPRIQQEEGQNQSFSNGLACLDMVLRIADVKEEDLLLQYDCLAL |
| | | NLHGLRRHTVRLSRKNPIDHHSIYCIIAVCSVFLMLINVLVIILKMFWIEA |
| | | TLLWRDIAKPYKTRNDGKLYDAYVVYPRNYKSSTDGASRVEHFVHQILPDV |
| | | LENKCGYTLCIYGRDMLPGEDVVTAVETNIRKSRRHIFILTPQITHNKEFA |
| | | YEQEVALHCALIQNDAKVILIEMEALSELDMLQAEALQDSLQHLMKVQGTI |
| | | KWREDHIANKRSLNSKFWKHVRYQMPVPSKIPRKASSLTPLAAQKQ |
| IL1RL2 | 4 | MWSLLLCGLSIALPLSVTADGCKDIFMKNEILSASQPFAFNCTFPPITSGE |
| | | VSVTWYKNSSKIPVSKIIQSRIHQDETWILFLPMEWGDSGVYQCVIKGRDS |
| | | CHRIHVNLTVFEKHWCDTSIGGLPNLSDEYKQILHLGKDDSLTCHLHFPKS |
| | | CVLGPIKWYKDCNEIKGERFTVLETRLLVSNVSAEDRGNYACQAILTHSGK |
| | | QYEVLNGITVSITERAGYGGSVPKIIYPKNHSIEVQLGTTLIVDCNVTDTK |
| | | DNTNLRCWRVNNTLVDDYYDESKRIREGVETHVSFREHNLYTVNITFLEVK |
| | | MEDYGLPFMCHAGVSTAYIILQLPAPDFRAYLIGGLIALVAVAVSVVYIYN |
| | | IFKIDIVLWYRSAFHSTETIVDGKLYDAYVLYPKPHKESQRHAVDALVLNI |
| | | LPEVLERQCGYKLFIFGRDEFPGQAVANVIDENVKLCRRLIVIVVPESLGF |
| | | GLLKNLSEEQIAVYSALIQDGMKVILIELEKIEDYTVMPESIQYIKQKHGA |
| | | IRWHGDFTEQSQCMKTKFWKTVRYHMPPRRCRPFPPVQLLQHTPCYRTAGP |
| | | ELGSRRKKCTLTTG |
| IL1RAP | 5 | MTLLWCVVSLYFYGILQSDASERCDDWGLDTMRQIQVFEDEPARIKCPLFE |
| | | HFLKFNYSTAHSAGLTLIWYWTRQDRDLEEPINFRLPENRISKEKDVLWFR |
| | | PTLLNDTGNYTCMLRNTTYCSKVAFPLEVVQKDSCFNSPMKLPVHKLYIEY |
| | | GIQRITCPNVDGYFPSSVKPTITWYMGCYKIQNFNNVIPEGMNLSFLIALI |
| | | SNNGNYTCVVTYPENGRTFHLTRTLTVKVVGSPKNAVPPVIHSPNDHVVYE |
| | | KEPGEELLIPCTVYFSFLMDSRNEVWWTIDGKKPDDITIDVTINESISHSR |
| | | TEDETRTQILSIKKVTSEDLKRSYVCHARSAKGEVAKAAKVKQKVPAPRYT |
| | | VELACGFGATVLLVVILIVVYHVYWLEMVLFYRAHFGTDETILDGKEYDIY |
| | | VSYARNAEEEEFVLLTLRGVLENEFGYKLCIFDRDSLPGGIVTDETLSFIQ |
| | | KSRRLLVVLSPNYVLQGTQALLELKAGLENMASRGNINVILVQYKAVKETK |
| | | VKELKRAKTVLTVIKWKGEKSKYPQGRFWKQLQVAMPVKKSPRRSSSDEQG |
| | | LSYSSLKNV |
| IL1RN | 399 | MEICRGLRSHLITLLLFLFHSETICRPSGRKSSKMQAFRIWDVNQKTFYLR |
| | | NNQLVAGYLQGPNVNLEEKIDVVPIEPHALFLGIHGGKMCLSCVKSGDETR |
| | | LQLEAVNITDLSENRKQDKRFAFIRSDSGPTTSFESAACPGWFLCTAMEAD |
| | | QPVSLTNMPDEGVMVTKFYFQEDE |
| IL-18 | 400 | MAAEPVEDNCINFVAMKFIDNTLYFIAEDDENLESDYFGKLESKLSVIRNL |
| | | NDQVLFIDQGNRPLFEDMTDSDCRDNAPRTIFIISMYKDSQPRGMAVTISV |
| | | KCEKISTLSCENKIISFKEMNPPDNIKDTKSDIIFFQRSVPGHDNKMQFES |
| | | SSYEGYFLACEKERDLFKLILKKEDELGDRSIMFTVQNED |
| IL-33 | 401 | MKPKMKYSTNKISTAKWKNTASKALCFKLGKSQQKAKEVCPMYFMKLRSGL |
| | | MIKKEACYFRRETTKRPSLKTGRKHKRHLVLAACQQQSTVECFAFGISGVQ |
| | | KYTRALHDSSITGISPITEYLASLSTYNDQSITFALEDESYEIYVEDLKKD |
| | | EKKDKVLLSYYESQHPSNESGDGVDGKMLMVTLSPTKDFWLHANNKEHSVE |
| | | LHKCEKPLPDQAFFVLHNMHSNCVSFECKTDPGVFIGVKDNHLALIKVDSS |
| | | ENLCTENILFKLSET |
| IL-36α | 402 | MEKALKIDTPQQGSIQDINHRVWVLQDQTLIAVPRKDRMSPVTIALISCRH |
| | | VETLEKDRGNPIYLGLNGLNLCLMCAKVGDQPTLQLKEKDIMDLYNQPEPV |
| | | KSFLFYHSQSGRNSTFESVAFPGWFIAVSSEGGCPLILTQELGKANTTDFG |
| | | LTMLF |
| IL-36β | 403 | MNPQREAAPKSYAIRDSRQMVWVLSGNSLIAAPLSRSIKPVTLHLIACRDT |
| | | EFSDKEKGNMVYLGIKGKDLCLFCAEIQGKPTLQLKLQGSQDNIGKDTCWK |
| | | LVGIHTCINLDVRESCFMGTLDQWGIGVGRKKWKSSFQHHHLRKKDKDFSS |
| | | MRTNIGMPGRM |
| IL-36γ | 404 | MRGTPGDADGGGRAVYQSMCKPITGTINDLNQQVWTLQGQNLVAVPRSDSV |
| | | TPVTVAVITCKYPEALEQGRGDPIYLGIQNPEMCLYCEKVGEQPTLQLKEQ |
| | | KIMDLYGQPEPVKPFLFYRAKTGRTSTLESVAFPDWFIASSKRDQPIILTS |
| | | ELGKSYNTAFELNIND |
| IL36RN | 405 | MVLSGALCFRMKDSALKVLYLHNNQLLAGGLHAGKVIKGEEISVVPNRWLD |
| | | ASLSPVILGVQGGSQCLSCGVGQEPTLTLEPVNIMELYLGAKESKSFTFYR |
| | | RDMGLTSSFESAAYPGWFLCTVPEADQPVRLTQLPENGGWNAPITDFYFQQ |
| | | CD |
| IL-37 | 406 | MSFVGENSGVKMGSEDWEKDEPQCCLEDPAGSPLEPGPSLPTMNFVHTSPK |
| | | VKNLNPKKFSIHDQDHKVLVLDSGNLIAVPDKNYIRPEIFFALASSLSSAS |
| | | AEKGSPILLGVSKGEFCLYCDKDKGQSHPSLQLKKEKLMKLAAQKESARRP |
| | | FIFYRAQVGSWNMLESAAHPGWFICTSCNCNEPVGVTDKFENRKHIEFSFQ |
| | | PVCKAEMSPSEVSD |
| IL-38 | 407 | MCSLPMARYYIIKYADQKALYTRDGQLLVGDPVADNCCAEKICILPNRGLA |
| | | RTKVPIFLGIQGGSRCLACVETEEGPSLQLEDVNIEELYKGGEEATRFTFF |
| | | QSSSGSAFRLEAAAWPGWFLCGPAEPQQPVQLTKESEPSARTKFYFEQSW |

In some embodiments, multispecific antibodies described herein bind to an amino acid sequence that is at least 60%, at least 70%, at least 75%, at least 80%, at least 85%, at least 90%, at least 95%, at least 99% or 100% identical to any one of the sequences recited in TABLE 1. In some embodiments, multispecific molecules described herein bind to an amino acid sequence that is at least 60%, at least 70%, at least 75%, at least 80%, at least 85%, at least 90%, at least 95%, at least 99% or 100% identical to any one of SEQ ID NOs: 1-5 and 399-407. In some embodiments, multispecific molecules described herein can bind: (a) an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407; and (b) TREM1, a functional fragment thereof, a variant thereof, or a combination thereof. In some embodiments, multispecific molecules described herein can bind: (a) an amino acid sequence that is at least 60%, at least 70%, at least 75%, at least 80%, at least 85%, at least 90%, at least 95%, at least 99% or 100% identical to any one of SEQ ID NOs: 1-5 and 400-401; and (b) TREM1, a functional fragment thereof, a variant thereof, or a combination thereof.

In some embodiments, multispecific molecules described herein comprise at least one of CDR-Hs described in TABLE 2 or a variant thereof, wherein the multispecific molecule is capable of binding an amino acid sequence that is at least 60%, at least 70%, at least 75%, at least 80%, at least 85%, at least 90%, 95%, at least 99% or 100% identical to any one of SEQ ID NOs: 1-5 and 399-407. In some embodiments, multispecific molecules described herein comprise any one of CDR-H1 described in TABLE 2 or a variant thereof, any one of CDR-H2 described in TABLE 2 or a variant thereof, and any one of CDR-H3 described in TABLE 2 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407.

In some embodiments, multispecific molecules described herein comprise any one of combinations of CDR-Hs or variants thereof described in TABLE 2, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407. In some embodiments, multispecific molecules described herein comprise at least one of CDR-Hs described in TABLE 2 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407. In some embodiments, a CDR-H variant comprises at least one, at least two or at least three substitutions, deletions, additions or combinations thereof relative to a corresponding parent CDR-H sequence described in TABLE 2. In some embodiments, a CDR-H or a variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 2.

In some embodiments, a multispecific molecule described herein comprises an IL-1 binding domain and a TREM1 binding domain, wherein (a) the IL-1 binding domain comprises any one of combinations of CDR-H1, CDR-H2 and CDR-H3 described in TABLE 2, (b) the IL-1 binding domain can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding domain can bind TREM1, a functional fragment thereof, a variant thereof, or a combination thereof.

In some embodiments, an engineered protein construct described herein comprises an IL-1 binding moiety, wherein (a) the IL-1 binding moiety comprises any one of combinations of CDR-H1, CDR-H2 and CDR-H3 described in TABLE 2, and (b) the IL-1 binding moiety can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401. In some embodiments, an engineered protein construct described herein comprises an IL-1 binding region and a TREM1 binding region, wherein (a) the IL-1 binding region comprises any one of combinations of CDR-H1, CDR-H2 and CDR-H3 described in TABLE 2, (b) the IL-1 binding region can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding region can bind TREM1, a functional fragment thereof, a variant thereof, or a combination thereof.

TABLE 2
Combinations of CDR-Hs for binding to proteins from IL-1 family

| | | SEQ | | SEQ | | SEQ | |
|---|---|---|---|---|---|---|---|
| Comb. | | ID | | ID | | ID | |
| No. | Target | NO: | CDR-H1 | NO: | CDR-H2 | NO: | CDR-H3 |
| 1 | IL-1α | 6 | GFTFSMFG | 16 | VSYDGSNK | 26 | ARGRPKVVIPAPLAH |
| 2 | IL-1α | 7 | GFIFSRYD | 17 | ISHGGAGT | 27 | ARGGVTKGYFDV |
| 3 | IL-1α | 8 | GGRFTNYA | 18 | IIPIFDET | 28 | ATGSNSYYGLY |
| 1 | IL-1β | 6 | GFTFSMFG | 16 | VSYDGSNK | 26 | ARGRPKVVIPAPLAH |
| 4 | IL-1β | 9 | GFTFSVYG | 19 | IWYDGDNQ | 29 | ARDLRTGPFDY |
| 5 | IL-1β | 10 | GFSLSTSGMG | 20 | IWWDGDE | 30 | ARNRYDPPWFVD |
| 6 | IL1RL1 | 11 | GYSFTNYW | 21 | IYPGNSDT | 31 | ARHGTSSDYYGLDV |
| 7 | IL1RL1 | 12 | GFTFSIYD | 22 | IRGEGGGT | 32 | ARDPWSTEGSFFVLDY |
| 8 | IL1RL2 | 13 | GYTFTNYW | 23 | FHPTGDVT | 33 | ARTTSMIIGGFAY |
| 9 | IL1RL2 | 14 | GYSFTSSW | 24 | INPGNVRT | 34 | TVVFYGEPYFPY |
| 10 | IL1RAP | 15 | GYAFTSSW | 25 | IYPGDGNT | 35 | GEGYLDPMDY |
| 275 | IL-18 | 408 | GGSISADGYY | 412 | LYYSGST | 417 | ARTPAYFGQDRTDFFDV |
| 276 | IL-33 | 215 | GYTFTSYW | 413 | IYPRNSNT | 418 | ARPLYYYLTSPPTLF |
| 277 | IL-33 | 409 | GFTFSRSA | 414 | ISGSGGRT | 419 | AKDSYTTSWYGGMDV |
| 278 | IL-33 | 410 | GFTFSFYA | 415 | ISGSGGST | 420 | ARTIHGIRAAYDAFII |
| 279 | IL-33 | 411 | GFTFSSYA | 416 | ISAIDQST | 421 | ARQKFMQLWGGGLRYPFGY |

In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 3, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407.

In some embodiments, a multispecific molecule described herein comprises at least one VH sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407. In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 3, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 400-401. In some embodiments, a multispecific molecule described herein comprises an IL-1 binding domain and a TREM1 binding domain, wherein (a) the IL-1 binding domain comprises an amino acid sequence that is at least 60%, at least 65%, at least 70%, at least 75%, at least 80%, at least 85%, at least 90%, at least 95% or 100% identical to any one of VH sequences described in TABLE 3, (b) the IL-1 binding domain can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding domain can bind TREM1, a functional fragment thereof, a variant thereof, or a combination thereof.

In some embodiments, an engineered protein construct described herein comprises an IL-1 binding moiety, wherein (a) the IL-1 binding moiety comprises an amino acid sequence that is at least 60%, at least 65%, at least 70%, at least 75%, at least 80%, at least 85%, at least 90%, at least 95% or 100% identical to any one of VH sequences described in TABLE 3, and (b) the IL-1 binding moiety can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401. In some embodiments, an engineered protein construct described herein comprises an IL-1 binding region and a TREM1 binding region, wherein (a) the IL-1 binding region comprises an amino acid sequence that is at least 60%, at least 65%, at least 70%, at least 75%, at least 80%, at least 85%, at least 90%, at least 95% or 100% identical to any one of VH sequences described in TABLE 3, (b) the IL-1 binding region can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding region can bind TREM1, a functional fragment thereof, a variant thereof, or a combination thereof.

TABLE 3
Exemplary VH sequence for binding to proteins from IL-1 family

| SEQ ID NO: | Target | VH Sequences |
|---|---|---|
| 36 | IL-1α | QVQLVESGGGVVQPGRSLRLSCTASGFTFSMFGVHWVRQAPGKGLEW |
| | | VAAVSYDGSNKYYAESVKGRFTISRDNSKNILFLQMDSLRLEDTAVY |
| | | YCARGRPKVVIPAPLAHWGQGTLVTFSS |
| 37 | IL-1α | EVQLVESGGGVVQPGRSLRLSCSASGFIFSRYDMSWVRQAPGKGLEW |
| | | VAYISHGGAGTYYPDSVKGRFTISRDNSKNTLFLQMDSLRPEDTGVY |
| | | FCARGGVTKGYFDVWGQGTPVTVSS |
| 38 | IL-1α | QVQLVQSGAEVKKPGSSVKVSCKASGGRFTNYAILWVRQAPGQGLQW |
| | | LGGIIPIFDETDHAQDFQDRLTITVDESMTTAYMELSSLRPEDTAIY |
| | | YCATGSNSYYGLYWGQGTLVTVSS |
| 36 | IL-1β | QVQLVESGGGVVQPGRSLRLSCTASGFTFSMFGVHWVRQAPGKGLEW |
| | | VAAVSYDGSNKYYAESVKGRFTISRDNSKNILFLQMDSLRLEDTAVY |
| | | YCARGRPKVVIPAPLAHWGQGTLVTFSS |
| 39 | IL-1β | QVQLVESGGGVVQPGRSLRLSCAASGFTFSVYGMNWVRQAPGKGLEW |
| | | VAIIWYDGDNQYYADSVKGRFTISRDNSKNTLYLQMNGLRAEDTAVY |
| | | YCARDLRTGPFDYWGQGTLVTVSS |
| 40 | IL-1β | QVQLQESGPGLVKPSQTLSLTCSFSGFSLSTSGMGVGWIRQPSGKGL |
| | | EWLAHIWWDGDESYNPSLKSRLTISKDTSKNQVSLKITSVTAADTAV |
| | | YFCARNRYDPPWFVDWGQGTLVTVSS |
| 41 | IL1RL1 | EVQLVQSGAEVKKPGESLKISCKGSGYSFTNYWIGWVRQMPGKGLEW |
| | | MGIIYPGNSDTRFSPSFQGQVTISADKSITTAYLQWSSLKASDTAMY |
| | | YCARHGTSSDYYGLDVWGQGTTVTVSS |
| 42 | IL1RL1 | EVQLLESGGGLVQPGGSLRLSCAASGFTFSIYDMIWVRQAPGKGLEW |
| | | VSSIRGEGGGTYYADSVKGRFTISRDNSKNTLYLQMNSLRAEDTAVY |
| | | YCARDPWSTEGSFFVLDYWGQGTLVTVSS |
| 43 | IL1RL2 | QVQLVQSGAEVKKPGASVKVSCKASGYTFTNYWMNWVRQAPRQGLEW |
| | | MGMFHPTGDVTRLNQKFKDRVTMTRDTSTSTVYMELSSLRSEDTAVY |
| | | YCARTTSMIIGGFAYWGQGTLVTVSS |
| 44 | IL1RL2 | QVQLVQSGAEVKKPGASVKVSCKASGYSFTSSWIHWVKQAPGQGLEW |
| | | MGEINPGNVRTNYNENFRNKVTMTVDTSISTAYMELSRLRSDDTAVY |
| | | YCTVVFYGEPYFPYWGQGTLVTVSS |
| 45 | IL1RAP | QVQLVQSGAEVKKPGSSVKVSCKASGYAFTSSWMNWVRQAPGQGLEW |
| | | MGRIYPGDGNTHYAQKFQGRVTLTADKSTSTAYMELSSLRSEDTAVY |
| | | YCGEGYLDPMDYWGQGTLVTVSS |
| 422 | IL-18 | QVQLQESGPGLVKPSETLSLTCTVSGGSISADGYYWSWIRQPPGKGL |
| | | EWIGSLYYSGSTYYNPSLKGRVTISGDTSKNQFSLKLSSVTAADTAV |
| | | YYCARTPAYFGQDRTDFFDVWGRGTLVTVSS |
| 423 | IL-33 | QVQLMQSGAEVKKPGASVKVSCKASGYTFTSYWMHWVRQAPGQGLEW |
| | | MGTIYPRNSNTDYNQKFKARVTMTRDTSTSTVYMELSSLRSEDTAVY |
| | | YCARPLYYYLTSPPTLFWGQGTLVTVSS |
| 424 | IL-33 | EVQLVESGGNLEQPGGSLRLSCTASGFTFSRSAMNWVRRAPGKGLEW |
| | | VSGISGSGGRTYYADSVKGRFTISRDNSKNTLYLQMNSLSAEDTAAY |
| | | YCAKDSYTTSWYGGMDVWGHGTTVTVSS |
| 425 | IL-33 | EVQLVETGGGLIQPGGSLRLSCAASGFTFSFYAMSWVRQAPGKGLEW |
| | | VSAISGSGGSTYYADSVKGRFTISRDNSKNTLYLQMNSLRAEDTAVY |
| | | YCARTIHGIRAAYDAFIIWGQGTLVTVSS |
| 426 | IL-33 | EVQLLESGGGLVQPGGSLRLSCAASGFTFSSYAMSWVRQAPGKGLEW |
| | | VSGISAIDQSTYYADSVKGRFTISRDNSKNTLYLQMNSLRAEDTAVY |
| | | YCARQKFMQLWGGGLRYPFGYWGQGTMVTVSS |

In some embodiments, a multispecific molecule described herein comprises at least one heavy chain (HC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407. In some embodiments, a multispecific molecule described herein comprises an IL-1 binding region, wherein the IL-1 binding region comprises a HC region. In some embodiments, a HC region of an IL-1 binding region comprises a VH sequence that is at least 60%, at least 65%, at least 70%, at least 75%, at least 80%, at least 85%, at least 90%, at least 95% or 100% identical to any one of amino acid sequences described in TABLE 3.

In some embodiments, a HC region of an IL-1 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVMHEALHNHYTQ KSLSLSPG (SEQ ID NO: 481), wherein C-terminus of the VH sequence is linked to N-terminus of the amino acid sequence.

In some embodiments, a HC region of an IL-1 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVCTLPPSREEMTKNQVSLSCAVKGFYPS DIAVEWESNGQPENNYKTTPPVLDSDGSFFLVSKLTVDKSRWQQGNVFSCSVMHEALHNHYTQ KSLSLSPG (SEQ ID NO: 482), wherein C-terminus of the VH sequence is linked to N-terminus of the amino acid sequence.

In some embodiments, multispecific molecules described herein comprise at least one of CDR-Ls described in TABLE 4 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407. In some embodiments, multispecific molecules described herein comprise any one of CDR-L1 described in TABLE 4 or a variant thereof, any one of CDR-L2 described in TABLE 4 or a variant thereof, and any one of CDR-L3 described in TABLE 4 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407.

In some embodiments, multispecific molecules described herein comprise any one of combinations of CDR-Ls or variants thereof described in TABLE 4, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407. In some embodiments, a multispecific molecule described herein comprises at least one of CDR-Ls described in TABLE 4 or a variant thereof, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407. In some embodiments, a CDR-L variant comprises at least one, at least two or at least three substitutions, deletions, additions or combinations thereof relative to a corresponding parent CDR-L sequence described in TABLE 4. In some embodiments, a CDR-L or a variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 4.

In some embodiments, an engineered protein construct described herein comprises an IL-1 binding moiety, wherein (a) the IL-1 binding moiety comprises any one of combinations of CDR-L1, CDR-L2 and CDR-L3 described in TABLE 4, and (b) the IL-1 binding moiety can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401. In some embodiments, an engineered protein construct described herein comprises an IL-1 binding region and a TREM1 binding region, wherein (a) the IL-1 binding region comprises any one of combinations of CDR-L1, CDR-L2 and CDR-L3 described in TABLE 4, (b) the IL-1 binding region can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding region can bind TREM1, a functional fragment thereof, a variant thereof, or a combination thereof. In some embodiments, a multispecific molecule described herein comprises an IL-1 binding domain and a TREM1 binding domain, wherein (a) the IL-1 binding domain comprises any one of combinations of CDR-L1, CDR-L2 and CDR-L3 described in TABLE 4, (b) the IL-1 binding domain can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding domain can bind TREM1, a functional fragment thereof, a variant thereof, or a combination thereof.

TABLE 4
Combinations of CDR-Ls for binding to proteins from IL-1 family

| | | SEQ | | SEQ | | SEQ | |
|---|---|---|---|---|---|---|---|
| Comb. | | ID | | ID | | ID | |
| No. | Target | NO: | CDR-L1 | NO: | CDR-L2 | NO: | CDR-L3 |
| 11 | IL-1α | 46 | QGISSW | 55 | EAS | 63 | QQTSSFLLS |
| 12 | IL-1α | 47 | GNIHNY | 56 | NAK | 64 | QHFWSIPYT |
| 13 | IL-1α | 48 | QSVLYSSNNKNY | 57 | WAS | 65 | QQYYSTPST |
| 11 | IL-1β | 46 | QGISSW | 55 | EAS | 63 | QQTSSFLLS |
| 14 | IL-1β | 49 | QSIGSS | 58 | YAS | 66 | HQSSSLPFT |
| 15 | IL-1β | 50 | QDISNY | 59 | YTS | 67 | LQGKMLPWT |
| 16 | IL1RL1 | 50 | QDISNY | 60 | DAS | 68 | QQDDNFPLT |
| 17 | IL1RL1 | 51 | QSVDDD | 60 | DAS | 69 | QQYITAPLT |
| 18 | IL1RL2 | 52 | KSLLHRNAITY | 61 | QMS | 70 | AQNLELPLT |
| 19 | IL1RL2 | 53 | SSVSSSY | 62 | RTS | 71 | HQFHRSPLT |
| 20 | IL1RAP | 54 | QGINNY | 59 | YTS | 72 | QQYSILPWT |
| 280 | IL-18 | 46 | QGISSW | 174 | KAS | 432 | QQSHHPPWT |
| 281 | IL-33 | 427 | QDVGTA | 57 | WAS | 433 | QQAKTYPFT |
| 282 | IL-33 | 428 | QGIFSW | 173 | AAS | 434 | QQANSVPIT |
| 283 | IL-33 | 429 | QSVGIN | 177 | GAS | 435 | HQYSQPPPFT |
| 284 | IL-33 | 430 | GMGDKY | 431 | RDT | 436 | GVIQDNTGV |

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 5.

TABLE 5
Exemplary CDR Combinations for Antibody
targeting proteins from IL-1 family

| Comb. | | CDR- | CDR- | CDR- | CDR- | CDR- | CDR- |
|---|---|---|---|---|---|---|---|
| No. | Target | H1 | H2 | H3 | L1 | L2 | L3 |
| 21 | IL-1α | 6 | 16 | 26 | 46 | 55 | 63 |
| 22 | IL-1α | 7 | 17 | 27 | 47 | 56 | 64 |
| 23 | IL-1α | 8 | 18 | 28 | 48 | 57 | 65 |
| 21 | IL-1β | 6 | 16 | 26 | 46 | 55 | 63 |
| 24 | IL-1β | 9 | 19 | 29 | 49 | 58 | 66 |
| 25 | IL-1β | 10 | 20 | 30 | 50 | 59 | 67 |
| 26 | IL1RL1 | 11 | 21 | 31 | 50 | 60 | 68 |
| 27 | IL1RL1 | 12 | 22 | 32 | 51 | 60 | 69 |
| 28 | IL1RL2 | 13 | 23 | 33 | 52 | 61 | 70 |
| 29 | IL1RL2 | 14 | 24 | 34 | 53 | 62 | 71 |
| 30 | IL1RAP | 15 | 25 | 35 | 54 | 59 | 72 |
| 285 | IL-18 | 408 | 412 | 417 | 46 | 174 | 432 |
| 286 | IL-33 | 215 | 413 | 418 | 427 | 57 | 433 |
| 287 | IL-33 | 409 | 414 | 419 | 428 | 173 | 434 |
| 288 | IL-33 | 410 | 415 | 420 | 429 | 177 | 435 |
| 289 | IL-33 | 411 | 416 | 421 | 430 | 431 | 436 |

In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 6, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 399-407.

In some embodiments, a multispecific molecule described herein comprises at least one VL sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407. In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 6, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 1-5 and 400-401. In some embodiments, an engineered protein construct described herein comprises an IL-1 binding moiety, wherein (a) the IL-1 binding moiety comprises an amino acid sequence that is at least 60%, at least 65%, at least 70%, at least 75%, at least 80%, at least 85%, at least 90%, at least 95% or 100% identical to any one of VL sequences described in TABLE 6, and (b) the IL-1 binding moiety can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401. In some embodiments, an engineered protein construct described herein comprises an IL-1 binding region and a TREM1 binding region, wherein (a) the IL-1 binding region comprises an amino acid sequence that is at least 60%, at least 65%, at least 70%, at least 75%, at least 80%, at least 85%, at least 90%, at least 95% or 100% identical to any one of VL sequences described in TABLE 6, (b) the IL-1 binding region can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding region can bind TREM1, a functional fragment thereof, a variant thereof, or a combination thereof. In some embodiments, a multispecific molecule described herein comprises an IL-1 binding domain and a TREM1 binding domain, wherein (a) the IL-1 binding domain comprises an amino acid sequence that is at least 60%, at least 65%, at least 70%, at least 75%, at least 80%, at least 85%, at least 90%, at least 95% or 100% identical to any one of VL sequences described in TABLE 6, (b) the IL-1 binding domain can bind an amino acid sequence that is at least 60%, at least 70%, at least 75%, at least 80%, at least 85%, at least 90%, at least 95%, at least 99% or 100% identical to any one of SEQ ID NOs: 1-5 and 400-401, and (c) the TREM1 binding domain can bind TREM1, a functional fragment thereof, a variant thereof, or a combination thereof.

TABLE 6
Exemplary VL sequence for binding to proteins from IL-1 family

| SEQ ID NO: | Target | VL Sequences |
|---|---|---|
| 73 | IL-1α | DIQMTQSPSSVSASVGDRVTITCRASQGISSWLAWYQQKPGKAP |
| | | KLLIYEASNLETGVPSRFSGSGSGSDFTLTISSLQPEDFATYYC |
| | | QQTSSFLLSFGGGTKVEHK |
| 74 | IL-1α | DIQMTQSPSSLSASVGDRVTITCRASGNIHNYLTWYQQTPGKAP |
| | | KLLIYNAKTLADGVPSRFSGSGSGTDYTFTISSLQPEDIATYYC |
| | | QHFWSIPYTFGQGTKLQIT |
| 75 | IL-1α | DIVMTQSPDSLAVSLGERATINCKSSQSVLYSSNNKNYLAWYQQ |
| | | KPGQPPKLLIYWASTRESGVPDRFSGSGSGTDFTLTISSLQAED |
| | | VAVYYCQQYYSTPSTFGQGTKVEIK |
| 73 | IL-1β | DIQMTQSPSSVSASVGDRVTITCRASQGISSWLAWYQQKPGKAP |
| | | KLLIYEASNLETGVPSRFSGSGSGSDFTLTISSLQPEDFATYYC |
| | | QQTSSFLLSFGGGTKVEHK |
| 76 | IL-1β | EIVLTQSPDFQSVTPKEKVTITCRASQSIGSSLHWYQQKPDQSP |
| | | KLLIKYASQSFSGVPSRFSGSGSGTDFTLTINSLEAEDAAAYYC |
| | | HQSSSLPFTFGPGTKVDIK |
| 77 | IL-1β | DIQMTQSTSSLSASVGDRVTITCRASQDISNYLSWYQQKPGKAV |
| | | KLLIYYTSKLHSGVPSRFSGSGSGTDYTLTISSLQQEDFATYFC |
| | | LQGKMLPWTFGQGTKLEIK |
| 78 | IL1RL1 | DIQMTQSPSSLSASVGDRVTITCQASQDISNYLNWYQQKPGKAP |
| | | KLLIYDASNLETGVPSRFSGSGSGTDFTFTISSLQPEDIATYYC |
| | | QQDDNFPLTFGGGTKVEIK |
| 79 | IL1RL1 | EIVLTQSPATLSLSPGERATLSCRASQSVDDDLAWYQQKPGQAP |
| | | RLLIYDASNRATGIPARFSGSGSGTDFTLTISSLEPEDFAVYYC |
| | | QQYITAPLTFGQGTKVEIK |
| 80 | IL1RL2 | DIVMTQTPLSLSVTPGQPASISCRSSKSLLHRNAITYFYWYLHK |
| | | PGQPPQLLIYQMSNLASGVPDRFSGSGSGTDFTLKISRVEAEDV |
| | | GVYYCAQNLELPLTFGGGTKVEIK |
| 81 | IL1RL2 | QIVLTQSPGTLSLSPGERATMTCTASSSVSSSYFHWYQQKPGQA |
| | | PRLWIYRTSRLASGVPDRFSGSGSGTDFTLTISRLEPEDAATYY |
| | | CHQFHRSPLTFGAGTKLEIK |
| 82 | IL1RAP | DIQMTQSPSSLSASVGDRVTITCQASQGINNYLNWYQQKPGKAP |
| | | KLLIHYTSGLHAGVPSRFSGSGSGTDYTLTISSLEPEDVATYYC |
| | | QQYSILPWTFGGGTKVEIK |
| 437 | IL-18 | DIQMTQSPSTLSASVGDRVTITCRASQGISSWLAWYQQKPGKAP |
| | | KVLIYKASTLESGVPSRFSGSGSGTEFTLTISSLQPDDFATYYC |
| | | QQSHHPPWTFGQGTKLEIK |
| 438 | IL-33 | DIQLTQSPSFLSASVGDRVTITCKASQDVGTAVAWYQQKPGKAP |
| | | KLLIYWASTRHTGVPSRFSGSGSGTEFTLTISSLQPEDFATYYC |
| | | QQAKTYPFTFGSGTKLEIK |
| 439 | IL-33 | DIQMTQSPSSVSASVGDRVTITCRASQGIFSWLAWYQQKPGKAP |
| | | KLLIYAASSLQSGVPSRFSGSGSGTDFTLTISSLQPEDFAIYYC |
| | | QQANSVPITFGQGTRLEIK |
| 440 | IL-33 | EIVLTQSPGTLSLSPGERATLSCRASQSVGINLSWYQQKPGQAP |
| | | RLLIYGASHRLTGIPDRFSGSGSGTDFTLTISRLEPEDFAVYYC |
| | | HQYSQPPPFTFGGGTKVEIK |
| 441 | IL-33 | SYVLTQPPSVSVSPGQTASITCSGEGMGDKYAAWYQQKPGQSPV |
| | | LVIYRDTKRPSGIPERFSGSNSGNTATLTISGTQAMDEADYYCG |
| | | VIQDNTGVFGGGTKLTVL |

In some embodiments, a multispecific molecule described herein comprises at least one light chain (LC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 1-5, 400-404 and 406-407. In some embodiments, a multispecific molecule described herein comprises an IL-1 binding region, wherein the IL-1 binding region comprises a LC region. In some embodiments, a LC region of an IL-1 binding region comprises a VL sequence that is at least 60%, at least 65%, at least 70%, at least 75%, at least 80%, at least 85%, at least 90%, at least 95% or 100% identical to any one of amino acid sequences described in TABLE 6.

In some embodiments, a LC region of an IL-1 binding region comprises a VL sequence, and an amino acid sequence that is at least 60%, at least 65%, at least 70%, at least 75%, at least 80%, at least 85%, at least 90%, at least 95% or 100% identical to RTVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 483), wherein C-terminus of the VL sequence is linked to N-terminus of the amino acid sequence.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 3; and (b) a VL sequence that is at least 60%, at least 65%, at least 70%, at least 75%, at least 80%, at least 85%, at least 90%, at least 95% or 100% identical to any one of amino acid sequences described in TABLE 6, wherein the multispecific molecule comprises the VH sequence and the VL sequence according to any one of combinations described in TABLE 7.

TABLE 7
Exemplary Combinations of VH sequences and VL sequences
for binding to proteins from IL-1 family

| Comb. No: | Target | VH (SEQ ID NO:) | VL (SEQ ID NO:) |
|---|---|---|---|
| 31 | IL1A | 36 | 73 |
| 32 | IL1A | 37 | 74 |
| 33 | IL1A | 38 | 75 |
| 31 | IL1B | 36 | 73 |
| 34 | IL1B | 39 | 76 |
| 35 | IL1B | 40 | 77 |
| 36 | IL1RL1 | 41 | 78 |
| 37 | ILIRL1 | 42 | 79 |
| 38 | IL1RL2 | 43 | 80 |
| 39 | IL1RL2 | 44 | 81 |
| 40 | IL1RAP | 45 | 82 |
| 290 | IL-18 | 422 | 437 |
| 291 | IL-33 | 423 | 438 |
| 292 | IL-33 | 424 | 439 |
| 293 | IL-33 | 425 | 440 |
| 294 | IL-33 | 426 | 441 |

### IL-6 Family

The IL-6 family, as described herein, includes Interleukin-6 cytokine (IL-6), Interleukin-11 cytokine (IL-11), a fragment thereof, a variant thereof, a multimeric form thereof or a combination thereof. In some embodiments, IL-6 family proteins bind to Interleukin-6 receptor (IL-6R). Amino acid sequences of IL-6, IL-11 and IL-6R are recited in TABLE 8.

TABLE 8
Amino Acid Sequences for Proteins from IL-6 Family

| | SEQ | |
|---|---|---|
| | ID | |
| Name | NO: | Amino Acid Sequences |
| IL-6 | 83 | MNSFSTSAFGPVAFSLGLLLVLPAAFPAPVPPGEDSKDVAAPHRQPLTSSERIDKQ |
| | | IRYILDGISALRKETCNKSNMCESSKEALAENNLNLPKMAEKDGCFQSGFNEETCL |
| | | VKIITGLLEFEVYLEYLQNRFESSEEQARAVQMSTKVLIQFLQKKAKNLDAITTPD |
| | | PTTNASLLTKLQAQNQWLQDMTTHLILRSFKEFLQSSLRALRQM |
| IL-6R | 84 | MLAVGCALLAALLAAPGAALAPRRCPAQEVARGVLTSLPGDSVTLTCPGVEPEDNA |
| | | TVHWVLRKPAAGSHPSRWAGMGRRLLLRSVQLHDSGNYSCYRAGRPAGTVHLLVDV |
| | | PPEEPQLSCFRKSPLSNVVCEWGPRSTPSLTTKAVLLVRKFQNSPAEDFQEPCQYS |
| | | QESQKFSCQLAVPEGDSSFYIVSMCVASSVGSKFSKTQTFQGCGILQPDPPANITV |
| | | TAVARNPRWLSVTWQDPHSWNSSFYRLRFELRYRAERSKTFTTWMVKDLQHHCVIH |
| | | DAWSGLRHVVQLRAQEEFGQGEWSEWSPEAMGTPWTESRSPPAENEVSTPMQALTT |
| | | NKDDDNILFRDSANATSLPVQDSSSVPLPTFLVAGGSLAFGTLLCIAIVLRFKKTW |
| | | KLRALKEGKTSMHPPYSLGQLVPERPRPTPVLVPLISPPVSPSSLGSDNTSSHNRP |
| | | DARDPRSPYDISNTDYFFPR |
| IL-11 | 442 | MNCVCRLVLVVLSLWPDTAVAPGPPPGPPRVSPDPRAELDSTVLLTRSLLADTRQL |
| | | AAQLRDKFPADGDHNLDSLPTLAMSAGALGALQLPGVLTRLRADLLSYLRHVQWLR |
| | | RAGGSSLKTLEPELGTLQARLDRLLRRLQLLMSRLALPQPPPDPPAPPLAPPSSAW |
| | | GGIRAAHAILGGLHLTLDWAVRGLLLLKTRL |

In some embodiments, multispecific molecules described herein bind to an amino acid sequence that is at least 60%, at least 70%, at least 75%, at least 80%, at least 85%, at least 90%, at least 95%, at least 99% or 100% identical to any one of the sequences recited in TABLE 8. In some embodiments, multispecific molecules described herein bind to an amino acid sequence that is at least 60%, at least 70%, at least 75%, at least 80%, at least 85%, at least 90%, at least 95%, at least 99% or 100% identical to any one of SEQ ID NOs: 83-84 and 442. In some embodiments, multispecific molecules described herein bind: (a) an amino acid sequence that is at least 60%, at least 70%, at least 75%, at least 80%, at least 85%, at least 90%, at least 95%, at least 99% or 100% identical to any one of SEQ ID NOs: 83-84 and 442; and (b) TREM1, a functional fragment thereof, a variant thereof, or a combination thereof.

In some embodiments, multispecific molecules described herein comprise at least one of CDR-Hs described in TABLE 9 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, multispecific molecules described herein comprise any one of CDR-H1 described in TABLE 9 or a variant thereof, any one of CDR-H2 described in TABLE 9 or a variant thereof, and any one of CDR-H3 described in TABLE 9 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, multispecific molecules described herein comprise any one of combinations of CDR-Hs or variants thereof described in TABLE 9, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, a CDR-H variant comprises at least one, at least two or at least three substitutions, deletions, additions or combinations thereof relative to a corresponding parent CDR-H sequence described in TABLE 9. In some embodiments, a CDR-H or a variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 9.

In some embodiments, an engineered protein construct described herein comprises an IL-6 binding moiety, wherein (a) the IL-6 binding moiety comprises any one of combinations of CDR-H1, CDR-H2 and CDR-H3 described in TABLE 9, and (b) the IL-6 binding moiety can bind an amino acid sequence that is at least 60%, at least 70%, at least 75%, at least 80%, at least 85%, at least 90%, at least 95%, at least 99% or 100% identical to any one of SEQ ID NOs: 83-84. In some embodiments, an engineered protein construct described herein comprises an IL-6 binding region and a TREM1 binding region, wherein (a) the IL-6 binding region comprises any one of combinations of CDR-H1, CDR-H2 and CDR-H3 described in TABLE 9, (b) the IL-6 binding region can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding region can bind TREM1, a functional fragment thereof, a variant thereof, or a combination thereof. In some embodiments, a multispecific molecule described herein comprises an IL-6 binding domain and a TREM1 binding domain, wherein (a) the IL-6 binding domain comprises any one of combinations of CDR-H1, CDR-H2 and CDR-H3 described in TABLE 9, (b) the IL-6 binding domain can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding domain can bind TREM1, a functional fragment thereof, a variant thereof, or a combination thereof.

TABLE 9
Combinations of CDR-Hs for binding to proteins from IL-6 family

| | | SEQ | | SEQ | | SEQ | |
|---|---|---|---|---|---|---|---|
| Comb. | | ID | | ID | | ID | |
| No. | Target | NO: | CDR-H1 | NO: | CDR-H2 | NO: | CDR-H3 |
| 41 | IL6 | 85 | GFSLSNYYV | 97 | IYGSDET | 108 | ARDDSSDWDAKFNL |
| 42 | IL6 | 86 | GFNFNDYF | 98 | MRNKNYQYGT | 109 | ARESYYGFTSY |
| 43 | IL6 | 87 | GFTFSSFA | 99 | ISSGGSYT | 110 | ARGLWGYYALDY |
| 44 | IL6 | 88 | GFTFSPFA | 100 | ISPGGSWT | 111 | ARQLWGYYALDI |
| 45 | IL6 | 89 | GYVLPNYL | 101 | TTPGGGTI | 112 | ARSRWDPLYYYALEY |
| 46 | IL6 | 90 | GFTISSNY | 102 | LYYYAGDT | 113 | ARWADDHPPWIDL |
| 47 | IL6 | 91 | SNYMI | 103 | DLYYYAGDTYYADSVKG | 114 | WADDHYYYIDV |
| 48 | IL6 | 92 | SNYMT | 104 | DLYYYAGDTYYADSVRG | 115 | WADGHYYYIDV |
| 49 | IL6 | 93 | SNYMV | 103 | DLYYYAGDTYYADSVKG | 116 | WADDHYYHIDV |
| 50 | IL6 | 92 | SNYMT | 103 | DLYYYAGDTYYADSVKG | 117 | WADGHYYYADV |
| 51 | IL6 | 91 | SNYMI | 103 | DLYYYAGDTYYADSVKG | 117 | WADGHYYYADV |
| 52 | IL6 | 91 | SNYMI | 103 | DLYYYAGDTYYADSVKG | 118 | WADDHPAWVDL |
| 53 | IL6 | 91 | SNYMI | 103 | DLYYYAGDTYYADSVKG | 119 | WADDHPRYIDH |
| 54 | IL6 | 91 | SNYMI | 103 | DLYYYAGDTYYADSVKG | 120 | WEEEGRGYIDV |
| 55 | IL6 | 91 | SNYMI | 103 | DLYYYAGDTYYADSVKG | 121 | WADDHNYPHIDV |
| 56 | IL6 | 91 | SNYMI | 103 | DLYYYAGDTYYADSVKG | 122 | WADDHPPYIDL |
| 57 | IL6 | 91 | SNYMI | 103 | DLYYYAGDTYYADSVKG | 123 | WADDHPPYIDM |
| 58 | IL6 | 91 | SNYMI | 103 | DLYYYAGDTYYADSVKG | 124 | WADDHPPWIDL |
| 59 | IL6 | 91 | SNYMI | 103 | DLYYYAGDTYYADSVKG | 125 | WADDHPSHLDI |
| 60 | IL6 | 91 | SNYMI | 103 | DLYYYAGDTYYADSVKG | 126 | WADDHPSHIDV |
| 61 | IL6 | 91 | SNYMI | 103 | DLYYYAGDTYYADSVKG | 127 | WADDHNNTYIDV |
| 62 | IL6 | 91 | SNYMI | 103 | DLYYYAGDTYYADSVKG | 128 | WADDHAPWVDL |
| 41 | IL6 | 85 | GFSLSNYYV | 97 | IYGSDET | 108 | ARDDSSDWDAKFNL |
| 43 | IL6 | 87 | GFTFSSFA | 99 | ISSGGSYT | 110 | ARGLWGYYALDY |
| 63 | IL6R | 94 | GFTFSSYY | 105 | IYSDGTTH | 129 | AKGAGPTWWYALDA |
| 64 | IL6R | 95 | RFTFDDYA | 106 | ISWNSGRI | 130 | AKGRDSFDI |
| 65 | IL6R | 96 | GHSISHDHA | 107 | FISYSGIT | 131 | ARSLARTTAMDY |

In some embodiments, a multispecific molecule described herein comprises at least one VH sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 10, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, an engineered protein construct described herein comprises an IL-6 binding moiety, wherein (a) the IL-6 binding moiety comprises an amino acid sequence that is at least 60%, at least 65%, at least 70%, at least 75%, at least 80%, at least 85%, at least 90%, at least 95% or 100% identical to any one of VH sequences described in TABLE 10, and (b) the IL-6 binding moiety can bind an amino acid sequence that is at least 60%, at least 70%, at least 75%, at least 80%, at least 85%, at least 90%, at least 95%, at least 99% or 100% identical to any one of SEQ ID NOs: 83-84. In some embodiments, an engineered protein construct described herein comprises an IL-6 binding region and a TREM1 binding region, wherein (a) the IL-6 binding region comprises an amino acid sequence that is at least 60%, at least 65%, at least 70%, at least 75%, at least 80%, at least 85%, at least 90%, at least 95% or 100% identical to any one of VH sequences described in TABLE 10, (b) the IL-6 binding region can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding region can bind TREM1, a functional fragment thereof, a variant thereof, or a combination thereof. In some embodiments, a multispecific molecule described herein comprises an IL-6 binding domain and a TREM1 binding domain, wherein (a) the IL-6 binding domain comprises an amino acid sequence that is at least 60%, at least 65%, at least 70%, at least 75%, at least 80%, at least 85%, at least 90%, at least 95% or 100% identical to any one of VH sequences described in TABLE 10, (b) the IL-6 binding domain can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding domain can bind TREM1, a functional fragment thereof, a variant thereof, or a combination thereof.

TABLE 10
Exemplary VH sequence for binding to proteins from IL-6 family

| SEQ ID NO: | Target | VH Sequences |
|---|---|---|
| 132 | IL6 | EVQLVESGGGLVQPGGSLRLSCAASGFSLSNYYVTWVRQAPGKGLEWVG |
| | | IIYGSDETAYATSAIGRFTISRDNSKNTLYLQMNSLRAEDTAVYYCARD |
| | | DSSDWDAKFNLWGQGTLVTVSS |
| 133 | IL6 | EVQLVESGGGLVQPGGSLRLSCAASGFNFNDYFMNWVRQAPGKGLEWVA |
| | | QMRNKNYQYGTYYAESLEGRFTISRDDSKNSLYLQMNSLKTEDTAVYYC |
| | | ARESYYGFTSYWGQGTLVTVSS |
| 134 | IL6 | EVQLVESGGKLLKPGGSLKLSCAASGFTFSSFAMSWFRQSPEKRLEWVA |
| | | EISSGGSYTYYPDTVTGRFTISRDNAKNTLYLEMSSLRSEDTAMYYCAR |
| | | GLWGYYALDYWGQGTSVTVSS |
| 135 | IL6 | EVQLVESGGGLVQPGGSLRLSCAASGFTFSPFAMSWVRQAPGKGLEWVA |
| | | KISPGGSWTYYSDTVTGRFTISRDNAKNSLYLQMNSLRAEDTAVYYCAR |
| | | QLWGYYALDIWGQGTTVTVSS |
| 136 | IL6 | QVQLVQSGAEVKKPGSSVKVSCKASGYVLPNYLIEWVRQAPGQGLEWMG |
| | | VTTPGGGTINYAQKFQGRVTITADESTSTAYMELSSLRSEDTAVYYCAR |
| | | SRWDPLYYYALEYWGQGTTVTVSS |
| 137 | IL6 | EVQLVESGGGLVQPGGSLRLSCAASGFTISSNYMIWVRQAPGKGLEWVS |
| | | DLYYYAGDTYYADSVKGRFTMSRDISKNTVYLQMNSLRAEDTAVYYCAR |
| | | WADDHPPWIDLWGRGTLVTVSS |
| 138 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHYYYIDVWGRGTLVTVSS |
| 139 | IL6 | EVQLVQSGGGLIQPGGSLRLSCAASGFTISSNYMTWVRQAPGKGLEWVS |
| | | DLYYYAGDTYYADSVRGRFTMSRDISKNTVYLQMDSLRAEDTGVYYCAR |
| | | WADGHYYYIDVWGGGTLVTVSS |
| 140 | IL6 | QVQLVQSGGGLIQPGGSLRLSCAASGFTISSNYMVWVRQAPGKGLEWVS |
| | | DLYYYAGDTYYADSVKGRFTVSRDISKNTVYLQMNSLRAEDTGVYYCAR |
| | | WADDHYYHIDVWGRGTLVTVSS |
| 141 | IL6 | EVQLVQSGGGLIQPGGSLRLSCAASGFTVSSNYMTWVRQAPGKGLEWVS |
| | | DLYYYAGDTYYADSVKGRFTMSRDISKNTVYLQMNSLRAEDTGVYYCAR |
| | | WADGHYYYADVWGRGTLVSVSS |
| 142 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADGHYYYADVWGRGTLVSVSS |
| 143 | IL6 | EVQLVESGGGLVQPGGSLRLSCAASGFTISSNYMIWVRQAPGKGLEWVS |
| | | DLYYYAGDTYYADSVKGRFTMSRDISKNTVYLQMNSLRAEDTAVYYCAR |
| | | WADDHPAWVDLWGRGTLVTVSS |
| 144 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHPRYIDHWGRGTLVTVSS |
| 145 | IL6 | EVQLVESGGGLVQPGGSLRLSCAASGFTISSNYMIWVRQAPGKGLEWVS |
| | | DLYYYAGDTYYADSVKGRFTMSRDISKNTVYLQMNSLRAEDTAVYYCAR |
| | | WEEEGRGYIDVWGRGTLVTVSS |
| 146 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHNYPHIDVWGRGTLVTVSS |
| 147 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHPPYIDLWGRGTLVTVSS |
| 148 | IL6 | EVQLVESGGGLVQPGGSLRLSCAASGFTISSNYMIWVRQAPGKGLEWVS |
| | | DLYYYAGDTYYADSVKGRFTMSRDISKNTVYLQMNSLRAEDTAVYYCAR |
| | | WADDHPPYIDMWGRGTLVTVSS |
| 149 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHPSHLDIWGRGTLVTVSS |
| 150 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHPSHIDVWGRGTLVTVSS |
| 151 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHNNTYIDVWGRGTLVTVSS |
| 152 | IL6 | EVQLVQSGGGLIQPGGSLRLSCAASGFTISSNYMIWVRQAPGKGLEWVS |
| | | DLYYYAGDTYYADSVKGRFTMSRDISKNTVYLQMNSLRAEDTGVYYCAR |
| | | WADDHAPWVDLWGRGTLVTVSS |
| 153 | IL6 | EVQLVESGGGLVQPGGSLRLSCAASGFSLSNYYVTWVRQAPGKGLEWVG |
| | | IIYGSDETAYATSAIGRFTISRDNSKNTLYLQMNSLRAEDTAVYYCARD |
| | | DSSDWDAKFNLWGQGTLVTVS |
| 154 | IL6 | EVQLVESGGGLVQPGGSLRLSCAASGSVFKINVMAWYRQAPGKGRELVA |
| | | GIISGGSTSYADSVKGRFTISRDNAKNTLYLQMNSLRPEDTAVYYCAFI |
| | | TTESDYDLGRRYWGQGTLVTVSS |
| 155 | IL6R | QVQLVQSGGGLVQPGGSLRLSCAASGFTFSSYYMSWVRQAPGKGLEWVS |
| | | GIYSDGTTHYGDSVKGRFTISRDNAKNTVYLQLNSLRAEDTAMYYCAKG |
| | | AGPTWWYALDAWGQGTLVTVSS |
| 156 | IL6R | EVQLVESGGGLVQPGRSLRLSCAASRFTFDDYAMHWVRQAPGKGLEWVS |
| | | GISWNSGRIGYADSVKGRFTISRDNAENSLFLQMNGLRAEDTALYYCAK |
| | | GRDSFDIWGQGTMVTVSS |
| 157 | IL6R | QVQLQESGPGLVKPSETLSLTCAVSGHSISHDHAWSWVRQPPGEGLEWI |
| | | GFISYSGITNYNPSLQGRVTISRDNSKNTLYLQMNSLRAEDTAVYYCAR |
| | | SLARTTAMDYWGEGTLVTVSS |
| 158 | IL6R | QVQLQESGPGLVRPSQTLSLTCTVSGYSITSDHAWSWVRQPPGRGLEWI |
| | | GYISYSGITTYNPSLKSRVTMLRDTSKNQFSLRLSSVTAADTAVYYCAR |
| | | SLARTTAMDYWGQGSLVTVSS |

In some embodiments, a multispecific molecule described herein comprises at least one heavy chain (HC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 83-84 and 442. In some embodiments, a multispecific molecule described herein comprises an IL-6 binding region, wherein the IL-6 binding region comprises a HC region. In some embodiments, a HC region of an IL-6 binding region comprises a VH sequence that is at least 60%, at least 65%, at least 70%, at least 75%, at least 80%, at least 85%, at least 90%, at least 95% or 100% identical to any one of amino acid sequences described in TABLE 10.

In some embodiments, a HC region of an IL-6 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVMHEALHNHYTQ KSLSLSPG (SEQ ID NO: 481), wherein C-terminus of the VH sequence is linked to N-terminus of the amino acid sequence.

In some embodiments, a HC region of an IL-6 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVCTLPPSREEMTKNQVSLSCAVKGFYPS DIAVEWESNGQPENNYKTTPPVLDSDGSFFLVSKLTVDKSRWQQGNVFSCSVMHEALHNHYTQ KSLSLSPG (SEQ ID NO: 482), wherein C-terminus of the VH sequence is linked to N-terminus of the amino acid sequence.

In some embodiments, multispecific molecules described herein comprise at least one of CDR-Ls described in TABLE 11 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, multispecific molecules described herein comprise any one of CDR-L1 described in TABLE 11 or a variant thereof, any one of CDR-L2 described in TABLE 11 or a variant thereof, and any one of CDR-L3 described in TABLE 11 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, multispecific molecules described herein comprise any one of combinations of CDR-Ls or variants thereof described in TABLE 11, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, a CDR-L variant comprises at least one, at least two or at least three substitutions, deletions, additions or combinations thereof relative to a corresponding parent CDR-L sequence described in TABLE 11. In some embodiments, a CDR-L or a variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 11.

In some embodiments, an engineered protein construct described herein comprises an IL-6 binding moiety, wherein the IL-6 binding moiety comprises any one of combinations of CDR-L1, CDR-L2 and CDR-L3 described in TABLE 11, and (b) the IL-6 binding moiety can bind an amino acid sequence that is at least 60%, at least 70%, at least 75%, at least 80%, at least 85%, at least 90%, at least 95%, at least 99% or 100% identical to any one of SEQ ID NOs: 83-84. In some embodiments, an engineered protein construct described herein comprises an IL-6 binding region and a TREM1 binding region, wherein (a) the IL-6 binding region comprises any one of combinations of CDR-L1, CDR-L2 and CDR-L3 described in TABLE 11, (b) the IL-6 binding region can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding region can bind TREM1, a functional fragment thereof, a variant thereof, or a combination thereof. In some embodiments, a multispecific molecule described herein comprises an IL-6 binding domain and a TREM1 binding domain, wherein (a) the IL-6 binding domain comprises any one of combinations of CDR-L1, CDR-L2 and CDR-L3 described in TABLE 11, (b) the IL-6 binding domain can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding domain can bind TREM1, a functional fragment thereof, a variant thereof, or a combination thereof.

TABLE 11
Combinations of CDR-Ls for binding to proteins from IL-6 family

| | | SEQ | | SEQ | | SEQ | |
|---|---|---|---|---|---|---|---|
| Comb. | | ID | | ID | | ID | |
| No. | Target | NO: | CDR-L1 | NO: | CDR-L2 | NO: | CDR-L3 |
| 66 | IL6 | 159 | QSINNE | 169 | RAS | 179 | QQGYSLRNIDNA |
| 67 | IL6 | 160 | QDIGIS | 170 | NAN | 180 | LQHNSAPYT |
| 68 | IL6 | 161 | SSVSY | 171 | DTS | 181 | QQWSGYPYT |
| 69 | IL6 | 162 | ISVSY | 172 | DMS | 182 | MQWSGYPYT |
| 70 | IL6 | 163 | ESVDNYGIPF | 173 | AAS | 183 | QQSEEVPLT |
| 71 | IL6 | 164 | QGISSW | 174 | KAS | 184 | QQSWLGGS |
| 72 | IL6 | 165 | RASQGISSWLA | 175 | KASTLES | 185 | QQSYSTPWT |
| 73 | IL6 | 166 | RASQGISSWLT | 175 | KASTLES | 186 | QQSYSAPWT |
| 72 | IL6 | 165 | RASQGISSWLA | 175 | KASTLES | 185 | QQSYSTPWT |
| 74 | IL6 | 165 | RASQGISSWLA | 175 | KASTLES | 186 | QQSYSAPWT |
| 74 | IL6 | 165 | RASQGISSWLA | 175 | KASTLES | 186 | QQSYSAPWT |
| 75 | IL6 | 165 | RASQGISSWLA | 175 | KASTLES | 184 | QQSWLGGS |
| 75 | IL6 | 165 | RASQGISSWLA | 175 | KASTLES | 184 | QQSWLGGS |
| 76 | IL6 | 165 | RASQGISSWLA | 175 | KASTLES | 187 | QQSWLGGS |
| 77 | IL6 | 165 | RASQGISSWLA | 175 | KASTLES | 188 | AAHYAAPWT |
| 75 | IL6 | 165 | RASQGISSWLA | 175 | KASTLES | 184 | QQSWLGGS |
| 75 | IL6 | 165 | RASQGISSWLA | 175 | KASTLES | 184 | QQSWLGGS |
| 75 | IL6 | 165 | RASQGISSWLA | 175 | KASTLES | 184 | QQSWLGGS |
| 75 | IL6 | 165 | RASQGISSWLA | 175 | KASTLES | 184 | QQSWLGGS |
| 76 | IL6 | 165 | RASQGISSWLA | 175 | KASTLES | 187 | QQSWLGGS |
| 77 | IL6 | 165 | RASQGISSWLA | 175 | KASTLES | 188 | AAHYAAPWT |
| 75 | IL6 | 165 | RASQGISSWLA | 175 | KASTLES | 184 | QQSWLGGS |
| 66 | IL6 | 159 | QSINNE | 169 | RAS | 179 | QQGYSLRNIDNA |
| 68 | IL6 | 161 | SSVSY | 171 | DTS | 181 | QQWSGYPYT |
| 78 | IL6R | 167 | QSVLSASNTY | 176 | YAS | 189 | QQAYRAPVT |
| 79 | IL6R | 164 | QGISSW | 177 | GAS | 190 | QQANSFPYT |
| 80 | IL6R | 168 | TDISSH | 178 | YGS | 191 | GQGNRLPYT |

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of combinations provided in TABLE 12.

TABLE 12
Exemplary CDR Combinations for Antibody
targeting to proteins from IL-6 family

| Comb. | | CDR- | CDR- | CDR- | CDR- | CDR- | CDR- |
|---|---|---|---|---|---|---|---|
| No. | Target | H1 | H2 | H3 | L1 | L2 | L3 |
| 81 | IL6 | 85 | 97 | 108 | 159 | 169 | 179 |
| 82 | IL6 | 86 | 98 | 109 | 160 | 170 | 180 |
| 83 | IL6 | 87 | 99 | 110 | 161 | 171 | 181 |
| 84 | IL6 | 88 | 100 | 111 | 162 | 172 | 182 |
| 85 | IL6 | 89 | 101 | 112 | 163 | 173 | 183 |
| 86 | IL6 | 90 | 102 | 113 | 164 | 174 | 184 |
| 87 | IL6 | 91 | 103 | 114 | 165 | 175 | 185 |
| 88 | IL6 | 92 | 104 | 115 | 166 | 175 | 186 |
| 89 | IL6 | 93 | 103 | 116 | 165 | 175 | 185 |
| 90 | IL6 | 92 | 103 | 117 | 165 | 175 | 186 |
| 91 | IL6 | 91 | 103 | 117 | 165 | 175 | 186 |
| 92 | IL6 | 91 | 103 | 118 | 165 | 175 | 184 |
| 93 | IL6 | 91 | 103 | 119 | 165 | 175 | 184 |
| 94 | IL6 | 91 | 103 | 120 | 165 | 175 | 187 |
| 95 | IL6 | 91 | 103 | 121 | 165 | 175 | 188 |
| 96 | IL6 | 91 | 103 | 122 | 165 | 175 | 184 |
| 97 | IL6 | 91 | 103 | 123 | 165 | 175 | 184 |
| 98 | IL6 | 91 | 103 | 124 | 165 | 175 | 184 |
| 99 | IL6 | 91 | 103 | 125 | 165 | 175 | 184 |
| 100 | IL6 | 91 | 103 | 126 | 165 | 175 | 187 |
| 101 | IL6 | 91 | 103 | 127 | 165 | 175 | 188 |
| 102 | IL6 | 91 | 103 | 128 | 165 | 175 | 184 |
| 81 | IL6 | 85 | 97 | 108 | 159 | 169 | 179 |
| 83 | IL6 | 87 | 99 | 110 | 161 | 171 | 181 |
| 103 | IL6R | 94 | 105 | 129 | 167 | 176 | 189 |
| 104 | IL6R | 95 | 106 | 130 | 164 | 177 | 190 |
| 105 | IL6R | 96 | 107 | 131 | 168 | 178 | 191 |

In some embodiments, a multispecific molecule described herein comprises at least one VL sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 13, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 83-84. In some embodiments, an engineered protein construct described herein comprises an IL-6 binding moiety, wherein (a) the IL-6 binding moiety comprises an amino acid sequence that is at least 60%, at least 65%, at least 70%, at least 75%, at least 80%, at least 85%, at least 90%, at least 95% or 100% identical to any one of VL sequences described in TABLE 13, and (b) the IL-6 binding moiety can bind an amino acid sequence that is at least 60%, at least 70%, at least 75%, at least 80%, at least 85%, at least 90%, at least 95%, at least 99% or 100% identical to any one of SEQ ID NOs: 83-84. In some embodiments, an engineered protein construct described herein comprises an IL-6 binding region and a TREM1 binding region, wherein (a) the IL-6 binding region comprises an amino acid sequence that is at least 60%, at least 65%, at least 70%, at least 75%, at least 80%, at least 85%, at least 90%, at least 95% or 100% identical to any one of VL sequences described in TABLE 13, (b) the IL-6 binding region can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding region can bind TREM1, a functional fragment thereof, a variant thereof, or a combination thereof. In some embodiments, a multispecific molecule described herein comprises an IL-6 binding domain and a TREM1 binding domain, wherein (a) the IL-6 binding domain comprises an amino acid sequence that is at least 60%, at least 65%, at least 70%, at least 75%, at least 80%, at least 85%, at least 90%, at least 95% or 100% identical to any one of VL sequences described in TABLE 13, (b) the IL-6 binding domain can bind an amino acid sequence that is at least 60%, at least 70%, at least 75%, at least 80%, at least 85%, at least 90%, at least 95%, at least 99% or 100% identical to any one of SEQ ID NOs: 83-84, and (c) the TREM1 binding domain can bind TREM1, a functional fragment thereof, a variant thereof, or a combination thereof.

TABLE 13
Exemplary VL sequence for binding to proteins from IL-6 family

| SEQ ID NO: | Target | VL Sequences |
|---|---|---|
| 192 | IL6 | IQMTQSPSSLSASVGDRVTITCQASQSINNELSWYQQKPGKAPKLL |
| | | IYRASTLASGVPSRFSGSGSGTDFTLTISSLQPDDFATYYCQQGYS |
| | | LRNIDNAFGGGTKVEIK |
| 193 | IL6 | DIQMTQSPSSLSASVGDRVTITCQASQDIGISLSWYQQKPGKAPKL |
| | | LIYNANNLADGVPSRFSGSGSGTDFTLTISSLQPEDFATYYCLQHN |
| | | SAPYTFGQGTKLEIK |
| 194 | IL6 | IVLIQSPAIMSASPGEKVTMTCSASSSVSYMYWYQQKPGSSPRLLI |
| | | YDTSNLASGVPVRFSGSGSGTSYSLTISRMEAEDAATYYCQQWSGY |
| | | PYTFGGGTKLEIK |
| 195 | IL6 | EIVLTQSPATLSLSPGERATLSCSASISVSYMYWYQQKPGQAPRLL |
| | | IYDMSNLASGIPARFSGSGSGTDFTLTISSLEPEDFAVYYCMQWSG |
| | | YPYTFGGGTKVEIK |
| 196 | IL6 | DIVMTQSPDSLAVSLGERATINCRASESVDNYGIPFMNWYQQKPGQ |
| | | PPKLLIYAASNRGSGVPDRFSGSGSGTDFTLTISSLQAEDVAVYYC |
| | | QQSEEVPLTFGQGTKLEIK |
| 197 | IL6 | DIQMTQSPSTLSASVGDRVTITCRASQGISSWLAWYQQKPGKAPKV |
| | | LIYKASTLESGVPSRFSGSGSGTEFTLTISSLQPDDFATYYCQQSW |
| | | LGGSFGQGTKLEIK |
| 198 | IL6 | DIVMTQSPSTLSASVGDRVTITCRASQGISSWLAWYQQKPGRAPKV |
| | | LIYKASTLESGVPSRFSGSGSGTDFTLTISSLQPEDFATYYCQQSY |
| | | STPWTFGQGTKLEIKR |
| 199 | IL6 | DIVMTQSPSTLSASVGDRVTITCRASQGISSWLTWYQQKPGRAPKV |
| | | LIYKASTLESGVPSRFSGSGSGTDFTLTISSLQPEDFATYYCQQSY |
| | | SAPWTFGQGTKLELKR |
| 200 | IL6 | DIVMTQSPSTLSASVGDRVTITCRASQGISSWLAWYQQKPGRAPKA |
| | | LIYKASTLESGVPSRFSGSGSGTDFTLTISSLQPEDFATYYCQQSY |
| | | STPWTFGQGTKLEIKR |
| 201 | IL6 | DIVMTQSPSTLSASVGDRVTITCRASQGISSWLAWYQQKPGRAPKV |
| | | LIYKASTLESGVPSRFSGSGSGTDFTLTISSLQPEDFASYYCQQSY |
| | | SAPWTFGQGTKLELKR |
| 202 | IL6 | DIVMTQSPPTLSASVGDRVTITCRASQGISSWLAWYQQKPGRAPKV |
| | | LIYKASTLESGVPSRFSGSGSGTDFTLTISSLQPEDFASYYCQQSY |
| | | SAPWTFGQGTKLEIKR |
| 203 | IL6 | DIQMTQSPSTLSASVGDRVTITCRASQGISSWLAWYQQKPGKAPKV |
| | | LIYKASTLESGVPSRFSGSGSGTEFTLTISSLQPDDFATYYCQQSW |
| | | LGGSFGQGTKLEIKR |
| 204 | IL6 | DIVMTQSPSTLSASVGDRVTITCRASQGISSWLAWYQQKPGRAPKV |
| | | LIYKASTLESGVPSRFSGSGSGTDFTLTISSLQPEDFATYYCAAHY |
| | | AAPWTFGQGTKLEIKR |
| 205 | IL6 | DIVMTQSPSTLSASVGDRVTITCRASQGISSWLAWYQQKPGRAPKV |
| | | LIYKASTLESGVPSRFSGSGSGTDFTLTISSLQPEDFATYYCQQSW |
| | | LGGSFGQGTKLEIKR |
| 206 | IL6 | AIQMTQSPSSLSASVGDRVTITCQASQSINNELSWYQQKPGKAPKL |
| | | LIYRASTLASGVPSRFSGSGSGTDFTLTISSLQPDDFATYYCQQGY |
| | | SLRNIDNAFGGGTKVEIK |
| 207 | IL6 | QIVLIQSPAIMSASPGEKVTMTCSASSSVSYMYWYQQKPGSSPRLL |
| | | IYDTSNLASGVPVRFSGSGSGTSYSLTISRMEAEDAATYYCQQWSG |
| | | YPYTFGGGTKLEIK |
| 208 | IL6R | DIQLTQSPSSVSVSVGERVTIDCKSSQSVLSASNTYLNWYQQKPGQ |
| | | APQLLIYYASTRESGVPDRFSGSGSGTDFTLTISSLQAEDAAVYYC |
| | | QQAYRAPVTFGQGTKLEIK |
| 209 | IL6R | DIQMTQSPSSVSASVGDRVTITCRASQGISSWLAWYQQKPGKAPKL |
| | | LIYGASSLESGVPSRFSGSGSGTDFTLTISSLQPEDFASYYCQQAN |
| | | SFPYTFGQGTKLEIK |
| 210 | IL6R | DIQMTQSPSSLSASVGDSVTITCQASTDISSHLNWYQQKPGKAPEL |
| | | LIYYGSHLLSGVPSRFSGSGSGTDFTFTISSLEAEDAATYYCGQGN |
| | | RLPYTFGQGTKVEIE |
| 211 | IL6R | DIQMTQSPSSLSASVGDRVTITCRASQDISSYLNWYQQKPGKAPKL |
| | | LIYYTSRLHSGVPSRFSGSGSGTDFTFTISSLQPEDIATYYCQQGN |
| | | TLPYTFGQGTKVEIK |

In some embodiments, a multispecific molecule described herein comprises at least one light chain (LC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 83-84 and 442. In some embodiments, a multispecific molecule described herein comprises an IL-6 binding region, wherein the IL-6 binding region comprises a LC region. In some embodiments, a LC region of an IL-6 binding region comprises a VL sequence that is at least 60%, at least 65%, at least 70%, at least 75%, at least 80%, at least 85%, at least 90%, at least 95% or 100% identical to any one of amino acid sequences described in TABLE 13.

In some embodiments, a LC region of an IL-6 binding region comprises a VL sequence, and an amino acid sequence that is at least 60%, at least 65%, at least 70%, at least 75%, at least 80%, at least 85%, at least 90%, at least 95% or 100% identical to RTVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 483), wherein C-terminus of the VL sequence is linked to N-terminus of the amino acid sequence.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 10; and (b) a VL sequence that is at least 60%, at least 65%, at least 70%, at least 75%, at least 80%, at least 85%, at least 90%, at least 95% or 100% identical to any one of amino acid sequences described in TABLE 13, wherein the multispecific molecule comprises the VH sequence and the VL sequence according to any one of combinations described in TABLE 14.

TABLE 14
Exemplary Combinations of VH sequences and VL sequences
for binding to proteins from IL-6 family

| Comb. No: | Target | VH (SEQ ID NO:) | VL (SEQ ID NO:) |
|---|---|---|---|
| 106 | IL6 | 132 | 192 |
| 107 | IL6 | 133 | 193 |
| 108 | IL6 | 134 | 194 |
| 109 | IL6 | 135 | 195 |
| 110 | IL6 | 136 | 196 |
| 111 | IL6 | 137 | 197 |
| 112 | IL6 | 138 | 198 |
| 113 | IL6 | 139 | 199 |
| 114 | IL6 | 140 | 200 |
| 115 | IL6 | 141 | 201 |
| 116 | IL6 | 142 | 202 |
| 117 | IL6 | 143 | 203 |
| 118 | IL6 | 144 | 203 |
| 119 | IL6 | 145 | 203 |
| 120 | IL6 | 146 | 204 |
| 121 | IL6 | 147 | 203 |
| 122 | IL6 | 148 | 203 |
| 123 | IL6 | 137 | 203 |
| 124 | IL6 | 149 | 205 |
| 125 | IL6 | 150 | 205 |
| 126 | IL6 | 151 | 204 |
| 127 | IL6 | 152 | 205 |
| 128 | IL6 | 153 | 206 |
| 129 | IL6 | 134 | 207 |
| 130 | IL6R | 155 | 208 |
| 131 | IL6R | 156 | 209 |
| 132 | IL6R | 157 | 210 |
| 133 | IL6R | 158 | 211 |

### IL-12 Family

The IL-12 family, as described herein, include Interleukin-12α cytokine (IL-12α), Interleukin-12β cytokine (IL-12β), Interleukin-12 receptor β1 (IL12Rβ1), Interleukin-12 receptor β2 (IL12Rβ2), Interleukin-23α (IL-23α), Interleukin-23 receptor (IL23R), Interleukin-27α (IL-27α), Interleukin-27β (IL-27β), a functional fragment thereof, a variant thereof, a multimeric form thereof or a combination thereof. Amino acid sequences of IL-12 family proteins are recited in TABLE 15.

TABLE 15
Amino Acid Sequences for Proteins from IL-12 Family

| | SEQ | |
|---|---|---|
| | ID | |
| Name | NO: | Amino Acid Sequences |
| IL- | 212 | MCPARSLLLVATLVLLDHLSLARNLPVATPDPGMFPCLHHSQNLLRAVSNMLQKA |
| 12α | | RQTLEFYPCTSEEIDHEDITKDKTSTVEACLPLELTKNESCLNSRETSFITNGSC |
| | | LASRKTSFMMALCLSSIYEDLKMYQVEFKTMNAKLLMDPKRQIFLDQNMLAVIDE |
| | | LMQALNFNSETVPQKSSLEEPDFYKTKIKLCILLHAFRIRAVTIDRVMSYLNAS |
| IL- | 213 | MCHQQLVISWFSLVFLASPLVAIWELKKDVYVVELDWYPDAPGEMVVLTCDTPEE |
| 12β | | DGITWTLDQSSEVLGSGKTLTIQVKEFGDAGQYTCHKGGEVLSHSLLLLHKKEDG |
| | | IWSTDILKDQKEPKNKTFLRCEAKNYSGRFTCWWLTTISTDLTFSVKSSRGSSDP |
| | | QGVTCGAATLSAERVRGDNKEYEYSVECQEDSACPAAEESLPIEVMVDAVHKLKY |
| | | ENYTSSFFIRDIIKPDPPKNLQLKPLKNSRQVEVSWEYPDTWSTPHSYFSLTFCV |
| | | QVQGKSKREKKDRVFTDKTSATVICRKNASISVRAQDRYYSSSWSEWASVPCS |
| IL- | 234 | MLGSRAVMLLLLLPWTAQGRAVPGGSSPAWTQCQQLSQKLCTLAWSAHPLVGHMD |
| 23α | | LREEGDEETTNDVPHIQCGDGCDPQGLRDNSQFCLQRIHQGLIFYEKLLGSDIFT |
| | | GEPSLLPDSPVGQLHASLLGLSQLLQPEGHHWETQQIPSLSPSQPWQRLLLRFKI |
| | | LRSLQAFVAVAARVFAHGAATLSP |
| IL- | 443 | MGQTAGDLGWRLSLLLLPLLLVQAGVWGFPRPPGRPQLSLQELRREFTVSLHLAR |
| 27α | | KLLSEVRGQAHRFAESHLPGVNLYLLPLGEQLPDVSLTFQAWRRLSDPERLCFIS |
| | | TTLQPFHALLGGLGTQGRWTNMERMQLWAMRLDLRDLQRHLRFQVLAAGFNLPEE |
| | | EEEEEEEEEEERKGLLPGALGSALQGPAQVSWPQLLSTYRLLHSLELVLSRAVRE |
| | | LLLLSKAGHSVWPLGFPTLSPQP |
| IL- | 444 | MTPQLLLALVLWASCPPCSGRKGPPAALTLPRVQCRASRYPIAVDCSWTLPPAPN |
| 27β | | STSPVSFIATYRLGMAARGHSWPCLQQTPTSTSCTITDVQLFSMAPYVLNVTAVH |
| | | PWGSSSSFVPFITEHIIKPDPPEGVRLSPLAERQLQVQWEPPGSWPFPEIFSLKY |
| | | WIRYKRQGAARFHRVGPIEATSFILRAVRPRARYYVQVAAQDLTDYGELSDWSLP |
| | | ATATMSLGK |

In some embodiments, multispecific molecules described herein bind to an amino acid sequence PP3, N that is at least 60%, at least 70%, at least 75%, at least 80%, at least 85%, at least 90%, at least 95%, at least 99% or 100% identical to any one of the sequences recited in TABLE 15. In some embodiments, multispecific molecules described herein bind to an amino acid sequence that is at least 60%, at least 70%, at least 75%, at least 80%, at least 85%, at least 90%, at least 95%, at least 99% or 100% identical to any one of SEQ ID NOs: 212-213, 234 and 443-444. In some embodiments, multispecific molecules described herein bind: (a) an amino acid sequence that is at least 60%, at least 70%, at least 75%, at least 80%, at least 85%, at least 90%, at least 95%, at least 99% or 100% identical to any one of SEQ ID NOs: 212-213, 234 and 443-444; and (b) TREM1, a functional fragment thereof, a variant thereof, or a combination thereof.

In some embodiments, multispecific molecules described herein comprise at least one of CDR-Hs described in TABLE 16 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, multispecific molecules described herein comprise any one of CDR-H1 described in TABLE 16 or a variant thereof, any one of CDR-H2 described in TABLE 16 or a variant thereof, and any one of CDR-H3 described in TABLE 16 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, multispecific molecules described herein comprise any one of combinations of CDR-Hs or variants thereof described in TABLE 16, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, a CDR-H variant comprises at least one, at least two or at least three substitutions, deletions, additions or combinations thereof relative to a corresponding parent CDR-H sequence described in TABLE 16. In some embodiments, a CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 16.

In some embodiments, an engineered protein construct described herein comprises an IL-12 binding moiety, wherein (a) the IL-12 binding moiety comprises any one of combinations of CDR-H1, CDR-H2 and CDR-H3 described in TABLE 16, and (b) the IL-12 binding moiety can bind an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, an engineered protein construct described herein comprises an IL-12 binding region and a TREM1 binding region, wherein (a) the IL-12 binding region comprises any one of combinations of CDR-H1, CDR-H2 and CDR-H3 described in TABLE 16, (b) the IL-12 binding region can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding region can bind TREM1, a functional fragment thereof, a variant thereof, or a combination thereof. In some embodiments, a multispecific molecule described herein comprises an IL-12 binding domain and a TREM1 binding domain, wherein (a) the IL-12 binding domain comprises any one of combinations of CDR-H1, CDR-H2 and CDR-H3 described in TABLE 16, (b) the IL-12 binding domain can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding domain can bind TREM1, a functional fragment thereof, a variant thereof, or a combination thereof.

TABLE 16
Combinations of CDR-Hs for binding to proteins from IL-12 family

| | | SEQ | | SEQ | | SEQ | |
|---|---|---|---|---|---|---|---|
| Comb. | | ID | | ID | | ID | |
| No. | Target | NO: | CDR-H1 | NO: | CDR-H2 | NO: | CDR-H3 |
| 134 | IL-12β | 214 | GFTFSSYG | 217 | IRYDGSNK | 219 | KTHGSHDN |
| 135 | IL-12β | 215 | GYTFTSYW | 218 | MSPVDSDI | 220 | ARRRPGQGYFDF |
| 136 | IL-12β | 216 | GYSFTTYW | 218 | MSPVDSDI | 220 | ARRRPGQGYFDF |
| 295 | IL-27α | 445 | GFTFRSYG | 446 | ISSSGSYI | 447 | ARDGGRTSYTAT |
| | | | | | | | AHNWFDP |

In some embodiments, a multispecific molecule described herein comprises at least one VH sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213, 234 and 443-444. In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 17, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, an engineered protein construct described herein comprises an IL-12 binding moiety, wherein (a) the IL-12 binding moiety comprises an amino acid sequence that is at least 60%, at least 65%, at least 70%, at least 75%, at least 80%, at least 85%, at least 90%, at least 95% or 100% identical to any one of VH sequences described in TABLE 17, and (b) the IL-12 binding moiety can bind an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, an engineered protein construct described herein comprises an IL-12 binding region and a TREM1 binding region, wherein (a) the IL-12 binding region comprises an amino acid sequence that is at least 60%, at least 65%, at least 70%, at least 75%, at least 80%, at least 85%, at least 90%, at least 95% or 100% identical to any one of VH sequences described in TABLE 17, (b) the IL-12 binding region can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding region can bind TREM1, a functional fragment thereof, a variant thereof, or a combination thereof. In some embodiments, a multispecific molecule described herein comprises an IL-12 binding domain and a TREM1 binding domain, wherein (a) the IL-12 binding domain comprises an amino acid sequence that is at least 60%, at least 65%, at least 70%, at least 75%, at least 80%, at least 85%, at least 90%, at least 95% or 100% identical to any one of VH sequences described in TABLE 17, (b) the IL-12 binding domain can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding domain can bind TREM1, a functional fragment thereof, a variant thereof, or a combination thereof.

TABLE 17
Exemplary VH sequence for binding to proteins from IL-12 family

| SEQ ID NO: | Target | VH Sequences |
|---|---|---|
| 221 | IL-12β | QVQLVESGGGVVQPGRSLRLSCAASGFTFSSYGMHWVRQAPGKGLEWVA |
| | | FIRYDGSNKYYADSVKGRFTISRDNSKNTLYLQMNSLRAEDTAVYYCKT |
| | | HGSHDNWGQGTMVTVSS |
| 222 | IL-12β | EVQLVQSGAEVKKPGESLKISCQSSGYTFTSYWIGWVRQMPGQGLEWIG |
| | | IMSPVDSDIRYNPMFRGQVTMSVDKSSSTAYLQWSSLKASDTAMYYCAR |
| | | RRPGQGYFDFWGQGTMVTVSS |
| 223 | IL-12β | EVQLVQSGAEVKKPGESLKISCKGSGYSFTTYWLGWVRQMPGKGLDWIG |
| | | IMSPVDSDIRYSPSFQGQVTMSVDKSITTAYLQWNSLKASDTAMYYCAR |
| | | RRPGQGYFDFWGQGTLVTVSS |
| 448 | IL-27α | EVQLVESGGGLVKPGGSLRLSCAASGFTFRSYGMNWVRQAPGKGLEWVS |
| | | GISSSGSYIYYADSVKGRFTISRDNAKNSLYLQMNSLRAEDTAVYYCAR |
| | | DGGRTSYTATAHNWFDPWGQGTLVTVSS |

In some embodiments, a multispecific molecule described herein comprises at least one heavy chain (HC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 212-213, 234 and 443-444. In some embodiments, a multispecific molecule described herein comprises an IL-12 binding region, wherein the IL-12 binding region comprises a HC region. In some embodiments, a HC region of an IL-12 binding region comprises a VH sequence that is at least 60%, at least 65%, at least 70%, at least 75%, at least 80%, at least 85%, at least 90%, at least 95% or 100% identical to any one of amino acid sequences described in TABLE 17.

In some embodiments, a HC region of an IL-12 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVMHEALHNHYTQ KSLSLSPG (SEQ ID NO: 481), wherein C-terminus of the VH sequence is linked to N-terminus of the amino acid sequence.

In some embodiments, a HC region of an IL-12 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVCTLPPSREEMTKNQVSLSCAVKGFYPS DIAVEWESNGQPENNYKTTPPVLDSDGSFFLVSKLTVDKSRWQQGNVFSCSVMHEALHNHYTQ KSLSLSPG (SEQ ID NO: 482), wherein C-terminus of the VH sequence is linked to N-terminus of the amino acid sequence.

In some embodiments, multispecific molecules described herein comprise at least one of CDR-Ls described in TABLE 18 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, multispecific molecules described herein comprise any one of CDR-L1 described in TABLE 18 or a variant thereof, any one of CDR-L2 described in TABLE 18 or a variant thereof, and any one of CDR-L3 described in TABLE 18 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, multispecific molecules described herein comprise any one of combinations of CDR-Ls or variants thereof described in TABLE 18, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, a CDR-L variant comprises at least one, at least two or at least three substitutions, deletions, additions or combinations thereof relative to a corresponding parent CDR-L sequence described in TABLE 18. In some embodiments, a CDR-L or a variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 18.

In some embodiments, an engineered protein construct described herein comprises an IL-12 binding moiety, wherein (a) the IL-12 binding moiety comprises any one of combinations of CDR-L1, CDR-L2 and CDR-L3 described in TABLE 18, and (b) the IL-12 binding moiety can bind an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, an engineered protein construct described herein comprises an IL-12 binding region and a TREM1 binding region, wherein (a) the IL-12 binding region comprises any one of combinations of CDR-L1, CDR-L2 and CDR-L3 described in TABLE 18, (b) the IL-12 binding region can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding region can bind TREM1, a functional fragment thereof, a variant thereof, or a combination thereof. In some embodiments, a multispecific molecule described herein comprises an IL-12 binding domain and a TREM1 binding domain, wherein (a) the IL-12 binding domain comprises any one of combinations of CDR-L1, CDR-L2 and CDR-L3 described in TABLE 18, (b) the IL-12 binding domain can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding domain can bind TREM1, a functional fragment thereof, a variant thereof, or a combination thereof.

TABLE 18
Combinations of CDR-Ls for binding to proteins from IL-12 family

| | | SEQ | | SEQ | | SEQ | |
|---|---|---|---|---|---|---|---|
| Comb. | | ID | | ID | | ID | |
| No. | Target | NO: | CDR-L1 | NO: | CDR-L2 | NO: | CDR-L3 |
| 137 | IL-12β | 224 | RSNIGSNT | 227 | YND | 229 | QSYDRYTHPALL |
| 138 | IL-12β | 225 | QSVGTW | 228 | AAS | 230 | QQYNIYPYT |
| 139 | IL-12β | 226 | QGISSW | 228 | AAS | 230 | QQYNIYPYT |
| 296 | IL-27α | 449 | QSVLFSSNNKNY | 57 | WAS | 450 | QQHASAPPT |

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of combinations provided in TABLE 19.

TABLE 19
Exemplary CDR Combinations for Antibody
targeting to proteins from IL-12 family

| Comb. | | CDR- | CDR- | CDR- | CDR- | CDR- | CDR- |
|---|---|---|---|---|---|---|---|
| No. | Target | H1 | H2 | H3 | L1 | L2 | L3 |
| 140 | IL-12β | 214 | 217 | 219 | 224 | 227 | 229 |
| 141 | IL-12β | 215 | 218 | 220 | 225 | 228 | 230 |
| 142 | IL-12β | 216 | 218 | 220 | 226 | 228 | 230 |
| 297 | IL-27α | 445 | 446 | 447 | 449 | 57 | 450 |

In some embodiments, a multispecific molecule described herein comprises at least one VL sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213, 234 and 443-444. In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 20, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, an engineered protein construct described herein comprises an IL-12 binding moiety, wherein (a) the IL-12 binding moiety comprises an amino acid sequence that is at least 60%, at least 65%, at least 70%, at least 75%, at least 80%, at least 85%, at least 90%, at least 95% or 100% identical to any one of VL sequences described in TABLE 20, and (b) the IL-12 binding moiety can bind an amino acid sequence that is at least 60%, at least 70%, at least 75%, at least 80%, at least 85%, at least 90%, at least 95%, at least 99% or 100% identical to any one of SEQ ID NOs: 212-213 and 443. In some embodiments, an engineered protein construct described herein comprises an IL-12 binding region and a TREM1 binding region, wherein (a) the IL-12 binding region comprises an amino acid sequence that is at least 60%, at least 65%, at least 70%, at least 75%, at least 80%, at least 85%, at least 90%, at least 95% or 100% identical to any one of VL sequences described in TABLE 20, (b) the IL-12 binding region can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding region can bind TREM1, a functional fragment thereof, a variant thereof, or a combination thereof. In some embodiments, a multispecific molecule described herein comprises an IL-12 binding domain and a TREM1 binding domain, wherein (a) the IL-12 binding domain comprises an amino acid sequence that is at least 60%, at least 65%, at least 70%, at least 75%, at least 80%, at least 85%, at least 90%, at least 95% or 100% identical to any one of VL sequences described in TABLE 20, (b) the IL-12 binding domain can bind an amino acid sequence that is at least 60%, at least 70%, at least 75%, at least 80%, at least 85%, at least 90%, at least 95%, at least 99% or 100% identical to any one of SEQ ID NOs: 212-213 and 443, and (c) the TREM1 binding domain can bind TREM1, a functional fragment thereof, a variant thereof, or a combination thereof.

TABLE 20
Exemplary VL sequence for binding to proteins from IL-12 family

| SEQ ID NO: | Target | VL Sequences |
|---|---|---|
| 231 | IL-12β | QSVLTQPPSVSGAPGQRVTISCSGSRSNIGSNTVKWYQQLPGTAP |
| | | KLLIYYNDQRPSGVPDRFSGSKSGTSASLAITGLQAEDEADYYCQ |
| | | SYDRYTHPALLFGTGTKVTVL |
| 232 | IL-12β | EIVLTQSPATLSASPGERATISCRASQSVGTWVAWYQQKPGQAPR |
| | | SLIYAASNLQSGIPARFSGSGSGTDFTLTISSLEPEDFAVYYCQQ |
| | | YNIYPYTFGQGTRLEIK |
| 233 | IL-12β | DIQMTQSPSSLSASVGDRVTITCRASQGISSWLAWYQQKPEKAPK |
| | | SLIYAASSLQSGVPSRFSGSGSGTDFTLTISSLQPEDFATYYCQQ |
| | | YNIYPYTFGQGTKLEIK |
| 451 | IL-27α | DIVMTQSPDSLAVSLGERATINCKSSQSVLFSSNNKNYLAWYQQK |
| | | PGQPPKLLIYWASTRESGVPDRFSGSGSGTDFTLTISSLQAEDVA |
| | | VYYCQQHASAPPTFGGGTKVEIK |

In some embodiments, a multispecific molecule described herein comprises at least one light chain (LC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 212-213, 234 and 443-444. In some embodiments, a multispecific molecule described herein comprises an IL-12 binding region, wherein the IL-12 binding region comprises a LC region. In some embodiments, a LC region of an IL-12 binding region comprises a VL sequence that is at least 60%, at least 65%, at least 70%, at least 75%, at least 80%, at least 85%, at least 90%, at least 95% or 100% identical to any one of amino acid sequences described in TABLE 20.

In some embodiments, a LC region of an IL-12 binding region comprises a VL sequence, and an amino acid sequence that is at least 60%, at least 65%, at least 70%, at least 75%, at least 80%, at least 85%, at least 90%, at least 95% or 100% identical to RTVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 483), wherein C-terminus of the VL sequence is linked to N-terminus of the amino acid sequence.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 17; and (b) a VL sequence that is at least 60%, at least 65%, at least 70%, at least 75%, at least 80%, at least 85%, at least 90%, at least 95% or 100% identical to any one of amino acid sequences described in TABLE 20, wherein the multispecific molecule comprises the VH sequence and the VL sequence according to any one of combinations described in TABLE 21.

TABLE 21
Exemplary Combinations of VH sequences and VL sequences
for binding to proteins from IL-12 family

| Comb. No: | Target | VH (SEQ ID NO:) | VL (SEQ ID NO:) |
|---|---|---|---|
| 143 | IL-12β | 221 | 231 |
| 144 | IL-12β | 222 | 232 |
| 145 | IL-12β | 223 | 233 |
| 298 | IL-27α | 448 | 451 |

### IL-23 Family

The IL-23 family, as described herein, include Interleukin-23at cytokine (IL-23at), a fragment thereof, a variant thereof, a multimeric form thereof or a combination thereof. In some embodiments, IL-23 family proteins bind to Interleukin-23 receptor (IL23R). An amino acid sequence of IL-23at and IL23R are recited in TABLE 22.

TABLE 22
Amino Acid Sequences for Proteins from IL-23 Family

| | SEQ | |
|---|---|---|
| | ID | |
| Name | NO: | Amino Acid Sequences |
| IL- | 234 | MLGSRAVMLLLLLPWTAQGRAVPGGSSPAWTQCQQLSQKLCTLAWSAHPLVGHMDL |
| 23α | | REEGDEETTNDVPHIQCGDGCDPQGLRDNSQFCLQRIHQGLIFYEKLLGSDIFTGE |
| | | PSLLPDSPVGQLHASLLGLSQLLQPEGHHWETQQIPSLSPSQPWQRLLLRFKILRS |
| | | LQAFVAVAARVFAHGAATLSP |
| IL23R | 452 | MNQVTIQWDAVIALYILFSWCHGGITNINCSGHIWVEPATIFKMGMNISIYCQAAI |
| | | KNCQPRKLHFYKNGIKERFQITRINKTTARLWYKNFLEPHASMYCTAECPKHFQET |
| | | LICGKDISSGYPPDIPDEVTCVIYEYSGNMTCTWNAGKLTYIDTKYVVHVKSLETE |
| | | EEQQYLTSSYINISTDSLQGGKKYLVWVQAANALGMEESKQLQIHLDDIVIPSAAV |
| | | ISRAETINATVPKTIIYWDSQTTIEKVSCEMRYKATTNQTWNVKEFDTNFTYVQQS |
| | | EFYLEPNIKYVFQVRCQETGKRYWQPWSSLFFHKTPETVPQVTSKAFQHDTWNSGL |
| | | TVASISTGHLTSDNRGDIGLLLGMIVFAVMLSILSLIGIFNRSFRTGIKRRILLLI |
| | | PKWLYEDIPNMKNSNVVKMLQENSELMNNNSSEQVLYVDPMITEIKEIFIPEHKPT |
| | | DYKKENTGPLETRDYPQNSLFDNTTVVYIPDLNTGYKPQISNFLPEGSHLSNNNEI |
| | | TSLTLKPPVDSLDSGNNPRLQKHPNFAFSVSSVNSLSNTIFLGELSLILNQGECSS |
| | | PDIQNSVEEETTMLLENDSPSETIPEQTLLPDEFVSCLGIVNEELPSINTYFPQNI |
| | | LESHFNRISLLEK |

In some embodiments, multispecific molecules described herein bind to an amino acid sequence PP4, N that is at least 60%, at least 70%, at least 75%, at least 80%, at least 85%, at least 90%, at least 95%, at least 99% or 100% identical to any one of the sequences recited in TABLE 22. In some embodiments, multispecific molecules described herein bind to an amino acid sequence that is at least 60%, at least 70%, at least 75%, at least 80%, at least 85%, at least 90%, at least 95%, at least 99% or 100% identical to SEQ ID NO: 234 or 452. In some embodiments, multispecific molecules described herein can bind: (a) an amino acid sequence that is at least 60%, at least 70%, at least 75%, at least 80%, at least 85%, at least 90%, at least 95%, at least 99% or 100% identical to any one of SEQ ID NOs: 234 and 452; and (b) TREM1, a functional fragment thereof, a variant thereof, or a combination thereof.

In some embodiments, multispecific molecules described herein comprise at least one of CDR-Hs described in TABLE 23 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, multispecific molecules described herein comprise any one of CDR-H1 described in TABLE 23 or a variant thereof, any one of CDR-H2 described in TABLE 23 or a variant thereof, and any one of CDR-H3 described in TABLE 23 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, multispecific antibodies described herein comprise any one of combinations of CDR-Hs or variants thereof described in TABLE 23, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, a CDR-H variant comprises at least one, at least two or at least three substitutions, deletions, additions or combinations thereof relative to a corresponding parent CDR-H sequence described in TABLE 23. In some embodiments, a CDR-H or a variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 23.

In some embodiments, an engineered protein construct described herein comprises an IL-23 binding moiety, wherein (a) the IL-23 binding moiety comprises any one of combinations of CDR-H1, CDR-H2 and CDR-H3 described in TABLE 23, and (b) the IL-23 binding moiety can bind an amino acid sequence that is at least 60%, at least 70%, at least 75%, at least 80%, at least 85%, at least 90%, at least 95%, at least 99% or 100% identical to SEQ ID NO: 234. In some embodiments, an engineered protein construct described herein comprises an IL-23 binding region and a TREM1 binding region, wherein (a) the IL-23 binding region comprises any one of combinations of CDR-H1, CDR-H2 and CDR-H3 described in TABLE 23, (b) the IL-23 binding region can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding region can bind TREM1, a functional fragment thereof, a variant thereof, or a combination thereof. In some embodiments, a multispecific molecule described herein comprises an IL-23 binding domain and a TREM1 binding domain, wherein (a) the IL-23 binding domain comprises any one of combinations of CDR-H1, CDR-H2 and CDR-H3 described in TABLE 23, (b) the IL-23 binding domain can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding domain can bind TREM1, a functional fragment thereof, a variant thereof, or a combination thereof.

TABLE 23
Combinations of CDR-Hs for binding to proteins from IL-23 family

| | | SEQ | | SEQ | | SEQ | |
|---|---|---|---|---|---|---|---|
| Comb. | | ID | | ID | | ID | |
| No. | Target | NO: | CDR-H1 | NO: | CDR-H2 | NO: | CDR-H3 |
| 146 | IL-23α | 235 | GFTFSSYG | 250 | IWYDGSNE | 270 | ARDRGYTSSWYPDA |
| | | | | | | | FDI |
| 147 | IL-23α | 236 | GYSFSNYW | 251 | IDPSNSYT | 271 | ARWYYKPFDV |
| 148 | IL-23α | 237 | GYKFTRYV | 252 | INPYNDGT | 272 | ARNWDTGL |
| 149 | IL-23α | 238 | GYTFTSYL | 253 | INPYNEGT | 273 | ARNWDLPY |
| 150 | IL-23α | 239 | GYTFTDQT | 254 | IYPRDDSP | 274 | AIPDRSGYAWFIY |
| 151 | IL-23α | 240 | GYIFITYW | 255 | IFPASGSA | 275 | ARGGGGFAY |
| 152 | IL-23 | 241 | SYGMH | 256 | VIWYDGSNEYYADSVKG | 276 | DRGYTSSWYPDA |
| 153 | IL-23 | 241 | SYGMH | 257 | VIWYDGSNKYYADSVKG | 277 | DRGYSSSWYPDAFD |
| 154 | IL-23 | 241 | SYGMH | 258 | VISFDGSLKYYADSVKG | 278 | ERTTLSGSYFDY |
| 155 | IL-23 | 242 | SYAMH | 259 | VISHDGSIKYYADSVKG | 278 | ERTTLSGSYFDY |
| 156 | IL-23 | 243 | SYSMN | 260 | YISSRSSTIYIADSVKG | 279 | RIAAAGGFHYYYAL |
| | | | | | | | DV |
| 157 | IL-23 | 244 | TYSMN | 261 | YISSSSSTRYHADSVKG | 280 | RIAAAGPWGYYYAM |
| | | | | | | | DV |
| 158 | IL-23 | 245 | SFSMN | 262 | YISSRSSTIYYADSVKG | 280 | RIAAAGPWGYYYAM |
| | | | | | | | DV |
| 159 | IL-23 | 246 | TYYWS | 263 | LIYTSGSTNYNPSLKS | 281 | DRGYYYGVDV |
| 160 | IL-23 | 247 | SGGYYWS | 264 | HIHYSGNTYYNPSLKS | 282 | NRGFYYGMDV |
| 161 | IL-23 | 247 | SGGYYWS | 265 | YIYYSGSSYYNPSLKS | 283 | DRGHYYGMDV |
| 162 | IL-23 | 247 | SGGYYWS | 266 | YIYYSGSTYYNPSLKS | 283 | DRGHYYGMDV |
| 163 | IL-23 | 248 | SYFWS | 267 | YIYYSGSTNYNPSLKS | 284 | DRGSYYGSDY |
| 164 | IL-23 | 249 | SGGYYWT | 268 | YIYYSGNTYYNPSLKS | 285 | NRGYYYGMDV |
| 165 | IL-23 | 247 | SGGYYWS | 266 | YIYYSGSTYYNPSLKS | 282 | NRGFYYGMDV |
| 161 | IL-23 | 247 | SGGYYWS | 265 | YIYYSGSSYYNPSLKS | 283 | DRGHYYGMDV |
| 166 | IL-23 | 241 | SYGMH | 269 | LIWYDGSNKYYADSVKG | 286 | ENTVTIYYNYGMDV |

In some embodiments, a multispecific molecule described herein comprises at least one VH sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 234 and 452. In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 24, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, an engineered protein construct described herein comprises an IL-23 binding moiety, wherein (a) the IL-23 binding moiety comprises an amino acid sequence that is at least 60%, at least 65%, at least 70%, at least 75%, at least 80%, at least 85%, at least 90%, at least 95% or 100% identical to any one of VH sequences described in TABLE 24, and (b) the IL-23 binding moiety can bind an amino acid sequence that is at least 60%, at least 70%, at least 75%, at least 80%, at least 85%, at least 90%, at least 95%, at least 99% or 100% identical to SEQ ID NO: 234. In some embodiments, an engineered protein construct described herein comprises an IL-23 binding region and a TREM1 binding region, wherein (a) the IL-23 binding region comprises an amino acid sequence that is at least 60%, at least 65%, at least 70%, at least 75%, at least 80%, at least 85%, at least 90%, at least 95% or 100% identical to any one of VH sequences described in TABLE 24, (b) the IL-23 binding region can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding region can bind TREM1, a functional fragment thereof, a variant thereof, or a combination thereof. In some embodiments, a multispecific molecule described herein comprises an IL-23 binding domain and a TREM1 binding domain, wherein (a) the IL-23 binding domain comprises an amino acid sequence that is at least 60%, at least 65%, at least 70%, at least 75%, at least 80%, at least 85%, at least 90%, at least 95% or 100% identical to any one of VH sequences described in TABLE 24, (b) the IL-23 binding domain can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding domain can bind TREM1, a functional fragment thereof, a variant thereof, or a combination thereof.

TABLE 24
Exemplary VH sequence for binding to proteins from IL-23 family

| SEQ ID | | |
|---|---|---|
| NO: | Target | VH Sequences |
| 287 | IL- | QVQLVESGGGVVQPGRSLRLSCAASGFTFSSYGMHWVRQAPGKGLEWVAVIWY |
| | 23α | DGSNEYYADSVKGRFTISRDNSKNTLYLQMNSLRAEDTAVYYCARDRGYTSSW |
| | | YPDAFDIWGQGTMVTVSS |
| 288 | IL- | EVQLVQSGAEVKKPGESLKISCKGSGYSFSNYWIGWVRQMPGKGLEWMGIIDP |
| | 23α | SNSYTRYSPSFQGQVTISADKSISTAYLQWSSLKASDTAMYYCARWYYKPFDV |
| | | WGQGTLVTVSS |
| 289 | IL- | QVQLVQSGAEVKKPGSSVKVSCKASGYKFTRYVMHWVRQAPGQGLEWMGYINP |
| | 23α | YNDGTNYNEKFKGRVTITADKSTSTAYMELSSLRSEDTAVYYCARNWDTGLWG |
| | | QGTTVTVSS |
| 290 | IL- | QVQLVQSGAEVKKPGASVKVSCKASGYTFTSYLMHWVRQAPGQGLEWMGYINP |
| | 23α | YNEGTNYAQKFQGRVTMTRDTSISTAYMELSRLRSDDTAVYYCARNWDLPYWG |
| | | QGTLVTVSS |
| 291 | IL- | QVQLVQSGAEVKKPGSSVKVSCKASGYTFTDQTIHWMRQAPGQGLEWIGYIYP |
| | 23α | RDDSPKYNENFKGKVTITADKSTSTAYMELSSLRSEDTAVYYCAIPDRSGYAW |
| | | FIYWGQGTLVTVSS |
| 292 | IL- | QVQLVQSGAEVKKPGASVKVSCKASGYIFITYWMTWVRQAPGQGLEWMGQIFP |
| | 23α | ASGSADYNEKFEGRVTMTTDTSTSTAYMELRSLRSDDTAVYYCARGGGGFAYW |
| | | GQGTLVTVSS |
| 293 | IL-23 | QVQLVESGGGWQPGRSLRLSCAASGFTFSSYGMHWVRQAPGKGLEWVAVIWYD |
| | | GSNEYYADSVKGRFTISRDNSKNTLYLQMNSLRAEDTAVYYCARDRGYTSSWY |
| | | PDAFDIWGQGTMVTVSS |
| 294 | IL-23 | QVQLVESGGGWQPGRSLRLSCAASGFTFSSYGMHWVRQAPGKGLEWVAVIWYD |
| | | GSNKYYADSVKGRFTISRDNSKNTLYLQMNSLRAEDTAVYYCARDRGYSSSWY |
| | | PDAFDIWGQGTMVTVSS |
| 295 | IL-23 | QVQLVESGGGWQPGRSLRLSCAASGFTFSSYGMHWVRQAPGKGLEWVAVISFD |
| | | GSLKYYADSVKGRFTISRDNSKNTLYLQMNSLRAEDTAVYYCARERTTLSGSY |
| | | FDYWGQGTLVTVSS |
| 296 | IL-23 | QVQLVESGGGWQPGRSLRLSCAASGFTFSSYAMHWVRQAPGKGLEWLSVISHD |
| | | GSIKYYADSVKGRFTISRDNSKNTLYLQMNSLRAEDTAVYYCARERTTLSGSY |
| | | FDYWGQGTLVTVSS |
| 297 | IL-23 | EVQLVESGGGLVQPGGSLRLSCAASGFTFSSYSMNWVRQAPGKGLEWVSYISS |
| | | RSSTIYIADSVKGRFTISRDNAKNSLYLQMNSLRDEDTAVYYCARRIAAAGGF |
| | | HYYYALDVWGQGTTVTVSS |
| 298 | IL-23 | EVQLVESGGGLVQPGGSLRLSCAASGFTFSTYSMNWVRQAPGKGLEWVSYISS |
| | | SSSTRYHADSVKGRFTISRDNAKNSLYLQMNSLRDEDTAVYYCARRIAAAGPW |
| | | GYYYAMDVWGQGTTVTVSS |
| 299 | IL-23 | EVQLVESGGGLVQPGGSLRLSCWSGFTFSSFSMNWVRQAPGKGLEWVSYISSR |
| | | SSTIYYADSVKGRFTISRDNAKNSLYLQMNSLRDEDTAVYYCARRIAAAGPWG |
| | | YYYAMDVWGQGTTVTVSS |
| 300 | IL-23 | QVQLQESGPGLVKPSETLSLTCTVSGGSISTYYWSWIRQPAGKGLEWIGLIYT |
| | | SGSTNYNPSLKSRVTMSLDTSKNQFSLRLTSVTAADTAVYYCARDRGYYYGVD |
| | | VWGQGTTVTVSS |
| 301 | IL-23 | QVQLQESGPGLVKPSQTLSLTCTVSGGSISSGGYYWSWIRQHPGKGLEWIGHI |
| | | HYSGNTYYNPSLKSRVTISVDTSKNQFSLKLSSVTAADTAVYYCAKNRGFYYG |
| | | MDVWGQGTTVTVSS |
| 302 | IL-23 | QVQLQESGPGLVKPSQTLSLTCTVSGGSINSGGYYWSWIRQHPGKGLEWIGYI |
| | | YYSGSSYYNPSLKSRVTISVDTSQNQFSLKLSSVTAADTAVYYCARDRGHYYG |
| | | MDVWGQGTTVTVSS |
| 303 | IL-23 | QVQLQESGPGLVKPSQTLSLTCTVSGGSISSGGYYWSWIRQHPGKGLEWIGYI |
| | | YYSGSTYYNPSLKSRVTISVDTSKNQFSLKLSSVTAADTAVYYCARDRGHYYG |
| | | MDVWGQGTTVTVSS |
| 304 | IL-23 | QVQLQESGPRLVKPSETLSLTCTVSGDSISSYFWSWIRQPPGKGLEWLGYIYY |
| | | SGSTNYNPSLKSRVTISIDTSKNQFSLKLSSVTAADTAVYYCTRDRGSYYGSD |
| | | YWGQGTLVTVSS |
| 305 | IL-23 | QVQLQESGPGLVKPSQTLSLTCTVSGGSISSGGYYWTWIRQHPGKGLEWIGYI |
| | | YYSGNTYYNPSLKSRITISVDTSKNQFSLSLSSVTAADTAVYYCARNRGYYYG |
| | | MDVWGQGTTVTVSS |
| 306 | IL-23 | QVQLQESGPGLVKPSQTLSLTCTVSGGSISSGGYYWSWIRQHPGKGLEWIGYI |
| | | YYSGSTYYNPSLKSRVTMSVDTSKNQFSLKLSSVTAADTAVYYCAKNRGFYYG |
| | | MDVWGQGTTVTVSS |
| 307 | IL-23 | QVQLQESGPGLVKPSQTLSLTCTVSGGSINSGGYYWSWIRQHPGKGLEWIGYI |
| | | YYSGSSYYNPSLKSRVTISVDTSKNQFSLKLSSVTAADTAVYYCARDRGHYYG |
| | | MDVWGQGTTVTVSS |
| 308 | IL-23 | QVQLVESGGGWQPGRSLRLSCAASGFTFSSYGMHWVRQAPGKGLEWVALIWYD |
| | | GSNKYYADSVKGRFTISRDNSKNTLYLQMNSLRAEDTAVYYCARENTVTIYYN |
| | | YGMDVWGQGTTVTVSS |

In some embodiments, a multispecific molecule described herein comprises at least one heavy chain (HC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 234 and 452. In some embodiments, a multispecific molecule described herein comprises an IL-23 binding region, wherein the IL-23 binding region comprises a HC region. In some embodiments, a HC region of an IL-23 binding region comprises a VH sequence that is at least 60%, at least 65%, at least 70%, at least 75%, at least 80%, at least 85%, at least 90%, at least 95% or 100% identical to any one of amino acid sequences described in TABLE 24.

In some embodiments, a HC region of an IL-23 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVMHEALHNHYTQ KSLSLSPG (SEQ ID NO: 481), wherein C-terminus of the VH sequence is linked to N-terminus of the amino acid sequence.

In some embodiments, a HC region of an IL-23 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVCTLPPSREEMTKNQVSLSCAVKGFYPS DIAVEWESNGQPENNYKTTPPVLDSDGSFFLVSKLTVDKSRWQQGNVFSCSVMHEALHNHYTQ KSLSLSPG (SEQ ID NO: 482), wherein C-terminus of the VH sequence is linked to N-terminus of the amino acid sequence.

In some embodiments, multispecific molecules described herein comprise at least one of CDR-Ls described in TABLE 25 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, multispecific molecules described herein comprise any one of CDR-L1 described in TABLE 25 or a variant thereof, any one of CDR-L2 described in TABLE 25 or a variant thereof, and any one of CDR-L3 described in TABLE 25 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, multispecific molecules described herein comprise any one of combinations of CDR-Ls or variants thereof described in TABLE 25, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, a CDR-L variant comprises at least one, at least two or at least three substitutions, deletions, additions or combinations thereof relative to a corresponding parent CDR-L sequence described in TABLE 25. In some embodiments, a CDR-L or a variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 25.

In some embodiments, an engineered protein construct described herein comprises an IL-23 binding moiety, wherein (a) the IL-23 binding moiety comprises any one of combinations of CDR-L1, CDR-L2 and CDR-L3 described in TABLE 25, and (b) the IL-23 binding moiety can bind an amino acid sequence that is at least 60%, at least 70%, at least 75%, at least 80%, at least 85%, at least 90%, at least 95%, at least 99% or 100% identical to SEQ ID NO: 234. In some embodiments, an engineered protein construct described herein comprises an IL-23 binding region and a TREM1 binding region, wherein (a) the IL-23 binding region comprises any one of combinations of CDR-L1, CDR-L2 and CDR-L3 described in TABLE 25, (b) the IL-23 binding region can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding region can bind TREM1, a functional fragment thereof, a variant thereof, or a combination thereof. In some embodiments, a multispecific molecule described herein comprises an IL-23 binding domain and a TREM1 binding domain, wherein (a) the IL-23 binding domain comprises any one of combinations of CDR-L1, CDR-L2 and CDR-L3 described in TABLE 25, (b) the IL-23 binding domain can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding domain can bind TREM1, a functional fragment thereof, a variant thereof, or a combination thereof.

TABLE 25
Combinations of CDR-Ls for binding to proteins from IL-23 family

| | | SEQ | | SEQ | | SEQ | |
|---|---|---|---|---|---|---|---|
| Comb. | | ID | | ID | | ID | |
| No. | Target | NO: | CDR-L1 | NO: | CDR-L2 | NO: | CDR-L3 |
| 167 | IL-23α | 309 | SSNTGAGYD | 328 | GSG | 341 | QSYDSSLSGWV |
| 168 | IL-23α | 310 | SSNIGSGYD | 329 | GNS | 342 | ASWTDGLSLVV |
| 169 | IL-23α | 311 | DHILKF | 330 | GAT | 343 | QMYWSTPFT |
| 170 | IL-23α | 312 | QSISDY | 331 | YAS | 344 | QQGHSFPFT |
| 171 | IL-23α | 313 | RDVAIA | 332 | WAS | 345 | HQYSSYPFT |
| 172 | IL-23α | 314 | ENIYSY | 333 | NAK | 346 | QHHYGIPFT |
| 173 | IL-23 | 315 | TGSSSNTGAGYDVH | 334 | GSGNRPS | 341 | QSYDSSLSGWV |
| 174 | IL-23 | 316 | TGSSSNIGAGYDVH | 335 | GSNNRPS | 341 | QSYDSSLSGWV |
| 175 | IL-23 | 317 | TLRSGINVGTYRIY | 336 | YKSDSDKQQGS | 347 | MIWHSSASV |
| 175 | IL-23 | 317 | TLRSGINVGTYRIY | 336 | YKSDSDKQQGS | 347 | MIWHSSASV |
| 176 | IL-23 | 318 | TLNSGYSDYKVD | 337 | VGTGGIVGSKGD | 348 | GADHGSGSNFVYV |
| 177 | IL-23 | 319 | TLSSGYSDYKVD | 338 | VGTGGIVGSKGE | 348 | GADHGSGSNFVYV |
| 178 | IL-23 | 319 | TLSSGYSDYKVD | 339 | VGTGGTVGSKGE | 348 | GADHGSGSNFVYV |
| 179 | IL-23 | 320 | RASQGIAGWLA | 340 | AASSLQS | 349 | QQADSFPPT |
| 180 | IL-23 | 321 | RASQVISSWLA | 340 | AASSLQS | 350 | QQANSFPFT |
| 181 | IL-23 | 322 | RASQGSSSWFA | 340 | AASSLQS | 350 | QQANSFPFT |
| 182 | IL-23 | 323 | RASQGISSWFA | 340 | AASSLQS | 350 | QQANSFPFT |
| 183 | IL-23 | 324 | RAGQVISSWLA | 340 | AASSLQS | 351 | QQATSFPLT |
| 184 | IL-23 | 325 | RASQGFSGWLA | 340 | AASSLQS | 351 | QQATSFPLT |
| 185 | IL-23 | 326 | RASQVISSWFA | 340 | AASSLQS | 351 | QQATSFPLT |
| 181 | IL-23 | 322 | RASQGSSSWFA | 340 | AASSLQS | 350 | QQANSFPFT |
| 186 | IL-23 | 327 | RASQGIRNDLG | 340 | AASSLQS | 352 | LQHNSYPPT |

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of combinations provided in TABLE 26.

TABLE 26
Exemplary CDR Combinations for Antibody
targeting to proteins from IL-23 family

| Comb. | | CDR- | CDR- | CDR- | CDR- | CDR- | CDR- |
|---|---|---|---|---|---|---|---|
| No. | Target | H1 | H2 | H3 | L1 | L2 | L3 |
| 187 | IL-23α | 235 | 250 | 270 | 309 | 328 | 341 |
| 188 | IL-23α | 236 | 251 | 271 | 310 | 329 | 342 |
| 189 | IL-23α | 237 | 252 | 272 | 311 | 330 | 343 |
| 190 | IL-23α | 238 | 253 | 273 | 312 | 331 | 344 |
| 191 | IL-23α | 239 | 254 | 274 | 313 | 332 | 345 |
| 192 | IL-23α | 240 | 255 | 275 | 314 | 333 | 346 |
| 193 | IL-23 | 241 | 256 | 276 | 315 | 334 | 341 |
| 194 | IL-23 | 241 | 257 | 277 | 316 | 335 | 341 |
| 195 | IL-23 | 241 | 258 | 278 | 317 | 336 | 347 |
| 196 | IL-23 | 242 | 259 | 278 | 317 | 336 | 347 |
| 197 | IL-23 | 243 | 260 | 279 | 318 | 337 | 348 |
| 198 | IL-23 | 244 | 261 | 280 | 319 | 338 | 348 |
| 199 | IL-23 | 245 | 262 | 280 | 319 | 339 | 348 |
| 200 | IL-23 | 246 | 263 | 281 | 320 | 340 | 349 |
| 201 | IL-23 | 247 | 264 | 282 | 321 | 340 | 350 |
| 202 | IL-23 | 247 | 265 | 283 | 322 | 340 | 350 |
| 203 | IL-23 | 247 | 266 | 283 | 323 | 340 | 350 |
| 204 | IL-23 | 248 | 267 | 284 | 324 | 340 | 351 |
| 205 | IL-23 | 249 | 268 | 285 | 325 | 340 | 351 |
| 206 | IL-23 | 247 | 266 | 282 | 326 | 340 | 351 |
| 202 | IL-23 | 247 | 265 | 283 | 322 | 340 | 350 |
| 207 | IL-23 | 241 | 269 | 286 | 327 | 340 | 352 |

In some embodiments, a multispecific molecule described herein comprises at least one VL sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 234 and 452. In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 27, wherein the multispecific molecule is capable of binding an amino acid sequence that is at least 60%, at least 70%, at least 75%, at least 80%, at least 85%, at least 90%, at least 95%, at least 99% or 100% identical to SEQ ID NO: 234. In some embodiments, an engineered protein construct described herein comprises an IL-23 binding moiety, wherein (a) the IL-23 binding moiety comprises an amino acid sequence that is at least 60%, at least 65%, at least 70%, at least 75%, at least 80%, at least 85%, at least 90%, at least 95% or 100% identical to any one of VL sequences described in TABLE 27, and (b) the IL-23 binding moiety can bind an amino acid sequence that is at least 60%, at least 70%, at least 75%, at least 80%, at least 85%, at least 90%, at least 95%, at least 99% or 100% identical to SEQ ID NO: 234. In some embodiments, an engineered protein construct described herein comprises an IL-23 binding region and a TREM1 binding region, wherein (a) the IL-23 binding region comprises an amino acid sequence that is at least 60%, at least 65%, at least 70%, at least 75%, at least 80%, at least 85%, at least 90%, at least 95% or 100% identical to any one of VL sequences described in TABLE 27, (b) the IL-23 binding region can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding region can bind TREM1, a functional fragment thereof, a variant thereof, or a combination thereof. In some embodiments, a multispecific molecule described herein comprises an IL-23 binding domain and a TREM1 binding domain, wherein (a) the IL-23 binding domain comprises an amino acid sequence that is at least 60%, at least 65%, at least 70%, at least 75%, at least 80%, at least 85%, at least 90%, at least 95% or 100% identical to any one of VL sequences described in TABLE 27, (b) the IL-23 binding domain can bind an amino acid sequence that is at least 60%, at least 70%, at least 75%, at least 80%, at least 85%, at least 90%, at least 95%, at least 99% or 100% identical to SEQ ID NO: 234, and (c) the TREM1 binding domain can bind TREM1, a functional fragment thereof, a variant thereof, or a combination thereof.

TABLE 27
Exemplary VL sequence for binding to proteins from IL-23 family

| SEQ ID NO: | Target | VL Sequences |
|---|---|---|
| 353 | IL-23α | QSVLTQPPSVSGAPGQRVTISCTGSSSNTGAGYDVHWYQQVPGT |
| | | APKLLIYGSGNRPSGVPDRFSGSKSGTSASLAITGLQAEDEADY |
| | | YCQSYDSSLSGWVFGGGTRLTVL |
| 354 | IL-23α | QSVLTQPPSVSGAPGQRVTISCTGSSSNIGSGYDVHWYQQLPGT |
| | | APKLLIYGNSKRPSGVPDRFSGSKSGTSASLAITGLQSEDEADY |
| | | YCASWTDGLSLVVFGGGTKLTVL |
| 355 | IL-23α | DIQMTQSPSSLSASVGDRVTITCKASDHILKFLTWYQQKPGKAP |
| | | KLLIYGATSLETGVPSRFSGSGSGTDFTLTISSLQPEDFATYYC |
| | | QMYWSTPFTFGGGTKVEIK |
| 356 | IL-23α | DIQMTQSPSSLSASVGDRVTITCRASQSISDYLHWYQQKPGKAP |
| | | KLLIKYASQSMSGVPSRFSGSGSGSDFTLTISSLQPEDFATYYC |
| | | QQGHSFPFTFGQGTKLEIK |
| 357 | IL-23α | DIQMTQSPSSLSASVGDRVTITCKASRDVAIAVAWYQQKPGKVP |
| | | KLLIYWASTRHTGVPSRFSGSGSRTDFTLTISSLQPEDVADYFC |
| | | HQYSSYPFTFGSGTKLEIK |
| 358 | IL-23α | DIQMTQSPSSLSASVGDRVTITCRTSENIYSYLAWYQQKPGKAP |
| | | KLLIYNAKTLAEGVPSRFSGSGSGTDFTLTISSLQPEDFATYYC |
| | | QHHYGIPFTFGQGTKVEIK |
| 353 | IL-23 | QSVLTQPPSVSGAPGQRVTISCTGSSSNTGAGYDVHWYQQVPGT |
| | | APKLLIYGSGNRPSGVPDRFSGSKSGTSASLAITGLQAEDEADY |
| | | YCQSYDSSLSGWVFGGGTRLTVL |
| 359 | IL-23 | QSVLTQPPSVSGAPGQRVTISCTGSSSNIGAGYDVHWYQQLPGT |
| | | APKLLIYGSNNRPSGVPDRFSGSKSGTSASLAITGLQAEDEADY |
| | | YCQSYDSSLSGWVFGGGTKLTVL |
| 360 | IL-23 | QAVLTQPSSLSASPGASASLTCTLRSGINVGTYRIYWYQQKPGS |
| | | PPQYLLRYKSDSDKQQGSGVPSRFSGSKDASANAGILLISGLQS |
| | | EDEADYYCMIWHSSASVFGGGTKLTVL |
| 361 | IL-23 | QPVLTQPPSASASLGASVTLTCTLNSGYSDYKVDWYQQRPGKGP |
| | | RFVMRVGTGGIVGSKGDGIPDRFSVLGSGLNRYLTIKNIQEEDE |
| | | SDYHCGADHGSGSNFVYVFGTGTKVTVL |
| 362 | IL-23 | QPVLTQPPSASASLGASVTLTCTLSSGYSDYKVDWYQQRPGKGP |
| | | RFVMRVGTGGIVGSKGEGIPDRFSVLGSGLNRYLTIKNIQEEDE |
| | | SDYHCGADHGSGSNFVYVFGTGTKVTVL |
| 363 | IL-23 | QPELTQPPSASASLGASVTLTCTLSSGYSDYKVDWYQLRPGKGP |
| | | RFVMRVGTGGTVGSKGEGIPDRFSVLGSGLNRSLTIKNIQEEDE |
| | | SDYHCGADHGSGSNFVYVFGTGTKVTVL |
| 364 | IL-23 | DIQLTPSPSSVSASVGDRVTITCRASQGIAGWLAWYQQKPGKAP |
| | | KLLIYAASSLQSGVPSRFSGSGSGTDFTLTISSLQPEDFATYYC |
| | | QQADSFPPTFGGGTKVEIK |
| 365 | IL-23 | DIQMTQSPSSVSASVGDRVTITCRASQVISSWLAWYQQKPGKAP |
| | | SLLIYAASSLQSGVPSRFSGSVSGTDFTLTISSLQPEDFATYYC |
| | | QQANSFPFTFGPGTKVDFK |
| 366 | IL-23 | DIQMTQSPSSVSASVGDRVTITCRASQGSSSWFAWYQQKPGKAP |
| | | KLLIYAASSLQSGVPSRFSGSGSGTDFTLTISSLQPEDFATYYC |
| | | QQANSFPFTFGPGTKVDIK |
| 367 | IL-23 | DSQMTQSPSSVSASVGDRVTITCRASQGISSWFAWYQQKPGQAP |
| | | NLLIYAASSLQSGVPSRFSGSGSGTEFTLTISSLQPEDFATYYC |
| | | QQANSFPFTFGPGTKVDIK |
| 368 | IL-23 | DIQMTQSPSSVSASVGDRVTITCRAGQVISSWLAWYQQKPGKAP |
| | | KLLIYAASSLQSGVPSRFSGSGSGTDFTLTISSLQPDDFATYYC |
| | | QQATSFPLTFGGGTKVEIK |
| 369 | IL-23 | DIQMTQSPSSVSASVGDRVTITCRASQGFSGWLAWYQQKPGKAP |
| | | KLLIYAASSLQSGVPSRFSGSGSGTDFTLTISSLQPEDFATYYC |
| | | QQATSFPLTFGPGTKVDIK |
| 370 | IL-23 | DIQLTQSPSSVSASVGDRVTITCRASQVISSWFAWYQQKPGKAP |
| | | NLLIYAASSLQSGVPSRFSGSGSGTDFTLTISSLQPADFATYFC |
| | | QQATSFPLTFGPGTKVDVK |
| 371 | IL-23 | DIQMTQSPSSLSASVGDRVTITCRASQGIRNDLGWYQQKPGKAP |
| | | KRLIYAASSLQSGVPSRFSGSGSGTEFTLTISSLQPEDFATYYC |
| | | LQHNSYPPTFGQGTKVEIE |

In some embodiments, a multispecific molecule described herein comprises at east one light chain (LC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 234 or 452. In some embodiments, a multispecific molecule described herein comprises an IL-23 binding region, wherein the IL-23 binding region comprises a LC region. In some embodiments, a LC region of an IL-23 binding region comprises a VL sequence that is at least 60%, at least 65%, at least 70%, at least 75%, at least 80%, at least 85%, at least 90%, at least 95% or 100% identical to any one of amino acid sequences described in TABLE 27.

In some embodiments, a LC region of an IL-23 binding region comprises a VL sequence, and an amino acid sequence that is at least 60%, at least 65%, at least 70%, at least 75%, at least 80%, at least 85%, at least 90%, at least 95% or 100% identical to RTVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 483), wherein C-terminus of the VL sequence is linked to N-terminus of the amino acid sequence.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 24; and (b) a VL sequence that is at least 60%, at least 65%, at least 70%, at least 75%, at least 80%, at least 85%, at least 90%, at least 95% or 100% identical to any one of amino acid sequences described in TABLE 27, wherein the multispecific molecule comprises the VH sequence and the VL sequence according to any one of combinations described in TABLE 28.

TABLE 28
Exemplary Combinations of VH sequences and VL sequences
for binding to proteins from IL-23 family

| Comb. No: | Target | VH (SEQ ID NO:) | VL (SEQ ID NO:) |
|---|---|---|---|
| 208 | IL23A | 287 | 353 |
| 209 | IL23A | 288 | 354 |
| 210 | IL23A | 289 | 355 |
| 211 | IL23A | 290 | 356 |
| 212 | IL23A | 291 | 357 |
| 213 | IL23A | 292 | 358 |
| 214 | IL23A | 293 | 353 |
| 215 | IL23 | 294 | 359 |
| 216 | IL23 | 295 | 360 |
| 217 | IL23 | 296 | 360 |
| 218 | IL23 | 297 | 361 |
| 219 | IL23 | 298 | 362 |
| 220 | IL23 | 299 | 363 |
| 221 | IL23 | 300 | 364 |
| 222 | IL23 | 301 | 365 |
| 223 | IL23 | 302 | 366 |
| 224 | IL23 | 303 | 367 |
| 225 | IL23 | 304 | 368 |
| 226 | IL23 | 305 | 369 |
| 227 | IL23 | 306 | 370 |
| 228 | IL23 | 307 | 366 |
| 229 | IL23 | 308 | 371 |

### TREM1

Triggering Receptor Expressed on Myeloid Cells 1 (TREM1, but also known as CD354, HGNC: 17760, Entrez Gene: 54210, UniProtKB: Q9NP99) belongs to the Ig superfamily of receptors and is highly expressed on subsets of myeloid cells including neutrophils, monocytes and macrophages. TREM1 is a cell surface receptor that is implicated in innate and adaptive immune function by amplifying inflammatory responses. TREM1 does not comprise its own signaling motifs and instead, receptor activation is mediated through the adapter DAP 12 (DNAX-activating protein 12). This can lead to amplification of inflammatory responses (Bouchon, et al (2000) J. Immunol. 164 (10): 4991-4995). Crosslinking of TREM1 induces expression of IL-8, myeloperoxidase, TNFa and MCP-1, and TREM1 expression can be up-regulated on myeloid cells in response to Toll-Like Receptor (TLR) stimulation (bacterial and fungi stimulation). TREM1 expression has also been shown to contribute to, and amplify the acute inflammatory response during septic shock and infection (Cohen, (2001) Lancet. 358: 776-778). In addition, TREM1 has been associated with other inflammatory diseases including but not limited to IBD (UC and Crohn's), NEC, RA, PsO, nephritis and SLE, and sepsis (see Colonna, M. The biology of TREM receptors. Nat Rev Immunol (2023). https://doi.org/10.1038/s41577-023-00837-1). In some embodiments, PGLYRP1 (peptidoglycan recognition protein 1) is targeted by a molecule described herein to bind TREM1. Five activating forms of TREM receptors exist including TREM 1, 2, 3, 4, and 5, with a soluble form of TREM1 (sTREM1) released during an infection. TREM1 consists of a single V-type immunoglobulin (Ig)-like domain (Ig-V) of about 108 amino acids, followed by a 70 amino acid stalk region. In some embodiments, a molecule described herein binds to sTREM1 or a fragment thereof. In some cases, a bispecific molecule described herein binds to a specific domain in TREM1 or sTREM1 for instance a Ig-V domain or a stalk region.

In some embodiments, multispecific molecules described herein are capable of binding a TREM1 protein including antibodies that disable non-stimulatory myeloid cells. In some embodiments, multispecific (e.g., bispecific, trispecific) molecules described herein that bind to a mammalian TREM1 sequence. In some cases, TREM1 is a murine homolog.

An amino acid sequence of a human TREM1 protein is recited in TABLE 29.

TABLE 29
Amino Acid Sequence of human TREM1 protein

| SEQ | |
|---|---|
| ID | |
| NO: | Amino Acid Sequences |
| 398 | RKTRLWGLLWMLFVSELRAATKLTEEKYELKEGQTLDVKCDYTLEKFASSQKAWQI |
| | IRDGEMPKTLACTERPSKNSHPVQVGRIILEDYHDHGLLRVRMVNLQVEDSGLYQC |
| | VIYQPPKEPHMLFDRIRLVVTKGFSGTPGSNENSTQNVYKIPPTTTKALCPLYTSP |
| | RTVTQAPPKSTADVSTPDSEINLTNVTDIIRVPVFNIVILLAGGFLSKSLVFSVLF |
| | AVTLRSFVP |
| 456 | RKTRLWGLLWMLFVSELRAATKLTEEKYELKEGQTLDVKCDYTLEKFASSQKAWQIIRDGE |
| | MPKTLACTERPSKNSHPVQVGRIILEDYHDHGLLRVRMVNLQVEDSGLYQCVIYQPPKEPH |
| | MLFDRIRLVVTKGPSGTPGSNENSTQNVYKIPPTTTKALCPLYTSPRTVTQAPPKSTADVS |
| | TPDSEINLTNVTDIIRVPVFNIVILLAGGEDSKSLVFSVLFAVTLRSEVP |
| 457 | RKTRLWGLLWMDEVSELRAATKLTEEKYELKEGQTLDVKCDYTLEKFASSQKAWQIIRDGE |
| | MPKTLACTERPSKNSHPVQVGRIILEDYHDHGLLRVRMVNLQVEDSGLYQCVIYQPPKEPH |
| | MLFDRIRLVVTKGFSGTPGSNENSTQNVYKIPPTTTKALCPLYTSPRTVTQAPPKSTADVS |
| | TPDSEINLTNVTDIIRYSFQVPGPLVWTLSPLFPSLCAERM |
| 458 | MRKTRLWGLLWMLFVSELRAATKLTEEKYELKEGQTLDVKCDYTLEKFASSQKAWQIIRDG |
| | EMPKTLACTERPSKNSHPVQVGRIILEDYHDHGLLRVRMVNLQVEDSGLYQCVIYQPPKEP |
| | HMLFDRIRLVVTKGFRCSTLSPSWLVDS |

In some embodiments, multispecific molecules described herein bind to an amino acid sequence that is at least 60%, at least 70%, at least 75%, at least 80%, at least 85%, at least 90%, at least 95%, at least 99% or 100% identical to any one of the sequences recited in TABLE 29. In some embodiments, multispecific molecules described herein bind to an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, multispecific molecules described herein bind: (a) an amino acid sequence that is at least 60%, at least 70%, at least 75%, at least 80%, at least 85%, at least 90%, at least 95%, at least 99% or 100% identical to any one of SEQ ID NOs: 398 and 456-458; and (b) a protein selected from any one of IL-1 family, IL-6 family, IL-12 family and IL-23 family, a variant thereof, a functional fragment thereof, or a combination thereof.

In some embodiments, multispecific molecules described herein comprise at least one of CDR-Hs described in TABLE 30 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, multispecific molecules described herein comprise any one of CDR-H1 described in TABLE 30 or a variant thereof, any one of CDR-H2 described in TABLE 30 or a variant thereof, and any one of CDR-H3 described in TABLE 30 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, multispecific molecules described herein comprise any one of combinations of CDR-Hs or variants thereof described in TABLE 30.1, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, a CDR-H variant comprises at least one, at least two or at least three substitutions, deletions, additions or combinations thereof relative to a corresponding parent CDR-H sequence described in TABLE 30. In some embodiments, a CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 30.

TABLE 30
CDR-Hs for binding to TREM1

| SEQ | | |
|---|---|---|
| ID | CDR- | AMINO ACID |
| NO: | H | SEQUENCE |
| 372 | CDR1 | GYTFTDYVIN |
| 459 | CDR1 | GFTFSTYA |
| 460 | CDR1 | GFSLSSYA |
| 484 | CDR1 | TYAMH |
| 491 | CDR1 | TYAQH |
| 492 | CDR1 | TYALH |
| 534 | CDR1 | GTFSSYAIS |
| 535 | CDR1 | YTFTSYYMH |
| 536 | CDR1 | GSISSSSYYWG |
| 537 | CDR1 | FTFSSYSMN |
| 538 | CDR1 | FTFSSYGMH |
| 539 | CDR1 | FTFDDYAMH |
| 540 | CDR1 | GSISSYYWS |
| 541 | CDR1 | FTFSDHHMD |
| 542 | CDR1 | FTFSSYWMS |
| 543 | CDR1 | YTFTSYYIH |
| 544 | CDR1 | GSISSGGYYWS |
| 545 | CDR1 | FTFSNYGMH |
| 546 | CDR1 | LTFSSYGMH |
| 547 | CDR1 | FTFSTYAMS |
| 548 | CDR1 | GTFSNYAIS |
| 549 | CDR1 | YSISSGYYWA |
| 550 | CDR1 | YSISSGYYWG |
| 551 | CDR1 | GSISSSDYYWG |
| 552 | CDR1 | YTFTGYYMH |
| 553 | CDR1 | YTFTSYGIH |
| 554 | CDR1 | YSFTTYWIG |
| 555 | CDR1 | YTFTSYGIS |
| 556 | CDR1 | FTFGDYAMH |
| 557 | CDR1 | FTFSSYAMS |
| 558 | CDR2 | GIIPIFGTANYAQKFQG |
| 559 | CDR2 | VINPSGGSTSYAQKFQG |
| 560 | CDR2 | IINPSGGSTSYAQKFQG |
| 561 | CDR2 | SIYYSGSTYYNPSLKS |
| 562 | CDR2 | SISSSSNYIYYADSVKG |
| 563 | CDR2 | VISYDGSNKYYADSVKG |
| 564 | CDR2 | SISSSSSYIYYADSVKG |
| 565 | CDR2 | GISWNSGSIGYADSVKG |
| 566 | CDR2 | SIYYSGSTNYNPSLKS |
| 567 | CDR2 | GISWNSGDIGYADSVKG |
| 568 | CDR2 | RTRNKANSYTTEYAASVKG |
| 569 | CDR2 | NIKQDGSEKYYVDSVKG |
| 570 | CDR2 | YISSSSSTIYYADSVKG |
| 571 | CDR2 | HIYYSGSTNYNPSLKS |
| 572 | CDR2 | GITWNSGSIGYADSVKG |
| 573 | CDR2 | YIYYSGSTYYNPSLKS |
| 574 | CDR2 | VIWYDGSNKYYADSVKG |
| 575 | CDR2 | LIWYDGSNKYYADSVKG |
| 576 | CDR2 | AISGSGGSTYYADSVKG |
| 577 | CDR2 | VIWYDGSNKGYADSVKG |
| 578 | CDR2 | VINPGGGSTSYAQKFQG |
| 579 | CDR2 | IINPGGGSTSYAQKFQG |
| 580 | CDR2 | SIIPIFGTANYAQKFQG |
| 581 | CDR2 | SIYHSGSTYYNPSLKS |
| 582 | CDR2 | SIYHSGNTYYNPSLKS |
| 583 | CDR2 | SISYSGSTYYNPSLKS |
| 584 | CDR2 | WINPNSGGTKYAQKFQG |
| 585 | CDR2 | WISAYNGNTNYAQKLQG |
| 586 | CDR2 | IIYPGDSDTRYSPSFQG |
| 373 | CDR2 | EIYPGSGSTF |
| 461 | CDR2 | IRTKSSNYAT |
| 462 | CDR2 | IYAGGSP |
| 485 | CDR2 | RIRTKSSNYATYYADSVKD |
| 487 | CDR2 | RIRTKSSNYATYYAASVKG |
| 587 | CDR3 | ARGQGSDHYYYGMDV |
| 588 | CDR3 | AREGGPRGASFNWFDP |
| 589 | CDR3 | ARDVGSMYFDI |
| 590 | CDR3 | ARHYYYGYAYFDL |
| 591 | CDR3 | ARESDGIDSYFDY |
| 592 | CDR3 | ARESGHSYVSSFDP |
| 593 | CDR3 | ARGLIYGDAFDY |
| 594 | CDR3 | AREVSMTAASLDV |
| 595 | CDR3 | AREAGYDISSAFDI |
| 596 | CDR3 | AREGSGSWETLDV |
| 597 | CDR3 | ARSGEYGFDL |
| 598 | CDR3 | ARGGGYPWEAFDY |
| 599 | CDR3 | ARGRYRRTGSLDV |
| 600 | CDR3 | ARRSSGDYLDV |
| 601 | CDR3 | ARRGGSYDAFQH |
| 602 | CDR3 | AKGPRMSGWWAD |
| 603 | CDR3 | ARGAPGGRHNWFDP |
| 604 | CDR3 | AKGPRMVTHLDV |
| 605 | CDR3 | ARGPLGYKL |
| 606 | CDR3 | ARDAPQLGLDV |
| 607 | CDR3 | ARGGPLGYGDYKGMDV |
| 608 | CDR3 | ARDAGRYYGSSSSWYFDL |
| 609 | CDR3 | AKGPRLLSALDV |
| 610 | CDR3 | AKGGSRYSHFDY |
| 611 | CDR3 | ARDSAQETYYYGMDV |
| 612 | CDR3 | ARDSSIAGRATLSFDY |
| 613 | CDR3 | ARGPSQYYYDSSAIEAFDI |
| 614 | CDR3 | ARDGGGTAQADGAYYYGMDV |
| 615 | CDR3 | ARGRKAAAGIDEAEYFQH |
| 616 | CDR3 | ARDRRMWDPYGMDV |
| 617 | CDR3 | ARDAPAVVGESPAFDI |
| 618 | CDR3 | AKGSTHRGSAYGMDV |
| 619 | CDR3 | ARRPDDRRGLFQH |
| 620 | CDR3 | ARPDYYSSRGVFDI |
| 621 | CDR3 | AKGDYLDPLFDY |
| 622 | CDR3 | ARERGTYYYASGWAN |
| 623 | CDR3 | ARRGGSSSTGLLY |
| 624 | CDR3 | ARTRIDDSFDI |
| 625 | CDR3 | AKSKHSTTSLDV |
| 626 | CDR3 | ARELMVTSGGWLYGMDV |
| 627 | CDR3 | AREAGNYYDIESAFDI |
| 628 | CDR3 | AREGSGYDESMDV |
| 629 | CDR3 | ARGRGIAFDI |
| 630 | CDR3 | AREAGQTSSALDV |
| 631 | CDR3 | AREAGSWLISTAFDI |
| 632 | CDR3 | AREAGTMSSAFDI |
| 633 | CDR3 | ARSGGYSSSWYGTGYDY |
| 634 | CDR3 | ARDRGQYSSSWYGRMDV |
| 635 | CDR3 | ARESGYHVSTAFDI |
| 636 | CDR3 | ARHWYALGSFDI |
| 637 | CDR3 | ARGADYYAGFDY |
| 638 | CDR3 | AKGPRLLGYFDL |
| 639 | CDR3 | AKGPRYSKPYFDY |
| 640 | CDR3 | ARQEYGDGYFDL |
| 641 | CDR3 | ARDLGGYEGAFDP |
| 642 | CDR3 | ARHDDYLSSFDP |
| 643 | CDR3 | ARGPSWIDV |
| 644 | CDR3 | ARELYAYSSPMFYGMDV |
| 645 | CDR3 | ARYYSPYGMDV |
| 646 | CDR3 | ARDSGQYTGSLDV |
| 647 | CDR3 | ARERHSSLGYAY |
| 648 | CDR3 | ARGRPSSSWGNWFDP |
| 649 | CDR3 | ARGSPWDGRLFDI |
| 650 | CDR3 | ARGAGMYDGSPLGMDV |
| 651 | CDR3 | ARAGTIYGRLDL |
| 652 | CDR3 | AKGPRRTSHLDI |
| 653 | CDR3 | AKGPRMTHSYFDL |
| 654 | CDR3 | AKAPRMYGYFDL |
| 655 | CDR3 | AKGPRTRGYFDL |
| 656 | CDR3 | AKAPRTRWTYFDY |
| 657 | CDR3 | ARARRGALAGMDV |
| 658 | CDR3 | ARGGPYPWSGWFDP |
| 659 | CDR3 | ARDLGQYEGYFDL |
| 660 | CDR3 | ARLGDGYRIWADY |
| 661 | CDR3 | ARELIVGATGGLTYYYGM |
| | | DV |
| 374 | CDR3 | RMAAMDY |
| 375 | CDR3 | RIAAMDY |
| 376 | CDR3 | REAAMDY |
| 377 | CDR3 | RLAAMDY |
| 378 | CDR3 | RQAAMDY |
| 463 | CDR3 | TRDMGIRRQFAY |
| 464 | CDR3 | ARGTGDTVYTYFNI |
| 486 | CDR3 | DMGQRRQFAY |
| 488 | CDR3 | DMGIRRQFAY |
| 489 | CDR3 | DQGIRRQFAY |
| 490 | CDR3 | DLGIRRQFAY |
| 493 | CDR1 | SSYWS |
| 494 | CDR2 | YTHYSGISNYNPSLKS |
| 495 | CDR3 | EGYDILTGYEYYGMDV |
| 496 | CDR1 | NYYWT |
| 497 | CDR2 | YIYDSGYTNYNPSLKS |
| 498 | CDR3 | GVLWFGELLPLLDY |
| 503 | CDR1 | SSAIS |
| 499 | CDR1 | SSAVS |
| 500 | CDR1 | TYAIS |
| 501 | CDR1 | IYVIS |
| 502 | CDR1 | RHAIS |
| 504 | CDR1 | SYAFT |
| 505 | CDR1 | RYAIS |
| 506 | CDR1 | RYAFS |
| 507 | CDR1 | TYDIN |
| 508 | CDR2 | GITPIFGTADYAQKFQG |
| 509 | CDR2 | GINPIFGTANYAQKFQG |
| 510 | CDR2 | GIIPLFGTPNYAQRFQD |
| 511 | CDR2 | GIIPLFGTPNYAQQFQD |
| 512 | CDR2 | GIIPLFGTANYAQQFQD |
| 513 | CDR2 | GIIPLFGTANYAQEFQG |
| 514 | CDR2 | GIIPLFGTANYAQKFQG |
| 515 | CDR2 | GIIPIFSTGNYAQKFQG |
| 516 | CDR2 | GIIPIFRTANYAQKFQG |
| 517 | CDR2 | GIIPIFGTSNYAQKFQG |
| 518 | CDR2 | GIIPIFGTPNYAQKFQG |
| 519 | CDR2 | GIIPIFGTADSAQKFQG |
| 520 | CDR2 | GIIPIFGTANYAQKFQG |
| 521 | CDR2 | WVNPNSGNTGYAQKFQD |
| 522 | CDR3 | TPRYRGSSHHYYYALGV |
| 523 | CDR3 | GGAVGFAY |
| 524 | CDR3 | GHGPGSSHYSYYGLDV |
| 525 | CDR3 | SYFYGSGSSNYYYYGLDV |
| 526 | CDR3 | STRVRGVSHYYYYGLDV |
| 527 | CDR3 | SHFSGSGSSHYYYYGMHV |
| 528 | CDR3 | GGNSWTTSLYYYGMDV |
| 529 | CDR3 | SHFYGSGSSHFYYYGMHV |
| 530 | CDR3 | SHFYGSGSSNYYYYGLDV |
| 531 | CDR3 | TPRYRGSSHHYFYALGV |
| 532 | CDR3 | ASQSRSSNYYYYGLDV |
| 533 | CDR3 | DGLNMVRGVHNYYGMDV |

In some embodiments, an engineered protein construct described herein comprises a TREM1 binding moiety, wherein (a) the TREM1 binding moiety comprises any one of combinations of CDR-H1, CDR-H2 and CDR-H3 described in TABLE 30.1, and (b) the TREM1 binding moiety can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding region and an interleukin binding region, wherein (a) the TREM1 binding region comprises any one of combinations of CDR-H1, CDR-H2 and CDR-H3 described in TABLE 30.1, (b) the TREM1 binding region can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding region can bind a protein selected from any one of IL-1 family, IL-6 family, IL-12 family and IL-23 family, a variant thereof, a functional fragment thereof, or a combination thereof. In some embodiments, a multispecific molecule described herein comprises a TREM11 binding domain and an interleukin binding domain, wherein (a) the TREM1 binding domain comprises any one of combinations of CDR-H1, CDR-H2 and CDR-H3 described in TABLE 30.1, (b) the TREM1 binding domain can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding domain can bind a protein selected from any one of IL-1 family, IL-6 family, IL-12 family and IL-23 family, a variant thereof, a functional fragment thereof, or a combination thereof.

TABLE 30.1
Combinations of CDR-Hs for binding to TREM1

| | SEQ | | SEQ | | SEQ | |
|---|---|---|---|---|---|---|
| Comb. | ID | | ID | | ID | |
| No. | NO: | CDR-H1 | NO: | CDR-H2 | NO: | CDR-H3 |
| 230 | 372 | GYTFTDYVIN | 373 | EIYPGSGSTF | 374 | RMAAMDY |
| 231 | 372 | GYTFTDYVIN | 373 | EIYPGSGSTF | 375 | RIAAMDY |
| 232 | 372 | GYTFTDYVIN | 373 | EIYPGSGSTF | 376 | REAAMDY |
| 233 | 372 | GYTFTDYVIN | 373 | EIYPGSGSTF | 377 | RLAAMDY |
| 234 | 372 | GYTFTDYVIN | 373 | EIYPGSGSTF | 378 | RQAAMDY |
| 301 | 459 | GFTFSTYA | 461 | IRTKSSNYAT | 463 | TRDMGIRRQFAY |
| 302 | 460 | GFSLSSYA | 462 | IYAGGSP | 464 | ARGTGDTVYTYFNI |
| 303 | 484 | TYAMH | 485 | RIRTKSSNYATYYADSVK | 486 | DMGQRRQFAY |
| | | | | D | | |
| 304 | 484 | TYAMH | 487 | RIRTKSSNYATYYAASVK | 488 | DMGIRRQFAY |
| | | | | G | | |
| 305 | 484 | TYAMH | 487 | RIRTKSSNYATYYAASVK | 489 | DQGIRRQFAY |
| | | | | G | | |
| 306 | 484 | TYAMH | 487 | RIRTKSSNYATYYAASVK | 490 | DLGIRRQFAY |
| | | | | G | | |
| 307 | 491 | TYAQH | 487 | RIRTKSSNYATYYAASVK | 488 | DMGIRRQFAY |
| | | | | G | | |
| 308 | 492 | TYALH | 487 | RIRTKSSNYATYYAASVK | 488 | DMGIRRQFAY |
| | | | | G | | |
| 309 | 493 | SSYWS | 494 | YTHYSGISNYNPSLKS | 495 | EGYDILTGYEYYGM |
| | | | | | | DV |
| 310 | 496 | NYYWT | 497 | YIYDSGYTNYNPSLKS | 498 | GVLWFGELLPLLDY |
| 311 | 499 | SSAVS | 508 | GITPIFGTADYAQKFQG | 522 | TPRYRGSSHHYYYA |
| | | | | | | LGV |
| 312 | 500 | TYAIS | 509 | GINPIFGTANYAQKFQG | 523 | GGAVGFAY |
| 313 | 501 | IYVIS | 510 | GIIPLFGTPNYAQRFQD | 524 | GHGPGSSHYSYYGL |
| | | | | | | DV |
| 314 | 501 | IYVIS | 511 | GIIPLFGTPNYAQQFQD | 524 | GHGPGSSHYSYYGL |
| | | | | | | DV |
| 315 | 501 | IYVIS | 512 | GIIPLFGTANYAQQFQD | 524 | GHGPGSSHYSYYGL |
| | | | | | | DV |
| 316 | 502 | RHAIS | 513 | GIIPLFGTANYAQEFQG | 525 | SYFYGSGSSNYYYY |
| | | | | | | GLDV |
| 317 | 50 | RHAIS | 514 | GIIPLFGTANYAQKFQG | 525 | SYFYGSGSSNYYYY |
| | | | | | | GLDV |
| 318 | 501 | IYVIS | 514 | GIIPLFGTANYAQKFQG | 524 | GHGPGSSHYSYYGL |
| | | | | | | DV |
| 319 | 503 | SSAIS | 515 | GIIPIFSTGNYAQKFQG | 526 | STRVRGVSHYYYYG |
| | | | | | | LDV |
| 320 | 504 | SYAFT | 516 | GIIPIFRTANYAQKFQG | 527 | SHFSGSGSSHYYYY |
| | | | | | | GMHV |
| 321 | 505 | RYAIS | 517 | GIIPIFGTSNYAQKFQG | 528 | GGNSWTTSLYYYGM |
| | | | | | | DV |
| 322 | 506 | RYAFS | 518 | GIIPIFGTPNYAQKFQG | 529 | SHFYGSGSSHFYYY |
| | | | | | | GMHV |
| 323 | 506 | RYAFS | 518 | GIIPIFGTPNYAQKFQG | 530 | SHFYGSGSSNYYYY |
| | | | | | | GLDV |
| 324 | 503 | SSAIS | 519 | GIIPIFGTADSAQKFQG | 531 | TPRYRGSSHHYFYA |
| | | | | | | LGV |
| 325 | 503 | SSAIS | 520 | GIIPIFGTANYAQKFQG | 532 | ASQSRSSNYYYYGL |
| | | | | | | DV |
| 326 | 507 | TYDIN | 521 | WVNPNSGNTGYAQKFQD | 533 | DGLNMVRGVHNYYG |
| | | | | | | MDV |

In some embodiments, a multispecific molecule described herein comprises at least one VH sequence, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 31, wherein the multispecific molecule is capable of binding an amino acid sequence that is at least 60%, 70%, 75%, 80%, 85%, 90%, 95%, 99% or 100% identical to any one of SEQ ID NOs: 398 and 456-458. In some embodiments, an engineered protein construct described herein comprises a TREM1 binding moiety, wherein (a) the TREM1 binding moiety comprises an amino acid sequence that is at least 60%, at least 65%, at least 70%, at least 75%, at least 80%, at least 85%, at least 90%, at least 95% or 100% identical to any one of VH sequences described in TABLE 31, and (b) the TREM1 binding moiety can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding region and an interleukin binding region, wherein (a) the TREM1 binding region comprises an amino acid sequence that is at least 60%, at least 65%, at least 70%, at least 75%, at least 80%, at least 85%, at least 90%, at least 95% or 100% identical to any one of VH sequences described in TABLE 31, (b) the TREM1 binding region can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding region can bind a protein selected from any one of IL-1 family, IL-6 family, IL-12 family and IL-23 family, a variant thereof, a functional fragment thereof, or a combination thereof. In some embodiments, a multispecific molecule described herein comprises a TREM1 binding domain and an interleukin binding domain, wherein (a) the TREM1 binding domain comprises an amino acid sequence that is at least 60%, at least 65%, at least 70%, at least 75%, at least 80%, at least 85%, at least 90%, at least 95% or 100% identical to any one of VH sequences described in TABLE 31, (b) the TREM1 binding domain can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding domain can bind a protein selected from any one of IL-1 family, IL-6 family, IL-12 family and IL-23 family, a variant thereof, a functional fragment thereof, or a combination thereof.

TABLE 31
Exemplary VH sequence for binding to TREM1

| SEQ ID NO: | VH Sequences |
|---|---|
| 379 | QVQLVQSGAEVKKPGASVKVSCKASGYTFTDYVINWVRQAPGQGLEWMGEI |
| | YPGSGSTFYAQKFQGRVTMTRDTSTSTVYMELSSLRSEDTAVYYCARRMAA |
| | MDYWGQGTLVTVSS |
| 380 | QVQLVQSGAEVKKPGASVKVSCKASGYTFTDYVINWVRQAPGQGLEWMGEI |
| | YPGSGSTFYAQKFQGRVTMTADTSTSTVYMELSSLRSEDTAVYYCTRRMAA |
| | MDYWGQGTLVTVSS |
| 381 | QVQLVQSGAEVKKPGASVKMSCKASGYTFTDYVINWVRQAPGQGLEWIGEI |
| | YPGSGSTFYAQKFQGRATLTADTSTSTAYMEVSSLRSEDTAVYYCTRRMAA |
| | MDYWGQGTLVTVSS |
| 382 | QVQLVQSGAEVKKPGASVKMSCKASGYTFTDYVINWVRQAPGQGLEWIGEI |
| | YPGSGSTFYAQKFQGRATLTADKSTSTAYMEVSSLRSEDTAVYYCTRRMAA |
| | MDYWGQGTLVTVSS |
| 383 | QVQLVQSGAEVKKPGASVKVSCKASGYTFTDYVINWVRQAPGQGLEWMGEI |
| | YPGSGSTFYAQKFQGRVTMTRDTSTSTVYMELSSLRSEDTAVYYCARRLAAM |
| | DYWGQGTLVTVSS |
| 384 | QVQLVQSGAEVKKPGASVKVSCKASGYTFTDYVINWVRQAPGQGLEWMGEI |
| | YPGSGSTFYAQKFQGRVTMTADTSTSTVYMELSSLRSEDTAVYYCTRRLAAM |
| | DYWGQGTLVTVSS |
| 385 | QVQLVQSGAEVKKPGASVKMSCKASGYTFTDYVINWVRQAPGQGLEWIGEI |
| | YPGSGSTFYAQKFQGRATLTADTSTSTAYMEVSSLRSEDTAVYYCTRRLAAM |
| | DYWGQGTLVTVSS |
| 386 | QVQLVQSGAEVKKPGASVKMSCKASGYTFTDYVINWVRQAPGQGLEWIGEI |
| | YPGSGSTFYAQKFQGRATLTADKSTSTAYMEVSSLRSEDTAVYYCTRRLAAM |
| | DYWGQGTLVTVSS |
| 387 | QVQLVQSGAEVKKPGASVKVSCKASGYTFTDYVINWVRQAPGQGLEWMGEI |
| | YPGSGSTFYAQKFQGRVTMTRDTSTSTVYMELSSLRSEDTAVYYCARRQAA |
| | MDYWGQGTLVTVSS |
| 388 | QVQLVQSGAEVKKPGASVKVSCKASGYTFTDYVINWVRQAPGQGLEWMGEI |
| | YPGSGSTFYAQKFQGRVTMTADTSTSTVYMELSSLRSEDTAVYYCTRRQAA |
| | MDYWGQGTLVTVSS |
| 389 | QVQLVQSGAEVKKPGASVKMSCKASGYTFTDYVINWVRQAPGQGLEWIGEI |
| | YPGSGSTFYAQKFQGRATLTADTSTSTAYMEVSSLRSEDTAVYYCTRRQAAM |
| | DYWGQGTLVTVSS |
| 390 | QVQLVQSGAEVKKPGASVKMSCKASGYTFTDYVINWVRQAPGQGLEWIGEI |
| | YPGSGSTFYAQKFQGRATLTADKSTSTAYMEVSSLRSEDTAVYYCTRRQAAM |
| | DYWGQGTLVTVSS |
| 465 | EVQLVESGGGLVQPGGSLKLSCAASGFTFSTYAMHWVRQASGKGLEWVGRI |
| | RTKSSNYATYYAASVKGRFTISRDDSKNTAYLQMNSLKTEDTAVYYCTRDM |
| | GIRRQFAYWGQGTLVTVSS |
| 466 | EVQLVESGGGLVQPGGSLRLSCAASGFSLSSYAMTWVRQAPGKGLEWIGIIY |
| | AGGSPSYASWAKGRFTISKDNSKNTLYLQMNSLRAEDTAVYYCARGTGDTV |
| | YTYFNIWGQGTLVTVSS |
| 662 | EVQLVESGGGLVQPGGSLKLSCAASGFTFSTYAMHWVRQASGKGLEWVGRI |
| | RTKSSNYATYYAASVKGRFTISRDDSKNTAYLQMNSLKTEDTAVYYCTRDM |
| | GIRRQFAYWGQGTLVTVSS |
| 663 | EVQLVESGGGLVQPGGSLRLSCAASGFSLSSYAMTWVRQAPGKGLEWIGIIY |
| | AGGSPSYASWAKGRFTISKDNSKNTLYLQMNSLRAEDTAVYYCARGTGDTV |
| | YTYFNIWGQGTLVTVSS |
| 664 | EVQLVESGGGLVQPGGSLKLSCAASGFTFSTYAMHWVRQASGKGLEWVGRI |
| | RTKSSNYATYYAASVKGRFTISRDDSKNTAYLQMNSLKTEDTAVYYCTRDQ |
| | GIRRQFAYWGQGTLVTVSS |
| 665 | EVQLVESGGGLVQPGGSLKLSCAASGFTFSTYAMHWVRQASGKGLEWVGRI |
| | RTKSSNYATYYAASVKGRFTISRDDSKNTAYLQMNSLKTEDTAVYYCTRDLG |
| | IRRQFAYWGQGTLVTVSS |
| 666 | EVQLVESGGGLVQPGGSLKLSCAASGFTFSTYAQHWVRQASGKGLEWVGRI |
| | RTKSSNYATYYAASVKGRFTISRDDSKNTAYLQMNSLKTEDTAVYYCTRDM |
| | GIRRQFAYWGQGTLVTVSS |
| 667 | EVQLVESGGGLVQPGGSLKLSCAASGFTFSTYALHWVRQASGKGLEWVGRIR |
| | TKSSNYATYYAASVKGRFTISRDDSKNTAYLQMNSLKTEDTAVYYCTRDMGI |
| | RRQFAYWGQGTLVTVSS |
| 668 | QVQLVQSGAEVKKPGSSVKVSCKASGGTFSSYAISWVRQAPGQGLEWMGGII |
| | PIFGTANYAQKFQGRVTITADESTSTAYMELSSLRSEDTAVYYCARGQGSDHY |
| | YYGMDVWGQGTTVTVSS |
| 669 | QVQLVQSGAEVKKPGSSVKVSCKASGGTFSSYAISWVRQAPGQGLEWMGGII |
| | PIFGTANYAQKFQGRVTITADESTSTAYMELSSLRSEDTAVYYCAREGGPRGA |
| | SFNWFDPWGQGTLVTVSS |
| 670 | QVQLVQSGAEVKKPGSSVKVSCKASGGTFSSYAISWVRQAPGQGLEWMGGII |
| | PIFGTANYAQKFQGRVTITADESTSTAYMELSSLRSEDTAVYYCARDVGSMYF |
| | DIWGQGTMVTVSS |
| 671 | QVQLVQSGAEVKKPGSSVKVSCKASGGTFSSYAISWVRQAPGQGLEWMGGII |
| | PIFGTANYAQKFQGRVTITADESTSTAYMELSSLRSEDTAVYYCARHYYYGY |
| | AYFDLWGRGTLVTVSS |
| 672 | QVQLVQSGAEVKKPGASVKVSCKASGYTFTSYYMHWVRQAPGQGLEWMG |
| | VINPSGGSTSYAQKFQGRVTMTRDTSTSTVYMELSSLRSEDTAVYYCARESD |
| | GIDSYFDYWGQGTLVTVSS |
| 673 | QVQLVQSGAEVKKPGASVKVSCKASGYTFTSYYMHWVRQAPGQGLEWMG |
| | VINPSGGSTSYAQKFQGRVTMTRDTSTSTVYMELSSLRSEDTAVYYCARESG |
| | HSYVSSFDPWGQGTLVTVSS |
| 674 | QVQLVQSGAEVKKPGASVKVSCKASGYTFTSYYMHWVRQAPGQGLEWMGII |
| | NPSGGSTSYAQKFQGRVTMTRDTSTSTVYMELSSLRSEDTAVYYCARGLIYG |
| | DAFDYWGQGTLVTVSS |
| 675 | QVQLVQSGAEVKKPGASVKVSCKASGYTFTSYYMHWVRQAPGQGLEWMGII |
| | NPSGGSTSYAQKFQGRVTMTRDTSTSTVYMELSSLRSEDTAVYYCAREVSMT |
| | AASLDVWGQGTMVTVSS |
| 676 | QVQLVQSGAEVKKPGASVKVSCKASGYTFTSYYMHWVRQAPGQGLEWMGII |
| | NPSGGSTSYAQKFQGRVTMTRDTSTSTVYMELSSLRSEDTAVYYCAREAGYD |
| | ISSAFDIWGQGTMVTVSS |
| 677 | QVQLVQSGAEVKKPGASVKVSCKASGYTFTSYYMHWVRQAPGQGLEWMGII |
| | NPSGGSTSYAQKFQGRVTMTRDTSTSTVYMELSSLRSEDTAVYYCAREGSGS |
| | WETLDVWGQGTMVTVSS |
| 678 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARSGEYGFDL |
| | WGRGTLVTVSS |
| 679 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARGGGYPWE |
| | AFDYWGKGTTVTVSS |
| 680 | EVQLVESGGGLVKPGGSLRLSCAASGFTFSSYSMNWVRQAPGKGLEWVSSIS |
| | SSSNYIYYADSVKGRFTISRDNAKNSLYLQMNSLRAEDTAVYYCARGRYRRT |
| | GSLDVWGQGTMVTVSS |
| 681 | QVQLVESGGGVVQPGRSLRLSCAASGFTFSSYGMHWVRQAPGKGLEWVAVI |
| | SYDGSNKYYADSVKGRFTISRDNSKNTLYLQMNSLRAEDTAVYYCARRSSGD |
| | YLDVWGQGTMVTVSS |
| 682 | EVQLVESGGGLVKPGGSLRLSCAASGFTFSSYSMNWVRQAPGKGLEWVSSIS |
| | SSSSYIYYADSVKGRFTISRDNAKNSLYLQMNSLRAEDTAVYYCARRGGSYD |
| | AFQHWGQGTLVTVSS |
| 683 | EVQLVESGGGLVQPGRSLRLSCAASGFTFDDYAMHWVRQAPGKGLEWVSGI |
| | SWNSGSIGYADSVKGRFTISRDNAKNSLYLQMNSLRAEDTALYYCAKGPRMS |
| | GWWADWGQGTLVTVSS |
| 684 | QVQLQESGPGLVKPSETLSLTCTVSGGSISSYYWSWIRQPPGKGLEWIGSIYYS |
| | GSTNYNPSLKSRVTISVDTSKNQFSLKLSSVTAADTAVYYCARGAPGGRHNW |
| | FDPWGQGTLVTVSS |
| 685 | EVQLVESGGGLVQPGRSLRLSCAASGFTFDDYAMHWVRQAPGKGLEWVSGI |
| | SWNSGDIGYADSVKGRFTISRDNAKNTLYLQMNSLRAEDTALYYCAKGPRM |
| | VTHLDVWGQGTMVTVSS |
| 686 | EVQLVESGGGLVQPGGSLRLSCAASGFTFSDHHMDWVRQAPGKGLEWVGRT |
| | RNKANSYTTEYAASVKGRFTISRDDSKNSLYLQMNSLKTEDTAVYYCARGPL |
| | GYKLWGQGTLVTVSS |
| 687 | EVQLVESGGGLVQPGGSLRLSCAASGFTFSSYWMSWVRQAPGKGLEWVANI |
| | KQDGSEKYYVDSVKGRFTISRDNAKNSLYLQMNSLRAEDTAVYYCARDAPQ |
| | LGLDVWGQGTMVTVSS |
| 688 | EVQLVESGGGLVQPGGSLRLSCAASGFTFSSYSMNWVRQAPGKGLEWVSYIS |
| | SSSSTIYYADSVKGRFTISRDNAKNSLYLQMNSLRAEDTAVYYCARGGPLGY |
| | GDYKGMDVWGQGTTVTVSS |
| 689 | QVQLQESGPGLVKPSETLSLTCTVSGGSISSYYWSWIRQPPGKGLEWIGHIYYS |
| | GSTNYNPSLKSRVTISVDTSKNQFSLKLSSVTAADTAVYYCARDAGRYYGSSS |
| | SWYFDLWGRGTLVTVSS |
| 690 | EVQLVESGGGLVQPGRSLRLSCAASGFTFDDYAMHWVRQAPGKGLEWVSGI |
| | TWNSGSIGYADSVKGRFTISRDNAKNSLYLQMNSLRAEDTALYYCAKGPRLL |
| | SALDVWGQGTMVTVSS |
| 691 | EVQLVESGGGLVQPGRSLRLSCAASGFTFDDYAMHWVRQAPGKGLEWVSGI |
| | SWNSGSIGYADSVKGRFTISRDNAKNSLYLQMNSLRAEDTALYYCAKGGSRY |
| | SHFDYWGQGTLVTVSS |
| 692 | QVQLVQSGAEVKKPGASVKVSCKASGYTFTSYYIHWVRQAPGQGLEWMGII |
| | NPSGGSTSYAQKFQGRVTMTRDTSTSTVYMELSSLRSEDTAVYYCARDSAQE |
| | TYYYGMDVWGQGTTVTVSS |
| 693 | QVQLQESGPGLVKPSQTLSLTCTVSGGSISSGGYYWSWIRQHPGKGLEWIGYI |
| | YYSGSTYYNPSLKSRVTISVDTSKNQFSLKLSSVTAADTAVYYCARDSSIAGR |
| | ATLSFDYWGQGTLVTVSS |
| 694 | EVQLVESGGGLVKPGGSLRLSCAASGFTFSSYSMNWVRQAPGKGLEWVSSIS |
| | SSSNYIYYADSVKGRFTISRDNAKNSLYLQMNSLRAEDTAVYYCARGPSQYY |
| | YDSSAIEAFDIWGQGTMVTVSS |
| 695 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARDGGGTAQ |
| | ADGAYYYGMDVWGQGTTVTVSS |
| 696 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARGRKAAAGI |
| | DEAEYFQHWGQGTLVTVSS |
| 697 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARDRRMWDP |
| | YGMDVWGQGTTVTVSS |
| 698 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARDAPAVVGE |
| | SPAFDIWGQGTMVTVSS |
| 699 | QVQLVESGGGVVQPGRSLRLSCAASGFTFSNYGMHWVRQAPGKGLEWVAVI |
| | WYDGSNKYYADSVKGRFTISRDNSKNTLYLQMNSLRAEDTAVYYCAKGSTH |
| | RGSAYGMDVWGQGTTVTVSS |
| 700 | EVQLVESGGGLVKPGGSLRLSCAASGFTFSSYSMNWVRQAPGKGLEWVSSIS |
| | SSSNYIYYADSVKGRFTISRDNAKNSLYLQMNSLKAEDTAVYYCARRPDDRR |
| | GLFQHWGQGTLVTVSS |
| 701 | QVQLVESGGGVVQPGRSLRLSCAASGFTFSSYGMHWVRQAPGKGLEWVAVI |
| | SYDGSNKYYADSVKGRFTISRDNSKNTLYLQMNSLRAEDTAVYYCARPDYY |
| | SSRGVFDIWGQGTMVTVSS |
| 702 | QVQLVESGGGVVQPGRSLRLSCAASGFTFSSYGMHWVRQAPGKGLEWVALI |
| | WYDGSNKYYADSVKGRFTISRDNSKNTLYLQMNSLRAEDTAVYYCAKGDYL |
| | DPLFDYWGQGTLVTVSS |
| 703 | QVQLVESGGGVVQPGRSLRLSCAASGLTFSSYGMHWVRQAPGKGLEWVAVI |
| | SYDGSNKYYADSVKGRFTISRDNSKNTLYLQMNSLRAEDTAVYYCARERGT |
| | YYYASGWANWGQGTLVTVSS |
| 704 | EVQLVESGGGLVKPGGSLRLSCAASGFTFSSYSMNWVRQAPGKGLEWVSSIS |
| | SSSNYIYYADSVKGRFTISRDNAKNSLYLQMNSLRAEDTAVYYCARRGGSSST |
| | GLLYWGQGTLVTVSS |
| 705 | EVQLVESGGGLVKPGGSLRLSCAASGFTFSSYSMNWVRQAPGKGLEWVSSIS |
| | SSSSYIYYADSVKGRFTISRDNAKNSLYLQMNSLRAEDTAVYYCARTRIDDSF |
| | DIWGQGTMVTVSS |
| 706 | EVQLLESGGGLVQPGGSLRLSCAASGFTFSTYAMSWVRQAPGKGLEWVSAIS |
| | GSGGSTYYADSVKGRFTISRDNSKNTLYLQMNSLRAEDTAVYYCAKSKHSTT |
| | SLDVWGQGTMVTVSS |
| 707 | QVQLVESGGGVVQPGRSLRLSCAASGFTFSSYGMHWVRQAPGKGLEWVAVI |
| | WYDGSNKGYADSVKGRFTISRDNSKNTLYLQMNSLRAEDTAVYYCARELMV |
| | TSGGWLYGMDVWGQGTTVTVSS |
| 708 | QVQLVQSGAEVKKPGASVKVSCKASGYTFTSYYMHWVRQAPGQGLEWMGII |
| | NPSGGSTSYAQKFQGRVTMTRDTSTSTVYMELSSLRSEDTAVYYCAREAGNY |
| | YDIESAFDIWGQGTMVTVSS |
| 709 | QVQLVQSGAEVKKPGASVKVSCKASGYTFTSYYMHWVRQAPGQGLEWMG |
| | VINPSGGSTSYAQKFQGRVTMTRDTSTSTVYMELSSLRSEDTAVYYCAREGS |
| | GYDESMDVWGQGTTVTVSS |
| 710 | QVQLVQSGAEVKKPGSSVKVSCKASGGTFSNYAISWVRQAPGQGLEWMGGII |
| | PIFGTANYAQKFQGRVTITADESTSTAYMELSSLRSEDTAVYYCARGRGIAFDI |
| | WGQGTMVTVSS |
| 711 | QVQLVQSGAEVKKPGASVKVSCKASGYTFTSYYMHWVRQAPGQGLEWMG |
| | VINPGGGSTSYAQKFQGRVTMTRDTSTSTVYMELSSLRSEDTAVYYCAREAG |
| | QTSSALDVWGQGTMVTVSS |
| 712 | QVQLVQSGAEVKKPGASVKVSCKASGYTFTSYYMHWVRQAPGQGLEWMGII |
| | NPSGGSTSYAQKFQGRVTMTRDTSTSTVYMELSSLRSEDTAVYYCAREAGSW |
| | LISTAFDIWGQGTMVTVSS |
| 713 | QVQLVQSGAEVKKPGASVKVSCKASGYTFTSYYMHWVRQAPGQGLEWMGII |
| | NPGGGSTSYAQKFQGRVTMTRDTSTSTVYMELSSLRSEDTAVYYCAREAGT |
| | MSSAFDIWGQGTMVTVSS |
| 714 | QVQLVQSGAEVKKPGSSVKVSCKASGGTFSSYAISWVRQAPGQGLEWMGSII |
| | PIFGTANYAQKFQGRVTITADESTSTAYMELSSLRSEDTAVYYCARSGGYSSS |
| | WYGTGYDYWGQGTLVTVSS |
| 715 | QVQLVQSGAEVKKPGSSVKVSCKASGGTFSSYAISWVRQAPGQGLEWMGSII |
| | PIFGTANYAQKFQGRVTITADESTSTAYMELSSLRSEDTAVYYCARDRGQYSS |
| | SWYGRMDVWGQGTTVTVSS |
| 716 | QVQLVQSGAEVKKPGASVKVSCKASGYTFTSYYIHWVRQAPGQGLEWMGII |
| | NPSGGSTSYAQKFQGRVTMTRDTSTSTVYMELSSLRSEDTAVYYCARESGYH |
| | VSTAFDIWGQGTMVTVSS |
| 717 | QVQLVQSGAEVKKPGSSVKVSCKASGGTFSSYAISWVRQAPGQGLEWMGGII |
| | PIFGTANYAQKFQGRVTITADESTSTAYMELSSLRSEDTAVYYCARHWYALG |
| | SFDIWGQGTMVTVSS |
| 718 | QVQLVQSGAEVKKPGASVKVSCKASGYTFTSYYMHWVRQAPGQGLEWMG |
| | VINPSGGSTSYAQKFQGRVTMTRDTSTSTVYMELSSLRSEDTAVYYCARGAD |
| | YYAGFDYWGQGTLVTVSS |
| 719 | EVQLVESGGGLVQPGRSLRLSCAASGFTFDDYAMHWVRQAPGKGLEWVSGI |
| | SWNSGSIGYADSVKGRFTISRDNAKNSLYLQMNSLRAEDTALYYCAKGPRLL |
| | GYFDLWGRGTLVTVSS |
| 720 | EVQLVESGGGLVQPGRSLRLSCAASGFTFDDYAMHWVRQAPGKGLEWVSGI |
| | TWNSGSIGYADSVKGRFTISRDNAKNSLYLQMNSLRAEDTALYYCAKGPRYS |
| | KPYFDYWGQGTLVTVSS |
| 721 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARQEYGDGYF |
| | DLWGRGTLVTVSS |
| 722 | QVQLQESGPGLVKPSETLSLTCAVSGYSISSGYYWAWIRQPPGKGLEWIGSIY |
| | HSGSTYYNPSLKSRVTISVDTSKNQFSLKLSSVTAADTAVYYCARDLGGYEG |
| | AFDPWGQGTLVTVSS |
| 723 | QVQLQESGPGLVKPSETLSLTCAVSGYSISSGYYWGWIRQPPGKGLEWIGSIY |
| | HSGSTYYNPSLKSRVTISVDTSKNQFSLKLSSVTAADTAVYYCARHDDYLSSF |
| | DPWGQGTLVTVSS |
| 724 | QVQLQESGPGLVKPSQTLSLTCTVSGGSISSGGYYWSWIRQHPGKGLEWIGYI |
| | YYSGSTYYNPSLKSRVTISVDTSKNQFSLKLSSVTAADTAVYYCARGPSWIDV |
| | WGQGTMVTVSS |
| 725 | QVQLQESGPGLVKPSETLSLTCAVSGYSISSGYYWGWIRQPPGKGLEWIGSIY |
| | HSGNTYYNPSLKSRVTISVDTSKNQFSLKLSSVTAADTAVYYCARELYAYSSP |
| | MFYGMDVWGRGTTVTVSS |
| 726 | QLQLQESGPGLVKPSETLSLTCTVSGGSISSSSYYWGWIRQPPGKGLEWIGSIS |
| | YSGSTYYNPSLKSRVTISVDTSKNQFSLKLSSVTAADTAVYYCARYYSPYGM |
| | DVWGQGTTVTVSS |
| 727 | QLQLQESGPGLVKPSETLSLTCTVSGGSISSSDYYWGWIRQPPGKGLEWIGSIY |
| | YSGSTYYNPSLKSRVTISVDTSKNQFSLKLSSVTAADTAVYYCARDSGQYTGS |
| | LDVWGQGTMVTVSS |
| 728 | QVQLVQSGAEVKKPGASVKVSCKASGYTFTGYYMHWVRQAPGQGLEWMG |
| | WINPNSGGTKYAQKFQGRVTMTRDTSISTAYMELSRLRSDDTAVYYCARERH |
| | SSLGYAYWGQGTLVTVSS |
| 729 | QVQLVQSGAEVKKPGASVKVSCKASGYTFTSYGIHWVRQAPGQGLEWMGW |
| | ISAYNGNTNYAQKLQGRVTMTTDTSTSTAYMELRSLRSDDTAVYYCARGRPS |
| | SSWGNWFDPWGQGTTVTVSS |
| 730 | EVQLVQSGAEVKKPGESLKISCKGSGYSFTTYWIGWVRQMPGKGLEWMGIIY |
| | PGDSDTRYSPSFQGQVTISADKSISTAYLQWSSLKASDTAMYYCARGSPWDG |
| | RLFDIWGQGTMVTVSS |
| 731 | QVQLVQSGAEVKKPGASVKVSCKASGYTFTSYGISWVRQAPGQGLEWMGWI |
| | SAYNGNTNYAQKLQGRVTMTTDTSTSTAYMELRSLRSDDTAVYYCARGAG |
| | MYDGSPLGMDVWGQGTTVTVSS |
| 732 | QVQLVQSGAEVKKPGASVKVSCKASGYTFTSYGIHWVRQAPGQGLEWMGW |
| | ISAYNGNTNYAQKLQGRVTMTTDTSTSTAYMELRSLRSDDTAVYYCARAGTI |
| | YGRLDLWGRGTLVTVSS |
| 733 | EVQLVESGGGLVQPGRSLRLSCAASGFTFGDYAMHWVRQAPGKGLEWVSGI |
| | SWNSGSIGYADSVKGRFTISRDNAKNSLYLQMNSLRAEDTALYYCAKGPRRT |
| | SHLDIWGQGTMVTVSS |
| 734 | EVQLVESGGGLVQPGRSLRLSCAASGFTFDDYAMHWVRQAPGKGLEWVSGI |
| | SWNSGDIGYADSVKGRFTISRDNAKNSLYLQMNSLRAEDTALYYCAKGPRM |
| | THSYFDLWGRGTLVTVSS |
| 735 | EVQLVESGGGLVQPGRSLRLSCAASGFTFDDYAMHWVRQAPGKGLEWVSGI |
| | SWNSGSIGYADSVKGRFTISRDNAKNSLYLQMNSLRAEDTALYYCAKAPRM |
| | YGYFDLWGRGTSVTVSS |
| 736 | EVQLVESGGGLVQPGRSLRLSCAASGFTFDDYAMHWVRQAPGKGLEWVSGI |
| | SWNSGSIGYADSVKGRFTISRDNAKNSLYLQMNSLRAEDTALYYCAKGPRTR |
| | GYFDLWGRGTLVTVSS |
| 737 | QVQLVESGGGLVQPGRSLRLSCAASGFTFDDYAMHWVRQAPGKGLEWVSGI |
| | SWNSGDIGYADSVKGRFTISRDNAKNSLYLQMNSLRAEDTALYYCAKAPRTR |
| | WTYFDYWGQGTLVTVSS |
| 738 | EVQLLESGGGLVQPGGSLRLSCAASGFTFSSYAMSWVRQAPGKGLEWVSAIS |
| | GSGGSTYYADSVKGRFTISRDNSKNTLYLQMNSLRAEDTAVYYCARARRGA |
| | LAGMDVWGQGTTVTVSS |
| 739 | QVQLQESGPGLVKPSETLSLTCAVSGYSISSGYYWAWIRQPPGKGLEWIGSIY |
| | HSGSTYYNPSLKSRVTISVDTSKNQFSLKLSSVTAADTAVYYCARGGPYPWSG |
| | WFDPWGQGTLVTVSS |
| 740 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARDLGQYEG |
| | YFDLWGRGTLVTVSS |
| 741 | QLQLQESGPGLVKPSETLSLTCTVSGGSISSSSYYWGWIRQPPGKGLEWIGSIY |
| | YSGSTYYNPSLKSRVTISVDTSKNQFSLKLSSVTAADTAVYYCARLGDGYRIW |
| | ADYWGQGTLVTVSS |
| 742 | QVQLVESGGGVVQPGRSLRLSCAASGFTFSSYGMHWVRQAPGKGLEWVALI |
| | WYDGSNKYYADSVKGRFTISRDNSKNTLYLQMNSLRAEDTAVYYCARELIV |
| | GATGGLTYYYGMDVWGQGTTVTVSS |
| 743 | QVQLQESGPGLVKPSETLSLTCTVSGGSISSSYWSWVRQPPGKGLEWIGYTHY |
| | SGISNYNPSLKSRVTISVDTSKNQFSLKLSSVTAADTAVYYCAREGYDILTGYE |
| | YYGMDVWGQGTTVTVSS |
| 744 | QVQLQESGPGLVKPSETLSLTCTVSGGSITNYYWTWIRQPPGKGLEWIGYIYD |
| | SGYTNYNPSLKSRVTLSIDTSKNQFSLKLSSVTAADTAVYYCARGVLWFGELL |
| | PLLDYWGQGTLVTVSS |
| 745 | QVQLVQSGAEVKRPGSSVKVSCKASGGTFSSSAISWVRQAPGQGLEWMGGII |
| | PIFGTTNGAQKFQGRVTITADESTSTAYMELSSLRSEDTAVYYCAAMVRGNY |
| | FYFYGMDVWGQGTTVTVSS |
| 746 | QVQLVESGGGVVQPGRSLRLSCAATEFTFSNYGMHWVRQAPGKGLEWVAVI |
| | WYDGSNKYYADSVKGRFTISRDNSKNTLYLQLNSLSAEDSAVYYCARDGRH |
| | YYGSTSYFGMDVWGQGTTVTVSS |
| 747 | QVQLVQSGAEVKKPGSSVKVSCKASGGTFINSEAINWVRQAPGQGLEWMGGI |
| | IPIFDITNYAQKFQGRVTITADESMSTAYMELSSLRSEDTAVYYCAKTYYDILT |
| | YHYHYGMDVWGQGTTVTVSS |
| 748 | QVQLVQSGAEVKKPGSSVKVSCKTSGGTFSSSAVSWVRQAPGQGLEWMGGI |
| | TPIFGTADYAQKFQGRVTITADASTSTGYMELSSLRSEDTAVYYCAFTPRYRG |
| | SSHHYYYALGVWGQGTTVTVSS |
| 749 | QVQLVQSGAEVKKPGSSVKVSCNPSGGTFSTYAISWVRQAPGQGLEWMGGI |
| | NPIFGTANYAQKFQGRVTITADESTSPGYLELSSLRSEDTAVYYCARGGAVGF |
| | AYWGQGTLVTVSS |
| 750 | QVQLVQSGAEVKRPGSSVKVSCKASGGTFSIYVISWVRQAPGQGLEWMGGII |
| | PLFGTPNYAQRFQDRVTITADESTRTAYMELSSLRSEDTAVYYCARGHGPGSS |
| | HYSYYGLDVWGQGTTVTVSS |
| 751 | QVQLVQSGAEVKRPGSSVKVSCKASGGTFSIYVISWVRQAPGQGLEWMGGII |
| | PLFGTPNYAQQFQDRVTITADESTRTAYMELNSLKSEDTAVYYCARGHGPGS |
| | SHYSYYGLDVWGQGTTVTVSS |
| 752 | QVQLVQSGAEVKRPGSSVKVSCKASGGTFSIYVISWVRQAPGQGLEWMGGII |
| | PLFGTPNYAQQFQDRVTITADESTRTAYMELSSLRSEDTAVYYCARGHGPGSS |
| | HYSYYGLDVWGQGTTVTVSS |
| 753 | QVQLVQSGAEVKKPGSSVKVSCKASGGTFSIYVISWVRQAPGQGLEWMGGII |
| | PLFGTANYAQQFQDRVTITADESTRTAYMELNSLKSEDTAVYYCARGHGPGS |
| | SHYSYYGLDVWGQGTTVTVSS |
| 754 | QVQLVQSGAEVKKPGSSVKVSCKASGGTFNRHAISWVRQAPGQGLEWMGGI |
| | IPLFGTANYAQEFQGRVTIAADEPTSTTYMELRSLRSEDTAVYYCASSYFYGS |
| | GSSNYYYYGLDVWGQGTTVTVSS |
| 755 | QVQLVQSGAEVKKPGSSVKVSCKASGGTFNRHAISWVRQAPGQGLEWMGGI |
| | IPLFGTANYAQKFQGRVTIAADEPTSTTYMELRSLRSEDTAVYYCASSYFYGS |
| | GSSNYYYYGLDVWGQGTTVTVSS |
| 756 | QVQLVQSGAEVKRPGSSVKVSCKASGGTFSIYVISWVRQAPGQGLEWMGGII |
| | PLFGTANYAQKFQGRVTITADESTNTAYMELSSLRSEDTAVYYCARGHGPGS |
| | SHYSYYGLDVWGQGTTVTVSS |
| 757 | QVQLVQSGAEVKKPGSSVKVSCKASGGTFSSSAISWVRQAPGQGLEWMGGII |
| | PIFSTGNYAQKFQGRVTITADESTNTAYMDLSSLRSEDTAVYYCARSTRVRGV |
| | SHYYYYGLDVWGQGTTVTVSS |
| 758 | QVQLVQSGAEVKKPGSSVKVSCKSSGGTFSSYAFTWVRQAPGQGLEWMGGII |
| | PIFRTANYAQKFQGRVTITADESTSTAYMELSSLRSEDTAVYYCASSHFSGSGS |
| | SHYYYYGMHVWGQGTTVTVSS |
| 759 | QVQLVQSGAEVKKPGSSVKVSCKASGGTFSRYAISWVRQAPGQGLEWMGGII |
| | PIFGTSNYAQKFQGRVTIKADESTSTAYMELSSLRSEDTAVYYCARGGNSWTT |
| | SLYYYGMDVWGQGTTVTVSS |
| 760 | QVQLVQSGAEVKKPGSSVKVSCKASGGTFNRYAFSWVRQAPGQGLEWMGGI |
| | IPIFGTPNYAQKFQGRVTITADESTSTAYMELSSLISEDTAVYYCASSHFYGSGS |
| | SHFYYYGMHVWGQGTTVTVSS |
| 761 | QVQLVQSGAEVKKPGSSVKVSCKASGGTFNRYAFSWVRQAPGQGLEWMGGI |
| | IPIFGTPNYAQKFQGRVTITADESTSTAYMELSSLISEDTAVYYCASSHFYGSGS |
| | SNYYYYGLDVWGQGTTVTVSS |
| 762 | QVQLVQSGAEVKKPGSSVKVSCKASGGTFSSSAISWVRQAPGQGLEWMGGII |
| | PIFGTADSAQKFQGRVTITADESTSTAYMELNSLRSEDTAVYYCAFTPRYRGS |
| | SHHYFYALGVWGQGTTVTVSS |
| 763 | QVQLVQSGAEVKKPGSSVKVSCKASGGTFSSSAISWVRQAPGQGLEWMGGII |
| | PIFGTANYAQKFQGRVTITADESTSTAYMELSSLRSEDTAVYYCARASQSRSS |
| | NYYYYGLDVWGQGTTVTVSS |
| 999 | QVQLVQSGAEVKKPGASVKVSCKASGYTFPTYDINWVRQATGQGLEWMGW |
| | VNPNSGNTGYAQKFQDRVTMTRNTSISTAYMELSSLRSEDTAVYYCASDGLN |
| | MVRGVHNYYGMDVWGQGTTVTVSS |

In some embodiments, a multispecific molecule described herein comprises at least one heavy chain (HC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, a multispecific molecule described herein comprises a TREM1 binding region, wherein the TREM1 binding region comprises a HC region. In some embodiments, a HC region of a TREM1 binding region comprises a VH sequence that is at least 60%, at least 65%, at least 70%, at least 75%, at least 80%, at least 85%, at least 90%, at least 95% or 100% identical to any one of amino acid sequences described in TABLE 31.

In some embodiments, a HC region of a TREM1 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVYTLPPSREEMTKNQVSLTCLVKGFYPS DIAVEWESNGQPENNYKTTPPVLDSDGSFFLYSKLTVDKSRWQQGNVFSCSVMHEALHNHYTQ KSLSLSPG (SEQ ID NO: 481), wherein C-terminus of the VH sequence is linked to N-terminus of the amino acid sequence.

In some embodiments, a HC region of a TREM1 binding region comprises a VH sequence, and an amino acid sequence that is at least 60%, at least 65%, at least 70%, at least 75%, at least 80%, at least 85%, at least 90%, at least 95% or 100% identical to ASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKRVEPKSCDKTHTCPPCPAPEAAGGPSVFLFPPKPK DTLMISRTPEVTCVVVDVSHEDPEVKFNWYVDGVEVHNAKTKPREEQYNSTYRVVSVLTVLHQ DWLNGKEYKCKVSNKALAAPIEKTISKAKGQPREPQVCTLPPSREEMTKNQVSLSCAVKGFYPS DIAVEWESNGQPENNYKTTPPVLDSDGSFFLVSKLTVDKSRWQQGNVFSCSVMHEALHNHYTQ KSLSLSPG (SEQ ID NO: 482), wherein C-terminus of the VH sequence is linked to N-terminus of the amino acid sequence.

In some embodiments, multispecific molecules described herein comprise at least one of CDR-Ls described in TABLE 32 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, multispecific molecules described herein comprise any one of CDR-L1 described in TABLE 32 or a variant thereof, any one of CDR-L2 described in TABLE 32 or a variant thereof, and any one of CDR-L3 described in TABLE 32 or a variant thereof, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, multispecific molecules described herein comprise any one of combinations of CDR-Ls or variants thereof described in TABLE 32.1, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NO: 398 and 456-458. In some embodiments, a CDR-L variant comprises at least one, at least two or at least three substitutions, deletions, additions or combinations thereof relative to a corresponding parent CDR-L sequence described in TABLE 32. In some embodiments, a CDR-L or a variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 32.

TABLE 32
CDR-Ls for binding to TREM1

| SEQ | | |
|---|---|---|
| ID | CDR- | AMINO ACID |
| NO: | L | SEQUENCE |
| 391 | CDR1 | SASSSVSYMH |
| 467 | CDR1 | ESVDTFDYSF |
| 470 | CDR1 | QNIGSD |
| 764 | CDR1 | RASESVDTFDYSFLH |
| 766 | CDR1 | RASQSVDTFDYSFLH |
| 780 | CDR1 | QASQDISNYLN |
| 781 | CDR1 | RASQSVSSSYLA |
| 782 | CDR1 | KSSQSVLYSSNNKNYLA |
| 783 | CDR1 | RASQSVSSNLA |
| 784 | CDR1 | RSSQSLLHSNGYNYLD |
| 785 | CDR1 | KSSQSVLFSSNNKNYLA |
| 770 | CDR1 | RASQSVSSYLA |
| 786 | CDR1 | RASQSVSSSFLA |
| 768 | CDR1 | RASQGISSWLA |
| 787 | CDR1 | RASQSISSWLA |
| 788 | CDR1 | RASQSISSFLN |
| 789 | CDR1 | RASQSIGSWLA |
| 790 | CDR1 | RASQSISSYLN |
| 791 | CDR1 | RASQSVGSNLA |
| 792 | CDR1 | RASQSISRYLN |
| 793 | CDR1 | RASQSINSWLA |
| 794 | CDR1 | RASQDISSWLA |
| 795 | CDR1 | RASQGIDSWLA |
| 796 | CDR1 | RSSQSLLHRNGYNYLD |
| 797 | CDR2 | DASNLET |
| 798 | CDR2 | GASSRAT |
| 799 | CDR2 | WASTRES |
| 800 | CDR2 | GASTRAT |
| 801 | CDR2 | LGSNRAS |
| 802 | CDR2 | DASNRAT |
| 769 | CDR2 | AASSLQS |
| 803 | CDR2 | DASNLAT |
| 804 | CDR2 | KASSLES |
| 805 | CDR2 | AASNLQS |
| 806 | CDR2 | DASSLES |
| 807 | CDR2 | LGSHRAS |
| 808 | CDR2 | DSSNRAT |
| 392 | CDR2 | TTSNLAS |
| 468 | CDR2 | RAS |
| 471 | CDR2 | KAA |
| 765 | CDR2 | RASNLES |
| 809 | CDR3 | QQVYVLPFT |
| 810 | CDR3 | QQYLGFPPT |
| 811 | CDR3 | QQSFLTPWT |
| 812 | CDR3 | QQFNNHPIT |
| 813 | CDR3 | VQARQTPLT |
| 814 | CDR3 | MQARDAPWT |
| 815 | CDR3 | QQLASYPYT |
| 816 | CDR3 | MQARQTPFT |
| 817 | CDR3 | MQARQAPWT |
| 818 | CDR3 | MQARQVPPWT |
| 819 | CDR3 | MQARQAFT |
| 820 | CDR3 | QQYTSWPLT |
| 821 | CDR3 | QQLDSHPPT |
| 822 | CDR3 | QQYDVDPLT |
| 823 | CDR3 | QQAFISPPT |
| 824 | CDR3 | QQADTLPIT |
| 825 | CDR3 | QQSDIHPRT |
| 826 | CDR3 | QQDSIYPIT |
| 827 | CDR3 | QQANSFPLT |
| 828 | CDR3 | QQYKSFSPFT |
| 829 | CDR3 | QQSYSDLT |
| 830 | CDR3 | QQYLIPPIT |
| 831 | CDR3 | QQHQSFSPT |
| 832 | CDR3 | QQRSVLPLT |
| 833 | CDR3 | QQIFSTPLT |
| 834 | CDR3 | QQSFYDPIT |
| 835 | CDR3 | QQYLYFPLT |
| 836 | CDR3 | QQGVNYPFT |
| 837 | CDR3 | QQVISFPT |
| 838 | CDR3 | QQYDDFPPIT |
| 839 | CDR3 | QQSLDLPFT |
| 840 | CDR3 | QQINDHPFT |
| 841 | CDR3 | QQYGPYPYT |
| 842 | CDR3 | QQSHSTPLT |
| 843 | CDR3 | QQLASQPPT |
| 844 | CDR3 | QQYAYWPLT |
| 845 | CDR3 | QQDFSLPYT |
| 846 | CDR3 | QQSLTHPT |
| 847 | CDR3 | QQYDLLPYT |
| 848 | CDR3 | QQAVIHPPYT |
| 849 | CDR3 | QQYNVHPPRT |
| 850 | CDR3 | MQSRNAPWT |
| 851 | CDR3 | MQARHGFT |
| 852 | CDR3 | MQAREVPFT |
| 853 | CDR3 | MQARHVPPLT |
| 854 | CDR3 | QQHDSAPYT |
| 855 | CDR3 | MQGRQVPFT |
| 856 | CDR3 | MQARGTPWT |
| 857 | CDR3 | MQSRRAPPWT |
| 858 | CDR3 | QQFQSYPFT |
| 859 | CDR3 | QQSSADSPFT |
| 860 | CDR3 | MQARQLPWT |
| 861 | CDR3 | QQHDVWPIT |
| 862 | CDR3 | MQTRHTPT |
| 863 | CDR3 | MQDFARPPT |
| 864 | CDR3 | QQRAVFPPT |
| 865 | CDR3 | QQDATGIT |
| 866 | CDR3 | QQLASFPWT |
| 867 | CDR3 | QQLAFTPWT |
| 869 | CDR3 | QQDHSFIT |
| 870 | CDR3 | QQDVSDFT |
| 871 | CDR3 | QQLYHAPPIT |
| 872 | CDR3 | QQYDSLPFT |
| 873 | CDR3 | QQVYLFPWT |
| 874 | CDR3 | QQFFLAPPT |
| 875 | CDR3 | QQAVSLPWT |
| 876 | CDR3 | QQFDNLPYT |
| 877 | CDR3 | QQATAHPPT |
| 878 | CDR3 | QQAVSHPLT |
| 879 | CDR3 | QQATSLPLT |
| 880 | CDR3 | MQRLQAWT |
| 881 | CDR3 | QQYRTYPT |
| 882 | CDR3 | QQHSLLSIT |
| 883 | CDR3 | QHYNLWRT |
| 884 | CDR3 | QQHSTYSWT |
| 885 | CDR3 | QQHDVWPYT |
| 886 | CDR3 | QQYFSTPPT |
| 887 | CDR3 | QQYALTPYT |
| 888 | CDR3 | QQDHDRPLT |
| 889 | CDR3 | HQWSGYPT |
| 469 | CDR3 | QQSNEDPYT |
| 472 | CDR3 | QQYYYGSAGADTDT |
| 767 | CDR3 | QQSNQDPYT |
| 890 | CDR1 | RASQSVSSSYLA |
| 771 | CDR2 | GASSRAT |
| 891 | CDR3 | QQYGSSPT |
| 892 | CDR1 | RASQGISSALA |
| 893 | CDR2 | DASSLES |
| 894 | CDR3 | QQFNSYPYT |
| 895 | CDR1 | DLGIRRQFAY |
| 779 | CDR3 | QQYGSSPLT |
| 775 | CDR3 | QQYNSYPLT |
| 772 | CDR3 | QQYNSYPYT |
| 773 | CDR3 | QQANSFPFT |
| 774 | CDR3 | QQYNSYPWT |
| 776 | CDR3 | QQYNSYPIT |
| 777 | CDR3 | QQFNSYPLT |
| 778 | CDR3 | QQYNSYPPT |

In some embodiments, an engineered protein construct described herein comprises a TREM1 binding moiety, wherein (a) the TREM1 binding moiety comprises any one of combinations of CDR-L1, CDR-L2 and CDR-L3 described in TABLE 32.1, and (b) the TREM1 binding moiety can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding region and an interleukin binding region, wherein (a) the TREM1 binding region comprises any one of combinations of CDR-L1, CDR-L2 and CDR-L3 described in TABLE 32.1, (b) the TREM1 binding region can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding region can bind a protein selected from any one of IL-1 family, IL-6 family, IL-12 family and IL-23 family, a variant thereof, a functional fragment thereof, or a combination thereof. In some embodiments, a multispecific molecule described herein comprises a TREM1 binding domain and an interleukin binding domain, wherein (a) the TREM1 binding domain comprises any one of combinations of CDR-L1, CDR-L2 and CDR-L3 described in TABLE 32.1, (b) the TREM1 binding domain can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding domain can bind a protein selected from any one of IL-1 family, IL-6 family, IL-12 family and IL-23 family, a variant thereof, a functional fragment thereof, or a combination thereof.

TABLE 32.1
Combinations of CDR-Ls for binding to TREM1

| | SEQ | | SEQ | | SEQ | |
|---|---|---|---|---|---|---|
| Comb. | ID | | ID | | ID | |
| No. | NO: | CDR-L1 | NO: | CDR-L2 | NO: | CDR-L3 |
| 235 | 391 | SASSSVSYMH | 392 | TTSNLAS | 393 | HQWSGYPT |
| 299 | 467 | ESVDTFDYSF | 468 | RAS | 469 | QQSNEDPYT |
| 300 | 470 | QNIGSD | 471 | KAA | 472 | QQYYYGSAGADTDT |
| 327 | 764 | RASESVDTFDYSFLH | 765 | RASNLES | 469 | QQSNEDPYT |
| 328 | 766 | RASQSVDTFDYSFLH | 765 | RASNLES | 767 | QQSNQDPYT |
| 329 | 768 | RASQGISSWLA | 769 | AASSLQS | 772 | QQYNSYPYT |
| 330 | 768 | RASQGISSWLA | 769 | AASSLQS | 773 | QQANSFPFT |
| 331 | 768 | RASQGISSWLA | 769 | AASSLQS | 774 | QQYNSYPWT |
| 332 | 768 | RASQGISSWLA | 769 | AASSLQS | 775 | QQYNSYPLT |
| 333 | 768 | RASQGISSWLA | 769 | AASSLQS | 772 | QQYNSYPYT |
| 334 | 768 | RASQGISSWLA | 769 | AASSLQS | 776 | QQYNSYPIT |
| 335 | 768 | RASQGISSWLA | 769 | AASSLQS | 777 | QQFNSYPLT |
| 336 | 768 | RASQGISSWLA | 769 | AASSLQS | 778 | QQYNSYPPT |
| 337 | 770 | RASQSVSSYLA | 771 | GASSRAT | 779 | QQYGSSPLT |

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 33.

TABLE 33
Exemplary CDR Combinations for Antibody targeting TREM1

| Comb. | CDR- | CDR- | CDR- | CDR- | CDR- | CDR- |
|---|---|---|---|---|---|---|
| No. | H1 | H2 | H3 | L1 | L2 | L3 |
| 236 | 372 | 373 | 374 | 391 | 392 | 393 |
| 237 | 372 | 373 | 377 | 391 | 392 | 393 |
| 238 | 372 | 373 | 378 | 391 | 392 | 393 |
| 338 | 459 | 461 | 463 | 467 | 468 | 469 |
| 339 | 460 | 462 | 464 | 470 | 471 | 472 |
| 340 | 484 | 485 | 486 | 764 | 765 | 469 |
| 341 | 484 | 487 | 488 | 764 | 765 | 469 |
| 342 | 484 | 487 | 488 | 766 | 765 | 767 |
| 343 | 499 | 508 | 522 | 768 | 769 | 772 |
| 344 | 500 | 509 | 523 | 768 | 769 | 773 |
| 345 | 501 | 510 | 524 | 768 | 769 | 774 |
| 346 | 501 | 511 | 524 | 768 | 769 | 775 |
| 347 | 501 | 511 | 524 | 768 | 769 | 772 |
| 348 | 501 | 512 | 524 | 768 | 769 | 775 |
| 349 | 502 | 513 | 525 | 768 | 769 | 775 |
| 350 | 502 | 514 | 525 | 768 | 769 | 772 |
| 351 | 502 | 514 | 525 | 768 | 769 | 776 |
| 352 | 502 | 514 | 525 | 768 | 769 | 775 |
| 353 | 501 | 514 | 524 | 768 | 769 | 772 |
| 354 | 503 | 515 | 526 | 768 | 769 | 772 |
| 355 | 504 | 516 | 527 | 768 | 769 | 772 |
| 356 | 505 | 517 | 528 | 768 | 769 | 775 |
| 357 | 506 | 518 | 529 | 768 | 769 | 774 |
| 358 | 506 | 518 | 530 | 768 | 769 | 772 |
| 359 | 503 | 519 | 531 | 768 | 769 | 777 |
| 360 | 503 | 520 | 532 | 768 | 769 | 775 |
| 361 | 507 | 521 | 533 | 768 | 769 | 778 |
| 362 | 507 | 521 | 533 | 768 | 769 | 772 |
| 363 | 501 | 511 | 524 | 770 | 771 | 779 |

In some embodiments, a multispecific molecule described herein comprises at least one VL sequence, wherein the multispecific molecule is capable of binding an amino acid sequence that is at least 60%, at least 70%, at least 75%, at least 80%, at least 85%, at least 90%, at least 95%, at least 99% or 100% identical to any one of amino acid sequences of TABLE 29. In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 34, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding moiety, wherein (a) the TREM1 binding moiety comprises an amino acid sequence that is at least 60%, at least 65%, at least 70%, at least 75%, at least 80%, at least 85%, at least 90%, at least 95% or 100% identical to any one of VL sequences described in TABLE 34, and (b) the TREM1 binding moiety can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding region and an interleukin binding region, wherein (a) the TREM binding region comprises an amino acid sequence that is at least 60%, at least 65%, at least 70%, at least 75%, at least 80%, at least 85%, at least 90%, at least 95% or 100% identical to any one of VL sequences described in TABLE 34, (b) the TREM1 binding region can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding region can bind a protein selected from any one of IL-1 family, IL-6 family, IL-12 family and IL-23 family, a variant thereof, a functional fragment thereof, or a combination thereof. In some embodiments, a multispecific molecule described herein comprises a TREM 1 binding domain and an interleukin binding domain, wherein (a) the TREM1 binding domain comprises an amino acid sequence that is at least 60%, at least 65%, at least 70%, at least 75%, at least 80%, at least 85%, at least 90%, at least 95% or 100% identical to any one of VL sequences described in TABLE 34, (b) the TREM1 binding domain can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding domain can bind a protein selected from any one of IL-1 family, IL-6 family, IL-12 family and IL-23 family, a variant thereof, a functional fragment thereof, or a combination thereof.

TABLE 34
Exemplary VL sequence for binding to TREM1

| SEQ | |
|---|---|
| ID | |
| NO: | VL Sequences |
| 394 | DIQMTQSPSSLSASVGDRVTITCSASSSVSYMHWYQQKPGKAPKLLIYTTSNLASGVPS |
| | RFSGSGSGTDFTLTISSLQPEDFATYYCHQWSGYPTFGQGTKLEIK |
| 395 | DIQLTQSPSSLSASVGDRVTITCSASSSVSYMHWYQQKPGKAPKLLLYTTSNLASGVPS |
| | RFSGSGSGTDYTLTISSLQPEDFATYYCHQWSGYPTFGQGTKLEIK |
| 396 | DIQLTQSPSSLSASVGDRITLTCSASSSVSYMHWYQQKPGKAPKLLLYTTSNLASGVPS |
| | RFSGSGSGTDYTLTISSVQPEDFATYYCHQWSGYPTFGQGTKLEIK |
| 397 | DIQLTQSPSSLSASVGDRITLTCSASSSVSYMHWYQQKPGKAPKLLLYTTSNLASGVPS |
| | RFSGSGSGTDYTLTISSVQPEDAATYYCHQWSGYPTFGQGTKLEIK |
| 473 | DIVLTQSPDSLAVSLGERATINCRASESVDTFDYSFLHWYQQKPGQPPKLLIYRASNLE |
| | SGVPDRFSGSGSGTDFTLTISSLQAEDVAVYYCQQSNEDPYTFGQGTKLEIK |
| 474 | DIQLTQSPSFLSASVGDRVTITCQASQNIGSDLAWYQQKPGKAPKLLIYKAATLASGVP |
| | SRFSGSGSGTEFTLTISSLQPEDFATYYCQQYYYGSAGADTDTFGGGTKVEIK |
| 896 | DIVLTQSPDSLAVSLGERATINCRASQSVDTFDYSFLHWYQQKPGQPPKLLIYRASNLESGVP |
| | DRFSGSGSGTDFTLTISSLQAEDVAVYYCQQSNQDPYTFGQGTKLEIK |
| 897 | DIQMTQSPSSLSASVGDRVTITCQASQDISNYLNWYQQKPGKAPKLLIYDASNLETGVPSRFS |
| | GSGSGTDFTFTISSLQPEDIATYYCQQVYVLPFTFGGGTKVEIK |
| 898 | EIVLTQSPGTLSLSPGERATLSCRASQSVSSSYLAWYQQKPGQAPRLLIYGASSRATGIPDRF |
| | SGSGSGTDFTLTISRLEPEDFAVYYCQQYLGFPPTFGGGTKVEIK |
| 899 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQSFLTPWTFGGGTKVEIK |
| 900 | EIVMTQSPATLSVSPGERATLSCRASQSVSSNLAWYQQKPGQAPRLLIYGASTRATGIPARFS |
| | GSGSGTEFTLTISSLQSEDFAVYYCQQFNNHPITFGGGTKVEIK |
| 901 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCVQARQTPLTFGGGTKVEIK |
| 902 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARDAPWTFGGGTKVEIK |
| 903 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQLASYPYTFGGGTKVEIK |
| 904 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARQTPFTFGGGTKVEIK |
| 905 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARQAPWTFGGGTKVEIK |
| 906 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARQVPPWTFGGGTKVEIK |
| 907 | DIVMTQSPLSLPVTPGEPASISCRSSQSLLHSNGYNYLDWYLQKPGQSPQLLIFLGSNRASGV |
| | PDRFSGSGSGTDFTLKISRVEAEDVGVYYCMQARQAFTFGGGTKVEIK |
| 908 | EIVLTQSPATLSLSPGERATLSCRASQSVSSYLAWYQQKPGQAPRLLIYDASNRATGIPARFS |
| | GSGSGTDFTLTISSLEPEDFAVYYCQQYTSWPLTFGGGTKVEIK |
| 909 | EIVLTQSPGTLSLSPGERATLSCRASQSVSSSFLAWYQQKPGQAPRLLIYGASSRATGIPDRF |
| | SGSGSGTDFTLTISRLEPEDFAVYYCQQLDSHPPTFGGGTKVEIK |
| 910 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQYDVDPLTFGGGTKVEIK |
| 911 | EIVLTQSPGTLSLSPGERATLSCRASQSVSSSYLAWYQQKPGQAPRLLIYGASSRATGIPDRF |
| | SGSGSGTDFTLTISRLEPEDFAVYYCQQAFISPPTFGGGTKVEIK |
| 912 | DIQMTQSPSSVSASVGDRVTITCRASQGISSWLAWYQQKPGKAPKLLIYAASSLQSGVPSRFS |
| | GSGSGTDFTLTISSLQPEDFATYYCQQADTLPITFGGGTKVEIK |
| 913 | DIQMTQSPSSLSASVGDRVTITCQASQDISNYLNWYQQKPGKAPKLLIYDASNLATGVPSRFS |
| | GSGSGTDFTFTISSLQPEDIATYYCQQSDIHPRTFGGGTKVEIK |
| 914 | DIQMTQSPSTLSASVGDRVTITCRASQSISSWLAWYQQKPGKAPKLLIYKASSLESGVPSRFS |
| | GSGSGTEFTLTISSLQPDDFATYYCQQDSIYPITFGGGTKVEIK |
| 915 | DIQMTQSPSSVSASVGDRVTITCRASQGISSWLAWYQQKPGKAPKLLIYAASNLQSGVPSRFS |
| | GSGSGTDFTLTISSLQPEDFATYYCQQANSFPLTFGGGTKVEIK |
| 916 | DIQMTQSPSTLSASVGDRVTITCRASQSISSWLAWYQQKPGKAPKLLIYKASSLESGVPSRFS |
| | GSGSGTEFTLTISSLQPDDFATYYCQQYKSFSPFTFGGGTKVEIK |
| 917 | DIQLTQSPSSLSASVGDRVTITCRASQSISSFLNWYQQKPGKAPKLLIYAASSLQSGVPSRFS |
| | GSGSGTDFTLTISSLQPEDFATYYCQQSYSDLTFGGGTKVEIK |
| 918 | EIVLTQSPGTLSLSPGERATLSCRASQSVSSSYLAWYQQKPGQAPRLLIYGASSRATGIPDRF |
| | SGSGSGTDFTLTISRLEPEDFAVYYCQQYLIPPITFGGGTKVEIK |
| 919 | DIQMTQSPSTLSASVGDRVTITCRASQSIGSWLAWYQQKPGKAPKLLIYKASSLESGVPSRFS |
| | GSGSGTEFTLTISSLQPDDFATYYCQQHQSFSPTFGGGTKVEIK |
| 920 | EIVLTQSPATLSLSPGERATLSCRASQSVSSYLAWYQQKPGQAPRLLIYDASNRATGIPARFS |
| | GSGSGTDFTLTISSLEPEDFAVYYCQQRSVLPLTFGGGTKVEIK |
| 921 | DIQMTQSPSSLSASVGDRVTITCRASQSISSYLNWYQQKPGKAPKLLIYAASSLQSGVPSRFS |
| | GSGSGTDFTLTISSLQPEDFATYYCQQIFSTPLTFGGGTKVEIK |
| 922 | DIQMTQSPSSLSASVGDRVTITCRASQSISSYLNWYQQKPGKAPKLLIYAASSLQSGVPSRFS |
| | GSGSGTDFTLTISSLQPEDFATYYCQQSFYDPITFGGGTKVEIK |
| 923 | EIVLTQSPATLSVSPGERATLSCRASQSVGSNLAWYQQKPGQAPRLLIYGASTRATGIPARFS |
| | GSGSGTEFTLTISSLQSEDFAVYYCQQYLYFPLTFGGGTKVEIK |
| 924 | EIVLTQSPATLSLSPGERATLSCRASQSVSSYLAWYQQKPGQAPRLLIYDASNRATGIPARFS |
| | GSGSGTDFTLTISSLEPEDFAVYYCQQGVNYPFTFGGGTKVEIK |
| 925 | DIQMTQSPSSVSASVGDRVTITCRASQGISSWLAWYQQKPGKAPKLLIYAASSLQSGVPSRFS |
| | GSGSGTDFTLTISSLQPEDFATYYCQQVISFPTFGGGTKVEIK |
| 926 | DIQMTQSPSSLSASVGDRVTITCQASQDISNYLNWYQQKPGKAPKLLIYDASNLETGVPSRFS |
| | GSGSGTDFTFTISSLQPEDIATYYCQQYDDFPPITFGGGTKVEIK |
| 927 | DIQMTQSPSSLSASVGDRVTITCRASQSISRYLNWYQQKPGKAPKLLIYAASSLQSGVPSRFS |
| | GSGSGTDFTLTISSLQPEDFATYYCQQSLDLPFTFGGGTKVEIK |
| 928 | DIQMTQSPSSVSASVGDRVTITCRASQGISSWLAWYQQKPGKAPKLLIYAASSLQSGVPSRFS |
| | GSGSGTDFTLTISSLQPEDFATYYCQQINDHPFTFGGGTKVEIK |
| 929 | DIQMTQSPSTLSASVGDRVTITCRASQSINSWLAWYQQKPGKAPKLLISDASSLESGVPSRFS |
| | GSGSGTEFTLTISSLQPDDFATYYCQQYGPYPYTFGGGTKVEIK |
| 930 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQSHSTPLTFGGGTKVEIK |
| 931 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQLASQPPTFGGGTKVEIK |
| 932 | EIVMTQSPATLSVSPGERATLSCRASQSVSSNLAWYQQKPGQAPRLLIYGASTRATGIPARFS |
| | GSGSGTEFTLTISSLQSEDFAVYYCQQYAYWPLTFGGGTKVEIK |
| 933 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQDFSLPYTFGGGTKVEIK |
| 934 | DIQLTQSPSSVSASVGDRVTITCRASQDISSWLAWYQQKPGKAPKLLIYAASSLQSGVPSRFS |
| | GSGSGTDFTLTISSLQPEDFATYYCQQSLTHPTFGGGTKVEIK |
| 935 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQYDLLPYTFGGGTKVEIK |
| 936 | DIQLTQSPSSVSASVGDRVTITCRASQDISSWLAWYQQKPGKAPKLLIYAASSLQSGVPSRFS |
| | GSGSGTDFTLTISSLQPEDFATYYCQQAVIHPPYTFGGGTKVEIK |
| 937 | EIVMTQSPATLSVSPGERATLSCRASQSVSSNLAWYQQKPGQAPRLLIYGASTRATGIPARFS |
| | GSGSGTEFTLTISSLQSEDFAVYYCQQYNVHPPRTFGGGTKVEIK |
| 938 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQSRNAPWTFGGGTKVEIK |
| 939 | DIVMTQSPLSLPVTPGEPASISCRSSQSLLHSNGYNYLDWYLQKPGQSPQVLIYLGSNRASGV |
| | PDRFSGSGSGTDFTLKISRVEAEDVGVYYCMQARHGFTFGGGTKVEIK |
| 940 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQAREVPFTFGGGTKVEIK |
| 941 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARHVPPLTFGGGTKVEIK |
| 942 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQHDSAPYTFGGGTKVEIK |
| 943 | DIVMTQSPLSLPVTPGEPASISCRSSQSLLHSNGYNYLDWYLQKPGQSPQLLIYLGSHRASGV |
| | PDRFSGSGSGTDFTLKISRVEAEDVGVYYCMQGRQVPFTFGGGTKVEIK |
| 944 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARGTPWTFGGGTKVEIK |
| 945 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQSRRAPPWTFGGGTKVEIK |
| 946 | DIQMTQSPSTLSASVGDRVTITCRASQSISSWLAWYQQKPGKAPKLLIYKASSLESGVPSRFS |
| | GSGSGTEFTLTISSLQPDDFATYYCQQFQSYPFTFGGGTKVEIK |
| 947 | DIQMTQSPSTLSASVGDRVTITCRASQSISSWLAWYQQKPGKAPKLLIYKASSLESGVPSRFS |
| | GSGSGTEFTLTISSLQPDDFATYYCQQSSADSPFTFGGGTKVEIK |
| 948 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQARQLPWTFGGGTKVEIK |
| 949 | EIVMTQSPATLSLSPGERATLSCRASQSVSSYLAWYQQKPGQAPRLLIYDSSNRATGIPARFS |
| | GSGSGTDFTLTISSLEPEDFAVYYCQQHDVWPITFGGGTKVEIK |
| 950 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQTRHTPTFGGGTKVEIK |
| 951 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQDFARPPTFGGGTKVEIK |
| 952 | DIQMTQSPSSVSASVGDRVTITCRASQGIDSWLAWYQQKPGKAPKLLIYAASSLQSGVPSRFS |
| | GSGSGTDFTLTISSLQPEDFATYYCQQRAVFPPTFGGGTKVEIK |
| 953 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQDATGITFGGGTKVEIK |
| 954 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQLASFPWTFGGGTKVEIK |
| 955 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQLAFTPWTFGGGTKVEIK |
| 956 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQDHSFITFGGGTKVEIK |
| 957 | EIVLTQSPGTLSLSPGERATLSCRASQSVSSSYLAWYQQKPGQAPRLLIYGASSRATGIPDRF |
| | SGSGSGTDFTLTISRLEPEDFAVYYCQQDVSDFTFGGGTKVEIK |
| 958 | DIQMTQSPSSLSASVGDRVTITCRASQSISRYLNWYQQKPGKAPKLLIYAASSLQSGVPSRFS |
| | GSGSGTDFTLTISSLQPEDFATYYCQQLYHAPPITFGGGTKVEIK |
| 959 | DIVMTQSPDSLAVSLGERATINCKSSQSVLFSSNNKNYLAWYQQKPGQPPKLLIYWASTRESG |
| | VPDRFSGSGSGTDFTLTISSLEPEDVAVYYCQQYDSLPFTFGGGTKVEIK |
| 960 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQVYLFPWTFGGGTKVEIK |
| 961 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQFFLAPPTFGGGTKVEIK |
| 962 | EIVLTQSPGTLSLSPGERATLSCRASQSVSSSFLAWYQQKPGQAPRLLIYGASSRATGIPDRF |
| | SGSGSGTDFTLTISRLEPEDFAVYYCQQAVSLPWTFGGGTKVEIK |
| 963 | EIVLTQSPATLSLSPGERATLSCRASQSVSSYLAWYQQKPGQAPRLLIYDASNRATGIPARFS |
| | GSGSGTDFTLTISSLEPEDFAVYYCQQFDNLPYTFGGGTKVEIK |
| 964 | DIQMTQSPSSVSASVGDRVTITCRASQGISSWLAWYQQKPGKAPKLLIYAASNLQSGVPSRFS |
| | GSGSGTDFTLTISSLQPEDFATYYCQQATAHPPTFGGGTKVEIK |
| 965 | DIQLTQSPSSVSASVGDRVTITCRASQDISSWLAWYQQKPGKAPKLLIYAASSLQSGVPSRFS |
| | GSGSGTDFTLTISSLQPEDFATYYCQQAVSHPLTFGGGTKVEIK |
| 966 | DIQMTQSPSSVSASVGDRVTITCRASQGIDSWLAWYQQKPGKAPKLLIYAASSLQSGVPSRFS |
| | GSGSGTDFTLTISSLQPEDFATYYCQQATSLPLTFGGGTKVEIK |
| 967 | DIVMTQSPLSLPVTPGEPASISCRSSQSLLHRNGYNYLDWYLQKPGQSPQLLIYLGSNRASGV |
| | PDRFSGSGSGTDFTLKISRVEAEDVGVYYCMQRLQAWTFGGGTKVEIK |
| 968 | DIQMTQSPSTLSASVGDRVTITCRASQSISSWLAWYQQKPGKAPKLLIYKASSLESGVPSRFS |
| | GSGSGTEFTLTISSLQPDDFATYYCQQYRTYPTFGGGTKVEIK |
| 969 | DIQMTQSPSTLSASVGDRVTITCRASQSISSWLAWYQQKPGKAPKLLIYKASSLESGVPSRFS |
| | GSGSGTEFTLTISSLQPDDFATYYCQQHSLLSITFGGGTKVEIK |
| 970 | EIVLTQSPATLSLSPGERATLSCRASQSVSSYLAWYQQKPGQAPRLLIYDASNRATGIPARFS |
| | GSGSGTDFTLTISSLEPEDFAVYYCQHYNLWRTFGGGTKVEIK |
| 971 | DIQMTQSPSTLSASVGDRVTITCRASQSISSWLAWYQQKPGKAPKLLIYDASSLESGVPSRFS |
| | GSGSGTEFTLTISSLQPDDFATYYCQQHSTYSWTFGGGTKVEIK |
| 972 | EIVMTQSPATLSVSPGERATLSCRASQSVGSNLAWYQQKPGQAPRLLIYGASTRATGIPARFS |
| | GSGSGTEFTLTISSLQSEDFAVYYCQQHDVWPYTFGGGTKVEIK |
| 973 | DIVMTQSPDSLAVSLGERATINCKSSQSVLFSSNNKNYLAWYQQKPGQPPKLLIYWASTRESG |
| | VPDRFSGSGSGTDFTLTISSLQAEDVAVYYCQQYFSTPPTFGGGTKVEIK |
| 974 | DIVMTQSPDSLAVSLGERATINCKSSQSVLYSSNNKNYLAWYQQKPGQPPKLLIYWASTRESG |
| | VPDRFSGSGSGTDFTLTISSLQAEDVAVYYCQQYALTPYTFGGGTKVEIK |
| 975 | EIVMTQSPATLSVSPGERATLSCRASQSVGSNLAWYQQKPGQAPRLLIYGASTRATGIPARFS |
| | GSGSGTEFTLTISSLQSEDFAVYYCQQDHDRPLTFGGGTKVEIK |
| 976 | EIVLTQSPGTLSLSPGERATLSCRASQSVSSSYLAWYQQKPGQAPRLLIYGASSRATGIPDRF |
| | SGSGSGTDFTLTISRLEPEDFAVYYCQQYGSSPTFGGGTKVEIK |
| 977 | AIQLTQSPSSLSASVGDRVTITCRASQGISSALAWYQQKPGKAPKLLIYDASSLESGVPSRFS |
| | GSGSGTDFTLTISSLQPEDFATYYCQQFNSYPYTFGQGTKLEIK |
| 978 | EIVLTQSPGTLSLSPGERATLSCRASQSVSSSYLAWYQQKPGQAPRLLIYGASSRATGIPERF |
| | SGSGSGTDFTLTISRLEPEDFAVYYCQQYGSSPLTFGGGTKVEIK |
| 979 | AIQLTQSPSSLSASVGDRVTITCRASQGISSALAWYQQKPGKAPKLLIYAASSLQSGVPSRFS |
| | GSGSGTDFTLTISSLQPEDFATYYCQQYNSYPLTFGGGTKVEIK |
| 980 | AIQLTQSPSSLSASVGDRVTITCRASQGISSALAWYQQKPGKAPKLLIYDASSLESGVPSRFS |
| | GSGSGTDFTLTISSLQPEDFATYYCQQFNSYPITFGQGTRLEIK |
| 981 | AIQLTQSPSSLSASVGDRVTITCRASQGISSALAWYQQKPGKAPKLLIYDASSLESGVPSRFS |
| | GSGSGTDFTLTISSLQPEDFATYYCQQYNSYPITFGQGTRLEIK |
| 982 | DIQMTQSPTSLSASVGDRVTITCRASQGISSWLAWYQQKPEKAPKSLIYAASSLQSGVPSRFS |
| | GSGSGTDFTLTISSLQPEDFATYYCQQYNSYPYTFGQGTKLEIK |
| 983 | DIQMTQSPSSVSASVGDRVTITCRASQGISSWLAWYQHKPGKAPKLLIYAASSLQSGVPSRFS |
| | GSGSGTDFTLTISSLQPEDFATYYCQQANSFPFTFGGGTKVEIK |
| 984 | DIQMTQSPTSLSASVGDRVTITCRASQGISSWLAWYQHKPGKAPKLLIYAASSLQSGVPSRFS |
| | GSGSGTDFTLTISSLQPEDFATYYCQQYNSYPWTFGQGTKVEIK |
| 985 | DIQMTQSPTSLSASVGDRVTITCRASQGISSWLAWYQQKPEKAPKSLIYAASSLQSGVPSRFS |
| | GSGSGTDFTLTISSLQPEDFATYYCQQYNSYPLTFGGGTKVEIK |
| 986 | DIQMTQSPTSLSASVGDRVTITCRASQGISSWLAWYQHKPGKAPKLLIYAASSLQSGVPSRFS |
| | GSGSGTDFTLTISSLQPEDFATYYCQQYNSYPYTFGQGTKLEIK |
| 987 | DIQMTQSPTSLSASVGDRVTITCRASQGISSWLAWYQHKPGKAPKSLIYAASSLQSGVPSRFS |
| | GSGSGTDFTLTISSLQPEDFATYYCQQYNSYPYTFGQGTKLEIK |
| 988 | DIQMTQSPTSLSASVGDRVTITCRASQGISSWLAWYQQKPGKAPKSLIYAASSLQSGVPSRFS |
| | GSGSGTDFTLTISSLQPEDFATYYCQQYNSYPLTFGGGTKVEIK |
| 989 | DIQMTQSPTSLSASVGDRVTITCRASQGISSWLAWYQQKPEKAPKSLIYAASSLQSGVPSRFS |
| | GGGSGTDFTLTISSLQPEDFATYYCQQYNSYPLTFGGGTKVEIK |
| 990 | DIQMTQSPTSLSASVGDRVTITCRASQGISSWLAWYQQKPEKAPKSLIYAASSLQSGVPSRFS |
| | GSGSGTDFTLTISSLQPDDFATYYCQQYNSYPYTFGQGTKLEIK |
| 991 | DIQMTQSPTSLSASVGDRVTITCRASQGISSWLAWYQQKPEKAPKSLIYAASSLQSGVPSRFS |
| | GSGSGTDFTLTISSLQPEDFATYYCQQYNSYPITFGQGTRLEIK |
| 992 | DIQMTQSPTSLSASVGDRVTITCRASQGISSWLAWYQHKPGKAPKSLIYAASSLQSGVPSRFS |
| | GSGSGTDFTLTISSLQPEDFATYYCQQYNSYPLTFGGGTKVEIK |
| 993 | DIQMTQSPTSLSASVGDRVTITCRASQGISSWLAWYQQKPEKAPKSLIHAASSLQSGVPSRFS |
| | GSGSGTDFTLTISSLQPEDFATYYCQQYNSYPLTFGGGTKVEIK |
| 994 | DIQMTQSPTSLSASVGDRVTITCRASQGISSWLAWYQQKPEKAPKSLIYAASSLQSGVPSRFS |
| | GSGSGTDYTLTISSLQPEDFATYYCQQYNSYPLTFGGGTKVEIK |
| 995 | DIQMTQSPTSLSASVGDRVTITCRASQGISSWLAWYQQKPEKAPKSLIYAASSLQSGVPSRFS |
| | GSGSGTDFTLTISSLQPEDFATYYCQQYNSYPWTFGQGTKVEIK |
| 996 | DIQMTQSPTSLSASVGDRVTITCRASQGISSWLAWYQQKPEKAPKSLIYAASSLQSGVPSRFS |
| | GSGSGTDFTLTISSLQPEDFATYYCQQFNSYPLTFGGGTKVEIK |
| 997 | DIQMTQSPTSLSASVGDRVTITCRASQGISSWLAWYQQKPEKAPKSLIYAASSLQSGVPSRFS |
| | GSGSGTDFTLTISSLQPEDFATYYCQQYNSYPPTFGQGTKLEIK |
| 998 | EIVLTQSPGTLSLSPGERATLSCRASQSVSSYLAWYQQKPGQAPRLLIYGASSRATGIPDRFS |
| | GSGSGTDFTLTISRLEPEDFAVYYCQQYGSSPLTFGGGTKVEIK |

In some embodiments, a multispecific molecule described herein comprises at least one light chain (LC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, a multispecific molecule described herein comprises a TREM1 binding region, wherein the TREM1 binding region comprises a LC region. In some embodiments, a LC region of a TREM1 binding region comprises a VL sequence that is at least 60%, at least 65%, at least 70%, at least 75%, at least 80%, at least 85%, at least 90%, at least 95% or 100% identical to any one of amino acid sequences described in TABLE 34.

In some embodiments, a LC region of a TREM1 binding region comprises a VL sequence, and an amino acid sequence that is at least 60%, at least 65%, at least 70%, at least 75%, at least 80%, at least 85%, at least 90%, at least 95% or 100% identical to RTVAAPSVFIFPPSDEQLKSGTASVVCLLINNFYPREAKVQWKVDNALQSGNSQESVTEQDSKDS TYSLSSTLTLSKADYEKHKVYACEVTHQGLSSPVTKSFNRGEC (SEQ ID NO: 483), wherein C-terminus of the VL sequence is linked to N-terminus of the amino acid sequence.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 31; and (b) a VL sequence that is at least 60%, at least 65%, at least 70%, at least 75%, at least 80%, at least 85%, at least 90%, at least 95% or 100% identical to any one of amino acid sequences described in TABLE 34, wherein the multispecific molecule comprises the VH sequence and the VL sequence according to the combination described in TABLE 35.

TABLE 35
Exemplary Combinations of VH sequences
and VL sequences for binding to TREM1

| | VH Amino | VL Amino | | VH Amino | VL Amino |
|---|---|---|---|---|---|
| Comb. | Acid | Acid | Comb. | Acid | Acid |
| NO: | Sequence | Sequence | NO: | Sequence | Sequence |
| 239 | 380 | 394 | 257 | 385 | 396 |
| 240 | 380 | 395 | 258 | 385 | 397 |
| 241 | 380 | 396 | 259 | 386 | 394 |
| 242 | 380 | 397 | 260 | 386 | 395 |
| 243 | 381 | 394 | 261 | 386 | 396 |
| 244 | 381 | 395 | 262 | 386 | 397 |
| 245 | 381 | 396 | 263 | 388 | 394 |
| 246 | 381 | 397 | 264 | 388 | 395 |
| 247 | 382 | 394 | 265 | 388 | 396 |
| 248 | 382 | 395 | 266 | 388 | 397 |
| 249 | 382 | 396 | 267 | 389 | 394 |
| 250 | 382 | 397 | 268 | 389 | 395 |
| 251 | 384 | 394 | 269 | 389 | 396 |
| 252 | 384 | 395 | 270 | 389 | 397 |
| 253 | 384 | 396 | 271 | 390 | 394 |
| 254 | 384 | 397 | 272 | 390 | 395 |
| 255 | 385 | 394 | 273 | 390 | 396 |
| 256 | 385 | 395 | 274 | 390 | 397 |
| 364 | 465 | 473 | 383 | 466 | 474 |
| 365 | 465 | 896 | 384 | 755 | 982 |
| 366 | 743 | 976 | 385 | 755 | 990 |
| 367 | 744 | 977 | 386 | 755 | 991 |
| 368 | 744 | 978 | 387 | 755 | 992 |
| 369 | 745 | 979 | 388 | 755 | 993 |
| 370 | 746 | 977 | 389 | 755 | 985 |
| 371 | 747 | 980 | 390 | 756 | 982 |
| 372 | 747 | 981 | 391 | 756 | 987 |
| 373 | 748 | 982 | 392 | 757 | 982 |
| 374 | 749 | 983 | 393 | 758 | 982 |
| 375 | 750 | 984 | 394 | 759 | 994 |
| 376 | 751 | 985 | 395 | 760 | 995 |
| 377 | 751 | 986 | 396 | 761 | 982 |
| 378 | 751 | 982 | 397 | 762 | 996 |
| 379 | 752 | 986 | 398 | 763 | 985 |
| 380 | 751 | 987 | 399 | 999 | 997 |
| 381 | 753 | 988 | 400 | 999 | 982 |
| 382 | 754 | 989 | 401 | 751 | 998 |

In some embodiments, a multispecific molecule described herein comprises at least one heavy chain (HC) region, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, a multispecific molecule described herein comprises an amino acid sequence that is at least 60%, at least 65%, at least 70%, at least 75%, at least 80%, at least 85%, at least 90%, at least 95% or 100% identical to any one of amino acid sequences of HC region described in TABLE 36, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding moiety, wherein (a) the TREM1 binding moiety comprises an amino acid sequence that is at least 60%, at least 65%, at least 70%, at least 75%, at least 80%, at least 85%, at least 90%, at least 95% or 100% identical to any one of amino acid sequences of HC region described in TABLE 36, (b) the TREM1 binding moiety can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding region and an interleukin binding region, wherein (a) the TREM1 binding region comprises an amino acid sequence that is at least 60%, at least 65%, at least 70%, at least 75%, at least 80%, at least 85%, at least 90%, at least 95% or 100% identical to any one of amino acid sequences of HC region described in TABLE 36, (b) the TREM1 binding region can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding region can bind a protein selected from any one of IL-1 family, IL-6 family, IL-12 family and IL-23 family, a variant thereof, a functional fragment thereof, or a combination thereof. In some embodiments, a multispecific molecule described herein comprises a TREM1 binding domain and an interleukin binding domain, wherein (a) the TREM1 binding domain comprises an amino acid sequence that is at least 60%, at least 65%, at least 70%, at least 75%, at least 80%, at least 85%, at least 90%, at least 95% or 100% identical to any one of amino acid sequences of HC region described in TABLE 36, (b) the TREM1 binding domain can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding domain can bind a protein selected from any one of IL-1 family, IL-6 family, IL-12 family and IL-23 family, a variant thereof, a functional fragment thereof, or a combination thereof.

TABLE 36
Exemplary HC region of TREM1 binding domain

| SEQ | |
|---|---|
| ID | |
| NO: | HC Amino Acid Sequences |
| 475 | EVQLVESGGGLVQPGGSLKLSCAASGFTFSTYAMHWVRQASGKGLEWVGRIRTKSSNYATYYA |
| | ASVKGRFTISRDDSKNTAYLQMNSLKTEDTAVYYCTRDMGIRRQFAYWGQGTLVTVSSASTKG |
| | PSVFPLAPSSKSTSGGTAALGCLVKDYFPEPVTVSWNSGALTSGVHTFPAVLQSSGLYSLSSV |
| | VTVPSSSLGTQTYICNVNHKPSNTKVDKRVEPKSCDKTHTCPPCPAPEAAGGPSVFLFPPKPK |
| | DTLMISRTPEVTCVVVDVSHEDPEVKFNWYVDGVEVHNAKTKPREEQYNSTYRVVSVLTVLHQ |
| | DWLNGKEYKCKVSNKALAAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSCSVMHEALHNHYT |
| | QKSLSLSPG |
| 476 | EVQLVESGGGLVQPGGSLRLSCAASGFSLSSYAMTWVRQAPGKGLEWIGIIYAGGSPSYASWA |
| | KGRFTISKDNSKNTLYLQMNSLRAEDTAVYYCARGTGDTVYTYFNIWGQGTLVTVSSASTKGP |
| | SVFPLAPSSKSTSGGTAALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPEAAGGPSVFLFPPKPKD |
| | TLMISRTPEVTCVVVDVSHEDPEVKFNWYVDGVEVHNAKTKPREEQYNSTYRVVSVLTVLHQD |
| | WLNGKEYKCKVSNKALAAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVMHEALHNHYTQ |
| | KSLSLSPG |
| 477 | EVQLVESGGGLVQPGGSLKLSCAASGFTFSTYAMHWVRQASGKGLEWVGRIRTKSSNYATYYA |
| | ASVKGRFTISRDDSKNTAYLQMNSLKTEDTAVYYCTRDMGIRRQFAYWGQGTLVTVSSASTKG |
| | PSVFPLAPSSKSTSGGTAALGCLVKDYFPEPVTVSWNSGALTSGVHTFPAVLQSSGLYSLSSV |
| | VTVPSSSLGTQTYICNVNHKPSNTKVDKRVEPKSCDKTHTCPPCPAPEAAGGPSVFLFPPKPK |
| | DTLMISRTPEVTCVVVDVSHEDPEVKFNWYVDGVEVHNAKTKPREEQYNSTYRVVSVLTVLHQ |
| | DWLNGKEYKCKVSNKALAAPIEKTISKAKGQPREPQVCTLPPSREEMTKNQVSLSCAVKGFYP |
| | SDIAVEWESNGQPENNYKTTPPVLDSDGSFFLVSKLTVDKSRWQQGNVFSCSVMHEALHNHYT |
| | QKSLSLSPG |
| 478 | EVQLVESGGGLVQPGGSLRLSCAASGFSLSSYAMTWVRQAPGKGLEWIGIIYAGGSPSYASWA |
| | KGRFTISKDNSKNTLYLQMNSLRAEDTAVYYCARGTGDTVYTYFNIWGQGTLVTVSSASTKGP |
| | SVFPLAPSSKSTSGGTAALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPEAAGGPSVFLFPPKPKD |
| | TLMISRTPEVTCVVVDVSHEDPEVKFNWYVDGVEVHNAKTKPREEQYNSTYRVVSVLTVLHQD |
| | WLNGKEYKCKVSNKALAAPIEKTISKAKGQPREPQVCTLPPSREEMTKNQVSLSCAVKGFYPS |
| | DIAVEWESNGQPENNYKTTPPVLDSDGSFFLVSKLTVDKSRWQQGNVFSCSVMHEALHNHYTQ |
| | KSLSLSPG |

In some embodiments, a multispecific molecule described herein comprises at least one light chain (LC) sequence, wherein the multispecific molecule can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, a multispecific molecule described herein comprises an amino acid sequence that is at least 60%, at least 65%, at least 70%, at least 75%, at least 80%, at least 85%, at least 90%, at least 95% or 100% identical to any one of amino acid sequence of LC region described in TABLE 37, wherein the multispecific molecule is capable of binding an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding moiety, wherein (a) the TREM1 binding moiety comprises an amino acid sequence that is at least 60%, at least 65%, at least 70%, at least 75%, at least 80%, at least 85%, at least 90%, at least 95% or 100% identical to any one of amino acid sequences of LC region described in TABLE 37, and (b) the TREM1 binding moiety can bind an amino acid sequence that is at least 60%, at least 70%, at least 75%, at least 80%, at least 85%, at least 90%, at least 95%, at least 99% or 100% identical to any one of SEQ ID NOs: 398 and 456-458. In some embodiments, an engineered protein construct described herein comprises a TREM1 binding region and an interleukin binding region, wherein (a) the TREM1 binding region comprises an amino acid sequence that is at least 60%, at least 65%, at least 70%, at least 75%, at least 80%, at least 85%, at least 90%, at least 95% or 100% identical to any one of amino acid sequences of LC region described in TABLE 37, (b) the TREM1 binding region can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding region can bind a protein selected from any one of IL-1 family, IL-6 family, IL-12 family and IL-23 family, a variant thereof, a functional fragment thereof, or a combination thereof. In some embodiments, a multispecific molecule described herein comprises a TREM1 binding domain and an interleukin binding domain, wherein (a) the TREM1 binding domain comprises an amino acid sequence that is at least 60%, at least 65%, at least 70%, at least 75%, at least 80%, at least 85%, at least 90%, at least 95% or 100% identical to any one of amino acid sequences of LC region described in TABLE 37, (b) the TREM1 binding domain can bind an amino acid sequence that is at least 60%, at least 70%, at least 75%, at least 80%, at least 85%, at least 90%, at least 95%, at least 99% or 100% identical to any one of SEQ ID NOs: 398 and 456-458, and (c) the interleukin binding domain can bind a protein selected from any one of IL-1 family, IL-6 family, IL-12 family and IL-23 family, a variant thereof, a functional fragment thereof, or a combination thereof.

TABLE 37
Exemplary LC region of TREM1 binding domain

| SEQ | |
|---|---|
| ID | |
| NO: | LC Amino Acid Sequences |
| 479 | DIVLTQSPDSLAVSLGERATINCRASESVDTFDYSFLHWYQQKPGQPPKLLIYRASNLESGVP |
| | DRFSGSGSGTDFTLTISSLQAEDVAVYYCQQSNEDPYTFGQGTKLEIKRTVAAPSVFIFPPSD |
| | EQLKSGTASVVCLLNNFYPREAKVQWKVDNALQSGNSQESVTEQDSKDSTYSLSSTLTLSKAD |
| | YEKHKVYACEVTHQGLSSPVTKSFNRGEC |
| 480 | DIQLTQSPSFLSASVGDRVTITCQASQNIGSDLAWYQQKPGKAPKLLIYKAATLASGVPSRFS |
| | GSGSGTEFTLTISSLQPEDFATYYCQQYYYGSAGADTDTFGGGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSFNRGEC |

In some embodiments, multispecific antibodies described herein comprise: (a) a HC region that is at least 60%, at least 65%, at least 70%, at least 75%, at least 80%, at least 85%, at least 90%, at least 95% or 100% identical to any one of amino acid sequences described in TABLE 36; and (b) a LC region that is at least 60%, at least 65%, at least 70%, at least 75%, at least 80%, at least 85%, at least 90%, at least 95% or 100% identical to any one of amino acid sequences described in TABLE 37, wherein the HC and LC regions are selected according to the combination described in TABLE 38.

TABLE 38
Exemplary Combinations of HC and
LC regions for binding to TREM1

| Comb. NO: | HC Amino Acid Sequence | LC Amino Acid Sequence |
|---|---|---|
| 402 | 475 | 479 |
| 403 | 476 | 480 |

### Nucleotide Constructs for BsAb

Provided herein are nucleotide sequences encoding a TREM1 binding domain, a variant thereof, or a functional fragment thereof. In some embodiments, a TREM1 binding domain comprises a HC region, a LC region, or a combination thereof. In some embodiments, a nucleotide sequence encodes a HC region of TREM1 binding domain, wherein the HC region comprises any one of combinations of CDR-Hs or variants thereof described in TABLE 30.1, wherein the variant comprises an amino acid sequence that is at least 60%, at least 65%, at least 70%, at least 75%, at least 80%, at least 85%, at least 90%, at least 95% or 100% identical to a corresponding parent CDR-H sequence described in TABLE 30.1. In some embodiments, a nucleotide sequence encodes a HC region of TREM1 binding domain, wherein the HC region comprises a VH sequence, a variant thereof or a functional fragment thereof, wherein the VH sequence comprises any one of sequences described in TABLE 31, and wherein the variant comprises an amino acid sequence that is at least 60%, at least 65%, at least 70%, at least 75%, at least 80%, at least 85%, at least 90%, at least 95% or 100% identical to a corresponding parent VH sequence described in TABLE 31. In some embodiments, a nucleotide sequence encodes a HC region of TREM1 binding domain, wherein the HC region comprises an amino acid sequence described in TABLE 36, wherein the variant comprises an amino acid sequence that is at least 60%, at least 65%, at least 70%, at least 75%, at least 80%, at least 85%, at least 90%, at least 95% or 100% identical to a corresponding parent HC sequence described in TABLE 36. In some embodiments, a nucleotide sequence encodes a LC region of TREM1 binding domain, wherein the LC region comprises any one of combinations of CDR-Ls or variants thereof described in TABLE 32.1, wherein the variant comprises an amino acid sequence that is at least 60%, at least 65%, at least 70%, at least 75%, at least 80%, at least 85%, at least 90%, at least 95% or 100% identical to a corresponding parent CDR-L sequence described in TABLE 32.1. In some embodiments, a nucleotide sequence encodes a LC region of TREM1 binding domain, wherein the LC region comprises a VL sequence, a variant thereof or a functional fragment thereof, wherein the VL sequence comprises any one of sequences described in TABLE 34, and wherein the variant comprises an amino acid sequence that is at least 60%, at least 65%, at least 70%, at least 75%, at least 80%, at least 85%, at least 90%, at least 95% or 100% identical to a corresponding parent VL sequence described in TABLE 34. In some embodiments, a nucleotide sequence encodes a LC region of TREM1 binding domain, wherein the LC region comprises an amino acid sequence described in TABLE 37, wherein the variant comprises an amino acid sequence that is at least 60%, at least 65%, at least 70%, at least 75%, at least 80%, at least 85%, at least 90%, at least 95% or 100% identical to a corresponding parent HC sequence described in TABLE 37.

TABLE 39 provides exemplary nucleotide sequences for some of the proteins described herein or portions thereof. In some embodiments, a nucleotide sequence encoding BsAb or a portion thereof comprises a sequence that is at least 60%, at least 65%, at least 70%, at least 75%, at least 80%, at least 85%, at least 90%, at least 95% or 100% identical to any one of nucleotide sequence described in TABLE 39.

TABLE 39
Exemplary Nucleotide Sequences encoding BsAb or a portion thereof

| Amino | | |
|---|---|---|
| Acid | Nucleotide | |
| Sequence | Sequence | |
| (SEQ ID NO: | SEQ ID NO: | Nucleotide Sequence |
| 475 | 1000 | GAAGTGCAGCTGGTTGAATCCGGTGGGGGGCTTGTACAACCAGG |
| | | GGGCTCTCTTAAACTTTCATGTGCTGCCTCCGGTTTTACGTTTA |
| | | GCACCTACGCAATGCACTGGGTTAGGCAGGCGTCCGGCAAGGGC |
| | | CTGGAGTGGGTCGGTCGGATTCGCACGAAGTCAAGCAATTACGC |
| | | GACGTATTATGCGGCATCTGTCAAGGGAAGGTTCACTATCTCTA |
| | | GAGACGATTCTAAGAACACCGCTTATCTTCAGATGAACTCTCTG |
| | | AAAACAGAAGATACAGCTGTATATTACTGTACTCGGGATATGGG |
| | | CATACGAAGGCAGTTTGCGTATTGGGGGCAAGGGACACTCGTAA |
| | | CTGTGAGCAGTGCTAGCACTAAAGGGCCTTCTGTATTTCCCTTG |
| | | GCCCCGTCCAGCAAATCGACCTCGGGAGGGACAGCCGCCCTGGG |
| | | TTGCCTTGTGAAAGATTATTTCCCTGAGCCAGTTACCGTAAGTT |
| | | GGAACAGTGGGGCGCTGACAAGTGGTGTGCACACGTTTCCTGCC |
| | | GTCCTGCAATCATCGGGCTTGTATAGCCTCAGCTCTGTGGTCAC |
| | | TGTCCCAAGTTCATCGCTGGGCACTCAGACGTATATTTGCAATG |
| | | TGAACCACAAACCTTCAAATACAAAAGTGGATAAACGCGTAGAA |
| | | CCGAAATCGTGTGATAAAACTCACACATGCCCGCCATGCCCGGC |
| | | ACCTGAAGCAGCTGGTGGTCCCAGCGTGTTCCTGTTCCCGCCGA |
| | | AGCCTAAAGATACTCTAATGATCAGCCGTACGCCAGAGGTGACA |
| | | TGTGTCGTGGTTGACGTGTCCCACGAAGATCCCGAAGTTAAGTT |
| | | CAATTGGTATGTTGATGGTGTAGAGGTACACAATGCTAAGACTA |
| | | AACCTCGCGAGGAGCAGTACAATTCGACCTATCGTGTCGTGAGC |
| | | GTTCTGACCGTCCTTCACCAAGATTGGCTTAACGGCAAAGAATA |
| | | TAAGTGCAAGGTAAGCAATAAAGCACTTGCAGCCCCAATCGAGA |
| | | AAACCATTTCCAAGGCCAAAGGTCAACCAAGAGAACCCCAGGTG |
| | | TATACTCTTCCGCCTTCTCGTGAGGAAATGACTAAAAATCAAGT |
| | | ATCCCTTACGTGTCTGGTTAAAGGTTTTTATCCTAGCGATATTG |
| | | CTGTTGAATGGGAATCGAACGGTCAGCCGGAGAATAATTATAAA |
| | | ACAACGCCACCCGTCCTGGATAGCGACGGCTCATTTTTTCTGTA |
| | | TAGCAAACTGACTGTAGATAAATCACGGTGGCAGCAGGGCAATG |
| | | TATTCAGTTGCTCCGTTATGCATGAAGCGTTACATAATCACTAC |
| | | ACGCAGAAATCTCTTAGTCTTTCACCCGGT |
| 479 | 1001 | GACATTGTGCTCACCCAAAGCCCGGACAGCCTTGCGGTCTCCCT |
| | | TGGAGAAAGAGCAACTATCAATTGTAGGGCGTCTGAAAGTGTTG |
| | | ACACCTTTGATTACTCCTTCCTGCACTGGTATCAACAGAAGCCA |
| | | GGTCAACCGCCAAAACTCCTGATCTATAGAGCTTCAAATTTGGA |
| | | GTCTGGTGTCCCCGACCGATTTAGCGGAAGCGGTAGCGGGACTG |
| | | ATTTTACGCTCACCATTTCCTCTCTGCAAGCTGAAGATGTGGCA |
| | | GTTTACTACTGTCAACAAAGTAACGAGGACCCATATACATTTGG |
| | | GCAGGGAACTAAATTGGAGATCAAACGTACGGTAGCTGCCCCTT |
| | | CAGTTTTTATCTTTCCGCCGTCTGACGAGCAGTTAAAATCCGGG |
| | | ACCGCTTCTGTAGTTTGCCTGCTGAATAATTTTTATCCGCGTGA |
| | | GGCTAAAGTACAATGGAAAGTCGACAATGCTTTGCAGTCGGGAA |
| | | ATTCACAGGAAAGTGTTACGGAGCAGGATTCTAAAGATTCCACA |
| | | TATTCACTCAGCTCCACCCTTACACTGAGCAAAGCCGACTATGA |
| | | AAAACATAAAGTTTACGCATGTGAGGTGACGCACCAAGGATTAT |
| | | CCAGTCCGGTCACAAAATCGTTTAACCGCGGTGAGTGT |
| 476 | 1002 | GAGGTGCAACTTGTAGAGTCTGGGGGGGGACTCGTCCAGCCAGG |
| | | AGGATCCCTCAGACTCAGTTGTGCAGCGTCAGGCTTCAGTCTCT |
| | | CCTCCTACGCAATGACGTGGGTTAGGCAAGCACCCGGTAAAGGT |
| | | CTGGAATGGATCGGGATTATTTACGCTGGGGGGTCCCCAAGTTA |
| | | CGCGAGCTGGGCTAAAGGTCGATTTACAATAAGTAAAGATAATT |
| | | CCAAGAACACCTTGTATCTGCAGATGAACAGCTTGAGGGCGGAG |
| | | GACACTGCAGTTTATTATTGTGCGCGGGGTACGGGCGATACAGT |
| | | CTATACTTATTTTAACATTTGGGGCCAAGGAACCTTGGTGACCG |
| | | TGTCCTCAGCTAGCACTAAAGGGCCTTCTGTATTTCCCTTGGCC |
| | | CCGTCCAGCAAATCGACCTCGGGAGGGACAGCCGCCCTGGGTTG |
| | | CCTTGTGAAAGATTATTTCCCTGAGCCAGTTACCGTAAGTTGGA |
| | | ACAGTGGGGCGCTGACAAGTGGTGTGCACACGTTTCCTGCCGTC |
| | | CTGCAATCATCGGGCTTGTATAGCCTCAGCTCTGTGGTCACTGT |
| | | CCCAAGTTCATCGCTGGGCACTCAGACGTATATTTGCAATGTGA |
| | | ACCACAAACCTTCAAATACAAAAGTGGATAAACGCGTAGAACCG |
| | | AAATCGTGTGATAAAACTCACACATGCCCGCCATGCCCGGCACC |
| | | TGAAGCAGCTGGTGGTCCCAGCGTGTTCCTGTTCCCGCCGAAGC |
| | | CTAAAGATACTCTAATGATCAGCCGTACGCCAGAGGTGACATGT |
| | | GTCGTGGTTGACGTGTCCCACGAAGATCCCGAAGTTAAGTTCAA |
| | | TTGGTATGTTGATGGTGTAGAGGTACACAATGCTAAGACTAAAC |
| | | CTCGCGAGGAGCAGTACAATTCGACCTATCGTGTCGTGAGCGTT |
| | | CTGACCGTCCTTCACCAAGATTGGCTTAACGGCAAAGAATATAA |
| | | GTGCAAGGTAAGCAATAAAGCACTTGCAGCCCCAATCGAGAAAA |
| | | CCATTTCCAAGGCCAAAGGTCAACCAAGAGAACCCCAGGTGTAT |
| | | ACTCTTCCGCCTTCTCGTGAGGAAATGACTAAAAATCAAGTATC |
| | | CCTTACGTGTCTGGTTAAAGGTTTTTATCCTAGCGATATTGCTG |
| | | TTGAATGGGAATCGAACGGTCAGCCGGAGAATAATTATAAAACA |
| | | ACGCCACCCGTCCTGGATAGCGACGGCTCATTTTTTCTGTATAG |
| | | CAAACTGACTGTAGATAAATCACGGTGGCAGCAGGGCAATGTAT |
| | | TCAGTTGCTCCGTTATGCATGAAGCGTTACATAATCACTACACG |
| | | CAGAAATCTCTTAGTCTTTCACCCGGT |
| 480 | 1003 | GACATACAACTGACTCAATCACCAAGTTTTTTGTCAGCATCAGT |
| | | CGGCGACAGAGTAACGATAACTTGTCAGGCGAGTCAGAATATCG |
| | | GTAGTGACTTGGCTTGGTATCAACAAAAACCGGGGAAGGCACCG |
| | | AAGCTCCTCATCTACAAAGCTGCTACGCTCGCATCAGGCGTCCC |
| | | CTCACGCTTTTCCGGCAGCGGAAGCGGCACAGAATTCACGCTCA |
| | | CCATCAGTAGCCTTCAGCCAGAAGACTTTGCTACTTATTACTGC |
| | | CAACAATATTACTACGGCAGCGCGGGTGCAGATACGGACACCTT |
| | | TGGAGGAGGGACCAAAGTGGAAATTAAACGTACGGTAGCTGCCC |
| | | CTTCAGTTTTTATCTTTCCGCCGTCTGACGAGCAGTTAAAATCC |
| | | GGGACCGCTTCTGTAGTTTGCCTGCTGAATAATTTTTATCCGCG |
| | | TGAGGCTAAAGTACAATGGAAAGTCGACAATGCTTTGCAGTCGG |
| | | GAAATTCACAGGAAAGTGTTACGGAGCAGGATTCTAAAGATTCC |
| | | ACATATTCACTCAGCTCCACCCTTACACTGAGCAAAGCCGACTA |
| | | TGAAAAACATAAAGTTTACGCATGTGAGGTGACGCACCAAGGAT |
| | | TATCCAGTCCGGTCACAAAATCGTTTAACCGCGGTGAGTGT |
| 477 | 1004 | GAAGTGCAGCTGGTTGAATCCGGTGGGGGGCTTGTACAACCAGG |
| | | GGGCTCTCTTAAACTTTCATGTGCTGCCTCCGGTTTTACGTTTA |
| | | GCACCTACGCAATGCACTGGGTTAGGCAGGCGTCCGGCAAGGGC |
| | | CTGGAGTGGGTCGGTCGGATTCGCACGAAGTCAAGCAATTACGC |
| | | GACGTATTATGCGGCATCTGTCAAGGGAAGGTTCACTATCTCTA |
| | | GAGACGATTCTAAGAACACCGCTTATCTTCAGATGAACTCTCTG |
| | | AAAACAGAAGATACAGCTGTATATTACTGTACTCGGGATATGGG |
| | | CATACGAAGGCAGTTTGCGTATTGGGGGCAAGGGACACTCGTAA |
| | | CTGTGAGCAGTGCTAGCACTAAAGGGCCTTCTGTATTTCCCTTG |
| | | GCCCCGTCCAGCAAATCGACCTCGGGAGGGACAGCCGCCCTGGG |
| | | TTGCCTTGTGAAAGATTATTTCCCTGAGCCAGTTACCGTAAGTT |
| | | GGAACAGTGGGGCGCTGACAAGTGGTGTGCACACGTTTCCTGCC |
| | | GTCCTGCAATCATCGGGCTTGTATAGCCTCAGCTCTGTGGTCAC |
| | | TGTCCCAAGTTCATCGCTGGGCACTCAGACGTATATTTGCAATG |
| | | TGAACCACAAACCTTCAAATACAAAAGTGGATAAACGCGTAGAA |
| | | CCGAAATCGTGTGATAAAACTCACACATGCCCGCCATGCCCGGC |
| | | ACCTGAAGCAGCTGGTGGTCCCAGCGTGTTCCTGTTCCCGCCGA |
| | | AGCCTAAAGATACTCTAATGATCAGCCGTACGCCAGAGGTGACA |
| | | TGTGTCGTGGTTGACGTGTCCCACGAAGATCCCGAAGTTAAGTT |
| | | CAATTGGTATGTTGATGGTGTAGAGGTACACAATGCTAAGACTA |
| | | AACCTCGCGAGGAGCAGTACAATTCGACCTATCGTGTCGTGAGC |
| | | GTTCTGACCGTCCTTCACCAAGATTGGCTTAACGGCAAAGAATA |
| | | TAAGTGCAAGGTAAGCAATAAAGCACTTGCAGCCCCAATCGAGA |
| | | AAACCATTTCCAAGGCCAAAGGTCAACCAAGAGAACCCCAGGTG |
| | | TGTACTCTTCCGCCTTCTCGTGAGGAAATGACTAAAAATCAAGT |
| | | ATCCCTTTCCTGTGCTGTTAAAGGTTTTTATCCTAGCGATATTG |
| | | CTGTTGAATGGGAATCGAACGGTCAGCCGGAGAATAATTATAAA |
| | | ACAACGCCACCCGTCCTGGATAGCGACGGCTCATTTTTTCTGGT |
| | | AAGCAAACTGACTGTAGATAAATCACGGTGGCAGCAGGGCAATG |
| | | TATTCAGTTGCTCCGTTATGCATGAAGCGTTACATAATCACTAC |
| | | ACGCAGAAATCTCTTAGTCTTTCACCCGGT |
| 478 | 1005 | GAGGTGCAACTTGTAGAGTCTGGGGGCGGACTCGTCCAGCCAGG |
| | | AGGATCCCTCAGACTCAGTTGTGCAGCGTCAGGCTTCAGTCTCT |
| | | CCTCCTACGCAATGACGTGGGTTAGGCAAGCACCCGGTAAAGGT |
| | | CTGGAATGGATCGGGATTATTTACGCTGGGGGGTCCCCAAGTTA |
| | | CGCGAGCTGGGCTAAAGGTCGATTTACAATAAGTAAAGATAATT |
| | | CCAAGAACACCTTGTATCTGCAGATGAACAGCTTGAGGGCGGAG |
| | | GACACTGCAGTTTATTATTGTGCGCGGGGTACGGGCGATACAGT |
| | | CTATACTTATTTTAACATTTGGGGCCAAGGAACCTTGGTGACCG |
| | | TGTCCTCAGCTAGCACTAAAGGGCCTTCTGTATTTCCCTTGGCC |
| | | CCGTCCAGCAAATCGACCTCGGGAGGGACAGCCGCCCTGGGTTG |
| | | CCTTGTGAAAGATTATTTCCCTGAGCCAGTTACCGTAAGTTGGA |
| | | ACAGTGGGGCGCTGACAAGTGGTGTGCACACGTTTCCTGCCGTC |
| | | CTGCAATCATCGGGCTTGTATAGCCTCAGCTCTGTGGTCACTGT |
| | | CCCAAGTTCATCGCTGGGCACTCAGACGTATATTTGCAATGTGA |
| | | ACCACAAACCTTCAAATACAAAAGTGGATAAACGCGTAGAACCG |
| | | AAATCGTGTGATAAAACTCACACATGCCCGCCATGCCCGGCACC |
| | | TGAAGCAGCTGGTGGTCCCAGCGTGTTCCTGTTCCCGCCGAAGC |
| | | CTAAAGATACTCTAATGATCAGCCGTACGCCAGAGGTGACATGT |
| | | GTCGTGGTTGACGTGTCCCACGAAGATCCCGAAGTTAAGTTCAA |
| | | TTGGTATGTTGATGGTGTAGAGGTACACAATGCTAAGACTAAAC |
| | | CTCGCGAGGAGCAGTACAATTCGACCTATCGTGTCGTGAGCGTT |
| | | CTGACCGTCCTTCACCAAGATTGGCTTAACGGCAAAGAATATAA |
| | | GTGCAAGGTAAGCAATAAAGCACTTGCAGCCCCAATCGAGAAAA |
| | | CCATTTCCAAGGCCAAAGGTCAACCAAGAGAACCCCAGGTGTGT |
| | | ACTCTTCCGCCTTCTCGTGAGGAAATGACTAAAAATCAAGTATC |
| | | CCTTTCCTGTGCTGTTAAAGGTTTTTATCCTAGCGATATTGCTG |
| | | TTGAATGGGAATCGAACGGTCAGCCGGAGAATAATTATAAAACA |
| | | ACGCCACCCGTCCTGGATAGCGACGGCTCATTTTTTCTGGTAAG |
| | | CAAACTGACTGTAGATAAATCACGGTGGCAGCAGGGCAATGTAT |
| | | TCAGTTGCTCCGTTATGCATGAAGCGTTACATAATCACTACACG |
| | | CAGAAATCTCTTAGTCTTTCACCCGGT |

### BsAb

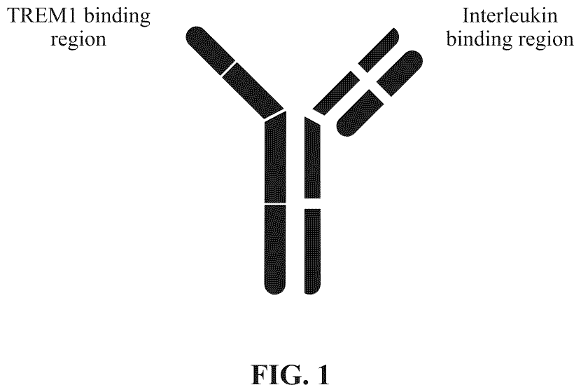

Disclosed herein is a bispecific antibody (BsAb) that binds to TREM1, and an interleukin (e.g., a protein selected from any one of IL-1 family, IL-6 family, IL-12 family and IL-23 family, a variant thereof or a functional fragment thereof). FIG. 1 depicts a bispecific antibody that binds TREM1 and an interleukin. In some embodiments, BsAb comprises a heavy chain region. In some embodiments, the BsAb comprises a light chain region. In some embodiments, the light chain region comprises a kappa light chain constant region. In some embodiments, the BsAb comprises an BsAb light chain variable region. In some embodiments, the BsAb comprises an BsAb heavy chain variable region. In some embodiments, the BsAb comprises an BsAb light chain variable region and an IgG heavy chain variable region. In some embodiments, the BsAb can be a humanized antibody. In some embodiments, the BsAb can be a chimeric antibody. In some embodiments, the BsAb can be a human antibody. In some embodiments, the BsAb comprises a common light chain (L chain). In some embodiments, the L chain acts against a specific antigen. The use of a common light chain can prioritize heterodimerization in the Fc region. In some embodiments, the L chain comprises a knob mutation. In some embodiments, the L chain comprises a hole mutation. In some embodiments, preferential heterodimer formation is preferred upon binding of the BsAb comprising a common L chain. In some embodiments, the formations of heterodimeric pairs are assembled using glutathione disulfide exchange.

Disclosed herein are engineered protein molecules. In some embodiments, an engineered protein molecule described herein comprises a BsAb described herein. In some embodiments, a BsAb described herein is an engineered BsAb that comprises one or more modifications of amino acids that can result in pH-dependent target binding activity. In some embodiments, an engineered BsAb described herein can exhibit pH-dependent target binding activity for a target peptide selected from TREM1, an interleukin (e.g., a protein IL-1 family, IL-6 family, IL-12 family, IL-23 family), a variant thereof and a functional fragment thereof. In some embodiments, an engineered BsAb as described herein can readily bind to a target peptide at a neutral pH and dissociates from the target peptide at an acidic pH. Accordingly, upon administration to a subject the engineered BsAb can bind the target peptide in plasma on account of its neutral pH, while remaining dissociated from the target peptide in endosomes which have an acidic pH. Dissociation of the engineered BsAb from the target peptide in endosomes can facilitate recycling of the engineered BsAb into plasma through FcRn, whereas the target peptide can be trafficked to lysosome and degraded. Such characteristics of the engineered BsAb can allow sweeping of a target peptide from the plasma. Accordingly, in some embodiments, the engineered BsAb can comprise a target peptide sweeping activity for a target peptide that is selected from TREM1, an interleukin (e.g., a protein IL-1 family, IL-6 family, IL-12 family, IL-23 family), a variant thereof and a functional fragment thereof.

In some embodiments, a BsAb described herein is an engineered BsAb that comprises one or more modifications of amino acids that can result in increased FcRn binding at neutral pH. In such embodiments, the engineered BsAb can have increased ability to repeatedly bind to FcRn and remove target peptide from plasma. Alternatively, in some embodiments, a BsAb described herein is an engineered BsAb that comprises one or more modifications of amino acids that can result in increased FcRn binding at acidic pH. In such embodiments, the engineered BsAb can have increased recycling efficiency from endosomes to plasma resulting in improving plasma retention of the engineered BsAb. Accordingly, in some embodiments, a constant domain of an engineered BsAb as described herein can be further modified for increasing FcRn binding activity at neutral pH and/or acidic pH.

In some embodiments, a BsAb described herein is an engineered BsAb that comprises one or more modifications of amino acids that can result in change of isoelectric point of the BsAb. In some embodiments, the one or more modifications of amino acids can result in change of isoelectric point of a VH sequence of the engineered BsAb. In some embodiments, the one or more modifications of amino acids can result in change of isoelectric point of a VL sequence of the engineered BsAb. In some embodiments, the one or more amino acid modifications can increase isoelectric point of the engineered BsAb. In some embodiments, increased isoelectric point can results in increased elimination rate of target peptides from plasma.

In some embodiments, a BsAb described herein is an engineered BsAb that comprises one or more modifications of amino acids that can result in pH-dependent target binding activity and/or increased FcRn binding activity. In some embodiments, the one or more modifications of amino acids comprise at least one, at least two, at least three, at least four, at least five, at least six, at least seven, at least eight, at least nine, at least ten, at least eleven, at least twelve, at least thirteen, at least fourteen, or at least fifteen amino acid modifications. In some embodiments, the one or more modifications of amino acids are in at least one of a Fab region, a scFv region, and a Fc region. In some embodiments, one or more modifications of amino acids are in a VL sequence of a BsAb, a VH sequence of a BsAb, or a combination thereof. In some embodiments, a modification of an amino acid is a deletion, a substitution, or an addition of the amino acid.

In some embodiments, a BsAb can be a monospecific antibody, including but not limited to an antibody wherein both arms target different epitopes of the same antigen. In some embodiments, a BsAb can be a bi-specific antibody. In some embodiments, a BsAb can be a tri-specific antibody. In some embodiments, a BsAb can be a multi-specific antibody.

In some embodiments, a BsAb described herein induces degradation of TREM1, cleavage of TREM1, internalization of TREM1, shedding of TREM1, downregulation of TREM1 expression, or combinations thereof. In some embodiments, a BsAb described herein inhibits interaction (e.g., binding) between TREM1 and one or more TREM1 ligands. In some embodiments, a BsAb described herein transiently activates, and then induces one or more of degradation of TREM1, cleavage of TREM1, internalization of TREM1, shedding of TREM1, downregulation of TREM1 expression, decreased expression of TREM1.

In some embodiments, a BsAb described herein binds proinflammatory receptors and proinflammatory cytokines. Proinflammatory receptors amplify immune response. For example, TREM1 is a proinflammatory receptor that, upon activation, induces production of—proinflammatory cytokines, such as IL-1, IL-2, IL-6, IL-8, IL-12, IL-23 and TNF-α; chemokines, such as MIP-1α, membrane cofactor protein-1 and -2, and GM-CSF; and costimulatory molecules, such as CD1a, CD86, and MHC class II. Accordingly, TREM1 is associated with the occurrence of immune-related inflammatory disease. Reduced TREM1 activity may result in reduced/attenuated proinflammatory cytokine production. Accordingly, in some embodiments, a treatment with a BsAb described herein advantageously reduces inflammatory effects by two independent mechanisms: (1) direct inhibition of inflammatory activity by binding to proinflammatory cytokines, and (2) indirect inhibition of inflammatory activity by reducing proinflammatory cytokine production by binding to TREM1.

In some embodiments, administration of a BsAb described herein that comprises an interleukin binding region results in reduced TREM-1 expression in subjects with an autoimmune condition. Accordingly, in some embodiments, administering a BsAb described herein to a subject with an autoimmune condition can advantageously reduce TREM1 activity by two independent mechanisms: (1) direct inhibition of TREM1 activity by binding to TREM1, and (2) indirect inhibition of TREM1 activity by reducing TREM1 expression by reducing activity of interleukins.

In some embodiments, administration of a BsAb described herein that comprises an TREM-1 binding region results in increased expression of a TREM-1 associated gene. In some embodiments, the TREM-1 associated gene comprises nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), Interferon lambda receptor 1 (IFNLR1), or combinations thereof.

High activity of TREM1 in peripheral myeloid and/or microglia can be responsible for age-associated inflammation and cognitive decline. TREM1 suppresses expression of genes associated with critical enzymes of pentose phosphate pathway, which in turns creates deficiency for ribose-5-phosphate. Ribose-5-phosphate is essential intermediate for purine and pyrimidine synthesis. The deficiency can be corrected by reducing TREM1 activity. Accordingly, in some embodiments, administration of a BsAb described herein that comprises an TREM-1 binding region restores pentose phosphate pathway (PPP). In some embodiments, administration of a BsAb described herein that comprises an TREM-1 binding region restores expression of glycolytic transcripts hexokinase and pyruvate kinase efficiencies. In some embodiments, administration of a BsAb described herein prevents cognitive decline. Moreover, TREM1 expression/activity is also associated with amyloid and tau pathologies in subjects having Alzheimer's disease. Accordingly, in some embodiments, a BsAb described herein comprises neuroprotective activity.

In some embodiments, a BsAb described herein comprises an anti-inflammatory activity that is at least as much as a combined anti-inflammatory activity of a monospecific antibody that binds TREM1 and a monospecific antibody that binds an interleukin (e.g., a protein selected from IL-1 family, IL-6 family, IL-12 family or IL-23 family, a variant thereof, or a functional fragment thereof). In some embodiments, the BsAb advantageously reduces need of administration of two separate antibodies. In some embodiments, a subject being treated with the BsAb advantageously tolerates higher dose relative to a combination of monospecific antibodies.

In some embodiments, a BsAb binds TREM1 and an interleukin (e.g., a protein selected from IL-1 family, IL-6 family, IL-12 family or IL-23 family, a variant thereof, or a functional fragment thereof), wherein the BsAb comprises an anti-inflammatory activity that is at least 10%, at least 20%, at least 30%, at least 40%, at least 50%, at least 60%, at least 70%, at least 80%, at least 90%, at least 100% or more relative to a combined anti-inflammatory activity of a monospecific antibody that binds TREM1 and a monospecific antibody that binds the interleukin.

Moreover, a BsAb described herein comprise two therapeutic domains (TREM1 binding domain and interleukin binding domain) for every Fc region. In contrast, a monospecific antibody comprised one therapeutic domain (TREM1 binding domain or interleukin binding domain) for every Fc region. Accordingly, administration of the BsAb advantageously reduces amount of Fc region being administered. In some embodiments, the BsAb advantageously show less adverse effects relative to the administration of two separate antibodies. In some embodiments, a BsAb described herein advantageously comprises more anti-inflammatory activity relative to combined anti-inflammatory activity of a monospecific antibody that binds TREM1 and a monospecific antibody that binds an interleukin (e.g., IL-1 family, IL-6 family, IL-12 family, IL-23 family).

A treatment with an interleukin binding antibody for reducing the interleukin mediated inflammatory response in a subject may can cause upper respiratory tract infection, headache, nausea, vomiting, rectal bleeding, sore throat, candida infection, hypersensitivity reaction, hypertension, diarrhea, back pain, cough, malignancy, and major adverse cardiovascular events. For example, inhibition of interleukin activity in a subject for treating inflammatory condition may cause candidiasis. In some embodiments, administration of a BsAb described herein in a subject for treating an inflammatory condition (or symptom) experiences lower incidence (or occurrence) of one or more adverse effects associated with reduced interleukin activity relative to a subject being administered with a monospecific interleukin binding antibody that reduces interleukin activity. For example, in some embodiments, a treatment of an inflammatory condition in a subject with a BsAb described herein lowers incidence of candida infection in the subject relative to the subject being treated with a monospecific antibody that binds interleukin and/or reduces activity of the interleukin.

In some embodiments, a BsAb described herein are engineered to comprise pH-dependent target binding activity. In some embodiments, the BsAb comprises such engineered BsAb. In some embodiments, the engineered BsAb comprises a pH-dependent target binding activity for at least one target, wherein the at least one target is selected from TREM1, and an interleukin. In some embodiments, the engineered BsAb readily binds to the at least one target at a neutral pH and dissociates from the at least one target in an acidic pH. Accordingly, the engineered BsAb binds the at least one target in plasma having a neutral pH, and dissociated from the at least one target in endosomes having an acidic pH. Dissociation of the engineered BsAb from the at least one target in endosomes allows recycling of the engineered BsAb into plasma through FcRn. However, the at least one target is trafficked to lysosome and degraded. Such characteristics of the engineered BsAb allows sweeping of at least one target from the plasma. Accordingly, in some embodiments, the engineered BsAb comprises a target sweeping activity for at least one target, wherein the at least one target is selected from TREM1, and an interleukin.

In some embodiments, a BsAb described herein comprises an interleukin binding region and a TREM1 (TREM1 and/or sTREM1) binding region. In some embodiments, the interleukin binding region comprises a first HC region and the TREM1 binding region comprises a second HC region. In some embodiments, the first HC region comprises any one of combinations of CDR-Hs or variants thereof described in TABLE 2, TABLE 9, TABLE 16, and TABLE 23, wherein the variant comprises an amino acid sequence that is at least 60%, at least 65%, at least 70%, at least 75%, at least 80%, at least 85%, at least 90%, at least 95% or 100% identical to a corresponding parent CDR-H sequence described in TABLE 2, TABLE 9, TABLE 16, and TABLE 23, respectively. In some embodiments, the first HC region comprises any one of VH sequences or a variant thereof described in TABLE 3, TABLE 10, TABLE 17, and TABLE 24, wherein the variant comprises an amino acid sequence that is at least 60%, at least 65%, at least 70%, at least 75%, at least 80%, at least 85%, at least 90%, at least 95% or 100% identical to a corresponding parent VH sequence described in TABLE 3, TABLE 10, TABLE 17, and TABLE 24, respectively. In some embodiments, the second HC region comprises any one of combinations of CDR-Hs or variants thereof described in TABLE 30.1, wherein the variant comprises an amino acid sequence that is at least 60%, at least 65%, at least 70%, at least 75%, at least 80%, at least 85%, at least 90%, at least 95% or 100% identical to a corresponding parent CDR-H sequence described in TABLE 30.1. In some embodiments, the second HC region comprises any one of VH sequence or a variant thereof described in TABLE 31, wherein the variant comprises an amino acid sequence that is at least 60%, at least 65%, at least 70%, at least 75%, at least 80%, at least 85%, at least 90%, at least 95% or 100% identical to a corresponding parent VH sequence described in TABLE 31.

In some embodiments, a BsAb described herein comprises an interleukin binding region comprising a first LC, a TREM1 binding region comprising a second LC, or a combination thereof. In some embodiments, the first LC region comprises any one of combinations of CDR-Ls or variants thereof described in TABLE 4, TABLE 11, TABLE 18, and TABLE 25, wherein the variant comprises an amino acid sequence that is at least 60%, at least 65%, at least 70%, at least 75%, at least 80%, at least 85%, at least 90%, at least 95% or 100% identical to a corresponding parent CDR-L sequence described in TABLE 4, TABLE 11, TABLE 18, and TABLE 25, respectively. In some embodiments, the first LC region comprises any one of VL sequence or a variant thereof described in TABLE 6, TABLE 13, TABLE 20, and TABLE 27, wherein the variant comprises an amino acid sequence that is at least 60%, at least 65%, at least 70%, at least 75%, at least 80%, at least 85%, at least 90%, at least 95% or 100% identical to a corresponding parent CDR-L sequence described in TABLE 6, TABLE 13, TABLE 20, and TABLE 27, respectively. In some embodiments, the second LC region comprises any one of combinations of CDR-Ls or variants thereof described in TABLE 32.1, wherein the variant comprises an amino acid sequence that is at least 60%, at least 65%, at least 70%, at least 75%, at least 80%, at least 85%, at least 90%, at least 95% or 100% identical to a corresponding parent CDR-L sequence described in TABLE 32.1. In some embodiments, the second LC region comprises any one of VL sequence or a variant thereof described in TABLE 34, wherein the variant comprises an amino acid sequence that is at least 60%, at least 65%, at least 70%, at least 75%, at least 80%, at least 85%, at least 90%, at least 95% or 100% identical to a corresponding parent CDR-L sequence described in TABLE 34.

In some embodiments, bispecific antibodies described herein comprise at least one constant region. In some embodiments, bispecific antibodies described herein comprise two constant regions. In some embodiments, the two constant regions are derived from a human IgG1 heavy constant chain. In some embodiments, the two constant regions are a first constant region and a second constant region. In some embodiments, the first constant region is engineered to comprise a knob, and the second constant region is engineered to comprise a hole. Accordingly, in some embodiments, the first constant region comprises a mutation at a position T366, per EU numbering, and the second constant region comprises a mutation at a position Y407, per EU numbering. In some embodiments, the first constant region comprises a mutation at a position T366, per EU numbering, and the second constant region comprises mutations at positions T366 and Y407, per EU numbering. In some embodiments, the first constant region comprises a mutation at a position T366, per EU numbering, and the second constant region comprises mutations at positions T366, L368 and Y407, per EU numbering. In some embodiments, the first constant region comprises S354C mutation, T366W mutation, or a combination thereof, per EU numbering, and the second constant region comprises Y349C mutation, T366S mutation, Y407V mutation, or a combination thereof, per EU numbering.

Alternatively, in some embodiments, bispecific antibodies described herein comprise two constant regions are derived from a human IgG1 heavy constant chain, wherein the two constant regions comprise a first constant region and a second constant region, wherein the first constant region comprises at least two mutations and the second constant region comprises at least one mutation. For example, in some embodiments, the first constant region comprises at least two mutations selected from positions L351, F405 and Y407, per EU numbering, and the second constant region comprises at least one mutation selected from positions T366, K392 and T394, per EU numbering. In some embodiments, the mutation at position L351 comprises L351Y and L351A substitutions. In some embodiments, the mutation at position F405 comprises F405A, F405S, F405T and F405V substitutions. In some embodiments, the mutation at position Y407 comprises Y407A, Y407V, Y407S and Y407I substitutions. In some embodiments, the mutation at position T366 comprises T366L, T366M, T366V and T366I substitutions. In some embodiments, the mutation at position K392 comprises K392C, K392M, K392L, K3921, K392E, K392D and K392F substitutions. In some embodiments, the mutation at position T394 comprises T394D, T394W, T394V and T394S substitutions. In some embodiments, the at least two mutations of the first constant region further comprises one or more mutations at positions Q347, Y349, T350, K370, G371, D399 and S400, per EU numbering. In some embodiments, the mutation at position Q347 comprises Q347R, Q347E and Q347K substitutions. In some embodiments, the mutation at position Y349 comprises Y349C substitution. In some embodiments, the mutation at position T350 comprises T350V substitution. In some embodiments, the mutation at position K370 comprises K370T substitution. In some embodiments, the mutation at position G371 comprises G371D and G371S substitutions. In some embodiments, the mutation at position D399 comprises D399C, D399R and D399K substitutions. In some embodiments, the mutation at position S400 comprises S400D, S400K, S400E and S400R substitutions. In some embodiments, the at least two mutations of the second constant region further comprises one or more mutations at positions T350, S354, E357, K360, Q362E, S364, N390, K409 and T411, per EU numbering. In some embodiments, the mutation at position T350 comprises T350V substitution. In some embodiments, the mutation at position S354 comprises S354C substitution. In some embodiments, the mutation at position E357 comprises E357Q substitution. In some embodiments, the mutation at position K360 comprises K360D and K360E substitutions. In some embodiments, the mutation at position Q362 comprises Q362E substitution. In some embodiments, the mutation at position S364 comprises S364R substitution. In some embodiments, the mutation at position N390 comprises N390K, N390R, N390D and N390E substitutions. In some embodiments, the mutation at position K409 comprises K409L, K409M, K409F and K409W substitutions. In some embodiments, the mutation at position T411 comprises T411R, T411D, T4111, T411K, T411E, T411N, T411S and T411L substitutions.

Alternatively, in some embodiments, bispecific antibodies described herein comprise two constant regions are derived from a human IgG1 heavy constant chain, wherein the two constant regions comprise a first constant region and a second constant region, wherein the first constant region and the second constant region are engineered to electrostatically interact with each other. In some embodiments, the first constant region comprises a substitution at K370, per EU numbering, with a negatively charged amino acid residue (e.g., aspartic acid, glutamic acid), and the second constant region comprises a substitution at E357, per EU numbering, with a positively charged amino acid residue (e.g., arginine, lysine, histidine). In some embodiments, the first constant region comprises a substitution at K392 or K409, per EU numbering, with a negatively charged amino acid residue (e.g., aspartic acid, glutamic acid), and the second constant region comprises a substitution at D399, per EU numbering, with a positively charged amino acid residue (e.g., arginine, lysine, histidine). In some embodiments, the first constant region comprises a substitution at K439, per EU numbering, with a negatively charged amino acid residue (e.g., aspartic acid, glutamic acid), and the second constant region comprises a substitution at D356, per EU numbering, with a positively charged amino acid residue (e.g., arginine, lysine, histidine).

In some embodiments, a treatment with the BsAbs described herein reduces at least one inflammatory marker level in serum of a subject by at least 10%, at least 20%, at least 30%, at least 40%, at least 50% more relative to the at least one inflammatory marker (e.g., biomarker) level following a treatment with a corresponding combination of the two mono-specific antibodies. Accordingly, a treatment with a BsAb that binds to TREM1 and an interleukin (e.g., IL-1 family, IL-6 family, IL-12 family, IL-23 family) reduces at least one biomarker level in serum of a subject by 10% or more relative to the at least one biomarker level following a treatment with a corresponding combination of a mono-specific antibody that binds TREM1 and a mono-specific antibody that binds the interleukin. In some embodiments, the at least one biomarker comprises matrix metalloproteinases 1 protein (MMP1), matrix metalloproteinases 2 protein (MMP2), matrix metalloproteinases 7 protein (MMP7), matrix metalloproteinases 10 protein (MMP10), granulocyte-macrophage colony stimulating factor (GM-CSF), tumor necrosis factor-alpha (TNFα), Tumor necrosis factor superfamily member 15 (TNFSF15), IL-17, IL-1α, IL-1β, IL-6, IL-8, IL-12, IL-23 subunit p19, IL-24, IL-36γ, IL-1RA, monocyte chemoattractant protein-1 (MCP-1), chemokine (C-C motif) ligand 1 (CCL1), chemokine (C-C motif) ligand 3 (CCL3), chemokine (C-C motif) ligand 20 (CCL20), chitinase-3-like protein 1 (CHI3L1), prostaglandin-endoperoxide synthase 2 (PTGS2), secretogranin V (SCG5), Inhibin beta-A (INHBA), osteoclast stimulatory membrane protein (OCSTA MP), tissue factor pathway inhibitor 2 (TFPI2), coagulation factor III, regulator of G-protein signaling 16 (RGS16), and TREM1.

### BiTE Antibody

In some embodiments, the molecule described herein comprises a bi-specific T-cell engager (BiTE) antibody construct and may be referred to herein as a “BiTE molecule”. A BiTe antibody construct is a type of fusion protein. In some embodiments, the BiTE molecule activates T-cell activity. In some embodiments, the BiTE molecule comprises two functional single-chain variable fragments. In some embodiments, the BiTE molecule comprises two binding domains. In some embodiments, bispecific antibodies described herein comprise a TREM1 binding domain. In some embodiments, bispecific antibodies described herein further comprise an interleukin binding domain, wherein the interleukin binding domain is capable of binding to a protein selected from any one of IL-1 family, IL-6 family, IL-12 family and IL-23 family, a variant thereof or a functional fragment thereof.

### Chemical Cross-Linking.

The use of chemical cross-linking reagents to covalently link two antibodies is known in the art. Functional antibody fragments generated from their respective parent antibodies by enzymatic digestion or generated through recombinant technologies may be conjugated using bifunctional reagents (Glennie M J et al., J Exp Med 1992; 175:217-225).

### Quadromas

Quadromas and triomas can be generated by fusing either two hybridomas or one hybridoma with a B lymphocyte, respectively (Suresh M R et al., Methods Enzymol 1986; 121: 210-228). In this case the simultaneous expression of two heavy and two light chains leads to the random assembly of 10 antibody combinations and the desired BsAb represent only a small fraction of the secreted antibodies. The BsAb may be purified using a combination of chromatographic techniques.

### Recombinant Multispecific Antibodies

The majority of multispecific antibody formats can be generated by genetic engineering techniques using functional antibody fragment such as scFv or Fab fragments as building blocks connected via polypeptide linkers. Formats based on linked functional antibody fragments include tandem scFv (BiTE), diabodies and tandem-diabodies (Kipriyanov S M. Methods Mol Biol 2003; 207:323-333; Korn T et al, Int J Cancer 2002; 100:690-697). These formats include diabody-Fe, tandem diabody-Fe, tandem diabody-CH3, (scFv)4-Fc and DVD-Ig (Lu D et al, J Immunol Methods 2003; 279: 219-232; Lu D et al, J Biol Chem 2005; 280: 19665-19672; Lu D et al, J Biol Chem 2004; 279: 2856-2865; Wu C et al., Nat Biotechnol 2007 25: 1290-7). In some embodiments, the multispecific antibody is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. Additional types of multispecific antibodies and their construction are disclosed in Brinkmann & Kontermann (2017), mAbs, 9:2, 182-212, DOI: 10.1080/19420862.2016.1268307, the contents of which are incorporated by reference.

Strategies based on forcing the heterodimerization of two heavy chains have been explored. A first approach coined ‘knob into hole’ aims at forcing the pairing of two different IgG heavy chains by introducing mutations into the CH3 domains to modify the contact interface (Ridgway J B et al., Protein Eng 1996; 9: 617-621). On one chain amino acids with large side chains were introduced, to create a ‘knob’. Conversely, bulky amino acids were replaced by amino acids with short side chains to create a ‘hole’ into the other CH3 domain. By co-expressing these two heavy chains, more than 90% heterodimer formation was observed (‘knob-hole’) versus homodimers formation (‘hole-hole’ or ‘knob-knob’). A similar concept was developed using strand-exchange engineered domain (SEED) human CH3 domains based on human IgG and human IgA sequences (Davis J H et al., 2010, PEDS 23: 195-202). These engineered domains lead to the formation of heterodimeric molecules that can carry two different specificities.

Recently an improvement over the ‘knob into hole’ approach; “CrossMab” has been described in WO 2009/080253 A1. This method involves the exchange of some of the light chain and heavy chain domains in addition to the ‘knob into hole’ mutations.

### Single Domain Based Antibodies

The immune systems of camelids (lamas and camels) and cartilaginous fish (nurse sharks) use single V-domains fused to a Fe demonstrating that a single domain can confer high affinity binding to an antigen. Camelid, shark and even human V domains represent alternatives to antibodies, but they also be used for BsAbs generation. They can be reformatted into a classical IgG in which each arm has the potential to bind two targets either via its VH or VL domain.

Bispecific antibodies of the present disclosure can be made by any process disclosed in the application or otherwise known in the art.

### Dual Variable Domain Immunoglobulin

Exemplary bispecific antibodies can comprise individually encoded peptides or “segments” which, in a single continuous chain, would comprise a compact tertiary structure. The component peptides are chosen so as to be asymmetric in their assumed structure, so as not to self-associate to form homo-multimers, but rather to associate in a complementary fashion, adopting a stable complex which resembles the parent tertiary structure. On the genetic level, these segments may be encoded by interchangeable cassettes with suitable restriction sites. These standardized cassettes may be fused C- or N-terminally to different recombinant proteins via a linker or hinge in a suitable expression vector system. Polypeptide segments which do not have the ability to assemble as homodimers are derived by cutting a parental polypeptide which has a compact tertiary structure. These polypeptide segments can then be fused to one or more different functional domains at the genetic level. These distinct polypeptide segments which are now fused to one or more functional domains can be, for example, co-expressed resulting in the formation of a native like parental structure attached to functional domains. This parental structure is formed by the dimerization of the polypeptide segments which were derived from the original parental polypeptide. The resulting multifunctional construct would appear as a compact tertiary structure attached to the one or more functional domains. Once structural sub-domains are identified, the protein is dissected in such a way these sub-domains remain intact. As part of this disclosure, DNA sequences, vectors, preferably bicistronic vectors, vector cassettes, can be made and characterized in that they comprise a DNA sequence encoding an amino acid sequence and optionally at least one further (poly)peptide comprised in the multifunctional polypeptide of the invention, and additionally at least one, preferably singular cloning sites for inserting the DNA encoding at least one further functional domain or that they comprise DNA sequences encoding the amino acid sequences, and optionally the further (poly)peptide(s) comprised in the multifunctional polypeptide of the invention and suitable restriction sites for the cloning of DNA sequences encoding the functional domains, such that upon expression of the DNA sequences after the insertion of the DNA sequences encoding the functional domains into said restriction sites, in a suitable host the multifunctional polypeptide of the invention may be formed. Said vector cassette is characterized in that it comprises the inserted DNA sequence(s) encoding said functional domain(s) and host cells transformed with at least one vector or vector cassette of the invention which can be used for the preparation of said bispecific or multifunctional polypeptides. The host cell may be a mammalian, preferably human, yeast, insect, plant or bacterial, preferably E. coli cell. Bispecific antibodies can be prepared by a method which comprises culturing at least two host cells of the invention in a suitable medium, said host cells each producing only one of said first and said second amino acid sequences attached to at least one further functional domain, recovering the amino acid sequences, mixing thereof under mildly denaturing conditions and allowing in vitro folding of the multifunctional polypeptide of the invention from said amino acid sequences. The method may be characterized in that the further amino acid sequences attached to at least one further functional domain are/is produced by at least one further host cell not producing said first or second amino acid sequence. Additionally, the method may be characterized in that at least one further amino acid sequence attached to at least one further functional domain is produced by the host cell of the invention producing said first or second amino acid sequence.

When either the second or the first portion of an antibody construct described herein comprises two antibody variable domains, these two antibody variable domains can be a VH- and VL-domain which are associated with one another. However, it is also contemplated that the two antibody variable domains comprised in either the second or the first portion may be two VH domains or two VL regions which are associated with one another. In the event that the two antibody variable domains of the first or second portion are covalently associated with one another, the two antibody variable domains may be designed as an scFv fragment, meaning that the two domains are separated from one another by a peptide linker long enough to allow intermolecular association between these two domains. The design of linkers suitable for this purpose is described in the prior art, for example in the granted patents EP 623 679 B1, U.S. Pat. No. 5,258,498, EP 573 551 B1 and U.S. Pat. No. 5,525,491. In other words, a bispecific antibody may be a construct with a total of three antibody variable domains. One antibody variable domain specifically binds alone, i.e., without being paired with another antibody variable domain (a) either to a human immune effector cell by specifically binding to an effector antigen on the human immune effector cell or to a target cell, while the remaining two antibody variable domains together specifically bind (b) either to the target antigen on the target cell or to a human immune effector cell by specifically binding to an effector antigen on the human immune effector cell, respectively. In this case, the presence of three antibody variable domains in the bispecific antibody entails unique advantages. Often, an scFv exhibiting the desired binding specificity for a target antigen is already known and optimized, and omitting one of its two antibody variable domains would abolish or at least attenuate its binding characteristics. Such an scFv may make up part of an antibody construct described herein. Specifically, such a three-domain antibody may advantageously comprise an entire scFv as either its effector antigen- or target antigen-conferring portion. Effectively, then, this allows a bispecific antibody to be formed starting from a desired scFv by simple incorporation of only one additional antibody variable domain into the same polypeptide chain as the scFv, wherein the one additional antibody variable domain incorporated has an antigen binding specificity different than that of the scFv. The first and second portions of the bispecific antibody may be separated from one another by a synthetic polypeptide spacer moiety, which covalently (i.e., peptidically) links either the C-terminus of the first portion with the N-terminus of the second portion, or the C-terminus of the second portion with the N-terminus of the first portion. As such, the portions of these bispecific antibodies may be arranged, as either N-(first portion)-(second portion)-C or N-(second portion)-(first portion)-C. In some embodiments, binding sites of a second specificity are fused to the N- or C-terminus of the heavy or light chain, e.g., in the form of an scFv fragment or a variable single domain, resulting in bispecific, tetravalent molecules. Bispecific molecules generated through fusion of an scFv fragment to a mAb offer great flexibility. ScFv molecules can be linked to the N-terminus but also the C-terminus of the heavy or light chain variable domain of a mAb, generally without compromising productivity or antigen-binding activity. This group of bispecific molecules also includes DVD-Igs, where a second VH and VL domain is fused to the heavy and light chain, respectively, of a mAb, two-in-one antibodies, where a second specificity is introduced into the natural binding site of an IgG molecule, and mAb2 molecules, where a second specificity is built into the CH3 domain of the Fc region. A characteristic feature of all these molecules is a symmetry caused by dimeric assembly of two identical heavy chains, an intrinsic property of these chains.

Heavy chain heterodimerization can be achieved by engineering a charged CH3 interface to introduce an electrostatic steering effect or using the strand-exchange engineered domain technology (SEEDbody) with CH3 sequences composed of alternating segments from human IgA and IgG. In contrast to bispecific IgG-like molecules, these bispecific antibodies are bivalent with a size basically identical to that of IgG. Fc heterodimerization was recently applied to generate a trivalent, bispecific molecule fusing a VH and a VL domain to the C-termini of the engineered heavy chains (HA-TF Fc variant.) Bispecific antibodies with a molecular mass in the range of 50-100 kDa can be generated by combining the variable domains of two antibodies. For example, two scFv have been connected by a more or less flexible peptide linker in a tandem orientation (tandem scFv, taFv, tascFv), which can be extended further by additional scFv, e.g., generating bispecific or trispecific triple bodies (sctb). Diabodies are heterodimeric molecules composed of the variable domains of two antibodies arranged either in the order VHA-VLB and VHB-VLA (VH-VL orientation) or in the order VLA-VHB and VLB-VHA (VL-VH orientation). The linker connecting the two domains within one chain is approximately 5 residues leading, after co-expression of the two chains within one cell, to a head-to-tail assembly and hence formation of a compact molecule with two functional binding sites. The diabody (Db) format was further stabilized by introducing interchain disulfide bonds (dsDb, DART molecules) or by generating a single-chain derivative (scDb). ScDbs can be converted into tetravalent molecules by reducing the middle linker, resulting in homodimerzation of two chains. Small bispecific molecules have also been produced by fusing a scFv to the heavy or light chain of a Fab fragment. Furthermore, tandem scFv, diabodies and scDb have been fused to the Fe or a CH3 domain to generate tetravalent derivatives. Also, scFv can be combined with Fe or CH3 domains to generate tetravalent molecules, e.g., fusing scFvs to the N- and C-terminus of an Fc fragment, or using the knobs-into-holes approach to generate bivalent scFv-Fc or scFv-CH3 molecules. A different approach for the generation of bispecific antibodies of the present invention is the dock-and-lock method (DNL). Many of the established bispecific antibody formats can also be combined with additional proteins and components, e.g., drugs, toxins, enzymes and cytokines, enabling dual targeting and delivery of a fusion partner. In addition, fusion to plasma proteins such as serum albumin or albumin-binding moieties can be applied to extend the plasma half-life of bispecific antibodies.

### Structure of Bispecific Antibodies

In one example, a bispecific antibody may be a binding protein comprising a first polypeptide chain, wherein the polypeptide chain comprises VH1-(X1)n-VH2-C-(X2)n, wherein VH1 is a first heavy chain variable domain, VH2 is a second heavy chain variable domain, C is a constant domain, X1 represents a polypeptide linker, X2 represents an Fc region and n is 0 or 1. In some embodiments, the VH1 and VH2 in the binding protein may be heavy chain variable domains selected from the group consisting of a murine heavy chain variable domain, a human heavy chain variable domain, a CDR grafted heavy chain variable domain, and a humanized heavy chain variable domain. VH1 and VH2 may be capable of binding different antigens. C may be a heavy chain constant domain. For example, X1 is a linker peptide. For example, X1 is a linker listed herein. In an embodiment, X2 is an Fc region. In another embodiment, X2 is a variant Fc region. In some embodiments, VH1 is capable of binding a first antigen and VH2 is capable of binding a second antigen. In some embodiments, VH1 is capable of binding a second antigen and VH2 is capable of binding a first antigen.

In one example, a bispecific antibody may be a binding protein comprising a second polypeptide chain, wherein the polypeptide chain comprises VL1-(X1)n-VL2-C-(X2)n, wherein VL1 is a first light chain variable domain, VL2 is a second light chain variable domain, C is a constant domain, X1 represents a polypeptide linker, X2 represents an Fc region and n is 0 or 1. In some embodiments, the VL1 and VL2 in the binding protein may be light chain variable domains selected from the group consisting of a murine light chain variable domain, a human light chain variable domain, a CDR grafted light chain variable domain, and a humanized light chain variable domain. VL1 and VL2 may be capable of binding different antigens. C may be a heavy chain constant domain. For example, X1 is a linker peptide. For example, X1 is a linker listed herein. In an embodiment, X2 is an Fc region. In another embodiment, X2 is a variant Fc region. In some embodiments, VL1 is capable of binding a first antigen and VL2 is capable of binding a second antigen. In some embodiments, VL1 is capable of binding a first antigen and VL2 is capable of binding a second antigen. In some embodiments, a bispecific antibody construct comprises both the first polypeptide chain and the second polypeptide chain. Bispecific antibodies of the present disclosure can be a dual-variable domain immunoglobulin (DVD-Ig™) as described in Jakob 2013 which combines the target binding domains of two monoclonal antibodies via flexible naturally occurring linkers, which yields a tetravalent IgG-like molecule.

The present disclosure additionally provides a method of making a DVD-Ig binding protein by preselecting the parent antibodies against a first antigen and a second antigen. A method of making a Dual Variable Domain Immunoglobulin that binds two antigens comprises the steps of a) obtaining a first parent antibody, or antigen binding fragment thereof, that binds a first antigen; b) obtaining a second parent antibody or antigen binding fragment thereof, that binds a second antigen; c) constructing two copies of a first polypeptide chains, each of which comprises VH1-(X1)n-VH2-C-(X2)n, wherein, VH1 is a first heavy chain variable domain obtained from said first parent antibody, or antigen binding fragment thereof; VH2 is a second heavy chain variable domain obtained from said second parent antibody or antigen binding fragment thereof, which can be the same as or different from the first parent antibody; C is a heavy chain constant domain; (X1)n is a linker wherein said (X1)n is either present or absent; and (X2)n is an Fc region, d) constructing two copies of a second polypeptide chains each of which comprises VL1-(X1)n-VL2-C-(X2)n, wherein, VL1 is a first light chain variable domain obtained from said first parent antibody, or antigen binding fragment thereof; VL2 is a second light chain variable domain obtained from said second parent antibody, or antigen binding thereof, which can be the same as or different from the first parent antibody; C is a light chain constant domain; (X1)n is a linker, wherein said (X1)n is either present or absent; and (X2)n does not comprise an Fc region, wherein said (X2)n is either present or absent; and e) expressing two copies of said first and second polypeptide chains; such that a DVD-Ig binds said first antigen and said second antigen is generated.

Generation of First Antigen Binding and/or Second Antigen Binding Domains

Disclosed herein are dual variable domain immunoglobulins. The variable domains of the DVD binding protein can be obtained from parent antibodies, including polyclonal and mAbs that bind antigens of interest. These antibodies may be naturally occurring or may be generated by recombinant technology, or can be designed de novo. MAbs can be prepared using a wide variety of techniques known in the art including the use of hybridoma, recombinant, and phage display technologies, or a combination thereof. Monoclonal antibodies can be prepared by methods disclosed herein.

Disclosed herein are dual variable domain immunoglobulins comprising variable domains. The dual variable domain immunoglobulin (DVD-Ig) molecule is designed such that two different light chain variable domains (VL) from the two different parent monoclonal antibodies are linked in tandem directly or via a short linker by recombinant DNA techniques, followed by the light chain constant domain, and optionally, an Fc region. Similarly, the heavy chain comprises two different heavy chain variable domains (VH) linked in tandem, followed by the constant domain CH1 and Fc region. The variable domains can be obtained using recombinant DNA techniques from a parent antibody generated by any one of the methods described herein. The variable domain may be a murine heavy or light chain variable domain, a CDR a human heavy or light chain variable domain. The first and second variable domains may be linked directly to each other using recombinant DNA techniques, linked via a linker sequence, or the two variable domains are linked. The variable domains may bind the same antigen or may bind different antigens. The constant domain may be linked to the two linked variable domains using recombinant DNA techniques. Sequence comprising linked heavy chain variable domains may be linked to a heavy chain constant domain and sequence comprising linked light chain variable domains is linked to a light chain constant domain. The constant domains may also be human heavy chain constant domain and human light chain constant domain respectively. The DVD heavy chain may be further linked to an Fc region. The Fc region may be a native sequence Fc region, or a variant Fc region, or a human Fc region. Two heavy chain DVD polypeptides and two light chain DVD polypeptides may be combined to form a DVD-Ig molecule.

In some embodiments, a Fc region described herein is derived from an IgG heavy constant chain or an IgA heavy constant chain. In some embodiments, the IgG heavy constant chain comprises a IgG1 heavy chain constant chain. In some embodiments, the IgG1 heavy constant chain comprises a human IgG1 heavy chain constant chain. An amino acid sequence of wildtype human IgG1 heavy chain constant chain is provided in TABLE 23. In some embodiments, the Fc region is engineered to not bind to Fe gamma receptor (FcγR). In some embodiments, the FcγR comprises FcγRI, FcγRII and FcγRIII.

TABLE 40
Amino acid sequence of Fc region of human IgG1

| SEQ | |
|---|---|
| ID | |
| NO: | HC Amino Acid Sequences |
| 453 | ASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPELQLEESCAEAQDG |
| | ELDGLWTTITIFITLFLLSVCYSATVTFFKVKWIFSSVVDLKQTIIPDYRNMIGQGA |
| 454 | ASTKGPSVFPLAPCSRSTSESTAALGCLVKDYFPEPVTVSWNSGALTSGVHTFPAVL |
| | QSSGLYSLSSVVTVPSSNFGTQTYTCNVDHKPSNTKVDKTVERKCCVECPPCPAPPV |
| | AGPSVFLFPPKPKDTLMISRTPEVTCVVVDVSHEDPEVQFNWYVDGVEVHNAKTKPR |
| | EEQFNSTFRVVSVLTVVHQDWLNGKEYKCKVSNKGLPAPIEKTISKTKGQPREPQVY |
| | TLPPSREEMTKNQVSLTCLVKGFYPSDISVEWESNGQPENNYKTTPPMLDSDGSFFL |
| | YSKLTVDKSRWQQGNVFSCSVMHEALHNHYTQKSLSLSPELQLEESCAEAQDGELDG |
| | LWTTITIFITLFLLSVCYSATITFFKVKWIFSSVVDLKQTIVPDYRNMIRQGA |
| 455 | ASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPSCPAPEF |
| | LGGPSVFLFPPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLELQLEESCAEAQDGELD |
| | GLWTTITIFITLFLLSVCYSATVTFFKVKWIFSSVVDLKQTIVPDYRNMIRQGA |

In some embodiments, the Fc region comprises an amino acid sequence that is at least 60%, at least 65%, at least 70%, at least 75%, at least 80%, at least 85%, at least 90%, at least 95% or 100% identical to a corresponding parent sequence of the Fc region of IgG1 (SEQ ID NO: 453).

In some embodiments, the Fc region described herein comprises one or more mutations relative to a corresponding parent sequence of the Fc region of IgG1 (SEQ ID NO: 453). In some embodiments, the Fc region of IgG1 comprises at least one, at least two, at least three, at least four, at least five, at least six, at least seven, at least eight, at least nine, at least ten, at least eleven, at least twelve, at least thirteen, at least fourteen or at least fifteen mutations relative to a corresponding parent sequence of SEQ ID NO: 453. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen deletions, substitutions, additions or combinations thereof relative to a corresponding parent sequence of SEQ ID NO: 453. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen substitutions relative to a corresponding parent sequence of SEQ ID NO: 453.

In some embodiments, a Fc region described herein is derived from a human IgG1 heavy chain constant chain. In some embodiments, the human IgG1 heavy chain constant chain comprises at least one substitution, at least two substitutions, at least three substitutions, at least four substitutions, at least five substitutions, at least six substitutions or at least seven substitutions. In some embodiments, the at least one substitution is selected from the positions N297, C226, C229, E233, L234, L235, G236, G237, P238, F243, M252, 5254, T256, D265, 5267, H268, D270, P271, R292, Y300, K322, A327, L328, P329, A330, P331, and P396, per EU numbering. In some embodiments, the at least two substitutions are selected from the positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering. In some embodiments, the at least three substitutions are selected from the positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering. In some embodiments, the at least four substitutions are selected from the positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering. In some embodiments, the at least five substitutions are selected from the positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering. In some embodiments, the at least six substitutions are selected from the positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering. In some embodiments, the at least seven substitutions are selected from the positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering. In some embodiments, the substitutions at the position N297 comprises N297A and N297Q substitutions. In some embodiments, the substitutions at the position C226 comprises C226S substitution. In some embodiments, the substitutions at the position C229 comprises C229S substitution. In some embodiments, the substitutions at the position E233 comprises E233P and E233D substitutions. In some embodiments, the substitutions at the position L234 comprises L234A, L234D, L234E, L234G, L234H, L234K, L234Q, L234R, L234S, L234T and L234F substitutions. In some embodiments, the substitutions at the position L235 comprises L235A, L235S, L235T, L235H, L235K, L235Q, L235D, L235I, L235V, L235R, L235E and L235G substitutions. In some embodiments, the substitutions at the position G236 comprises G236R substitution. In some embodiments, the substitutions at the position G237 comprises G237A and G237D substitutions. In some embodiments, the substitutions at the position P238 comprises P238A, P238D and P238S substitutions. In some embodiments, the substitutions at the position F243 comprises F243L substitution. In some embodiments, the substitutions at the position M252 comprises M252Y substitution. In some embodiments, the substitutions at the position S254 comprises S254T substitution. In some embodiments, the substitutions at the position T256 comprises T256E substitution. In some embodiments, the substitutions at the position D265 comprises D265A substitution. In some embodiments, the substitutions at the position S267 comprises S267E substitution. In some embodiments, the substitutions at the position H268 comprises H268A and H268D substitutions. In some embodiments, the substitutions at the position D270 comprises D270A substitution. In some embodiments, the substitutions at the position P271 comprises P271G substitution. In some embodiments, the substitutions at the position R292 comprises R292P substitution. In some embodiments, the substitutions at the position Y300 comprises Y300L substitutions. In some embodiments, the substitutions at the position K322 comprises K322A and K322Q substitutions. In some embodiments, the substitutions at the position A327 comprises A327Q and A327G substitutions. In some embodiments, the substitutions at the position L328 comprises L328E and L328F substitutions. In some embodiments, the substitutions at the position P329 comprises P329G and P329A substitutions. In some embodiments, the substitutions at the position A330 comprises A330S, A330R and A330L substitutions. In some embodiments, the substitutions at the position P331 comprises P331S substitution. In some embodiments, the substitutions at the position P396 comprises P396L substitution.

In some embodiments, a human IgG1 heavy chain constant chain described herein comprises two substitutions. In some embodiments, the two substitutions are located at positions L234 and L235, per EU numbering. In some embodiments, the two substitutions are L234A and L235A substitutions.

In some embodiments, a human IgG1 heavy chain constant chain described herein comprises three substitutions. In some embodiments, the three substitutions are located at positions L234, L235 and P329, per EU numbering. In some embodiments, the two substitutions are L234A, L235A and P329A substitutions. In some embodiments, the three substitutions are located at positions M252, S254 and T256, per EU numbering. In some embodiments, the three substitutions are M252T, S254T and T256E substitutions.

In some embodiments, the Fc region comprises an amino acid sequence that is at least 60%, at least 65%, at least 70%, at least 75%, at least 80%, at least 85%, at least 90%, at least 95% or 100% identical to a corresponding parent sequence of the Fc region of IgG2 (SEQ ID NO: 454).

In some embodiments, the Fc region described herein comprises one or more mutations relative to a corresponding parent sequence of the Fc region of IgG2 (SEQ ID NO: 454). In some embodiments, the Fc region of IgG2 comprises at least one, at least two, at least three, at least four, at least five, at least six, at least seven, at least eight, at least nine, at least ten, at least eleven, at least twelve, at least thirteen, at least fourteen or at least fifteen mutations relative to a corresponding parent sequence of SEQ ID NO: 454. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen deletions, substitutions, additions or combinations thereof relative to a corresponding parent sequence of SEQ ID NO: 454. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen substitutions relative to a corresponding parent sequence of SEQ ID NO: 454.

In some embodiments, a Fc region described herein is derived from a human IgG2 heavy chain constant chain. In some embodiments, the human IgG2 heavy chain constant chain comprises at least one substitution, at least two substitutions, at least three substitutions, at least four substitutions, at least five substitutions, at least six substitutions or at least seven substitutions. In some embodiments, the at least one substitution is selected from the positions C232, C233, V234, G237, P238, M252, S254, T256, H268, N297, V309, A330, and P331, per EU numbering. In some embodiments, the at least two substitutions are selected from the positions C232, C233, V234, G237, P238, M252, S254, T256, H268, N297, V309, A330, and P331, per EU numbering. In some embodiments, the at least three substitutions are selected from the positions C232, C233, V234, G237, P238, M252, S254, T256, H268, N297, V309, A330, and P331, per EU numbering. In some embodiments, the at least four substitutions are selected from the positions C232, C233, V234, G237, P238, M252, S254, T256, H268, N297, V309, A330, and P331, per EU numbering. In some embodiments, the at least five substitutions are selected from the positions C232, C233, V234, G237, P238, M252, S254, T256, H268, N297, V309, A330, and P331, per EU numbering. In some embodiments, the at least six substitutions are selected from the positions C232, C233, V234, G237, P238, M252, S254, T256, H268, N297, V309, A330, and P331, per EU numbering. In some embodiments, the at least seven substitutions are selected from the positions C232, C233, V234, G237, P238, M252, S254, T256, H268, N297, V309, A330, and P331, per EU numbering. In some embodiments, the substitutions at the position C232 comprises C232S substitution. In some embodiments, the substitutions at the position C233 comprises C233S substitution. In some embodiments, the substitutions at the position V234 comprises V234A substitution. In some embodiments, the substitutions at the position G237 comprises G237A substitution. In some embodiments, the substitutions at the position P238 comprises P238S substitution. In some embodiments, the substitutions at the position M252 comprises M252Y substitution. In some embodiments, the substitutions at the position S254 comprises S254T substitution. In some embodiments, the substitutions at the position T256 comprises T256E substitution. In some embodiments, the substitutions at the position H268 comprises H268A, H268E and H268Q substitutions. In some embodiments, the substitutions at the position N297 comprises N297A and N297Q substitutions. In some embodiments, the substitutions at the position V309 comprises V309L substitution. In some embodiments, the substitutions at the position A330 comprises A330S substitution. In some embodiments, the substitutions at the position P331 comprises P331S substitution.

In some embodiments, the Fc region comprises an amino acid sequence that is at least 60%, at least 65%, at least 70%, at least 75%, at least 80%, at least 85%, at least 90%, at least 95% or 100% identical to a corresponding parent sequence of the Fc region of IgG4 (SEQ ID NO: 455).

In some embodiments, the Fc region described herein comprises one or more mutations relative to a corresponding parent sequence of the Fc region of IgG4 (SEQ ID NO: 455). In some embodiments, the Fc region of IgG4 comprises at least one, at least two, at least three, at least four, at least five, at least six, at least seven, at least eight, at least nine, at least ten, at least eleven, at least twelve, at least thirteen, at least fourteen or at least fifteen mutations relative to a corresponding parent sequence of SEQ ID NO: 455. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen deletions, substitutions, additions or combinations thereof relative to a corresponding parent sequence of SEQ ID NO: 455. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen substitutions relative to a corresponding parent sequence of SEQ ID NO: 455.

In some embodiments, a Fc region described herein is derived from a human IgG4 heavy chain constant chain. In some embodiments, the human IgG4 heavy chain constant chain comprises at least one substitution, at least two substitutions, at least three substitutions, at least four substitutions, at least five substitutions, at least six substitutions or at least seven substitutions. In some embodiments, the at least one substitution is selected from the positions S228, E233, F234, L235, L236, G237, S241, L248, M252, S254, T256, N297, E318, and T394, per EU numbering. In some embodiments, the at least two substitutions are selected from the positions S228, E233, F234, L235, L236, G237, S241, L248, M252, S254, T256, N297, E318, and T394, per EU numbering. In some embodiments, the at least three substitutions are selected from the positions S228, E233, F234, L235, L236, G237, S241, L248, M252, S254, T256, N297, E318, and T394, per EU numbering. In some embodiments, the at least four substitutions are selected from the positions S228, E233, F234, L235, L236, G237, S241, L248, M252, S254, T256, N297, E318, and T394, per EU numbering. In some embodiments, the at least five substitutions are selected from the positions S228, E233, F234, L235, L236, G237, S241, L248, M252, S254, T256, N297, E318, and T394, per EU numbering. In some embodiments, the at least six substitutions are selected from the positions S228, E233, F234, L235, L236, G237, S241, L248, M252, S254, T256, N297, E318, and T394, per EU numbering. In some embodiments, the at least seven substitutions are selected from the positions S228, E233, F234, L235, L236, G237, S241, L248, M252, S254, T256, N297, E318, and T394, per EU numbering. In some embodiments, the substitutions at the position S228 comprises S228P substitution. In some embodiments, the substitutions at the position E233 comprises E233P substitution. In some embodiments, the substitutions at the position F234 comprises F234V substitution. In some embodiments, the substitutions at the position L235 comprises L235A substitution. In some embodiments, the substitutions at the position G237 comprises G237A substitution. In some embodiments, the substitutions at the position S241 comprises S241P substitution. In some embodiments, the substitutions at the position L248 comprises L248E substitution. In some embodiments, the substitutions at the position M252 comprises M252Y substitution. In some embodiments, the substitutions at the position S254 comprises S254T substitution. In some embodiments, the substitutions at the position T256 comprises T256E substitution. In some embodiments, the substitutions at the position N297 comprises N297A and N297Q substitutions. In some embodiments, the substitutions at the position E318 comprises E318A substitution. In some embodiments, the substitutions at the position T394 comprises T394D substitution.

The design of the “dual-specific multivalent full length binding proteins” of the present disclosure leads to a dual variable domain light chain and a dual variable domain heavy chain which assemble primarily to the desired “dual-specific multivalent full length binding proteins”.

### Construction of DVD Molecules

Disclosed herein are dual variable domain immunoglobulins. The dual variable domain immunoglobulin (DVD-Ig) molecule is designed such that two different light chain variable domains (VL) from the two parent monoclonal antibodies, which can be the same or different, are linked in tandem directly or via a short linker by recombinant DNA techniques, followed by the light chain constant domain, and optionally, an Fc region. Similarly, the heavy chain comprises two different heavy chain variable domains (VH) linked in tandem, followed by the constant domain CH1 and Fc region

Disclosed herein are dual variable domain immunoglobulins comprising variable domains. The variable domains can be obtained using recombinant DNA techniques from a parent antibody generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

Disclosed herein are dual variable domain immunoglobulins comprising a first variable domain and a second variable domain. In one embodiment the first and second variable domains are linked directly to each other using recombinant DNA techniques. In another embodiment the variable domains are linked via a linker sequence. In an embodiment, two variable domains are linked. Three or more variable domains may also be linked directly or via a linker sequence. The variable domains may bind the same antigen or may bind different antigens. DVD-Ig molecules of the invention may include one immunoglobulin variable domain and one non-immunoglobulin variable domain, such as ligand binding domain of a receptor, or an active domain of an enzyme. DVD-Ig molecules may also comprise two or more non-Ig domains.

In an embodiment a constant domain is linked to the two linked variable domains using recombinant DNA techniques. In an embodiment, sequence comprising linked heavy chain variable domains is linked to a heavy chain constant domain and sequence comprising linked light chain variable domains is linked to a light chain constant domain. In an embodiment, the constant domains are human heavy chain constant domain and human light chain constant domain respectively. DVD-Ig molecules described herein may include a DVD heavy chain. In an embodiment, the DVD heavy chain is further linked to an Fc region. The Fc region may be a native sequence Fc region, or a variant Fc region. In another embodiment, the Fc region is a human.

In another embodiment two heavy chain DVD polypeptides and two light chain DVD polypeptides are combined to form a DVD-Ig molecule.

Binding proteins of the present invention may be produced by any of a number of techniques known in the art. For example, expression from host cells, wherein expression vector(s) encoding the DVD heavy and DVD light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD proteins of the invention in either prokaryotic or eukaryotic host cells, DVD proteins are expressed in eukaryotic cells, for example, mammalian host cells, because such eukaryotic cells (and in particular mammalian cells) are more likely than prokaryotic cells to assemble and secrete a properly folded and immunologically active DVD protein.

Exemplary mammalian host cells for expressing the recombinant antibodies of the invention include Chinese Hamster Ovary (CHO cells) (including dhfr-CHO cells, described in Urlaub and Chasm, (1980) Proc. Natl. Acad. Sci. USA 77:4216-4220, used with a DHFR selectable marker, e.g., as described in Kaufman, R. J. and Sharp, P. A. (1982) Mol. Biol. 159:601-621), NS0 myeloma cells, COS cells, SP2 and PER.C6 cells. When recombinant expression vectors encoding DVD proteins are introduced into mammalian host cells, the DVD proteins are produced by culturing the host cells for a period of time sufficient to allow for expression of the DVD proteins in the host cells or secretion of the DVD proteins into the culture medium in which the host cells are grown. DVD proteins can be recovered from the culture medium using standard protein purification methods.

In an exemplary system for recombinant expression of DVD proteins in constructs described herein, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. Still further the invention provides a method of synthesizing a DVD protein of the invention by culturing a host cell of the invention in a suitable culture medium until a DVD protein of the invention is synthesized. The method can further comprise isolating the DVD protein from the culture medium.

An important feature of DVD-Ig is that it can be produced and purified in a similar way as a conventional antibody. The production of DVD-Ig results in a homogeneous, single major product with desired dual-specific activity, without any sequence modification of the constant region or chemical modifications of any kind. Other previously described methods to generate “bi-specific”, “multi-specific”, and “multi-specific multivalent” full length binding proteins do not lead to a single primary product but instead lead to the intracellular or secreted production of a mixture of assembled inactive, mono-specific, multi-specific, multivalent, full length binding proteins, and multivalent full length binding proteins with combination of different binding sites. As an example, based on the design described by Miller and Presta (PCT Publication No. WO2001/077342(A1), there are 16 possible combinations of heavy and light chains. Consequently only 6.25% of protein is likely to be in the desired active form, and not as a single major product or single primary product compared to the other 15 possible combinations. Separation of the desired, fully active forms of the protein from inactive and partially active forms of the protein using standard chromatography techniques, typically used in large scale manufacturing, is yet to be demonstrated.

The design of the “dual-specific multivalent full length binding proteins” for use in constructs described herein leads to a dual variable domain light chain and a dual variable domain heavy chain which assemble primarily to the desired “dual-specific multivalent full length binding proteins”.

In some embodiments, at least 50%, at least 75% and at least 90% of the assembled, and expressed dual variable domain immunoglobulin molecules are the desired dual-specific tetravalent protein. This aspect of the invention particularly enhances the commercial utility of the invention. Therefore, the present invention includes a method to express a dual variable domain light chain and a dual variable domain heavy chain in a single cell leading to a single primary product of a “dual-specific tetravalent full length binding protein”.

Provided herein are methods of expressing a dual variable domain light chain and a dual variable domain heavy chain in a single cell leading to a “primary product” of a “dual-specific tetravalent full length binding protein,” where the “primary product” is more than 50% of all assembled protein, comprising a dual variable domain light chain and a dual variable domain heavy chain.

Provided herein are methods of expressing a dual variable domain light chain and a dual variable domain heavy chain in a single cell leading to a single “primary product” of a “dual-specific tetravalent full length binding protein,” where the “primary product” is more than 75% of all assembled protein, comprising a dual variable domain light chain and a dual variable domain heavy chain.

Provided herein are methods of expressing a dual variable domain light chain and a dual variable domain heavy chain in a single cell leading to a single “primary product” of a “dual-specific tetravalent full length binding protein,” where the “primary product” is more than 90% of all assembled protein, comprising a dual variable domain light chain and a dual variable domain heavy chain.

### Binding Affinity

Binding affinity is generally represented by the dissociation constant (K_{D}). K_{D} values for antibodies can be determined by any of the methods known in the art. Exemplary methods for determining K_{D} includes by using surface plasmon resonance (SPR), isothermal titration calorimetry (ITC), spectroscopic assays, biolayer interferometry (BLI), and grating-coupled interferometry (GCI).

In some embodiments, a multispecific antibody described herein comprises a binding affinity in a range of from 1 pM to 1 μM, from 10 pM to 1 μM, from 100 pM to 1 μM, from 1 nM to 1 μM, from 10 nM to 1 μM, from 100 nM to 1 μM, from 500 nM to 1 μM, from 1 pM to 500 nM, from 10 pM to 500 nM, from 100 pM to 500 nM, from 1 nM to 500 nM, from 10 nM to 500 nM, from 100 nM to 500 nM, from 1 pM to 100 nM, from 10 pM to 100 nM, from 100 pM to 100 nM, from 1 nM to 100 nM, from 10 nM to 100 nM, from 1 pM to 10 nM, from 10 pM to 10 nM, from 100 pM to 10 nM, from 1 nM to 10 nM, from 1 pM to 1 nM, from 10 pM to 1 nM, from 100 pM to 1 nM, from 1 pM to 100 pM, or from 10 pM to 100 pM.

In some embodiments, a BsAb antibody described herein comprises an average binding affinity for target antigen is in a range of from 1 pM to 1 μM, from 10 pM to 1 μM, from 100 pM to 1 μM, from 1 nM to 1 μM, from 10 nM to 1 μM, from 100 nM to 1 μM, from 500 nM to 1 μM, from 1 pM to 500 nM, from 10 pM to 500 nM, from 100 pM to 500 nM, from 1 nM to 500 nM, from 10 nM to 500 nM, from 100 nM to 500 nM, from 1 pM to 100 nM, from 10 pM to 100 nM, from 100 pM to 100 nM, from 1 nM to 100 nM, from 10 nM to 100 nM, from 1 pM to 10 nM, from 10 pM to 10 nM, from 100 pM to 10 nM, from 1 nM to 10 nM, from 1 pM to 1 nM, from 10 pM to 1 nM, from 100 pM to 1 nM, from 1 pM to 100 pM, or from 10 pM to 100 pM.

In some embodiments, a multispecific antibody described herein comprises at least two binding domains. In some embodiments, the at least two binding domains are a first binding domain and a second binding domain. In some embodiments, the first binding domain is TREM1 binding domain. In some embodiments, the second binding domain is an interleukin binding domain. In some embodiments, the binding affinity of multispecific antibody is measured with only one target molecule (e.g., TREM1, sTREM1, or an interleukin (e.g., any one of proteins from IL-1 family, IL-6 family, IL-12 family and IL-23 family)).

In some embodiments, a multispecific antibody described herein comprises a TREM1 binding region and an interleukin binding region. In some embodiments, the interleukin binding region binds an interleukin, wherein the interleukin comprises any one of proteins from IL-1 family, IL-6 family, IL-12 family and IL-23 family. In some embodiments, a binding affinity of the TREM1 binding region for TREM1 is lower than a binding affinity of the interleukin binding region for an interleukin. In some embodiments, the multispecific antibody comprises the binding affinity of the TREM1 binding region for TREM1 that is at least two times, at least three time, at least four times, at least five times, at least ten times, at least fifteen times, at least twenty times, at least forty times, at least sixty times, at least eight times, or at least hundred times higher than the binding affinity of the interleukin binding region for an interleukin. Alternatively, in some embodiments, a binding affinity of the interleukin binding region for an interleukin is lower than a binding affinity of the TREM1 binding region for TREM1. Accordingly, in some embodiments, the multispecific antibody comprises a binding affinity of the interleukin binding region for an interleukin that is at least two times, at least three time, at least four times, at least five times, at least ten times, at least fifteen times, at least twenty times, at least forty times, at least sixty times, at least eight times, or at least hundred times higher than the binding affinity of the TREM1 binding region for TREM1.

In some embodiments, a multispecific antibody described herein comprises a TREM1 binding region and an interleukin binding region that binds an interleukin (e.g., any one of proteins from IL-1 family, IL-6 family, IL-12 family, IL-23 family). In some embodiments, the interleukin binding region comprises an IL-1 binding region that binds an interleukin from IL-1 family, an IL-6 binding region that binds an interleukin from IL-6 family, an IL-12 binding region that binds an interleukin from IL-12 family, an IL-23 binding region that binds an interleukin from IL-23 family, or a combination thereof. In some embodiments, a binding affinity of the TREM1 binding region for sTREM1 is lower than a binding affinity of the interleukin binding region for an interleukin. In some embodiments, a binding affinity of the TREM1 binding region for sTREM1 is at least two times, at least three time, at least four times, at least five times, at least ten times, at least fifteen times, at least twenty times, at least forty times, at least sixty times, at least eight times, or at least hundred times higher than a binding affinity of the interleukin binding region for an interleukin. Alternatively, in some embodiments, a binding affinity of the interleukin binding region for an interleukin is lower than a binding affinity of the TREM1 binding region for sTREM1. Accordingly, in some embodiments, a binding affinity of the interleukin binding region for an interleukin is at least two times, at least three time, at least four times, at least five times, at least ten times, at least fifteen times, at least twenty times, at least forty times, at least sixty times, at least eight times, or at least hundred times higher than a binding affinity of the TREM1 binding region for sTREM1.

Alternatively, in some embodiments, multispecific antibodies described herein undergo cooperative binding event. For example, in some embodiments, a multispecific antibody undergoes a positive cooperative binding event, wherein binding of the multispecific antibody to a first target molecule results in increase in binding affinity for a second target molecule. For example, in some embodiments, a binding affinity of a TREM1 bound multispecific antibody for an interleukin is lower than a binding affinity of a TREM1 unbound multispecific antibody for the interleukin. In some embodiments, a binding affinity of an interleukin bound multispecific antibody for TREM1 is lower than a binding affinity of an interleukin unbound multispecific antibody for TREM1. Alternatively, in some embodiments, amultispecific antibody undergoes a negative cooperative binding event, wherein binding of the multispecific antibody to a first target molecule results in decrease in binding affinity for a second target molecule. For example, in some embodiments, a binding affinity of a TREM1 bound multispecific antibody for an interleukin is higher than a binding affinity of a TREM1 unbound multispecific antibody for the interleukin. In some embodiments, a binding affinity of an interleukin bound multispecific antibody for TREM1 is higher than a binding affinity of an interleukin unbound multispecific antibody for TREM1. In some embodiments, a binding affinity of sTREM1 bound multispecific antibody for an interleukin is lower than a binding affinity of a sTREM1 unbound multispecific antibody for the interleukin. In some embodiments, a binding affinity of an interleukin bound multispecific antibody for sTREM1 is lower than a binding affinity of an interleukin unbound multispecific antibody for sTREM1. Alternatively, in some embodiments, a multispecific antibody undergoes a negative cooperative binding event, wherein binding of the multispecific antibody to a first target molecule results in decrease in binding affinity for a second target molecule. For example, in some embodiments, a binding affinity of a sTREM1 bound multispecific antibody for an interleukin is higher than a binding affinity of a sTREM1 unbound multispecific antibody for the interleukin. In some embodiments, a binding affinity of an interleukin bound multispecific antibody for sTREM1 is higher than a binding affinity of the interleukin unbound multispecific antibody for sTREM1.

### Kappa-Lambda Antibodies

Provided herein are multispecific (e.g., bispecific, trispecific) antibodies in the kappa-lambda antibody format. Bispecific antibodies provided herein have a common heavy chain, two light chains—one Kappa (K), one Lambda (λ)—that each has a different specificity (i.e., two light chains, two specificities). The methods provided herein produce molecules having specific binding where diversity is restricted to the VL region. These methods produce bispecific antibodies through controlled co-expression of the three chains (one VH chains, two VL chains), and purification.

This type of molecule is composed of two copies of a unique heavy chain polypeptide, a first light chain variable region fused to a constant Kappa domain and a second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However, it is also possible to obtain bispecific antibodies of the invention by fusing a Kappa light chain variable domain to a constant Lambda domain for a first specificity and fusing a Lambda light chain variable domain to a constant Kappa domain for the second specificity.

An essential step of exemplary methods is the identification of two antibody Fv regions (each composed by a variable light chain and variable heavy chain domain) having different antigen specificities that share the same heavy chain variable domain. Numerous methods have been described for the generation of monoclonal antibodies and functional fragments thereof. (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al, 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies may be generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, antibodies may be obtained by screening a library that contains antibody or antigen binding domain sequences for binding to the target antigen. This library may be prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies may be prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Kappa-lambda antibodies having the same heavy chain variable domain can be generated by the use of antibody libraries in which the heavy chain variable domain is the same for all the library members and thus the diversity is confined to the light chain variable domain. Such libraries are described, for example, WO 2010/135558. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. Numerous methods for the modification of the Fc portion have been described and are applicable to antibodies of the invention, (see for example Strohl, W R Curr Opin Biotechnol 2009 (6):685-91).

Another step of exemplary embodiments is the optimization of co-expression of the common heavy chain and two different light chains into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

In some embodiments, purified bispecific antibodies described herein are characterized as follows. The flow-through and elution from each affinity purification step can be analyzed by SDS-PAGE. The specificity and affinity of d-bodies can be determined by ELISA and surface plasmon resonance. The methods of the invention can allow for the identification of antibodies with affinities in the sub-nanomolar to nanomolar range without optimization. This is not obvious as the diversity in antibody libraries described herein is restricted to the light chain which contributes less to the binding energy in standard antibodies.

To avoid the requirement of having access to two antibodies having light chain variable domains of the Kappa and Lambda type being perceived as a limitation to the instant invention, the methods described herein allow for the generation of hybrid light chain in which a Lambda variable domain can be fused to a Kappa constant domain and conversely a Kappa variable domain can be fused to a Lambda constant domain. In some embodiments, the methods of generating bispecific and/or multi-specific antibodies use a complete serum-free chemically defined process. These methods incorporate the most widely used mammalian cell line in pharmaceutical industry, the Chinese Hamster Ovary (CHO) cell line. The methods described therein are used to generate both semi-stable and stable cell lines. The methods can be used to manufacture bispecific and/or multi-specific antibodies of the invention at small scale (e.g., in an Erlenmeyer flask) and at mid-scale (e.g., in 25 L Wave bag). The methods are also readily adaptable for larger scale production of bispecific and/or multi-specific antibodies, as well as antibody mixtures of the invention.

### Methods of Treatment

Disclosed herein, in some embodiments, are methods of treating a subject in need thereof, comprising administering to the subject, an effective amount of one or more engineered protein molecules described herein. In some embodiments, an engineered protein molecule comprises a TREM1 binding engineered protein molecule described herein. In some embodiments, an engineered protein molecule comprises an interleukin binding engineered protein molecule described herein. In some embodiments, an engineered protein molecule comprises both, a TREM1 binding engineered protein molecule described herein, and an interleukin binding engineered protein molecule described herein. In some embodiments, an interleukin binding engineered protein molecule is selected from an IL-1 binding engineered protein molecule, an IL-6 binding engineered protein molecule, an IL-12 binding engineered protein molecule, an IL-23 binding engineered protein molecule, or a combination thereof.

Disclosed herein, in some embodiments, are methods of treating a subject in need thereof, comprising administering to the subject, a dose in an effective amount of one or more engineered protein molecules described herein. Disclosed herein, in some embodiments, are methods of treating a subject in need thereof, comprising administering to the subject, a dose in an effective amount of a multispecific (e.g., bispecific, trispecific) antibody or multispecific (e.g., bispecific, trispecific) molecule described herein or a pharmaceutical composition comprising said multispecific (e.g., bispecific, trispecific) antibody or multispecific (e.g., bispecific, trispecific) molecule described herein. In some embodiments, the subject has any one of the conditions selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, lupus, acute and chronic colitis, rheumatoid arthritis, atherosclerosis, wound healing, Crohn's disease, inflammatory bowel disease, ulcerative colitis, obesity, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, lupus, arthritis, multiple sclerosis, low bone density, osteoporosis, osteogenesis, osteopetrotic disease, and Paget's disease of bone. In some embodiments, the subject has an autoimmune condition. In some embodiments, the autoimmune condition comprises inflammatory bowel disease, Crohn's disease, ulcerative colitis, irritable bowel syndrome, rheumatoid arthritis, psoriasis, psoriatic arthritis, systemic lupus erythematosus, lupus nephritis, type I diabetes, Grave's disease, multiple sclerosis, autoimmune myocarditis, Kawasaki disease, coronary artery disease, chronic obstructive pulmonary disease, interstitial lung disease, autoimmune thyroiditis, scleroderma, systemic sclerosis, osteoarthritis, atopic dermatitis, vitiligo, graft versus host disease, Sjogrens's syndrome, autoimmune nephritis, Goodpasture's syndrome, chronic inflammatory demyelinating polyneuropathy, allergy, and asthma. In some embodiments, the subject has age-associated Alzheimer's disease. In some embodiments, the subject has a disease or condition selected from the group consisting of: an infectious disease, or an autoimmune disease. In some embodiments, the subject has: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis.

Inflammatory diseases or conditions are associated with increases activity and/or expression of TREM-1, an interleukin described herein, or combinations thereof in a subject relative to the subject not having (or prior to development of) the inflammatory diseases or conditions. Additionally, inflammatory diseases or conditions are also associated with increased activity or expression of one or more downstream inflammatory signaling proteins of TREM1, an interleukin described herein, or combinations thereof in a subject relative to the subject not having (or prior to development of) the inflammatory diseases or conditions. In some embodiments, the one or more inflammatory conditions comprise dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, lupus, acute and chronic colitis, rheumatoid arthritis, atherosclerosis, wound healing, Crohn's disease, inflammatory bowel disease, ulcerative colitis, obesity, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, lupus, arthritis, multiple sclerosis, low bone density, osteoporosis, osteogenesis, osteopetrotic disease, and Paget's disease of bone. In some embodiments, the one or more inflammatory conditions comprise an autoimmune condition. In some embodiments, the one or more inflammatory conditions comprise inflammatory bowel disease, Crohn's disease, ulcerative colitis, irritable bowel syndrome, rheumatoid arthritis, psoriasis, psoriatic arthritis, systemic lupus erythematosus, lupus nephritis, type I diabetes, Grave's disease, multiple sclerosis, autoimmune myocarditis, Kawasaki disease, coronary artery disease, chronic obstructive pulmonary disease, interstitial lung disease, autoimmune thyroiditis, scleroderma, systemic sclerosis, osteoarthritis, atopic dermatitis, vitiligo, graft versus host disease, Sjogrens's syndrome, autoimmune nephritis, Goodpasture's syndrome, chronic inflammatory demyelinating polyneuropathy, allergy, and asthma. In some embodiments, the one or more inflammatory conditions comprise inflammatory condition in central nervous system. In some embodiments, the one or more inflammatory conditions comprise age-associated Alzheimer's disease. In some embodiments, the one or more inflammatory conditions are selected from the group consisting of: an infectious disease, or an autoimmune disease. In some embodiments, the one or more inflammatory conditions comprise rheumatoid arthritis, juvenile arthritis, psoriatic arthritis, axial spondyloarthritis and/or ankylosing spondylitis. In some embodiments, the one or more inflammatory conditions comprise psoriasis and/or hidradenitis suppurativa. In some embodiments, the one or more inflammatory conditions comprise ulcerative colitis, Crohn's disease, necrotizing enterocolitis, sepsis and/or multiple sclerosis. In some embodiments, the one or more inflammatory conditions comprise sepsis and/or multiple sclerosis. Accordingly, in some embodiments, methods of treatment of the inflammatory diseases or conditions in a subject comprises administering a BsAb described herein, wherein the BsAb reduces activity and/or expression of TREM1, an interleukin described herein, one or more downstream signaling proteins thereof, or combinations thereof more in the subject relative to the subject being treated with a monospecific antibody targeting TREM1, or an interleukin described herein. In some embodiments, methods of treatment of the inflammatory diseases or conditions in a subject comprises administering a BsAb described herein, wherein the BsAb reduces activity and/or expression of TREM1, an interleukin described herein, one or more downstream signaling proteins thereof, or combinations thereof more in the subject relative to the subject being treated with a combination of two monospecific antibodies, wherein the first monospecific antibody targets TREM1 and the second monospecific antibody targets an interleukin described herein.

In some embodiments, the multispecific (e.g., bispecific, trispecific) antibody or multispecific (e.g., bispecific, trispecific) molecule is administered with one or more additional therapeutic agents. In some embodiments, the multispecific (e.g., bispecific, trispecific) antibody or multispecific (e.g., bispecific, trispecific) molecule and one or more additional therapeutic agents are co-administered. In some embodiments, the multispecific (e.g., bispecific, trispecific) antibody or multispecific (e.g., bispecific, trispecific) molecule and one or more additional therapeutic agents are sequentially administered.

### Methods of Diagnosis

Disclosed herein, in some embodiments, are methods of diagnosing a condition of a subject, comprising incubating a sample with an effective amount of a composition comprising one or more engineered protein molecules described herein. Disclosed herein, in some embodiments, are methods of diagnosing a condition of a subject, comprising incubating a sample with an effective amount of a composition comprising a multispecific (e.g., bispecific, trispecific) antibody or multispecific (e.g., bispecific, trispecific) molecule described herein. In some embodiments, the sample comprises a tissue, blood, serum, plasma, saliva, urine, or combinations thereof. In some embodiments, the diagnosis is based on the expression level of TREM1, wherein an elevated level of TREM1 in the subject relative to that in a healthy individual indicates that the individual suffers from an autoimmune condition. In some embodiments, the diagnosis is based on the expression level of TREM1, wherein an elevated level of TREM1 in the subject relative to level of TREM1 in the subject prior to development of an autoimmune condition. In some embodiments, the diagnosis is based on the expression level of sTREM1, wherein an elevated level of sTREM1 in the subject relative to that in a healthy individual indicates that the individual suffers from an infectious disease or an autoimmune condition. In some embodiments, In some embodiments, the autoimmune condition comprises inflammatory bowel disease, Crohn's disease, ulcerative colitis, irritable bowel syndrome, rheumatoid arthritis, psoriasis, psoriatic arthritis, systemic lupus erythematosus, lupus nephritis, type I diabetes, Grave's disease, multiple sclerosis, autoimmune myocarditis, Kawasaki disease, coronary artery disease, chronic obstructive pulmonary disease, interstitial lung disease, autoimmune thyroiditis, scleroderma, systemic sclerosis, osteoarthritis, atopic dermatitis, vitiligo, graft versus host disease, Sjogrens's syndrome, autoimmune nephritis, Goodpasture's syndrome, chronic inflammatory demyelinating polyneuropathy, allergy, and asthma. In some embodiments, the autoimmune condition comprises rheumatoid arthritis, juvenile arthritis, psoriatic arthritis, ankylosing spondylitis, axial spondyloarthritis, psoriasis, hidradenitis suppurativa, ulcerative colitis, Crohn's disease, necrotizing enterocolitis, sepsis, multiple sclerosis or a combination thereof.

### Compositions

Disclosed herein are compositions comprises one or more engineered protein constructs described herein. In some embodiments, an engineered protein construct comprises a TREM1 binding moiety described herein. In some embodiments, an engineered protein construct comprises an interleukin binding moiety described herein. In some embodiments, an engineered protein molecule comprises both, a TREM1 binding moiety described herein, and an interleukin binding moiety described herein. In some embodiments, an interleukin binding moiety is selected from an IL-1 binding moiety, an IL-6 binding moiety, an IL-12 binding moiety, an IL-23 binding moiety, or a combination thereof. In some embodiments, compositions described herein further comprise one or more additional therapeutic agents.

### Dosages

Provided herein are compositions comprising one or more engineered protein constructs for treatment (including prevention) of a disease (e.g., an infectious condition, disorder or disease, an autoimmune condition, disorder or disease, a dermatological condition, disorder or disease). In some embodiments, an engineered protein construct comprises a TREM1 binding moiety described herein. In some embodiments, an engineered protein construct comprises an interleukin binding moiety described herein. In some embodiments, an engineered protein construct comprises both, a TREM1 binding moiety described herein, and an interleukin binding moiety described herein. Also, provided herein are compositions comprising a multispecific (e.g., bispecific, trispecific) antibody or multispecific (e.g., bispecific, trispecific) molecule or antigen binding fragment thereof for treatment (including prevention) of a disease (e.g., an infectious condition, disorder or disease, an autoimmune condition, disorder or disease, a dermatological condition, disorder or disease). In some embodiments, the compositions are pharmaceutical compositions comprising a pharmaceutically acceptable carrier. The compositions are administered in an amount effective for treatment (including prophylaxis) of an infectious condition, disorder or disease, an autoimmune condition, disorder or disease, a dermatological condition, disorder or disease. In some embodiments, the compositions (e.g., the antibodies or the antigen binding fragment thereof or the nucleic acid molecules encoding said antibody or antigen binding fragment thereof) are administered in an amount effective for enhancing an immune response and/or increasing T cell activation in a subject. The compositions are to be used for in vivo administration to a subject by any available means, such as parenteral administration. For administration to a subject, a composition or medicament comprising the antibodies or antigen binding fragment thereof described herein can be sterile, which can readily be accomplished by filtration through sterile filtration membranes, or other methods known to those of skill in the art. In one embodiment, a composition or medicament has been treated to be free of pyrogens or endotoxins. Testing pharmaceutical compositions or medicaments for pyrogens or endotoxins and preparing pharmaceutical compositions or medicaments free of pyrogens or endotoxins or preparing pharmaceutical compositions or medicaments that have endotoxins at a clinically acceptable level, are well understood to one of ordinary skill in the art. Commercial kits are available to test pharmaceutical compositions or medicaments for pyrogens or endotoxins.

The compositions to be used for in vivo administration, such as parenteral administration, in the methods described herein can be sterile, which is readily accomplished by filtration through sterile filtration membranes, or other methods known to those of skill in the art.

In some embodiments, elevated level of TREM1 and/or sTREM1 is directly corelated with a condition. In some embodiments, the condition comprises the condition comprises rheumatoid arthritis, juvenile arthritis, psoriatic arthritis, ankylosing spondylitis, axial spondyloarthritis, psoriasis, hidradenitis suppurativa, ulcerative colitis, Crohn's disease, necrotizing enterocolitis, sepsis, multiple sclerosis or a combination thereof. In some embodiments, a dose for treating a condition in a subject is determined based on proportional increase in TREM1 and/or sTREM1 relative to that in a healthy individual. In some embodiments, a dose for treating a condition in a subject is determined based on proportional change in level of TREM1 and/or sTREM1 relative to level of TREM1 and/or sTREM1 prior to development of the condition.

### Pharmaceutical Compositions and Dosage Forms

Disclosed herein, in certain embodiments, are pharmaceutical compositions comprising one or more engineered protein constructs for administration in a subject. In some embodiments, an engineered protein construct comprises a TREM1 binding moiety described herein. In some embodiments, an engineered protein construct comprises an interleukin binding moiety described herein. In some embodiments, an engineered protein construct comprises both, a TREM1 binding moiety described herein, and an interleukin binding moiety described herein. Also, disclosed herein, in certain embodiments, are pharmaceutical compositions comprising an engineered protein construct, a multispecific (e.g., bispecific, trispecific) antibody or multispecific (e.g., bispecific, trispecific) molecule or a functional fragment thereof disclosed herein for administration in a subject.

In some embodiments, pharmaceutical compositions comprising an engineered protein construct, a multispecific (e.g., bispecific, trispecific) molecule or multispecific (e.g., bispecific, trispecific) antibody described herein are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, intraarterial, intrathecal, intralymphatic, intralesional, intracapsular, intraorbital, intracardiac, intradermal, transtracheal, subcuticular, subcapsular, subarachnoid, intraspinal, epidural, intrasternal, or intracranial), intranasal, buccal, sublingual, spinal or rectal administration routes. In some embodiments, the pharmaceutical composition is formulated for parenteral (e.g., intravenous, subcutaneous, intramuscular, intracerebral, intracerebroventricular, intra-articular, intraperitoneal, intraarterial, intrathecal, intralymphatic, intralesional, intracapsular, intraorbital, intracardiac, intradermal, transtracheal, subcuticular, subcapsular, subarachnoid, intraspinal, epidural, intrasternal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, pharmaceutical compositions described herein comprise one or more stabilizers, filling agents, surfactants, or combinations thereof. In some embodiments, the stabilizer comprises amino acids (e.g., glycine, alanine, lysine, arginine, and threonine), carbohydrates (e.g., glucose, sucrose, trehalose, raffinose, and maltose), polyols (e.g., glycerol, mannitol, sorbitol, cyclodextrins or dextran of any kind and molecular weight, and polyethylene glycol (PEG)). In some embodiments, the filling agent comprises lactose, sucrose, glucose, mannitol, sorbitol, calcium carbonate, and magnesium stearate. In some embodiments, the surfactant comprises anionic surfactant, cationic surfactant, zwitterionic surfactant, or nonionic surfactant. In some embodiments, the surfactant comprises alkyl ethoxylate, nonylphenol ethoxylate, amine ethoxylate, polyethylene oxide, polypropylene oxide, fatty alcohols (e.g., cetyl alcohol or oleyl alcohol), cocamide MEA, cocamide DEA, polysorbates, and dodecyl dimethylamine oxide. In some embodiments, the surfactant is polysorbate 20. In some embodiments, the surfactant is polysorbate 80.

In some embodiments, the pharmaceutical compositions described herein are formulated into capsules. In some embodiments, the pharmaceutical compositions are formulated into solutions (for example, for IV administration). In some embodiments, the pharmaceutical composition is formulated as an infusion. In some embodiments, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

The compositions disclosed herein, comprising an antibody or antigen binding fragment, described herein, can also contain more than one active compound as necessary for the particular indication being treated, preferably those with complementary activities that do not adversely affect each other. For example, the composition can further comprise retinoids, such as Acitretin (e.g., Soriatane®) and isotretinoin, immune system suppressants (e.g., rapamycin, Tcell blockers [e.g., Amevive® (alefacept) and Raptiva® [efalizumab]), cyclosporine, methotrexate, mycophenolate mofetil, mycophenolic acid, leflunomide, tacrolimus, etc.), hydroxyurea (e.g., Hydrea®), sulfasalazine, 6-thioguanine, fumarates (e.g., dimethylfumarate and fumaric acid esters), azathioprine, colchicine, alitretinoin, steroids, corticosteroids, certolizumab, aprelimast, mometasone, rosiglitazone, pioglitazone, botulinium toxin, triamcinolone, IFN-1 (InflaRx), bimekizumab (UCB), MaBpl (XBiotech), LY-3041658 (Eli Lilly), TE-2232 (Immunwork), NSAIDs, prescription narcotics, ketoprofen, codeine, gabapentin, pregabalin gentanyl, antibiotics (topical, oral, IV) (e.g., clindamycin, rifampin, tetracycline, sarecycline, doxycycline, minocycline, lymecycline, trimethoprim-sulfamethoxazole, erythromycin, ceftriaxone, moxifloxacin, metronidazole, separately or as combinations), corticosteroid (injectable or oral), antiandrogen/hormonal therapy (oral contraceptives, spironolactone, finasteride, dutasteride, progesterone IUD, cyproterone acetate, ethinyloestradiol, gestodene, norgestimate, desogestrel, drospirenone, spironolactone), Triamcinolone Acetonide, MEDI8968, hydroxychloroquine, dapsone, metformin, adapalene, azelaic acid and zinc. Such molecules are suitably present in combination in amounts that are effective for the purpose intended. The active ingredients of the compositions comprising an antibody or antigen binding fragment thereof described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microparticle, microemulsions, nano-particles and nanocapsules) or in macroemulsions. Such techniques are disclosed in Remington's Pharmaceutical Sciences (16th ed., Osol, ed., 1980). The pharmaceutical composition can be also delivered in a vesicle, in particular a liposome (see Langer 1990 Science 249:1527-1533; Treat et al. (1989) in Liposomes in the Therapy of Infectious Disease and Cancer, Lopez-Berestein and Fidler (eds.), Liss, New York, pp. 353-365; Lopez-Berestein, ibid., pp. 317-327; see generally ibid.). Liposomes include emulsions, foams, micelles, insoluble monolayers, phospholipid dispersions, lamellar layers and the like, and can serve as vehicles to target the M-CSF antibodies to a particular tissue as well as to increase the half-life of the composition. A variety of methods are available for preparing liposomes, as described in, e.g., U.S. Pat. Nos. 4,837,028 and 5,019,369, which patents are incorporated herein by reference.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. In some embodiments, pharmaceutical formulations and medicaments may be prepared as liquid suspensions or aqueous solutions, for example, using a sterile liquid, such as, but not limited to, an oil, water, an alcohol, and combinations of these. In some embodiments, pharmaceutical compositions can be prepared in a lyophilized form. The lyophilized preparations can comprise a cryoprotectant known in the art. The term “cryoprotectants” as used herein generally includes agents, which provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations. Pharmaceutically suitable surfactants, suspending agents, emulsifying agents, may be added for oral or par-enteral administration.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

In some embodiments, pharmaceutical compositions described herein comprises: (a) one or more antibodies described herein; (b) at least one of a buffering agent, a stabilizer, a pH modifier, a salt, and a surfactant.

### Kits

Provided herein are also kits, medicines, compositions, and unit dosage forms for use in any of the methods described herein. Provided herein is a kit comprising a therapeutically effective amount of at least one of an engineered protein construct, a multispecific (e.g., bispecific, trispecific) antibody, a multispecific (e.g., bispecific, trispecific) molecule, or antigen binding fragment thereof disclosed herein. In some embodiments, an engineered protein construct comprises a TREM1 binding moiety described herein. In some embodiments, an engineered protein construct comprises an interleukin binding moiety described herein. In some embodiments, an engineered protein construct comprises both, a TREM1 binding moiety described herein, and an interleukin binding moiety described herein. In some embodiments, the kit further comprises a second therapeutic agent (e.g., an immune system suppressant, immunomodulating agent, or other agent including but not limited to an agent disclosed herein). In some embodiments, the antibody or antigen binding fragment thereof is an aqueous form or a lyophilized form. The kit further comprises a diluent or a reconstitution solution.

Kits can include one or more containers comprising an antibody (or unit dosage forms and/or articles of manufacture). In some embodiments, a unit dosage is provided wherein the unit dosage contains a predetermined amount of a composition comprising an antibody (e.g., a therapeutically effective amount), with or without one or more additional agents. In some embodiments, such a unit dosage is supplied in single-use prefilled syringe for injection. In some embodiments, the composition comprising the antibody or antigen binding fragment thereof can comprise saline, sucrose, or the like; a buffer, such as phosphate, or the like; and/or be formulated within a stable and effective pH range. In some embodiments, the antibody or antigen binding fragment thereof can be provided as a lyophilized powder that may be reconstituted upon addition of an appropriate liquid, for example, sterile water. In some embodiments, an antibody or an antigen binding functional fragment thereof further comprises one or more substances that inhibit protein aggregation, including, but not limited to, sucrose and arginine. In some embodiments, an antibody or an antigen binding functional fragment further comprises heparin and/or a proteoglycan.

In some embodiments, kits further comprise instructions for use in the treatment of a disease or condition in accordance with any of the methods described herein. The kit may further comprise a description of selection an individual suitable or treatment. Instructions supplied in the kits are typically written instructions on a label or package insert (for example, a paper sheet included in the kit), but machine-readable instructions (for example, instructions carried on a magnetic or optical storage disk) are also acceptable. In some embodiments, the kit further comprises another therapeutic agent.

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### EXEMPLARY EMBODIMENTS

Disclosed herein are bispecific molecules comprising: a first domain and a second domain, wherein the first domain binds TREM1, a variant thereof or a functional fragment thereof, and wherein the second domain binds at least one protein from any one of IL-1 family, IL-6 family, IL-12 family and IL-23 family, a variant thereof or a functional fragment thereof. In some embodiments, a bispecific molecule is an antibody, a variant thereof, or a functional fragment thereof. In some embodiments, TREM1 comprises an IgV domain or a stalk region. In some embodiments, a bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, a bispecific molecule comprises a heterodimeric antibody or a functional fragment thereof. In some embodiments, a bispecific molecule comprises a constant region. In some embodiments, the molecule comprises a sequence knock out in a constant region. In some embodiments, the first domain comprises a TREM1-binding heavy chain variable domain. In some embodiments, the first domain comprises a TREM1-binding light chain variable domain. In some embodiments, the second domain comprises a n interleukin binding domain. In some embodiments, the interleukin binding domain comprising an interleukin binding heavy chain variable domain, an interleukin binding light chain variable domain, or a combination thereof. In some embodiments, at least one of the first domain and the second domain comprises a light chain constant domain and/or heavy chain constant domain.

Also disclosed herein are bispecific molecules for use in the treatment of a disease or condition. In some embodiments, the disease or condition is selected from the group consisting of: rheumatoid arthritis, juvenile arthritis, psoriatic arthritis, an axial spondyloarthritis, ankylosing spondylitis, psoriasis, hidradenitis suppurativa, ulcerative colitis, a Crohn's disease, necrotizing enterocolitis, sepsis, or multiple sclerosis. In some embodiments, a bispecific molecule comprises at least one of a Fc region and/or a Fab region.

Also disclosed herein are pharmaceutical compositions comprising a bispecific molecule described herein, and a pharmaceutically acceptable carrier. Also disclosed herein are methods of treating a disease or condition in a subject, the method comprising administering to the subject an effective amount of a bispecific molecule or a pharmaceutical composition described herein, thereby treating the disease or condition.

Also disclosed herein are methods of treating an infectious disease, autoimmune disease and/or dermatological disease in a subject, the method comprising administering to the subject an effective amount of a bispecific molecule that comprises: a TREM1 binding domain that comprises at least one of the heavy chain complementarity-determining regions (CDR-Hs) recited in TABLE 30, at least one of the light chain CDR-Ls recited in TABLE 31, a TREM1 binding variant thereof, or a combination thereof; and an interleukin binding domain that comprises at least one of the heavy chain complementarity-determining regions (CDR-Hs) recited in TABLE 2, TABLE 9, TABLE 16 and TABLE 23, at least one of the light chain CDR-Ls recited in TABLE 4, TABLE 11, TABLE 18 and TABLE 25, the interleukin binding variant thereof, or a combination thereof.

Also disclosed herein are compositions comprising a bispecific molecule comprising a first domain and a second domain, wherein the first domain binds TREM1, a variant thereof or a functional fragment thereof, wherein the second domain binds a protein selected from any one of IL-1 family, IL-6 family, IL-12 family and IL-23 family, a variant thereof or a functional fragment thereof, and wherein administration of an effective amount of the composition to a subject in need thereof results in treatment of a disease or condition. In some embodiments, a bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, a bispecific molecule comprises a heterodimeric antibody or a functional fragment thereof. In some embodiments, a bispecific molecule comprises a constant region. In some embodiments, a bispecific molecule comprises a sequence knock out in a constant region. Also disclosed herein are nucleic acids encoding at least a portion of any one of bispecific molecules disclosed herein.

### Additional Exemplary Embodiments

Embodiment 1: An engineered protein molecule comprising: a first region and a second region, wherein the first region binds TREM1, a variant thereof or a functional fragment thereof, and wherein the second region binds an interleukin, wherein the interleukin comprises a protein selected from any one of IL-1 family, IL-6 family, IL-12 family and IL-23 family, a variant thereof or a functional fragment thereof.

Embodiment 2: The engineered protein construct of Embodiment 1, wherein the engineered protein construct is an antibody, a variant thereof, or a functional fragment thereof.

Embodiment 3: The engineered protein construct of any one of the preceding Embodiments 1-2, wherein the engineered protein construct is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof.

Embodiment 4: The engineered protein construct of any one of Embodiments 1-3 the preceding Embodiments, wherein the engineered protein construct comprises a heterodimeric antibody or a functional fragment thereof.

Embodiment 5: The engineered protein construct of any one of Embodiments 1-4 the preceding Embodiments, wherein the engineered protein construct comprises a constant region.

Embodiment 6: The engineered protein construct of any one of Embodiments 1-5 the preceding Embodiments, wherein the first region comprises a TREM1-binding heavy chain variable domain.

Embodiment 7: The engineered protein construct of any one of Embodiments 1-6 the preceding Embodiments, wherein the first region comprises a TREM1-binding light chain variable domain.

Embodiment 8: The engineered protein construct of any one of Embodiments 1-7 the preceding Embodiments, wherein the second region comprises an interleukin binding heavy chain variable domain.

Embodiment 9: The engineered protein construct of any one of Embodiments 1-8 the preceding Embodiments, wherein the second region comprises an interleukin binding light chain variable domain.

Embodiment 10: The engineered protein construct of any one of Embodiments 1-9, wherein a binding affinity of the first region for TREM1 is lower than a binding affinity of the second region for the interleukin.

Embodiment 11: The engineered protein construct of Embodiment 10, wherein the binding affinity of second binding region for the interleukin is at least two times the binding affinity of the first region for TREM1.

Embodiment 12: The engineered protein construct of any one of Embodiments 1-9, wherein a binding affinity of the first region for TREM1 is higher than a binding affinity of the second region for the interleukin.

Embodiment 13: The engineered protein construct of Embodiment 12, wherein the binding affinity of the first region for TREM1 is at least two times the binding affinity of second binding region for the interleukin.

Embodiment 14: The engineered protein construct of any one of Embodiments 1-13 the preceding Embodiments, wherein the engineered protein construct comprises at least one of a Fc region and/or a Fab region.

Embodiment 15: The engineered protein construct of Embodiment 14, wherein the Fc region comprises an amino acid sequence that is at least 60%, at least 65%, at least 70%, at least 75%, at least 80%, at least 85%, at least 90%, at least 95% or 100% identical to any one of amino acid sequences of SEQ ID NO: 453-455.

Embodiment 16: The engineered protein construct of any one of Embodiments 1-15, wherein at least one of the first region and the second region comprises a light chain constant domain and/or a heavy chain constant domain.

Embodiment 17: The engineered protein construct of Embodiment 16, wherein the heavy chain constant domain of the first region comprises the Fc region having S354C mutation and T366W mutation, per EU numbering, and the heavy chain constant domain of the second region comprises the Fc region having Y349C mutation, T366S mutation and Y407V mutation, per EU numbering.

Embodiment 18: The engineered protein construct of Embodiment 16, the heavy chain constant domain of the second region comprises the Fc region having S354C mutation and T366W mutation, per EU numbering, and the heavy chain constant domain of the first region comprises the Fc region having Y349C mutation, T366S mutation and Y407V mutation, per EU numbering.

Embodiment 19: The engineered protein construct of Embodiment 16, wherein the Fc region comprises a human IgG1 heavy chain constant chain having at least one substitution is selected from positions N297, C226, C229, E233, L234, L235, G236, G237, P238, F243, M252, S254, T256, D265, S267, H268, D270, P271, R292, Y300, K322, A327, L328, P329, A330, P331, and P396, per EU numbering.

Embodiment 20: The engineered protein construct of Embodiment 16, wherein the Fc region comprises a human IgG2 heavy chain constant chain having at least one substitution is selected from positions C232, C233, V234, G237, P238, M252, S254, T256, H268, N297, V309, A330, and P331, per EU numbering.

Embodiment 21: The engineered protein construct of Embodiment 16, wherein the Fc region comprises a human IgG4 heavy chain constant chain having at least one substitution is selected from positions S228, E233, F234, L235, L236, G237, S241, L248, M252, S254, T256, N297, E318, and T394, per EU numbering.

Embodiment 22: The engineered protein construct of any one of Embodiments 1-21 exhibits a pH-dependent target binding activity for a target peptide, wherein the target peptide is selected from TREM1, an interleukin, a variant thereof and a functional fragment thereof, and wherein the interleukin is selected from IL-1 family of proteins, IL-6 family of proteins, IL-12 family of proteins, and IL-23 family of proteins.

Embodiment 23: The engineered protein construct of any one of Embodiments 1-22, wherein the engineered protein construct comprises anti-inflammatory activity that is at least 10%, at least 20%, at least 30%, at least 40%, at least 50%, at least 60%, at least 70%, at least 80%, at least 90%, at least 100%, or more relative to a combined anti-inflammatory activity of a monospecific antibody that binds TREM1 and a monospecific antibody that binds interleukin.

Embodiment 24: The engineered protein construct of any one of Embodiments 1-23 the preceding Embodiments, for use in the treatment of an inflammatory disease or condition.

Embodiment 25: The engineered protein construct of Embodiment 24, wherein the inflammatory disease or condition is selected from the group consisting of: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis.

Embodiment 26: A composition comprising the engineered protein construct of any one of Embodiments 1-25.

Embodiment 27: A composition for use in treating an inflammatory disease or condition comprising: an engineered protein construct comprising a first region and a second region, wherein the first region binds TREM1, a variant thereof or a functional fragment thereof, wherein the second region binds an interleukin, wherein the interleukin comprises a protein selected from any one of IL-1 family, IL-6 family, IL-12 family and IL-23 family, a variant thereof or a functional fragment thereof, and wherein administration of an effective amount of the composition to a subject in need thereof results in treatment of an inflammatory disease or condition.

Embodiment 28: The composition for use of Embodiment 27, wherein the inflammatory disease or condition is selected from the group consisting of: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis.

Embodiment 29: The composition for use of Embodiment 27, wherein the inflammatory disease or condition is rheumatoid arthritis, juvenile arthritis, psoriatic arthritis, axial spondyloarthritis or ankylosing spondylitis.

Embodiment 30: The composition for use of Embodiment 27, wherein the inflammatory disease or condition is psoriasis or hidradenitis suppurativa.

Embodiment 31: The composition for use of Embodiment 27, wherein the inflammatory disease or condition is ulcerative colitis, Crohn's disease, necrotizing enterocolitis, sepsis, or multiple sclerosis.

Embodiment 32: The composition for use of Embodiment 27, wherein the inflammatory disease or condition is sepsis.

Embodiment 33: The composition for use of Embodiment 27, wherein the inflammatory disease or condition is multiple sclerosis.

Embodiment 34: A nucleic acid encoding at least a portion of any one of the engineered protein constructs of Embodiments 1-26 disclosed herein.

Embodiment 35: A pharmaceutical composition comprising the engineered protein molecule of any one of Embodiments 1-26, and a pharmaceutically acceptable carrier.

Embodiment 36: A pharmaceutical composition for use in treating an inflammatory disease or condition, wherein the pharmaceutical composition comprises: a TREM1 binding moiety, an interleukin binding moiety and a pharmaceutically acceptable carrier, wherein the interleukin binding moiety comprises a protein selected from an IL-1 binding moiety, an IL-6 binding moiety, an IL-12 binding moiety, and an IL-23 binding moiety, and wherein administration of an effective amount of the composition to a subject in need thereof results in the treatment of inflammatory disease or condition.

Embodiment 37: The pharmaceutical composition of Embodiment 36, wherein the inflammatory disease or condition is selected from the group consisting of: a rheumatoid arthritis, a juvenile arthritis, a psoriatic arthritis, an ankylosing spondylitis, an axial spondyloarthritis, a psoriasis, a hidradenitis suppurativa, an ulcerative colitis, a Crohn's disease, a necrotizing enterocolitis, a sepsis, or a multiple sclerosis

Embodiment 38: A method of treating an inflammatory disease or condition in a subject, the method comprising administering to the subject an effective amount of the engineered protein construct of any one of Embodiments 1-25, the composition of any one of Embodiments 26-33, or the pharmaceutical composition of any one of Embodiments 35-37, thereby treating the disease or condition.

Embodiment 39: The method of Embodiment 38, wherein the inflammatory disease or condition is associated with increased activity and/or expression of TREM1, the interleukin, one or more downstream inflammatory signaling proteins thereof, or combinations thereof relative to a subject not having the inflammatory disease or condition.

Embodiment 40: The method of Embodiment 38 or 39, wherein the method reduces occurrence of candida infection in the subject relative to the subject being treated with a monospecific antibody that reduces interleukin activity.

Embodiment 41: A method of reducing an IL-1 associated inflammatory condition in a subject comprising administering to the subject an effective amount of a pharmaceutical composition comprising an IL-1 binding moiety, a TREM1 binding moiety, and a pharmaceutically acceptable carrier, thereby reducing IL-1 associated inflammatory condition in the subject relative to the IL-1 associated inflammatory condition in the subject prior to administration of the pharmaceutical composition.

Embodiment 42: A method of reducing an IL-6 associated inflammatory condition in a subject comprising administering to the subject an effective amount of a pharmaceutical composition comprising an IL-6 binding moiety, a TREM1 binding moiety and a pharmaceutically acceptable carrier, thereby reducing IL-6 associated inflammatory condition in the subject relative to the IL-6 associated inflammatory condition in the subject prior to administration of the pharmaceutical composition.

Embodiment 43: A method of reducing an IL-12 associated inflammatory condition in a subject comprising administering to the subject an effective amount of a pharmaceutical composition comprising an IL-12 binding moiety, a TREM1 binding moiety, and a pharmaceutically acceptable carrier, thereby reducing IL-12 associated inflammatory condition in the subject relative to the IL-12 associated inflammatory condition in the subject prior to administration of the pharmaceutical composition.

Embodiment 44: A method of reducing an IL-23 associated inflammatory condition in a subject comprising administering to the subject an effective amount of a pharmaceutical composition comprising an IL-1 binding moiety, a TREM1 binding moiety, and a pharmaceutically acceptable carrier, thereby reducing IL-23 associated inflammatory condition in the subject relative to the IL-23 associated inflammatory condition in the subject prior to administration of the pharmaceutical composition.

Embodiment 45: A method of reducing a TREM1 associated inflammatory condition in a subject comprising administering to the subject an effective amount of a pharmaceutical composition comprising a TREM1 binding moiety, an interleukin binding moiety, and a pharmaceutically acceptable carrier, thereby reducing TREM1 associated inflammatory condition in the subject relative to the TREM1 associated inflammatory condition in the subject prior to administration of the pharmaceutical composition.

Embodiment 46: The method of any one of Embodiments 38-45, wherein the method increases expression of at least one of nicotinamide phosphoribosyltransferase (NAMPT), dehydrogenase/reductase 9 (DHRS9), cyclin dependent kinase inhibitor 1A (CDKN1A), CD52 molecule (CD52), Myotubularin related protein 11 (MTMR11), EH domain containing 1 (EHD1), solute carrier family 27 member 3 (SLC27A3), Interleukin 24 (IL24), Pim-2 proto-oncogene serine/threonine kinase (PIM2), chitinase 3 like 1 (CHI3L1), polypeptide N-acetylgalactosaminyltransferase 6 (GALNT6), acyl-CoA thioesterase 7 (ACOT7), cytokine inducible SH2 containing protein (CISH), family with sequence similarity 129 member A (FAM129A), polo like kinase 3 (PLK3), major facilitator superfamily domain containing 12 (MFSD12), StAR related lipid transfer domain containing 4 (STARD4), c-type lectin domain family 12 member A (CLEC12A), CD55 molecule (Cromer blood group) (CD55), and Interferon lambda receptor 1 (IFNLR1).

Embodiment 47: The method of any one of Embodiments 38-46, wherein the method restores pentose phosphate pathway (PPP).

Embodiment 48: The method of any one of Embodiments 38-47, wherein the method restores pentose phosphate pathway (PPP).

### EXAMPLES

### Example 1. Bispecific Antibody Constructs

Bispecific antibody capable of binding two different targets are generated. The two different targets are (a) a protein selected from any one of IL-1 family, IL-6 family, IL-12 family and IL-23 family, a variant thereof or a functional fragment thereof, and (b) TREM1, a variant thereof or a functional fragment thereof. The bispecific antibody comprises a first target binding domain and a second target binding domain. The first target binding domain comprises a first VH sequence and a first VL sequence, wherein the first target binding domain is capable of binding to the protein from any one of IL-1 family, IL-6 family, IL-12 family and IL-23 family, a variant thereof or a functional fragment thereof. The second target binding domain comprises a second VH sequence and a second VL sequence, wherein the second target binding domain is capable of binding TREM1, a variant thereof or a functional fragment thereof. The first VH sequence comprises any one of amino acid sequences of TABLE 3, TABLE 10, TABLE 17 and TABLE 24. The first VL sequence comprises any one of amino acid sequences of TABLE 6, TABLE 13, TABLE 20 and TABLE 27. The second VH sequence comprises any one of amino acid sequences of TABLE 8. The second VL sequence comprises any one of amino acid sequences of TABLE 10.

### Example 2. Proof of Concept Phase 1 Trial for a Bispecific Antibody

Bispecific antibody capable of binding two different targets are generated as described in Example 1. Briefly, the two different targets are (a) a protein selected from any one of IL-1 family, IL-6 family, IL-12 family and IL-23 family, a variant thereof or a functional fragment thereof, and (b) TREM1, a variant thereof or a functional fragment thereof. The bispecific antibody is administered to a subject in need thereof. The subject is selected according to criteria described in TABLE 40.

TABLE 40
Eligibility Criteria:
Ages Eligible for Study: 18 Years to 80 Years (Adult, Older Adult)
Sexes Eligible for Study: All
Accepts Healthy Volunteers: No
Inclusion Criteria:
18-80 years age
Recent onset of autoimmune condition and have known doctor diagnosis ≤1 years and
symptoms for ≤2 years.
Clinically stable with no significant changes in health status within 2 weeks prior to
randomization
Exclusion Criteria:
Presence of active infection
History of chronic viral infections including Hepatitis B or C or HIV. Treated Hepatitis C is
allowed if the viral in non-detectable
Known chronic liver disease
Pregnant, breastfeeding, or desire to become pregnant or unwilling to practice birth control
during participation in the study and for twelve months after completing the study infusion,
unless surgically sterilized or postmenopausal during the study.
Active tuberculosis (TB) requiring treatment within 3 years prior to baseline
Latent TB diagnosed during screening that has not been appropriately treated
Chronic obstructive pulmonary disease or known lung disease except for mild asthma treated
with bronchodilators.
Use of an investigational agent within the 4-week period prior to screen
If Dimethyl sulfoxide (DMSO) is used in the preparation of MSCs then subjects with known
sensitivity to DMSO will be excluded
History of Transient Ischemic Attack
History of Cerebrovascular Accident (stroke)
Clinically significant heart disease (New York Heart Association, class III and class IV).

### Example 3. Effect of a Combination of Engineered Protein Molecules on Inflammatory Biomarkers

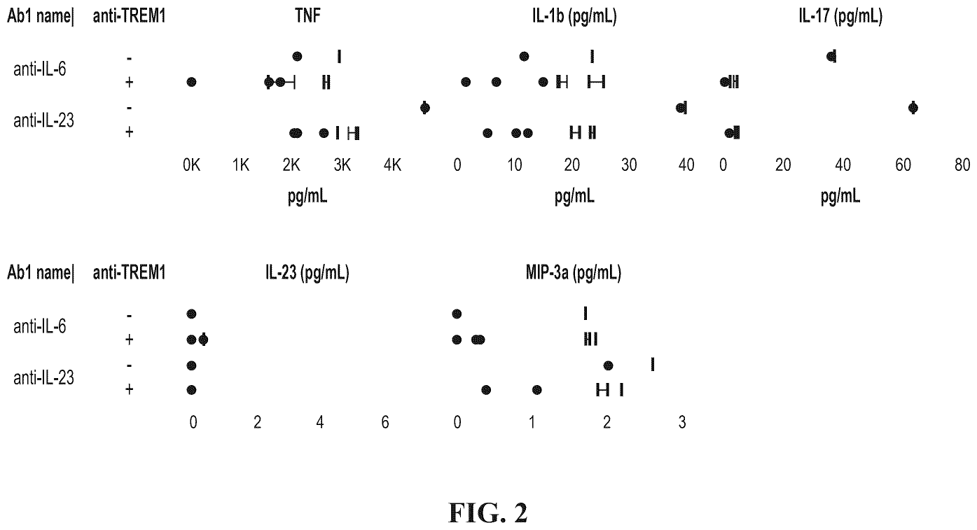

To determine activity of potential advantageous anti-inflammatory activity of a combination of engineered protein molecules, human peripheral blood mononuclear cells (PBMC) were used. Briefly, PBMC were stimulated for TREM1 activation with peptidoglycan recognition protein 1 (PGLYRP1) and peptidoglycan (PGN) at a concentration of 0.1 μg/ml and 0.3 μg/ml (at a ratio of 1:3), respectively. The PBMCs were then incubated with anti-cytokine (anti-CD3) at a concentration of 0.5 μg/ml for T cell activation. The stimulated cells were then incubated with a combination of an engineered TREM1 binding protein molecule and an engineered interleukin (IL-6 or IL-23) binding protein molecule at a concentration of 50 nM. The engineered IL-6 binding protein molecule comprised a VH sequence of SEQ ID NO: 134, and a VL sequence of SEQ ID NO: 207. The engineered IL-23 binding protein molecule comprised a VH sequence of SEQ ID NO: 288, and a VL sequence of SEQ ID NO: 354. The cells were then centrifuged, and supernatant was analyzed for the presence of tumor necrosis factor α (TNFα), IL-1β, IL-17, IL-23 and Macrophage Inflammatory Protein-3 Alpha (MIP-3a). The results of the presence of biomarker are shown in FIG. 2. For negative control, unstimulated cells were used.

### Example 4. Bispecific Antibody

### Bispecific Matrix Generation

Four different bispecific candidates are prepared each comprising a TREM-1 binding domain and an interleukin binding domain selected from an IL-1 binding domain, an IL-6 binding domain, an IL-12 binding domain and an IL-23 binding domain. Briefly, a nucleotide sequences encoding the TREM-1 binding domain and each of the four-interleukin binding domain is synthesized. The TREM-1 binding domain comprises a TREM-1 heavy chain sequence and a TREM-1 light chain sequence. The IL-1 binding domain comprises an IL-1 heavy chain sequence. The IL-6 binding domain comprises an IL-6 heavy chain sequence. The IL-12 binding domain comprises an IL-12 heavy chain sequence. The IL-23 binding domain comprises an IL-23 heavy chain sequence. Knob and Hole technology is used for designing the constructs. The nucleotide sequences are, optionally, codon optimized.

All constructs are cloned using Gibson Assembly via the NEBuilder HiFi DNA Assembly Cloning Kit (NEB). After Gibson Assembly the constructs are transformed using DH5a competent cells (NEB) and plated onto LB-CARB trays (Teknova) with growth o/n@37 C shaking. Single colonies are then picked from each construct on the LB-CARB trays for inoculation into LB-CARB Media (Teknova, prepared fresh) and grown o/n@37 C shaking in 15 mL falcon tubes, seeded at 5 mL LB-CARB media. The cultured cells tubes are then pelleted, discarding the media and plasmid purification is initiated using the Qiagen plasmid plus midiprep purification kits (for yield), using a vacuum manifold and centrifugation. Plasmid DNA is then confirmed for concentration (on the Nanodrop) and nucleotide sequences are confirmed by Sanger Sequencing.

### Bispecific Expression & Purification

Unique heavy and light chain pairs are cloned into vectors designed to express bispecifics and relevant controls in HEK293 cells under the control of a CMV promoter. Antibody expression vectors are complexed with polyethylenimine and transfected into HEK293 cultures. Knob and Hole technology is used to enrich for heterodimerization, and disfavor homodimerization, during expression. After 5 days of shaking at 37 C in 293 cell culture media, cultures are harvested and the supernatant containing the secreted antibodies is clarified and filtered. Antibodies are captured out of the supernatant via an agarose-based protein A resin on a FPLC. After several washes with PBS, antibodies are eluted in a phosphoric acid and saline solution (pH 3) and neutralized with a basic phosphate saline solution (pH 11) to pH˜5.5. Analytical SEC and SDS PAGE analysis are used for determining the optimal fractions from each protein A elution to move forward into secondary/polishing purification.

Automated cation exchange chromatography (CEX), again on the FPLC, using a bind and elute methodology is employed to further enrich the samples for heterodimer bispecifics or controls. After dilution, to lower the salt concentration, a strong cation exchange column (Capto S ImpACT) is used for capturing the material of interest in the protein A elute, followed by washing and elution with increasing levels of sodium chloride at a constant pH of 6.5. The combination of analytic SEC and SDS PAGE is used for identifying fractions from CEX that are suitable for pooling. Following pooling, samples are sterilized, and assayed for purity via analytical SEC and endotoxin by LAL test from Charles River.

### Bispecific Activity

To determine activity of the bispecific antibodies, human peripheral blood mononuclear cells (PBMC) are used. Briefly, PBMC are stimulated for TREM1 activation with peptidoglycan recognition protein 1 (PGLYRP1) and peptidoglycan (PGN) at a concentration of 0.1 μg/ml and 0.3 μg/ml (at a ratio of 1:3), respectively. The PBMCs are then incubated with anti-cytokine (anti-CD3) at a concentration of 0.5 μg/ml for T cell activation. The stimulated cells are then incubated with bispecific antibody candidates at a concentration of 50 nM. The cells are then centrifuged, and supernatant is analyzed for the presence of tumor necrosis factor α (TNFα), IL-1β, IL-17, IL-23 and Macrophage Inflammatory Protein-3 Alpha (MIP-3α). For negative control, unstimulated cells are used. The stimulated isotype cells are used as a control, where activity is measured in the presence of a non-targeting antibody containing a similar Fc-silencing mutation as the bispecific candidates.

What is claimed is:

1. A method of treating inflammatory bowel disease in a subject in need thereof, the method comprising:
administering to the subject in need thereof an effective amount of a pharmaceutical composition, wherein the pharmaceutical composition comprises an engineered protein construct, wherein the engineered protein construct comprises:
(a) a TREM1 binding heavy chain variable (VH) domain;
(b) an IL-23 binding heavy chain variable (VH) domain; and
(c) a heterodimeric Fc region that comprises a first constant region and a second constant region,
wherein the effective amount of the pharmaceutical composition is sufficient to treat inflammatory bowel disease in the subject in need thereof.

2. The method of claim 1, wherein the heterodimeric Fc region is operably linked to the TREM1 binding heavy chain variable (VH) domain and the IL-23 binding heavy chain variable (VH) domain.

3. The method of claim 1, wherein the first constant region and the second constant region each independently comprise an amino acid substitution selected from: a L234A amino acid substitution, a L235A amino acid substitution, a P329A amino substitution, or a combination thereof, per EU numbering relative to a corresponding IgG1 constant region sequence of SEQ ID NO: 453.

4. The method of claim 1, wherein the first constant region and the second constant region each independently comprise an amino acid substitution selected from: a M252Y amino acid substitution, a S254T amino acid substitution, a T256E amino acid substitution, or a combination thereof, per EU numbering relative to a corresponding IgG1 constant region sequence of SEQ ID NO: 453.

5. The method of claim 1, wherein:
the first constant region comprises one or more amino acid substitutions selected from: a Y349C amino acid substitution, a T366S amino acid substitution, a Y407V amino acid substitution, or a combination thereof, per EU numbering relative to a corresponding IgG1 constant region sequence of SEQ ID NO: 453; and
the second constant region comprises one or more amino acid substitutions selected from a S354C amino acid substitution, a T366W amino acid substitution, or a combination thereof, per EU numbering relative to a corresponding IgG1 constant region sequence of SEQ ID NO: 453.

6. The method of claim 1, wherein the first constant region and the second constant region each independently comprise at least one mutation that decreases binding affinity of the engineered protein construct to an FcRn relative to a binding affinity to an FcRn of a corresponding IgG1 constant region comprising SEQ ID NO: 453.

7. The method of claim 1, wherein the first constant region and the second constant region each independently comprise at least one mutation that increases isoelectric point of the engineered protein construct relative to an isoelectric point of a corresponding IgG1 constant region comprising SEQ ID NO: 453.

8. The method of claim 1, wherein upon binding to TREM1, the TREM1 binding VH domain inhibits binding of one or more TREM1 ligands to TREM1.

9. The method of claim 1, wherein under acidic pH conditions, the TREM1 binding VH domain dissociates from TREM1 and soluble TREM1, thereby facilitating recycling of the engineered protein construct into a plasma of a cell from endosomes.

10. The method of claim 1, wherein the engineered protein construct:
(a) directly inhibits TREM1 activity by binding to TREM1;
(b) indirectly inhibits TREM1 activity by reducing TREM1 expression by reducing IL-17 activity; or
(c) a combination thereof.

11. The method of claim 1, wherein administering the pharmaceutical composition increases anti-inflammatory activity in the subject in need thereof as compared to a subject that has been administered a combination of a monospecific antibody that binds to TREM1 and a monospecific antibody that binds to IL-23.

12. The method of claim 1, wherein administering the pharmaceutical composition reduces adverse effects in the subject in need thereof as compared to a subject that has been administered a combination of a monospecific antibody that binds to TREM1 and a monospecific antibody that binds IL-23.

13. The method of claim 1, wherein administering the pharmaceutical composition reduces the incidence of a Candida infection in the subject in need thereof as compared to a subject that has been administered a combination of a monospecific antibody that binds to TREM1 and a monospecific antibody that binds to IL-23.

14. The method of claim 1, wherein the pharmaceutical composition is formulated for oral delivery, subcutaneous delivery or intravenous delivery.

15. The method of claim 1, wherein the inflammatory bowel disease comprises Crohn's disease or ulcerative colitis.

16. The method of claim 1, wherein the engineered protein construct comprises a target sweeping activity for removing TREM1, IL-23, or both TREM1 and IL-23 from plasma of a cell.

17. The method of claim 1, wherein the subject in need thereof advantageously tolerates a higher effective amount of the pharmaceutical composition as compared to a combination of a monospecific antibody that binds to TREM1 and a monospecific antibody that binds to IL-23.

18. The method of claim 1, wherein a binding affinity of the engineered protein construct for a TREM1 epitope is higher than a binding affinity for IL-23, as determined by an in vitro assay.

19. The method of claim 1, wherein a binding affinity of the engineered protein construct for a TREM1 epitope is lower than a binding affinity for IL-23, as determined by an in vitro assay.

20. A method of treating at least one symptom associated with an inflammatory condition in a subject in need thereof, the method comprising:
administering to the subject in need thereof an effective amount of a pharmaceutical composition comprising an engineered protein construct, wherein the engineered protein construct comprises:
(a) a TREM1 binding heavy chain variable (VH) domain;
(b) an IL-23 binding heavy chain variable (VH) domain; and
(c) a heterodimeric Fc region that comprises a first constant region and a second constant region,
wherein the effective amount is sufficient to treat the inflammatory condition in the subject in need thereof,
and wherein the one or more symptoms associated with the inflammatory condition comprise: headache, nausea, vomiting, rectal bleeding, diarrhea, and back pain.


SEQUENCE LISTING (1005 sequences)

SEQ ID NO: 1 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 2 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 3 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 4 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 5 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 6 - protein, 8 aa, synthetic construct
GFTFSMFG

SEQ ID NO: 7 - protein, 8 aa, synthetic construct
GFIFSRYD

SEQ ID NO: 8 - protein, 8 aa, synthetic construct
GGRFTNYA

SEQ ID NO: 9 - protein, 8 aa, synthetic construct
GFTFSVYG

SEQ ID NO: 10 - protein, 10 aa, synthetic construct
GFSLSTSGMG

SEQ ID NO: 11 - protein, 8 aa, synthetic construct
GYSFTNYW

SEQ ID NO: 12 - protein, 8 aa, synthetic construct
GFTFSIYD

SEQ ID NO: 13 - protein, 8 aa, synthetic construct
GYTFTNYW

SEQ ID NO: 14 - protein, 8 aa, synthetic construct
GYSFTSSW

SEQ ID NO: 15 - protein, 8 aa, synthetic construct
GYAFTSSW

SEQ ID NO: 16 - protein, 8 aa, synthetic construct
VSYDGSNK

SEQ ID NO: 17 - protein, 8 aa, synthetic construct
ISHGGAGT

SEQ ID NO: 18 - protein, 8 aa, synthetic construct
IIPIFDET

SEQ ID NO: 19 - protein, 8 aa, synthetic construct
IWYDGDNQ

SEQ ID NO: 20 - protein, 7 aa, synthetic construct
IWWDGDE

SEQ ID NO: 21 - protein, 8 aa, synthetic construct
IYPGNSDT

SEQ ID NO: 22 - protein, 8 aa, synthetic construct
IRGEGGGT

SEQ ID NO: 23 - protein, 8 aa, synthetic construct
FHPTGDVT

SEQ ID NO: 24 - protein, 8 aa, synthetic construct
INPGNVRT

SEQ ID NO: 25 - protein, 8 aa, synthetic construct
IYPGDGNT

SEQ ID NO: 26 - protein, 15 aa, synthetic construct
ARGRPKVVIPAPLAH

SEQ ID NO: 27 - protein, 12 aa, synthetic construct
ARGGVTKGYFDV

SEQ ID NO: 28 - protein, 11 aa, synthetic construct
ATGSNSYYGLY

SEQ ID NO: 29 - protein, 11 aa, synthetic construct
ARDLRTGPFDY

SEQ ID NO: 30 - protein, 12 aa, synthetic construct
ARNRYDPPWFVD

SEQ ID NO: 31 - protein, 14 aa, synthetic construct
ARHGTSSDYYGLDV

SEQ ID NO: 32 - protein, 16 aa, synthetic construct
ARDPWSTEGSFFVLDY

SEQ ID NO: 33 - protein, 13 aa, synthetic construct
ARTTSMIIGGFAY

SEQ ID NO: 34 - protein, 12 aa, synthetic construct
TVVFYGEPYFPY

SEQ ID NO: 35 - protein, 10 aa, synthetic construct
GEGYLDPMDY

SEQ ID NO: 36 - protein, 122 aa, synthetic construct
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 37 - protein, 119 aa, synthetic construct
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSS

SEQ ID NO: 38 - protein, 118 aa, synthetic construct
QVQLVQSGAEVKKPGSSVKVSCKASGGRFTNYAILWVRQAPGQGLQWLGGIIPIFDETDHAQDFQDRLTITVDESMTTAYMELSSLRPEDTAIYYCATGSNSYYGLYWGQGTLVTVSS

SEQ ID NO: 39 - protein, 118 aa, synthetic construct [sequence omitted]

SEQ ID NO: 40 - protein, 120 aa, synthetic construct [sequence omitted]

SEQ ID NO: 41 - protein, 121 aa, synthetic construct
EVQLVQSGAEVKKPGESLKISCKGSGYSFTNYWIGWVRQMPGKGLEWMGIIYPGNSDTRFSPSFQGQVTISADKSITTAYLQWSSLKASDTAMYYCARHGTSSDYYGLDVWGQGTTVTVSS

SEQ ID NO: 42 - protein, 123 aa, synthetic construct
EVQLLESGGGLVQPGGSLRLSCAASGFTFSIYDMIWVRQAPGKGLEWVSSIRGEGGGTYYADSVKGRFTISRDNSKNTLYLQMNSLRAEDTAVYYCARDPWSTEGSFFVLDYWGQGTLVTVSS

SEQ ID NO: 43 - protein, 120 aa, synthetic construct
QVQLVQSGAEVKKPGASVKVSCKASGYTFTNYWMNWVRQAPRQGLEWMGMFHPTGDVTRLNQKFKDRVTMTRDTSTSTVYMELSSLRSEDTAVYYCARTTSMIIGGFAYWGQGTLVTVSS

SEQ ID NO: 44 - protein, 119 aa, synthetic construct [sequence omitted]

SEQ ID NO: 45 - protein, 117 aa, synthetic construct
QVQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVTVSS

SEQ ID NO: 46 - protein, 6 aa, synthetic construct
QGISSW

SEQ ID NO: 47 - protein, 6 aa, synthetic construct
GNIHNY

SEQ ID NO: 48 - protein, 12 aa, synthetic construct
QSVLYSSNNKNY

SEQ ID NO: 49 - protein, 6 aa, synthetic construct
QSIGSS

SEQ ID NO: 50 - protein, 6 aa, synthetic construct
QDISNY

SEQ ID NO: 51 - protein, 6 aa, synthetic construct
QSVDDD

SEQ ID NO: 52 - protein, 11 aa, synthetic construct
KSLLHRNAITY

SEQ ID NO: 53 - protein, 7 aa, synthetic construct
SSVSSSY

SEQ ID NO: 54 - protein, 6 aa, synthetic construct
QGINNY

SEQ ID NO: 55-62 - skipped (8 entries)

SEQ ID NO: 63 - protein, 9 aa, synthetic construct
QQTSSFLLS

SEQ ID NO: 64 - protein, 9 aa, synthetic construct
QHFWSIPYT

SEQ ID NO: 65 - protein, 9 aa, synthetic construct
QQYYSTPST

SEQ ID NO: 66 - protein, 9 aa, synthetic construct
HQSSSLPFT

SEQ ID NO: 67 - protein, 9 aa, synthetic construct
LQGKMLPWT

SEQ ID NO: 68 - protein, 9 aa, synthetic construct
QQDDNFPLT

SEQ ID NO: 69 - protein, 9 aa, synthetic construct
QQYITAPLT

SEQ ID NO: 70 - protein, 9 aa, synthetic construct
AQNLELPLT

SEQ ID NO: 71 - protein, 9 aa, synthetic construct
HQFHRSPLT

SEQ ID NO: 72 - protein, 9 aa, synthetic construct
QQYSILPWT

SEQ ID NO: 73 - protein, 107 aa, synthetic construct
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHK

SEQ ID NO: 74 - protein, 107 aa, synthetic construct
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQIT

SEQ ID NO: 75 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPSTFGQGTKVEIK

SEQ ID NO: 76 - protein, 107 aa, synthetic construct [sequence omitted]

SEQ ID NO: 77 - protein, 107 aa, synthetic construct [sequence omitted]

SEQ ID NO: 78 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDDNFPLTFGGGTKVEIK

SEQ ID NO: 79 - protein, 107 aa, synthetic construct
EIVLTQSPATLSLSPGERATLSCRASQSVDDDLAWYQQKPGQAPRLLIYDASNRATGIPARFSGSGSGTDFTLTISSLEPEDFAVYYCQQYITAPLTFGQGTKVEIK

SEQ ID NO: 80 - protein, 112 aa, synthetic construct
DIVMTQTPLSLSVTPGQPASISCRSSKSLLHRNAITYFYWYLHKPGQPPQLLIYQMSNLASGVPDRFSGSGSGTDFTLKISRVEAEDVGVYYCAQNLELPLTFGGGTKVEIK

SEQ ID NO: 81 - protein, 108 aa, synthetic construct [sequence omitted]

SEQ ID NO: 82 - protein, 107 aa, synthetic construct
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

SEQ ID NO: 83 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 84 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 85 - protein, 9 aa, synthetic construct
GFSLSNYYV

SEQ ID NO: 86 - protein, 8 aa, synthetic construct
GFNFNDYF

SEQ ID NO: 87 - protein, 8 aa, synthetic construct
GFTFSSFA

SEQ ID NO: 88 - protein, 8 aa, synthetic construct
GFTFSPFA

SEQ ID NO: 89 - protein, 8 aa, synthetic construct
GYVLPNYL

SEQ ID NO: 90 - protein, 8 aa, synthetic construct
GFTISSNY

SEQ ID NO: 91 - protein, 5 aa, synthetic construct
SNYMI

SEQ ID NO: 92 - protein, 5 aa, synthetic construct
SNYMT

SEQ ID NO: 93 - protein, 5 aa, synthetic construct
SNYMV

SEQ ID NO: 94 - protein, 8 aa, synthetic construct
GFTFSSYY

SEQ ID NO: 95 - protein, 8 aa, synthetic construct
RFTFDDYA

SEQ ID NO: 96 - protein, 9 aa, synthetic construct
GHSISHDHA

SEQ ID NO: 97 - protein, 7 aa, synthetic construct
IYGSDET

SEQ ID NO: 98 - protein, 10 aa, synthetic construct
MRNKNYQYGT

SEQ ID NO: 99 - protein, 8 aa, synthetic construct
ISSGGSYT

SEQ ID NO: 100 - protein, 8 aa, synthetic construct
ISPGGSWT

SEQ ID NO: 101 - protein, 8 aa, synthetic construct
TTPGGGTI

SEQ ID NO: 102 - protein, 8 aa, synthetic construct
LYYYAGDT

SEQ ID NO: 103 - protein, 17 aa, synthetic construct
DLYYYAGDTYYADSVKG

SEQ ID NO: 104 - protein, 17 aa, synthetic construct
DLYYYAGDTYYADSVRG

SEQ ID NO: 105 - protein, 8 aa, synthetic construct
IYSDGTTH

SEQ ID NO: 106 - protein, 8 aa, synthetic construct
ISWNSGRI

SEQ ID NO: 107 - protein, 8 aa, synthetic construct
FISYSGIT

SEQ ID NO: 108 - protein, 14 aa, synthetic construct
ARDDSSDWDAKFNL

SEQ ID NO: 109 - protein, 11 aa, synthetic construct
ARESYYGFTSY

SEQ ID NO: 110 - protein, 12 aa, synthetic construct
ARGLWGYYALDY

SEQ ID NO: 111 - protein, 12 aa, synthetic construct
ARQLWGYYALDI

SEQ ID NO: 112 - protein, 15 aa, synthetic construct
ARSRWDPLYYYALEY

SEQ ID NO: 113 - protein, 13 aa, synthetic construct
ARWADDHPPWIDL

SEQ ID NO: 114 - protein, 11 aa, synthetic construct
WADDHYYYIDV

SEQ ID NO: 115 - protein, 11 aa, synthetic construct
WADGHYYYIDV

SEQ ID NO: 116 - protein, 11 aa, synthetic construct
WADDHYYHIDV

SEQ ID NO: 117 - protein, 11 aa, synthetic construct
WADGHYYYADV

SEQ ID NO: 118 - protein, 11 aa, synthetic construct
WADDHPAWVDL

SEQ ID NO: 119 - protein, 11 aa, synthetic construct
WADDHPRYIDH

SEQ ID NO: 120 - protein, 11 aa, synthetic construct
WEEEGRGYIDV

SEQ ID NO: 121 - protein, 12 aa, synthetic construct
WADDHNYPHIDV

SEQ ID NO: 122 - protein, 11 aa, synthetic construct
WADDHPPYIDL

SEQ ID NO: 123 - protein, 11 aa, synthetic construct
WADDHPPYIDM

SEQ ID NO: 124 - protein, 11 aa, synthetic construct
WADDHPPWIDL

SEQ ID NO: 125 - protein, 11 aa, synthetic construct
WADDHPSHLDI

SEQ ID NO: 126 - protein, 11 aa, synthetic construct
WADDHPSHIDV

SEQ ID NO: 127 - protein, 12 aa, synthetic construct
WADDHNNTYIDV

SEQ ID NO: 128 - protein, 11 aa, synthetic construct
WADDHAPWVDL

SEQ ID NO: 129 - protein, 14 aa, synthetic construct
AKGAGPTWWYALDA

SEQ ID NO: 130 - protein, 9 aa, synthetic construct
AKGRDSFDI

SEQ ID NO: 131 - protein, 12 aa, synthetic construct
ARSLARTTAMDY

SEQ ID NO: 132 - protein, 120 aa, synthetic construct
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 133 - protein, 120 aa, synthetic construct [sequence omitted]

SEQ ID NO: 134 - protein, 119 aa, synthetic construct [sequence omitted]

SEQ ID NO: 135 - protein, 119 aa, synthetic construct
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 136 - protein, 122 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSS

SEQ ID NO: 137 - protein, 120 aa, synthetic construct
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSS

SEQ ID NO: 138 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHYYYIDVWGRGTLVTVSS

SEQ ID NO: 139 - protein, 120 aa, synthetic construct
EVQLVQSGGGLIQPGGSLRLSCAASGFTISSNYMTWVRQAPGKGLEWVSDLYYYAGDTYYADSVRGRFTMSRDISKNTVYLQMDSLRAEDTGVYYCARWADGHYYYIDVWGGGTLVTVSS

SEQ ID NO: 140 - protein, 120 aa, synthetic construct
QVQLVQSGGGLIQPGGSLRLSCAASGFTISSNYMVWVRQAPGKGLEWVSDLYYYAGDTYYADSVKGRFTVSRDISKNTVYLQMNSLRAEDTGVYYCARWADDHYYHIDVWGRGTLVTVSS

SEQ ID NO: 141 - protein, 120 aa, synthetic construct
EVQLVQSGGGLIQPGGSLRLSCAASGFTVSSNYMTWVRQAPGKGLEWVSDLYYYAGDTYYADSVKGRFTMSRDISKNTVYLQMNSLRAEDTGVYYCARWADGHYYYADVWGRGTLVSVSS

SEQ ID NO: 142 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADGHYYYADVWGRGTLVSVSS

SEQ ID NO: 143 - protein, 120 aa, synthetic construct
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPAWVDLWGRGTLVTVSS

SEQ ID NO: 144 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHPRYIDHWGRGTLVTVSS

SEQ ID NO: 145 - protein, 120 aa, synthetic construct
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWEEEGRGYIDVWGRGTLVTVSS

SEQ ID NO: 146 - protein, 121 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHNYPHIDVWGRGTLVTVSS

SEQ ID NO: 147 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHPPYIDLWGRGTLVTVSS

SEQ ID NO: 148 - protein, 120 aa, synthetic construct
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPYIDMWGRGTLVTVSS

SEQ ID NO: 149 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHPSHLDIWGRGTLVTVSS

SEQ ID NO: 150 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHPSHIDVWGRGTLVTVSS

SEQ ID NO: 151 - protein, 121 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHNNTYIDVWGRGTLVTVSS

SEQ ID NO: 152 - protein, 120 aa, synthetic construct
EVQLVQSGGGLIQPGGSLRLSCAASGFTISSNYMIWVRQAPGKGLEWVSDLYYYAGDTYYADSVKGRFTMSRDISKNTVYLQMNSLRAEDTGVYYCARWADDHAPWVDLWGRGTLVTVSS

SEQ ID NO: 153 - protein, 119 aa, synthetic construct
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVS

SEQ ID NO: 154 - protein, 121 aa, synthetic construct [sequence omitted]

SEQ ID NO: 155 - protein, 120 aa, synthetic construct
QVQLVQSGGGLVQPGGSLRLSCAASGFTFSSYYMSWVRQAPGKGLEWVSGIYSDGTTHYGDSVKGRFTISRDNAKNTVYLQLNSLRAEDTAMYYCAKGAGPTWWYALDAWGQGTLVTVSS

SEQ ID NO: 156 - protein, 116 aa, synthetic construct
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 157 - protein, 119 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 158 - protein, 119 aa, synthetic construct [sequence omitted]

SEQ ID NO: 159 - protein, 6 aa, synthetic construct
QSINNE

SEQ ID NO: 160 - protein, 6 aa, synthetic construct
QDIGIS

SEQ ID NO: 161 - protein, 5 aa, synthetic construct
SSVSY

SEQ ID NO: 162 - protein, 5 aa, synthetic construct
ISVSY

SEQ ID NO: 163 - protein, 10 aa, synthetic construct
ESVDNYGIPF

SEQ ID NO: 164 - protein, 6 aa, synthetic construct
QGISSW

SEQ ID NO: 165 - protein, 11 aa, synthetic construct
RASQGISSWLA

SEQ ID NO: 166 - protein, 11 aa, synthetic construct
RASQGISSWLT

SEQ ID NO: 167 - protein, 10 aa, synthetic construct
QSVLSASNTY

SEQ ID NO: 168 - protein, 6 aa, synthetic construct
TDISSH

SEQ ID NO: 169-174 - skipped (6 entries)

SEQ ID NO: 175 - protein, 7 aa, synthetic construct
KASTLES

SEQ ID NO: 176-178 - skipped (3 entries)

SEQ ID NO: 179 - protein, 12 aa, synthetic construct
QQGYSLRNIDNA

SEQ ID NO: 180 - protein, 9 aa, synthetic construct
LQHNSAPYT

SEQ ID NO: 181 - protein, 9 aa, synthetic construct
QQWSGYPYT

SEQ ID NO: 182 - protein, 9 aa, synthetic construct
MQWSGYPYT

SEQ ID NO: 183 - protein, 9 aa, synthetic construct
QQSEEVPLT

SEQ ID NO: 184 - protein, 8 aa, synthetic construct
QQSWLGGS

SEQ ID NO: 185 - protein, 9 aa, synthetic construct
QQSYSTPWT

SEQ ID NO: 186 - protein, 9 aa, synthetic construct
QQSYSAPWT

SEQ ID NO: 187 - protein, 8 aa, synthetic construct
QQSWLGGS

SEQ ID NO: 188 - protein, 9 aa, synthetic construct
AAHYAAPWT

SEQ ID NO: 189 - protein, 9 aa, synthetic construct
QQAYRAPVT

SEQ ID NO: 190 - protein, 9 aa, synthetic construct
QQANSFPYT

SEQ ID NO: 191 - protein, 9 aa, synthetic construct
GQGNRLPYT

SEQ ID NO: 192 - protein, 109 aa, synthetic construct
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 193 - protein, 107 aa, synthetic construct [sequence omitted]

SEQ ID NO: 194 - protein, 105 aa, synthetic construct
IVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIK

SEQ ID NO: 195 - protein, 106 aa, synthetic construct
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 196 - protein, 111 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIK

SEQ ID NO: 197 - protein, 106 aa, synthetic construct
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIK

SEQ ID NO: 198 - protein, 108 aa, synthetic construct
DIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKR

SEQ ID NO: 199 - protein, 108 aa, synthetic construct
DIVMTQSPSTLSASVGDRVTITCRASQGISSWLTWYQQKPGRAPKVLIYKASTLESGVPSRFSGSGSGTDFTLTISSLQPEDFATYYCQQSYSAPWTFGQGTKLELKR

SEQ ID NO: 200 - protein, 108 aa, synthetic construct
DIVMTQSPSTLSASVGDRVTITCRASQGISSWLAWYQQKPGRAPKALIYKASTLESGVPSRFSGSGSGTDFTLTISSLQPEDFATYYCQQSYSTPWTFGQGTKLEIKR

SEQ ID NO: 201 - protein, 108 aa, synthetic construct
DIVMTQSPSTLSASVGDRVTITCRASQGISSWLAWYQQKPGRAPKVLIYKASTLESGVPSRFSGSGSGTDFTLTISSLQPEDFASYYCQQSYSAPWTFGQGTKLELKR

SEQ ID NO: 202 - protein, 108 aa, synthetic construct
DIVMTQSPPTLSASVGDRVTITCRASQGISSWLAWYQQKPGRAPKVLIYKASTLESGVPSRFSGSGSGTDFTLTISSLQPEDFASYYCQQSYSAPWTFGQGTKLEIKR

SEQ ID NO: 203 - protein, 107 aa, synthetic construct
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIKR

SEQ ID NO: 204 - protein, 108 aa, synthetic construct
DIVMTQSPSTLSASVGDRVTITCRASQGISSWLAWYQQKPGRAPKVLIYKASTLESGVPSRFSGSGSGTDFTLTISSLQPEDFATYYCAAHYAAPWTFGQGTKLEIKR

SEQ ID NO: 205 - protein, 107 aa, synthetic construct
DIVMTQSPSTLSASVGDRVTITCRASQGISSWLAWYQQKPGRAPKVLIYKASTLESGVPSRFSGSGSGTDFTLTISSLQPEDFATYYCQQSWLGGSFGQGTKLEIKR

SEQ ID NO: 206 - protein, 110 aa, synthetic construct
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 207 - protein, 106 aa, synthetic construct [sequence omitted]

SEQ ID NO: 208 - protein, 111 aa, synthetic construct
DIQLTQSPSSVSVSVGERVTIDCKSSQSVLSASNTYLNWYQQKPGQAPQLLIYYASTRESGVPDRFSGSGSGTDFTLTISSLQAEDAAVYYCQQAYRAPVTFGQGTKLEIK

SEQ ID NO: 209 - protein, 107 aa, synthetic construct
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 210 - protein, 107 aa, synthetic construct
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIE

SEQ ID NO: 211 - protein, 107 aa, synthetic construct [sequence omitted]

SEQ ID NO: 212 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 213 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 214 - protein, 8 aa, synthetic construct
GFTFSSYG

SEQ ID NO: 215 - protein, 8 aa, synthetic construct
GYTFTSYW

SEQ ID NO: 216 - protein, 8 aa, synthetic construct
GYSFTTYW

SEQ ID NO: 217 - protein, 8 aa, synthetic construct
IRYDGSNK

SEQ ID NO: 218 - protein, 8 aa, synthetic construct
MSPVDSDI

SEQ ID NO: 219 - protein, 8 aa, synthetic construct
KTHGSHDN

SEQ ID NO: 220 - protein, 12 aa, synthetic construct
ARRRPGQGYFDF

SEQ ID NO: 221 - protein, 115 aa, synthetic construct [sequence omitted]

SEQ ID NO: 222 - protein, 119 aa, synthetic construct
EVQLVQSGAEVKKPGESLKISCQSSGYTFTSYWIGWVRQMPGQGLEWIGIMSPVDSDIRYNPMFRGQVTMSVDKSSSTAYLQWSSLKASDTAMYYCARRRPGQGYFDFWGQGTMVTVSS

SEQ ID NO: 223 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 224 - protein, 8 aa, synthetic construct
RSNIGSNT

SEQ ID NO: 225 - protein, 6 aa, synthetic construct
QSVGTW

SEQ ID NO: 226 - protein, 6 aa, synthetic construct
QGISSW

SEQ ID NO: 227-228 - skipped (2 entries)

SEQ ID NO: 229 - protein, 12 aa, synthetic construct
QSYDRYTHPALL

SEQ ID NO: 230 - protein, 9 aa, synthetic construct
QQYNIYPYT

SEQ ID NO: 231 - protein, 111 aa, synthetic construct [sequence omitted]

SEQ ID NO: 232 - protein, 107 aa, synthetic construct
EIVLTQSPATLSASPGERATISCRASQSVGTWVAWYQQKPGQAPRSLIYAASNLQSGIPARFSGSGSGTDFTLTISSLEPEDFAVYYCQQYNIYPYTFGQGTRLEIK

SEQ ID NO: 233 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 234 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 235 - protein, 8 aa, synthetic construct
GFTFSSYG

SEQ ID NO: 236 - protein, 8 aa, synthetic construct
GYSFSNYW

SEQ ID NO: 237 - protein, 8 aa, synthetic construct
GYKFTRYV

SEQ ID NO: 238 - protein, 8 aa, synthetic construct
GYTFTSYL

SEQ ID NO: 239 - protein, 8 aa, synthetic construct
GYTFTDQT

SEQ ID NO: 240 - protein, 8 aa, synthetic construct
GYIFITYW

SEQ ID NO: 241 - protein, 5 aa, synthetic construct
SYGMH

SEQ ID NO: 242 - protein, 5 aa, synthetic construct
SYAMH

SEQ ID NO: 243 - protein, 5 aa, synthetic construct
SYSMN

SEQ ID NO: 244 - protein, 5 aa, synthetic construct
TYSMN

SEQ ID NO: 245 - protein, 5 aa, synthetic construct
SFSMN

SEQ ID NO: 246 - protein, 5 aa, synthetic construct
TYYWS

SEQ ID NO: 247 - protein, 7 aa, synthetic construct
SGGYYWS

SEQ ID NO: 248 - protein, 5 aa, synthetic construct
SYFWS

SEQ ID NO: 249 - protein, 7 aa, synthetic construct
SGGYYWT

SEQ ID NO: 250 - protein, 8 aa, synthetic construct
IWYDGSNE

SEQ ID NO: 251 - protein, 8 aa, synthetic construct
IDPSNSYT

SEQ ID NO: 252 - protein, 8 aa, synthetic construct
INPYNDGT

SEQ ID NO: 253 - protein, 8 aa, synthetic construct
INPYNEGT

SEQ ID NO: 254 - protein, 8 aa, synthetic construct
IYPRDDSP

SEQ ID NO: 255 - protein, 8 aa, synthetic construct
IFPASGSA

SEQ ID NO: 256 - protein, 17 aa, synthetic construct
VIWYDGSNEYYADSVKG

SEQ ID NO: 257 - protein, 17 aa, synthetic construct
VIWYDGSNKYYADSVKG

SEQ ID NO: 258 - protein, 17 aa, synthetic construct
VISFDGSLKYYADSVKG

SEQ ID NO: 259 - protein, 17 aa, synthetic construct
VISHDGSIKYYADSVKG

SEQ ID NO: 260 - protein, 17 aa, synthetic construct
YISSRSSTIYIADSVKG

SEQ ID NO: 261 - protein, 17 aa, synthetic construct
YISSSSSTRYHADSVKG

SEQ ID NO: 262 - protein, 17 aa, synthetic construct
YISSRSSTIYYADSVKG

SEQ ID NO: 263 - protein, 16 aa, synthetic construct
LIYTSGSTNYNPSLKS

SEQ ID NO: 264 - protein, 16 aa, synthetic construct
HIHYSGNTYYNPSLKS

SEQ ID NO: 265 - protein, 16 aa, synthetic construct
YIYYSGSSYYNPSLKS

SEQ ID NO: 266 - protein, 16 aa, synthetic construct
YIYYSGSTYYNPSLKS

SEQ ID NO: 267 - protein, 16 aa, synthetic construct
YIYYSGSTNYNPSLKS

SEQ ID NO: 268 - protein, 16 aa, synthetic construct
YIYYSGNTYYNPSLKS

SEQ ID NO: 269 - protein, 17 aa, synthetic construct
LIWYDGSNKYYADSVKG

SEQ ID NO: 270 - protein, 17 aa, synthetic construct
ARDRGYTSSWYPDAFDI

SEQ ID NO: 271 - protein, 10 aa, synthetic construct
ARWYYKPFDV

SEQ ID NO: 272 - protein, 8 aa, synthetic construct
ARNWDTGL

SEQ ID NO: 273 - protein, 8 aa, synthetic construct
ARNWDLPY

SEQ ID NO: 274 - protein, 13 aa, synthetic construct
AIPDRSGYAWFIY

SEQ ID NO: 275 - protein, 9 aa, synthetic construct
ARGGGGFAY

SEQ ID NO: 276 - protein, 12 aa, synthetic construct
DRGYTSSWYPDA

SEQ ID NO: 277 - protein, 14 aa, synthetic construct
DRGYSSSWYPDAFD

SEQ ID NO: 278 - protein, 12 aa, synthetic construct
ERTTLSGSYFDY

SEQ ID NO: 279 - protein, 16 aa, synthetic construct
RIAAAGGFHYYYALDV

SEQ ID NO: 280 - protein, 16 aa, synthetic construct
RIAAAGPWGYYYAMDV

SEQ ID NO: 281 - protein, 10 aa, synthetic construct
DRGYYYGVDV

SEQ ID NO: 282 - protein, 10 aa, synthetic construct
NRGFYYGMDV

SEQ ID NO: 283 - protein, 10 aa, synthetic construct
DRGHYYGMDV

SEQ ID NO: 284 - protein, 10 aa, synthetic construct
DRGSYYGSDY

SEQ ID NO: 285 - protein, 10 aa, synthetic construct
NRGYYYGMDV

SEQ ID NO: 286 - protein, 14 aa, synthetic construct
ENTVTIYYNYGMDV

SEQ ID NO: 287 - protein, 124 aa, synthetic construct
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 288 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 289 - protein, 115 aa, synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSS

SEQ ID NO: 290 - protein, 115 aa, synthetic construct
QVQLVQSGAEVKKPGASVKVSCKASGYTFTSYLMHWVRQAPGQGLEWMGYINPYNEGTNYAQKFQGRVTMTRDTSISTAYMELSRLRSDDTAVYYCARNWDLPYWGQGTLVTVSS

SEQ ID NO: 291 - protein, 120 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 292 - protein, 116 aa, synthetic construct [sequence omitted]

SEQ ID NO: 293 - protein, 123 aa, synthetic construct
QVQLVESGGGWQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 294 - protein, 123 aa, synthetic construct
QVQLVESGGGWQPGRSLRLSCAASGFTFSSYGMHWVRQAPGKGLEWVAVIWYDGSNKYYADSVKGRFTISRDNSKNTLYLQMNSLRAEDTAVYYCARDRGYSSSWYPDAFDIWGQGTMVTVSS

SEQ ID NO: 295 - protein, 120 aa, synthetic construct
QVQLVESGGGWQPGRSLRLSCAASGFTFSSYGMHWVRQAPGKGLEWVAVISFDGSLKYYADSVKGRFTISRDNSKNTLYLQMNSLRAEDTAVYYCARERTTLSGSYFDYWGQGTLVTVSS

SEQ ID NO: 296 - protein, 120 aa, synthetic construct
QVQLVESGGGWQPGRSLRLSCAASGFTFSSYAMHWVRQAPGKGLEWLSVISHDGSIKYYADSVKGRFTISRDNSKNTLYLQMNSLRAEDTAVYYCARERTTLSGSYFDYWGQGTLVTVSS

SEQ ID NO: 297 - protein, 125 aa, synthetic construct
EVQLVESGGGLVQPGGSLRLSCAASGFTFSSYSMNWVRQAPGKGLEWVSYISSRSSTIYIADSVKGRFTISRDNAKNSLYLQMNSLRDEDTAVYYCARRIAAAGGFHYYYALDVWGQGTTVTVSS

SEQ ID NO: 298 - protein, 125 aa, synthetic construct
EVQLVESGGGLVQPGGSLRLSCAASGFTFSTYSMNWVRQAPGKGLEWVSYISSSSSTRYHADSVKGRFTISRDNAKNSLYLQMNSLRDEDTAVYYCARRIAAAGPWGYYYAMDVWGQGTTVTVSS

SEQ ID NO: 299 - protein, 124 aa, synthetic construct
EVQLVESGGGLVQPGGSLRLSCWSGFTFSSFSMNWVRQAPGKGLEWVSYISSRSSTIYYADSVKGRFTISRDNAKNSLYLQMNSLRDEDTAVYYCARRIAAAGPWGYYYAMDVWGQGTTVTVSS

SEQ ID NO: 300 - protein, 118 aa, synthetic construct
QVQLQESGPGLVKPSETLSLTCTVSGGSISTYYWSWIRQPAGKGLEWIGLIYTSGSTNYNPSLKSRVTMSLDTSKNQFSLRLTSVTAADTAVYYCARDRGYYYGVDVWGQGTTVTVSS

SEQ ID NO: 301 - protein, 120 aa, synthetic construct
QVQLQESGPGLVKPSQTLSLTCTVSGGSISSGGYYWSWIRQHPGKGLEWIGHIHYSGNTYYNPSLKSRVTISVDTSKNQFSLKLSSVTAADTAVYYCAKNRGFYYGMDVWGQGTTVTVSS

SEQ ID NO: 302 - protein, 120 aa, synthetic construct
QVQLQESGPGLVKPSQTLSLTCTVSGGSINSGGYYWSWIRQHPGKGLEWIGYIYYSGSSYYNPSLKSRVTISVDTSQNQFSLKLSSVTAADTAVYYCARDRGHYYGMDVWGQGTTVTVSS

SEQ ID NO: 303 - protein, 120 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDRGHYYGMDVWGQGTTVTVSS

SEQ ID NO: 304 - protein, 118 aa, synthetic construct
QVQLQESGPRLVKPSETLSLTCTVSGDSISSYFWSWIRQPPGKGLEWLGYIYYSGSTNYNPSLKSRVTISIDTSKNQFSLKLSSVTAADTAVYYCTRDRGSYYGSDYWGQGTLVTVSS

SEQ ID NO: 305 - protein, 120 aa, synthetic construct
QVQLQESGPGLVKPSQTLSLTCTVSGGSISSGGYYWTWIRQHPGKGLEWIGYIYYSGNTYYNPSLKSRITISVDTSKNQFSLSLSSVTAADTAVYYCARNRGYYYGMDVWGQGTTVTVSS

SEQ ID NO: 306 - protein, 120 aa, synthetic construct
QVQLQESGPGLVKPSQTLSLTCTVSGGSISSGGYYWSWIRQHPGKGLEWIGYIYYSGSTYYNPSLKSRVTMSVDTSKNQFSLKLSSVTAADTAVYYCAKNRGFYYGMDVWGQGTTVTVSS

SEQ ID NO: 307 - protein, 120 aa, synthetic construct
QVQLQESGPGLVKPSQTLSLTCTVSGGSINSGGYYWSWIRQHPGKGLEWIGYIYYSGSSYYNPSLKSRVTISVDTSKNQFSLKLSSVTAADTAVYYCARDRGHYYGMDVWGQGTTVTVSS

SEQ ID NO: 308 - protein, 122 aa, synthetic construct
QVQLVESGGGWQPGRSLRLSCAASGFTFSSYGMHWVRQAPGKGLEWVALIWYDGSNKYYADSVKGRFTISRDNSKNTLYLQMNSLRAEDTAVYYCARENTVTIYYNYGMDVWGQGTTVTVSS

SEQ ID NO: 309 - protein, 9 aa, synthetic construct
SSNTGAGYD

SEQ ID NO: 310 - protein, 9 aa, synthetic construct
SSNIGSGYD

SEQ ID NO: 311 - protein, 6 aa, synthetic construct
DHILKF

SEQ ID NO: 312 - protein, 6 aa, synthetic construct
QSISDY

SEQ ID NO: 313 - protein, 6 aa, synthetic construct
RDVAIA

SEQ ID NO: 314 - protein, 6 aa, synthetic construct
ENIYSY

SEQ ID NO: 315 - protein, 14 aa, synthetic construct
TGSSSNTGAGYDVH

SEQ ID NO: 316 - protein, 14 aa, synthetic construct
TGSSSNIGAGYDVH

SEQ ID NO: 317 - protein, 14 aa, synthetic construct
TLRSGINVGTYRIY

SEQ ID NO: 318 - protein, 12 aa, synthetic construct
TLNSGYSDYKVD

SEQ ID NO: 319 - protein, 12 aa, synthetic construct
TLSSGYSDYKVD

SEQ ID NO: 320 - protein, 11 aa, synthetic construct
RASQGIAGWLA

SEQ ID NO: 321 - protein, 11 aa, synthetic construct
RASQVISSWLA

SEQ ID NO: 322 - protein, 11 aa, synthetic construct
RASQGSSSWFA

SEQ ID NO: 323 - protein, 11 aa, synthetic construct
RASQGISSWFA

SEQ ID NO: 324 - protein, 11 aa, synthetic construct
RAGQVISSWLA

SEQ ID NO: 325 - protein, 11 aa, synthetic construct
RASQGFSGWLA

SEQ ID NO: 326 - protein, 11 aa, synthetic construct
RASQVISSWFA

SEQ ID NO: 327 - protein, 11 aa, synthetic construct
RASQGIRNDLG

SEQ ID NO: 328-333 - skipped (6 entries)

SEQ ID NO: 334 - protein, 7 aa, synthetic construct
GSGNRPS

SEQ ID NO: 335 - protein, 7 aa, synthetic construct
GSNNRPS

SEQ ID NO: 336 - protein, 11 aa, synthetic construct
YKSDSDKQQGS

SEQ ID NO: 337 - protein, 12 aa, synthetic construct
VGTGGIVGSKGD

SEQ ID NO: 338 - protein, 12 aa, synthetic construct
VGTGGIVGSKGE

SEQ ID NO: 339 - protein, 12 aa, synthetic construct
VGTGGTVGSKGE

SEQ ID NO: 340 - protein, 7 aa, synthetic construct
AASSLQS

SEQ ID NO: 341 - protein, 11 aa, synthetic construct
QSYDSSLSGWV

SEQ ID NO: 342 - protein, 11 aa, synthetic construct
ASWTDGLSLVV

SEQ ID NO: 343 - protein, 9 aa, synthetic construct
QMYWSTPFT

SEQ ID NO: 344 - protein, 9 aa, synthetic construct
QQGHSFPFT

SEQ ID NO: 345 - protein, 9 aa, synthetic construct
HQYSSYPFT

SEQ ID NO: 346 - protein, 9 aa, synthetic construct
QHHYGIPFT

SEQ ID NO: 347 - protein, 9 aa, synthetic construct
MIWHSSASV

SEQ ID NO: 348 - protein, 13 aa, synthetic construct
GADHGSGSNFVYV

SEQ ID NO: 349 - protein, 9 aa, synthetic construct
QQADSFPPT

SEQ ID NO: 350 - protein, 9 aa, synthetic construct
QQANSFPFT

SEQ ID NO: 351 - protein, 9 aa, synthetic construct
QQATSFPLT

SEQ ID NO: 352 - protein, 9 aa, synthetic construct
LQHNSYPPT

SEQ ID NO: 353 - protein, 111 aa, synthetic construct
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 354 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 355 - protein, 107 aa, synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIK

SEQ ID NO: 356 - protein, 107 aa, synthetic construct
DIQMTQSPSSLSASVGDRVTITCRASQSISDYLHWYQQKPGKAPKLLIKYASQSMSGVPSRFSGSGSGSDFTLTISSLQPEDFATYYCQQGHSFPFTFGQGTKLEIK

SEQ ID NO: 357 - protein, 107 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 358 - protein, 107 aa, synthetic construct [sequence omitted]

SEQ ID NO: 359 - protein, 111 aa, synthetic construct
QSVLTQPPSVSGAPGQRVTISCTGSSSNIGAGYDVHWYQQLPGTAPKLLIYGSNNRPSGVPDRFSGSKSGTSASLAITGLQAEDEADYYCQSYDSSLSGWVFGGGTKLTVL

SEQ ID NO: 360 - protein, 115 aa, synthetic construct
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSASVFGGGTKLTVL

SEQ ID NO: 361 - protein, 116 aa, synthetic construct
QPVLTQPPSASASLGASVTLTCTLNSGYSDYKVDWYQQRPGKGPRFVMRVGTGGIVGSKGDGIPDRFSVLGSGLNRYLTIKNIQEEDESDYHCGADHGSGSNFVYVFGTGTKVTVL

SEQ ID NO: 362 - protein, 116 aa, synthetic construct
QPVLTQPPSASASLGASVTLTCTLSSGYSDYKVDWYQQRPGKGPRFVMRVGTGGIVGSKGEGIPDRFSVLGSGLNRYLTIKNIQEEDESDYHCGADHGSGSNFVYVFGTGTKVTVL

SEQ ID NO: 363 - protein, 116 aa, synthetic construct
QPELTQPPSASASLGASVTLTCTLSSGYSDYKVDWYQLRPGKGPRFVMRVGTGGTVGSKGEGIPDRFSVLGSGLNRSLTIKNIQEEDESDYHCGADHGSGSNFVYVFGTGTKVTVL

SEQ ID NO: 364 - protein, 107 aa, synthetic construct
DIQLTPSPSSVSASVGDRVTITCRASQGIAGWLAWYQQKPGKAPKLLIYAASSLQSGVPSRFSGSGSGTDFTLTISSLQPEDFATYYCQQADSFPPTFGGGTKVEIK

SEQ ID NO: 365 - protein, 107 aa, synthetic construct
DIQMTQSPSSVSASVGDRVTITCRASQVISSWLAWYQQKPGKAPSLLIYAASSLQSGVPSRFSGSVSGTDFTLTISSLQPEDFATYYCQQANSFPFTFGPGTKVDFK

SEQ ID NO: 366 - protein, 107 aa, synthetic construct
DIQMTQSPSSVSASVGDRVTITCRASQGSSSWFAWYQQKPGKAPKLLIYAASSLQSGVPSRFSGSGSGTDFTLTISSLQPEDFATYYCQQANSFPFTFGPGTKVDIK

SEQ ID NO: 367 - protein, 107 aa, synthetic construct
DSQMTQSPSSVSASVGDRVTITCRASQGISSWFAWYQQKPGQAPNLLIYAASSLQSGVPSRFSGSGSGTEFTLTISSLQPEDFATYYCQQANSFPFTFGPGTKVDIK

SEQ ID NO: 368 - protein, 107 aa, synthetic construct
DIQMTQSPSSVSASVGDRVTITCRAGQVISSWLAWYQQKPGKAPKLLIYAASSLQSGVPSRFSGSGSGTDFTLTISSLQPDDFATYYCQQATSFPLTFGGGTKVEIK

SEQ ID NO: 369 - protein, 107 aa, synthetic construct
DIQMTQSPSSVSASVGDRVTITCRASQGFSGWLAWYQQKPGKAPKLLIYAASSLQSGVPSRFSGSGSGTDFTLTISSLQPEDFATYYCQQATSFPLTFGPGTKVDIK

SEQ ID NO: 370 - protein, 107 aa, synthetic construct
DIQLTQSPSSVSASVGDRVTITCRASQVISSWFAWYQQKPGKAPNLLIYAASSLQSGVPSRFSGSGSGTDFTLTISSLQPADFATYFCQQATSFPLTFGPGTKVDVK

SEQ ID NO: 371 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTFGQGTKVEIE

SEQ ID NO: 372 - protein, 10 aa, synthetic construct
GYTFTDYVIN

SEQ ID NO: 373 - protein, 10 aa, synthetic construct
EIYPGSGSTF

SEQ ID NO: 374 - protein, 7 aa, synthetic construct
RMAAMDY

SEQ ID NO: 375 - protein, 7 aa, synthetic construct
RIAAMDY

SEQ ID NO: 376 - protein, 7 aa, synthetic construct
REAAMDY

SEQ ID NO: 377 - protein, 7 aa, synthetic construct
RLAAMDY

SEQ ID NO: 378 - protein, 7 aa, synthetic construct
RQAAMDY

SEQ ID NO: 379 - protein, 116 aa, synthetic construct
QVQLVQSGAEVKKPGASVKVSCKASGYTFTDYVINWVRQAPGQGLEWMGEIYPGSGSTFYAQKFQGRVTMTRDTSTSTVYMELSSLRSEDTAVYYCARRMAAMDYWGQGTLVTVSS

SEQ ID NO: 380 - protein, 116 aa, synthetic construct
QVQLVQSGAEVKKPGASVKVSCKASGYTFTDYVINWVRQAPGQGLEWMGEIYPGSGSTFYAQKFQGRVTMTADTSTSTVYMELSSLRSEDTAVYYCTRRMAAMDYWGQGTLVTVSS

SEQ ID NO: 381 - protein, 116 aa, synthetic construct
QVQLVQSGAEVKKPGASVKMSCKASGYTFTDYVINWVRQAPGQGLEWIGEIYPGSGSTFYAQKFQGRATLTADTSTSTAYMEVSSLRSEDTAVYYCTRRMAAMDYWGQGTLVTVSS

SEQ ID NO: 382 - protein, 116 aa, synthetic construct
QVQLVQSGAEVKKPGASVKMSCKASGYTFTDYVINWVRQAPGQGLEWIGEIYPGSGSTFYAQKFQGRATLTADKSTSTAYMEVSSLRSEDTAVYYCTRRMAAMDYWGQGTLVTVSS

SEQ ID NO: 383 - protein, 116 aa, synthetic construct
QVQLVQSGAEVKKPGASVKVSCKASGYTFTDYVINWVRQAPGQGLEWMGEIYPGSGSTFYAQKFQGRVTMTRDTSTSTVYMELSSLRSEDTAVYYCARRLAAMDYWGQGTLVTVSS

SEQ ID NO: 384 - protein, 116 aa, synthetic construct
QVQLVQSGAEVKKPGASVKVSCKASGYTFTDYVINWVRQAPGQGLEWMGEIYPGSGSTFYAQKFQGRVTMTADTSTSTVYMELSSLRSEDTAVYYCTRRLAAMDYWGQGTLVTVSS

SEQ ID NO: 385 - protein, 116 aa, synthetic construct
QVQLVQSGAEVKKPGASVKMSCKASGYTFTDYVINWVRQAPGQGLEWIGEIYPGSGSTFYAQKFQGRATLTADTSTSTAYMEVSSLRSEDTAVYYCTRRLAAMDYWGQGTLVTVSS

SEQ ID NO: 386 - protein, 116 aa, synthetic construct
QVQLVQSGAEVKKPGASVKMSCKASGYTFTDYVINWVRQAPGQGLEWIGEIYPGSGSTFYAQKFQGRATLTADKSTSTAYMEVSSLRSEDTAVYYCTRRLAAMDYWGQGTLVTVSS

SEQ ID NO: 387 - protein, 116 aa, synthetic construct
QVQLVQSGAEVKKPGASVKVSCKASGYTFTDYVINWVRQAPGQGLEWMGEIYPGSGSTFYAQKFQGRVTMTRDTSTSTVYMELSSLRSEDTAVYYCARRQAAMDYWGQGTLVTVSS

SEQ ID NO: 388 - protein, 116 aa, synthetic construct
QVQLVQSGAEVKKPGASVKVSCKASGYTFTDYVINWVRQAPGQGLEWMGEIYPGSGSTFYAQKFQGRVTMTADTSTSTVYMELSSLRSEDTAVYYCTRRQAAMDYWGQGTLVTVSS

SEQ ID NO: 389 - protein, 116 aa, synthetic construct
QVQLVQSGAEVKKPGASVKMSCKASGYTFTDYVINWVRQAPGQGLEWIGEIYPGSGSTFYAQKFQGRATLTADTSTSTAYMEVSSLRSEDTAVYYCTRRQAAMDYWGQGTLVTVSS

SEQ ID NO: 390 - protein, 116 aa, synthetic construct
QVQLVQSGAEVKKPGASVKMSCKASGYTFTDYVINWVRQAPGQGLEWIGEIYPGSGSTFYAQKFQGRATLTADKSTSTAYMEVSSLRSEDTAVYYCTRRQAAMDYWGQGTLVTVSS

SEQ ID NO: 391 - protein, 10 aa, synthetic construct
SASSSVSYMH

SEQ ID NO: 392 - protein, 7 aa, synthetic construct
TTSNLAS

SEQ ID NO: 393 - protein, 8 aa, synthetic construct
HQWSGYPT

SEQ ID NO: 394 - protein, 105 aa, synthetic construct
DIQMTQSPSSLSASVGDRVTITCSASSSVSYMHWYQQKPGKAPKLLIYTTSNLASGVPSRFSGSGSGTDFTLTISSLQPEDFATYYCHQWSGYPTFGQGTKLEIK

SEQ ID NO: 395 - protein, 105 aa, synthetic construct
DIQLTQSPSSLSASVGDRVTITCSASSSVSYMHWYQQKPGKAPKLLLYTTSNLASGVPSRFSGSGSGTDYTLTISSLQPEDFATYYCHQWSGYPTFGQGTKLEIK

SEQ ID NO: 396 - protein, 105 aa, synthetic construct
DIQLTQSPSSLSASVGDRITLTCSASSSVSYMHWYQQKPGKAPKLLLYTTSNLASGVPSRFSGSGSGTDYTLTISSVQPEDFATYYCHQWSGYPTFGQGTKLEIK

SEQ ID NO: 397 - protein, 105 aa, synthetic construct
DIQLTQSPSSLSASVGDRITLTCSASSSVSYMHWYQQKPGKAPKLLLYTTSNLASGVPSRFSGSGSGTDYTLTISSVQPEDAATYYCHQWSGYPTFGQGTKLEIK

SEQ ID NO: 398 - protein, 233 aa, Homo sapiens
RKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 399 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 400 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 401 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 402 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 403 - protein, 164 aa, Homo sapiens
MNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 404 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 405 - protein, 155 aa, Homo sapiens
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 406 - protein, 218 aa, Homo sapiens
MSFVGENSGVKMGSEDWEKDEPQCCLEDPAGSPLEPGPSLPTMNFVHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 407 - protein, 152 aa, Homo sapiens
MCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 408 - protein, 10 aa, synthetic construct
GGSISADGYY

SEQ ID NO: 409 - protein, 8 aa, synthetic construct
GFTFSRSA

SEQ ID NO: 410 - protein, 8 aa, synthetic construct
GFTFSFYA

SEQ ID NO: 411 - protein, 8 aa, synthetic construct
GFTFSSYA

SEQ ID NO: 412 - protein, 7 aa, synthetic construct
LYYSGST

SEQ ID NO: 413 - protein, 8 aa, synthetic construct
IYPRNSNT

SEQ ID NO: 414 - protein, 8 aa, synthetic construct
ISGSGGRT

SEQ ID NO: 415 - protein, 8 aa, synthetic construct
ISGSGGST

SEQ ID NO: 416 - protein, 8 aa, synthetic construct
ISAIDQST

SEQ ID NO: 417 - protein, 17 aa, synthetic construct
ARTPAYFGQDRTDFFDV

SEQ ID NO: 418 - protein, 15 aa, synthetic construct
ARPLYYYLTSPPTLF

SEQ ID NO: 419 - protein, 15 aa, synthetic construct
AKDSYTTSWYGGMDV

SEQ ID NO: 420 - protein, 16 aa, synthetic construct
ARTIHGIRAAYDAFII

SEQ ID NO: 421 - protein, 19 aa, synthetic construct
ARQKFMQLWGGGLRYPFGY

SEQ ID NO: 422 - protein, 125 aa, synthetic construct
QVQLQESGPGLVKPSETLSLTCTVSGGSISADGYYWSWIRQPPGKGLEWIGSLYYSGSTYYNPSLKGRVTISGDTSKNQFSLKLSSVTAADTAVYYCARTPAYFGQDRTDFFDVWGRGTLVTVSS

SEQ ID NO: 423 - protein, 122 aa, synthetic construct
QVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 424 - protein, 122 aa, synthetic construct
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 425 - protein, 123 aa, synthetic construct
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTIHGIRAAYDAFIIWGQGTLVTVSS

SEQ ID NO: 426 - protein, 126 aa, synthetic construct
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 427 - protein, 6 aa, synthetic construct
QDVGTA

SEQ ID NO: 428 - protein, 6 aa, synthetic construct
QGIFSW

SEQ ID NO: 429 - protein, 6 aa, synthetic construct
QSVGIN

SEQ ID NO: 430 - protein, 6 aa, synthetic construct
GMGDKY

SEQ ID NO: 431 - skipped

SEQ ID NO: 432 - protein, 9 aa, synthetic construct
QQSHHPPWT

SEQ ID NO: 433 - protein, 9 aa, synthetic construct
QQAKTYPFT

SEQ ID NO: 434 - protein, 9 aa, synthetic construct
QQANSVPIT

SEQ ID NO: 435 - protein, 10 aa, synthetic construct
HQYSQPPPFT

SEQ ID NO: 436 - protein, 9 aa, synthetic construct
GVIQDNTGV

SEQ ID NO: 437 - protein, 107 aa, synthetic construct
DIQMTQSPSTLSASVGDRVTITCRASQGISSWLAWYQQKPGKAPKVLIYKASTLESGVPSRFSGSGSGTEFTLTISSLQPDDFATYYCQQSHHPPWTFGQGTKLEIK

SEQ ID NO: 438 - protein, 107 aa, synthetic construct
DIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIK

SEQ ID NO: 439 - protein, 107 aa, synthetic construct
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 440 - protein, 108 aa, synthetic construct
EIVLTQSPGTLSLSPGERATLSCRASQSVGINLSWYQQKPGQAPRLLIYGASHRLTGIPDRFSGSGSGTDFTLTISRLEPEDFAVYYCHQYSQPPPFTFGGGTKVEIK

SEQ ID NO: 441 - protein, 106 aa, synthetic construct
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 442 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 443 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 444 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 445 - protein, 8 aa, synthetic construct
GFTFRSYG

SEQ ID NO: 446 - protein, 8 aa, synthetic construct
ISSSGSYI

SEQ ID NO: 447 - protein, 19 aa, synthetic construct
ARDGGRTSYTATAHNWFDP

SEQ ID NO: 448 - protein, 126 aa, synthetic construct
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSS

SEQ ID NO: 449 - protein, 12 aa, synthetic construct
QSVLFSSNNKNY

SEQ ID NO: 450 - protein, 9 aa, synthetic construct
QQHASAPPT

SEQ ID NO: 451 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIK

SEQ ID NO: 452 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 453 - protein, 399 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 454 - protein, 395 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPELQLEESCAEAQDGELDGLWTTITIFITLFLLSVCYSATITFFKVKWIFSSVVDLKQTIVPDYRNMIRQGA

SEQ ID NO: 455 - protein, 396 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLELQLEESCAEAQDGELDGLWTTITIFITLFLLSVCYSATVTFFKVKWIFSSVVDLKQTIVPDYRNMIRQGA

SEQ ID NO: 456 - protein, 233 aa, Homo sapiens
RKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGPSGTPGSNENSTQNVYKIPPTTTKALCPLYTSPRTVTQAPPKSTADVSTPDSEINLTNVTDIIRVPVFNIVILLAGGEDSKSLVFSVLFAVTLRSEVP

SEQ ID NO: 457 - protein, 224 aa, Homo sapiens
RKTRLWGLLWMDEVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRYSFQVPGPLVWTLSPLFPSLCAERM

SEQ ID NO: 458 - protein, 150 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFRCSTLSPSWLVDS

SEQ ID NO: 459 - protein, 8 aa, synthetic construct
GFTFSTYA

SEQ ID NO: 460 - protein, 8 aa, synthetic construct
GFSLSSYA

SEQ ID NO: 461 - protein, 10 aa, synthetic construct
IRTKSSNYAT

SEQ ID NO: 462 - protein, 7 aa, synthetic construct
IYAGGSP

SEQ ID NO: 463 - protein, 12 aa, synthetic construct
TRDMGIRRQFAY

SEQ ID NO: 464 - protein, 14 aa, synthetic construct
ARGTGDTVYTYFNI

SEQ ID NO: 465 - protein, 121 aa, synthetic construct
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 466 - protein, 120 aa, synthetic construct
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSS

SEQ ID NO: 467 - protein, 10 aa, synthetic construct
ESVDTFDYSF

SEQ ID NO: 468 - skipped

SEQ ID NO: 469 - protein, 9 aa, synthetic construct
QQSNEDPYT

SEQ ID NO: 470 - protein, 6 aa, synthetic construct
QNIGSD

SEQ ID NO: 471 - skipped

SEQ ID NO: 472 - protein, 14 aa, synthetic construct
QQYYYGSAGADTDT

SEQ ID NO: 473 - protein, 111 aa, synthetic construct
DIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIK

SEQ ID NO: 474 - protein, 112 aa, synthetic construct
DIQLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIK

SEQ ID NO: 475 - protein, 450 aa, synthetic construct
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 476 - protein, 449 aa, synthetic construct
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 477 - protein, 450 aa, synthetic construct
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 478 - protein, 449 aa, synthetic construct
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 479 - protein, 218 aa, synthetic construct
DIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 480 - protein, 219 aa, synthetic construct
DIQLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 481 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 482 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 483 - protein, 107 aa, synthetic construct [sequence omitted]

SEQ ID NO: 484 - protein, 5 aa, synthetic construct
TYAMH

SEQ ID NO: 485 - protein, 19 aa, synthetic construct
RIRTKSSNYATYYADSVKD

SEQ ID NO: 486 - protein, 10 aa, synthetic construct
DMGQRRQFAY

SEQ ID NO: 487 - protein, 19 aa, synthetic construct
RIRTKSSNYATYYAASVKG

SEQ ID NO: 488 - protein, 10 aa, synthetic construct
DMGIRRQFAY

SEQ ID NO: 489 - protein, 10 aa, synthetic construct
DQGIRRQFAY

SEQ ID NO: 490 - protein, 10 aa, synthetic construct
DLGIRRQFAY

SEQ ID NO: 491 - protein, 5 aa, synthetic construct
TYAQH

SEQ ID NO: 492 - protein, 5 aa, synthetic construct
TYALH

SEQ ID NO: 493 - protein, 5 aa, synthetic construct
SSYWS

SEQ ID NO: 494 - protein, 16 aa, synthetic construct
YTHYSGISNYNPSLKS

SEQ ID NO: 495 - protein, 16 aa, synthetic construct
EGYDILTGYEYYGMDV

SEQ ID NO: 496 - protein, 5 aa, synthetic construct
NYYWT

SEQ ID NO: 497 - protein, 16 aa, synthetic construct
YIYDSGYTNYNPSLKS

SEQ ID NO: 498 - protein, 14 aa, synthetic construct
GVLWFGELLPLLDY

SEQ ID NO: 499 - protein, 5 aa, synthetic construct
SSAVS

SEQ ID NO: 500 - protein, 5 aa, synthetic construct
TYAIS

SEQ ID NO: 501 - protein, 5 aa, synthetic construct
IYVIS

SEQ ID NO: 502 - protein, 5 aa, synthetic construct
RHAIS

SEQ ID NO: 503 - protein, 5 aa, synthetic construct
SSAIS

SEQ ID NO: 504 - protein, 5 aa, synthetic construct
SYAFT

SEQ ID NO: 505 - protein, 5 aa, synthetic construct
RYAIS

SEQ ID NO: 506 - protein, 5 aa, synthetic construct
RYAFS

SEQ ID NO: 507 - protein, 5 aa, synthetic construct
TYDIN

SEQ ID NO: 508 - protein, 17 aa, synthetic construct
GITPIFGTADYAQKFQG

SEQ ID NO: 509 - protein, 17 aa, synthetic construct
GINPIFGTANYAQKFQG

SEQ ID NO: 510 - protein, 17 aa, synthetic construct
GIIPLFGTPNYAQRFQD

SEQ ID NO: 511 - protein, 17 aa, synthetic construct
GIIPLFGTPNYAQQFQD

SEQ ID NO: 512 - protein, 17 aa, synthetic construct
GIIPLFGTANYAQQFQD

SEQ ID NO: 513 - protein, 17 aa, synthetic construct
GIIPLFGTANYAQEFQG

SEQ ID NO: 514 - protein, 17 aa, synthetic construct
GIIPLFGTANYAQKFQG

SEQ ID NO: 515 - protein, 17 aa, synthetic construct
GIIPIFSTGNYAQKFQG

SEQ ID NO: 516 - protein, 17 aa, synthetic construct
GIIPIFRTANYAQKFQG

SEQ ID NO: 517 - protein, 17 aa, synthetic construct
GIIPIFGTSNYAQKFQG

SEQ ID NO: 518 - protein, 17 aa, synthetic construct
GIIPIFGTPNYAQKFQG

SEQ ID NO: 519 - protein, 17 aa, synthetic construct
GIIPIFGTADSAQKFQG

SEQ ID NO: 520 - protein, 17 aa, synthetic construct
GIIPIFGTANYAQKFQG

SEQ ID NO: 521 - protein, 17 aa, synthetic construct
WVNPNSGNTGYAQKFQD

SEQ ID NO: 522 - protein, 17 aa, synthetic construct
TPRYRGSSHHYYYALGV

SEQ ID NO: 523 - protein, 8 aa, synthetic construct
GGAVGFAY

SEQ ID NO: 524 - protein, 16 aa, synthetic construct
GHGPGSSHYSYYGLDV

SEQ ID NO: 525 - protein, 18 aa, synthetic construct
SYFYGSGSSNYYYYGLDV

SEQ ID NO: 526 - protein, 17 aa, synthetic construct
STRVRGVSHYYYYGLDV

SEQ ID NO: 527 - protein, 18 aa, synthetic construct
SHFSGSGSSHYYYYGMHV

SEQ ID NO: 528 - protein, 16 aa, synthetic construct
GGNSWTTSLYYYGMDV

SEQ ID NO: 529 - protein, 18 aa, synthetic construct
SHFYGSGSSHFYYYGMHV

SEQ ID NO: 530 - protein, 18 aa, synthetic construct
SHFYGSGSSNYYYYGLDV

SEQ ID NO: 531 - protein, 17 aa, synthetic construct
TPRYRGSSHHYFYALGV

SEQ ID NO: 532 - protein, 16 aa, synthetic construct
ASQSRSSNYYYYGLDV

SEQ ID NO: 533 - protein, 17 aa, synthetic construct
DGLNMVRGVHNYYGMDV

SEQ ID NO: 534 - protein, 9 aa, synthetic construct
GTFSSYAIS

SEQ ID NO: 535 - protein, 9 aa, synthetic construct
YTFTSYYMH

SEQ ID NO: 536 - protein, 11 aa, synthetic construct
GSISSSSYYWG

SEQ ID NO: 537 - protein, 9 aa, synthetic construct
FTFSSYSMN

SEQ ID NO: 538 - protein, 9 aa, synthetic construct
FTFSSYGMH

SEQ ID NO: 539 - protein, 9 aa, synthetic construct
FTFDDYAMH

SEQ ID NO: 540 - protein, 9 aa, synthetic construct
GSISSYYWS

SEQ ID NO: 541 - protein, 9 aa, synthetic construct
FTFSDHHMD

SEQ ID NO: 542 - protein, 9 aa, synthetic construct
FTFSSYWMS

SEQ ID NO: 543 - protein, 9 aa, synthetic construct
YTFTSYYIH

SEQ ID NO: 544 - protein, 11 aa, synthetic construct
GSISSGGYYWS

SEQ ID NO: 545 - protein, 9 aa, synthetic construct
FTFSNYGMH

SEQ ID NO: 546 - protein, 9 aa, synthetic construct
LTFSSYGMH

SEQ ID NO: 547 - protein, 9 aa, synthetic construct
FTFSTYAMS

SEQ ID NO: 548 - protein, 9 aa, synthetic construct
GTFSNYAIS

SEQ ID NO: 549 - protein, 10 aa, synthetic construct
YSISSGYYWA

SEQ ID NO: 550 - protein, 10 aa, synthetic construct
YSISSGYYWG

SEQ ID NO: 551 - protein, 11 aa, synthetic construct
GSISSSDYYWG

SEQ ID NO: 552 - protein, 9 aa, synthetic construct
YTFTGYYMH

SEQ ID NO: 553 - protein, 9 aa, synthetic construct
YTFTSYGIH

SEQ ID NO: 554 - protein, 9 aa, synthetic construct
YSFTTYWIG

SEQ ID NO: 555 - protein, 9 aa, synthetic construct
YTFTSYGIS

SEQ ID NO: 556 - protein, 9 aa, synthetic construct
FTFGDYAMH

SEQ ID NO: 557 - protein, 9 aa, synthetic construct
FTFSSYAMS

SEQ ID NO: 558 - protein, 17 aa, synthetic construct
GIIPIFGTANYAQKFQG

SEQ ID NO: 559 - protein, 17 aa, synthetic construct
VINPSGGSTSYAQKFQG

SEQ ID NO: 560 - protein, 17 aa, synthetic construct
IINPSGGSTSYAQKFQG

SEQ ID NO: 561 - protein, 16 aa, synthetic construct
SIYYSGSTYYNPSLKS

SEQ ID NO: 562 - protein, 17 aa, synthetic construct
SISSSSNYIYYADSVKG

SEQ ID NO: 563 - protein, 17 aa, synthetic construct
VISYDGSNKYYADSVKG

SEQ ID NO: 564 - protein, 17 aa, synthetic construct
SISSSSSYIYYADSVKG

SEQ ID NO: 565 - protein, 17 aa, synthetic construct
GISWNSGSIGYADSVKG

SEQ ID NO: 566 - protein, 16 aa, synthetic construct
SIYYSGSTNYNPSLKS

SEQ ID NO: 567 - protein, 17 aa, synthetic construct
GISWNSGDIGYADSVKG

SEQ ID NO: 568 - protein, 19 aa, synthetic construct
RTRNKANSYTTEYAASVKG

SEQ ID NO: 569 - protein, 17 aa, synthetic construct
NIKQDGSEKYYVDSVKG

SEQ ID NO: 570 - protein, 17 aa, synthetic construct
YISSSSSTIYYADSVKG

SEQ ID NO: 571 - protein, 16 aa, synthetic construct
HIYYSGSTNYNPSLKS

SEQ ID NO: 572 - protein, 17 aa, synthetic construct
GITWNSGSIGYADSVKG

SEQ ID NO: 573 - protein, 16 aa, synthetic construct
YIYYSGSTYYNPSLKS

SEQ ID NO: 574 - protein, 17 aa, synthetic construct
VIWYDGSNKYYADSVKG

SEQ ID NO: 575 - protein, 17 aa, synthetic construct
LIWYDGSNKYYADSVKG

SEQ ID NO: 576 - protein, 17 aa, synthetic construct
AISGSGGSTYYADSVKG

SEQ ID NO: 577 - protein, 17 aa, synthetic construct [sequence omitted]

SEQ ID NO: 578 - protein, 17 aa, synthetic construct
VINPGGGSTSYAQKFQG

SEQ ID NO: 579 - protein, 17 aa, synthetic construct
IINPGGGSTSYAQKFQG

SEQ ID NO: 580 - protein, 17 aa, synthetic construct
SIIPIFGTANYAQKFQG

SEQ ID NO: 581 - protein, 16 aa, synthetic construct
SIYHSGSTYYNPSLKS

SEQ ID NO: 582 - protein, 16 aa, synthetic construct
SIYHSGNTYYNPSLKS

SEQ ID NO: 583 - protein, 16 aa, synthetic construct
SISYSGSTYYNPSLKS

SEQ ID NO: 584 - protein, 17 aa, synthetic construct
WINPNSGGTKYAQKFQG

SEQ ID NO: 585 - protein, 17 aa, synthetic construct
WISAYNGNTNYAQKLQG

SEQ ID NO: 586 - protein, 17 aa, synthetic construct
IIYPGDSDTRYSPSFQG

SEQ ID NO: 587 - protein, 15 aa, synthetic construct
ARGQGSDHYYYGMDV

SEQ ID NO: 588 - protein, 16 aa, synthetic construct
AREGGPRGASFNWFDP

SEQ ID NO: 589 - protein, 11 aa, synthetic construct
ARDVGSMYFDI

SEQ ID NO: 590 - protein, 13 aa, synthetic construct
ARHYYYGYAYFDL

SEQ ID NO: 591 - protein, 13 aa, synthetic construct
ARESDGIDSYFDY

SEQ ID NO: 592 - protein, 14 aa, synthetic construct
ARESGHSYVSSFDP

SEQ ID NO: 593 - protein, 12 aa, synthetic construct
ARGLIYGDAFDY

SEQ ID NO: 594 - protein, 13 aa, synthetic construct
AREVSMTAASLDV

SEQ ID NO: 595 - protein, 14 aa, synthetic construct
AREAGYDISSAFDI

SEQ ID NO: 596 - protein, 13 aa, synthetic construct
AREGSGSWETLDV

SEQ ID NO: 597 - protein, 10 aa, synthetic construct
ARSGEYGFDL

SEQ ID NO: 598 - protein, 13 aa, synthetic construct
ARGGGYPWEAFDY

SEQ ID NO: 599 - protein, 13 aa, synthetic construct
ARGRYRRTGSLDV

SEQ ID NO: 600 - protein, 11 aa, synthetic construct
ARRSSGDYLDV

SEQ ID NO: 601 - protein, 12 aa, synthetic construct
ARRGGSYDAFQH

SEQ ID NO: 602 - protein, 12 aa, synthetic construct
AKGPRMSGWWAD

SEQ ID NO: 603 - protein, 14 aa, synthetic construct
ARGAPGGRHNWFDP

SEQ ID NO: 604 - protein, 12 aa, synthetic construct
AKGPRMVTHLDV

SEQ ID NO: 605 - protein, 9 aa, synthetic construct
ARGPLGYKL

SEQ ID NO: 606 - protein, 11 aa, synthetic construct
ARDAPQLGLDV

SEQ ID NO: 607 - protein, 16 aa, synthetic construct
ARGGPLGYGDYKGMDV

SEQ ID NO: 608 - protein, 18 aa, synthetic construct
ARDAGRYYGSSSSWYFDL

SEQ ID NO: 609 - protein, 12 aa, synthetic construct
AKGPRLLSALDV

SEQ ID NO: 610 - protein, 12 aa, synthetic construct
AKGGSRYSHFDY

SEQ ID NO: 611 - protein, 15 aa, synthetic construct
ARDSAQETYYYGMDV

SEQ ID NO: 612 - protein, 16 aa, synthetic construct
ARDSSIAGRATLSFDY

SEQ ID NO: 613 - protein, 19 aa, synthetic construct
ARGPSQYYYDSSAIEAFDI

SEQ ID NO: 614 - protein, 20 aa, synthetic construct
ARDGGGTAQADGAYYYGMDV

SEQ ID NO: 615 - protein, 18 aa, synthetic construct
ARGRKAAAGIDEAEYFQH

SEQ ID NO: 616 - protein, 14 aa, synthetic construct
ARDRRMWDPYGMDV

SEQ ID NO: 617 - protein, 16 aa, synthetic construct
ARDAPAVVGESPAFDI

SEQ ID NO: 618 - protein, 15 aa, synthetic construct
AKGSTHRGSAYGMDV

SEQ ID NO: 619 - protein, 13 aa, synthetic construct
ARRPDDRRGLFQH

SEQ ID NO: 620 - protein, 14 aa, synthetic construct
ARPDYYSSRGVFDI

SEQ ID NO: 621 - protein, 12 aa, synthetic construct
AKGDYLDPLFDY

SEQ ID NO: 622 - protein, 15 aa, synthetic construct
ARERGTYYYASGWAN

SEQ ID NO: 623 - protein, 13 aa, synthetic construct
ARRGGSSSTGLLY

SEQ ID NO: 624 - protein, 11 aa, synthetic construct
ARTRIDDSFDI

SEQ ID NO: 625 - protein, 12 aa, synthetic construct
AKSKHSTTSLDV

SEQ ID NO: 626 - protein, 17 aa, synthetic construct
ARELMVTSGGWLYGMDV

SEQ ID NO: 627 - protein, 16 aa, synthetic construct
AREAGNYYDIESAFDI

SEQ ID NO: 628 - protein, 13 aa, synthetic construct
AREGSGYDESMDV

SEQ ID NO: 629 - protein, 10 aa, synthetic construct
ARGRGIAFDI

SEQ ID NO: 630 - protein, 13 aa, synthetic construct
AREAGQTSSALDV

SEQ ID NO: 631 - protein, 15 aa, synthetic construct
AREAGSWLISTAFDI

SEQ ID NO: 632 - protein, 13 aa, synthetic construct
AREAGTMSSAFDI

SEQ ID NO: 633 - protein, 17 aa, synthetic construct
ARSGGYSSSWYGTGYDY

SEQ ID NO: 634 - protein, 17 aa, synthetic construct
ARDRGQYSSSWYGRMDV

SEQ ID NO: 635 - protein, 14 aa, synthetic construct
ARESGYHVSTAFDI

SEQ ID NO: 636 - protein, 12 aa, synthetic construct
ARHWYALGSFDI

SEQ ID NO: 637 - protein, 12 aa, synthetic construct
ARGADYYAGFDY

SEQ ID NO: 638 - protein, 12 aa, synthetic construct
AKGPRLLGYFDL

SEQ ID NO: 639 - protein, 13 aa, synthetic construct
AKGPRYSKPYFDY

SEQ ID NO: 640 - protein, 12 aa, synthetic construct
ARQEYGDGYFDL

SEQ ID NO: 641 - protein, 13 aa, synthetic construct
ARDLGGYEGAFDP

SEQ ID NO: 642 - protein, 12 aa, synthetic construct
ARHDDYLSSFDP

SEQ ID NO: 643 - protein, 9 aa, synthetic construct
ARGPSWIDV

SEQ ID NO: 644 - protein, 17 aa, synthetic construct
ARELYAYSSPMFYGMDV

SEQ ID NO: 645 - protein, 11 aa, synthetic construct
ARYYSPYGMDV

SEQ ID NO: 646 - protein, 13 aa, synthetic construct
ARDSGQYTGSLDV

SEQ ID NO: 647 - protein, 12 aa, synthetic construct
ARERHSSLGYAY

SEQ ID NO: 648 - protein, 15 aa, synthetic construct
ARGRPSSSWGNWFDP

SEQ ID NO: 649 - protein, 13 aa, synthetic construct
ARGSPWDGRLFDI

SEQ ID NO: 650 - protein, 16 aa, synthetic construct
ARGAGMYDGSPLGMDV

SEQ ID NO: 651 - protein, 12 aa, synthetic construct
ARAGTIYGRLDL

SEQ ID NO: 652 - protein, 12 aa, synthetic construct
AKGPRRTSHLDI

SEQ ID NO: 653 - protein, 13 aa, synthetic construct
AKGPRMTHSYFDL

SEQ ID NO: 654 - protein, 12 aa, synthetic construct
AKAPRMYGYFDL

SEQ ID NO: 655 - protein, 12 aa, synthetic construct
AKGPRTRGYFDL

SEQ ID NO: 656 - protein, 13 aa, synthetic construct
AKAPRTRWTYFDY

SEQ ID NO: 657 - protein, 13 aa, synthetic construct
ARARRGALAGMDV

SEQ ID NO: 658 - protein, 14 aa, synthetic construct
ARGGPYPWSGWFDP

SEQ ID NO: 659 - protein, 13 aa, synthetic construct
ARDLGQYEGYFDL

SEQ ID NO: 660 - protein, 13 aa, synthetic construct
ARLGDGYRIWADY

SEQ ID NO: 661 - protein, 20 aa, synthetic construct
ARELIVGATGGLTYYYGMDV

SEQ ID NO: 662 - protein, 121 aa, synthetic construct; identical to SEQ ID NO: 465

SEQ ID NO: 663 - protein, 120 aa, synthetic construct; identical to SEQ ID NO: 466

SEQ ID NO: 664 - protein, 121 aa, synthetic construct
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDQGIRRQFAYWGQGTLVTVSS

SEQ ID NO: 665 - protein, 121 aa, synthetic construct
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDLGIRRQFAYWGQGTLVTVSS

SEQ ID NO: 666 - protein, 121 aa, synthetic construct
EVQLVESGGGLVQPGGSLKLSCAASGFTFSTYAQHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 667 - protein, 121 aa, synthetic construct
EVQLVESGGGLVQPGGSLKLSCAASGFTFSTYALHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 668 - protein, 122 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQGSDHYYYGMDVWGQGTTVTVSS

SEQ ID NO: 669 - protein, 123 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREGGPRGASFNWFDPWGQGTLVTVSS

SEQ ID NO: 670 - protein, 118 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDVGSMYFDIWGQGTMVTVSS

SEQ ID NO: 671 - protein, 120 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHYYYGYAYFDLWGRGTLVTVSS

SEQ ID NO: 672 - protein, 120 aa, synthetic construct
QVQLVQSGAEVKKPGASVKVSCKASGYTFTSYYMHWVRQAPGQGLEWMGVINPSGGSTSYAQKFQGRVTMTRDTSTSTVYMELSSLRSEDTAVYYCARESDGIDSYFDYWGQGTLVTVSS

SEQ ID NO: 673 - protein, 121 aa, synthetic construct
QVQLVQSGAEVKKPGASVKVSCKASGYTFTSYYMHWVRQAPGQGLEWMGVINPSGGSTSYAQKFQGRVTMTRDTSTSTVYMELSSLRSEDTAVYYCARESGHSYVSSFDPWGQGTLVTVSS

SEQ ID NO: 674 - protein, 119 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGLIYGDAFDYWGQGTLVTVSS

SEQ ID NO: 675 - protein, 120 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREVSMTAASLDVWGQGTMVTVSS

SEQ ID NO: 676 - protein, 121 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREAGYDISSAFDIWGQGTMVTVSS

SEQ ID NO: 677 - protein, 120 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGSGSWETLDVWGQGTMVTVSS

SEQ ID NO: 678 - protein, 118 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARSGEYGFDLWGRGTLVTVSS

SEQ ID NO: 679 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGGGYPWEAFDYWGKGTTVTVSS

SEQ ID NO: 680 - protein, 120 aa, synthetic construct
EVQLVESGGGLVKPGGSLRLSCAASGFTFSSYSMNWVRQAPGKGLEWVSSISSSSNYIYYADSVKGRFTISRDNAKNSLYLQMNSLRAEDTAVYYCARGRYRRTGSLDVWGQGTMVTVSS

SEQ ID NO: 681 - protein, 118 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARRSSGDYLDVWGQGTMVTVSS

SEQ ID NO: 682 - protein, 119 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARRGGSYDAFQHWGQGTLVTVSS

SEQ ID NO: 683 - protein, 119 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGPRMSGWWADWGQGTLVTVSS

SEQ ID NO: 684 - protein, 120 aa, synthetic construct
QVQLQESGPGLVKPSETLSLTCTVSGGSISSYYWSWIRQPPGKGLEWIGSIYYSGSTNYNPSLKSRVTISVDTSKNQFSLKLSSVTAADTAVYYCARGAPGGRHNWFDPWGQGTLVTVSS

SEQ ID NO: 685 - protein, 119 aa, synthetic construct
EVQLVESGGGLVQPGRSLRLSCAASGFTFDDYAMHWVRQAPGKGLEWVSGISWNSGDIGYADSVKGRFTISRDNAKNTLYLQMNSLRAEDTALYYCAKGPRMVTHLDVWGQGTMVTVSS

SEQ ID NO: 686 - protein, 118 aa, synthetic construct
EVQLVESGGGLVQPGGSLRLSCAASGFTFSDHHMDWVRQAPGKGLEWVGRTRNKANSYTTEYAASVKGRFTISRDDSKNSLYLQMNSLKTEDTAVYYCARGPLGYKLWGQGTLVTVSS

SEQ ID NO: 687 - protein, 118 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDAPQLGLDVWGQGTMVTVSS

SEQ ID NO: 688 - protein, 123 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGPLGYGDYKGMDVWGQGTTVTVSS

SEQ ID NO: 689 - protein, 124 aa, synthetic construct
QVQLQESGPGLVKPSETLSLTCTVSGGSISSYYWSWIRQPPGKGLEWIGHIYYSGSTNYNPSLKSRVTISVDTSKNQFSLKLSSVTAADTAVYYCARDAGRYYGSSSSWYFDLWGRGTLVTVSS

SEQ ID NO: 690 - protein, 119 aa, synthetic construct
EVQLVESGGGLVQPGRSLRLSCAASGFTFDDYAMHWVRQAPGKGLEWVSGITWNSGSIGYADSVKGRFTISRDNAKNSLYLQMNSLRAEDTALYYCAKGPRLLSALDVWGQGTMVTVSS

SEQ ID NO: 691 - protein, 119 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGGSRYSHFDYWGQGTLVTVSS

SEQ ID NO: 692 - protein, 122 aa, synthetic construct
QVQLVQSGAEVKKPGASVKVSCKASGYTFTSYYIHWVRQAPGQGLEWMGIINPSGGSTSYAQKFQGRVTMTRDTSTSTVYMELSSLRSEDTAVYYCARDSAQETYYYGMDVWGQGTTVTVSS

SEQ ID NO: 693 - protein, 124 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDSSIAGRATLSFDYWGQGTLVTVSS

SEQ ID NO: 694 - protein, 126 aa, synthetic construct
EVQLVESGGGLVKPGGSLRLSCAASGFTFSSYSMNWVRQAPGKGLEWVSSISSSSNYIYYADSVKGRFTISRDNAKNSLYLQMNSLRAEDTAVYYCARGPSQYYYDSSAIEAFDIWGQGTMVTVSS

SEQ ID NO: 695 - protein, 128 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDGGGTAQADGAYYYGMDVWGQGTTVTVSS

SEQ ID NO: 696 - protein, 126 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGRKAAAGIDEAEYFQHWGQGTLVTVSS

SEQ ID NO: 697 - protein, 122 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDRRMWDPYGMDVWGQGTTVTVSS

SEQ ID NO: 698 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDAPAVVGESPAFDIWGQGTMVTVSS

SEQ ID NO: 699 - protein, 122 aa, synthetic construct
QVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCAKGSTHRGSAYGMDVWGQGTTVTVSS

SEQ ID NO: 700 - protein, 120 aa, synthetic construct
EVQLVESGGGLVKPGGSLRLSCAASGFTFSSYSMNWVRQAPGKGLEWVSSISSSSNYIYYADSVKGRFTISRDNAKNSLYLQMNSLKAEDTAVYYCARRPDDRRGLFQHWGQGTLVTVSS

SEQ ID NO: 701 - protein, 121 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARPDYYSSRGVFDIWGQGTMVTVSS

SEQ ID NO: 702 - protein, 119 aa, synthetic construct
QVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCAKGDYLDPLFDYWGQGTLVTVSS

SEQ ID NO: 703 - protein, 122 aa, synthetic construct
QVQLVESGGGVVQPGRSLRLSCAASGLTFSSYGMHWVRQAPGKGLEWVAVISYDGSNKYYADSVKGRFTISRDNSKNTLYLQMNSLRAEDTAVYYCARERGTYYYASGWANWGQGTLVTVSS

SEQ ID NO: 704 - protein, 120 aa, synthetic construct
EVQLVESGGGLVKPGGSLRLSCAASGFTFSSYSMNWVRQAPGKGLEWVSSISSSSNYIYYADSVKGRFTISRDNAKNSLYLQMNSLRAEDTAVYYCARRGGSSSTGLLYWGQGTLVTVSS

SEQ ID NO: 705 - protein, 118 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARTRIDDSFDIWGQGTMVTVSS

SEQ ID NO: 706 - protein, 119 aa, synthetic construct
EVQLLESGGGLVQPGGSLRLSCAASGFTFSTYAMSWVRQAPGKGLEWVSAISGSGGSTYYADSVKGRFTISRDNSKNTLYLQMNSLRAEDTAVYYCAKSKHSTTSLDVWGQGTMVTVSS

SEQ ID NO: 707 - protein, 124 aa, synthetic construct
QVQLVESGGGVVQPGRSLRLSCAASGFTFSSYGMHWVRQAPGKGLEWVAVIWYDGSNKGYADSVKGRFTISRDNSKNTLYLQMNSLRAEDTAVYYCARELMVTSGGWLYGMDVWGQGTTVTVSS

SEQ ID NO: 708 - protein, 123 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREAGNYYDIESAFDIWGQGTMVTVSS

SEQ ID NO: 709 - protein, 120 aa, synthetic construct
QVQLVQSGAEVKKPGASVKVSCKASGYTFTSYYMHWVRQAPGQGLEWMGVINPSGGSTSYAQKFQGRVTMTRDTSTSTVYMELSSLRSEDTAVYYCAREGSGYDESMDVWGQGTTVTVSS

SEQ ID NO: 710 - protein, 117 aa, synthetic construct
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGRGIAFDIWGQGTMVTVSS

SEQ ID NO: 711 - protein, 120 aa, synthetic construct
QVQLVQSGAEVKKPGASVKVSCKASGYTFTSYYMHWVRQAPGQGLEWMGVINPGGGSTSYAQKFQGRVTMTRDTSTSTVYMELSSLRSEDTAVYYCAREAGQTSSALDVWGQGTMVTVSS

SEQ ID NO: 712 - protein, 122 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREAGSWLISTAFDIWGQGTMVTVSS

SEQ ID NO: 713 - protein, 120 aa, synthetic construct
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCAREAGTMSSAFDIWGQGTMVTVSS

SEQ ID NO: 714 - protein, 124 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARSGGYSSSWYGTGYDYWGQGTLVTVSS

SEQ ID NO: 715 - protein, 124 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARDRGQYSSSWYGRMDVWGQGTTVTVSS

SEQ ID NO: 716 - protein, 121 aa, synthetic construct
QVQLVQSGAEVKKPGASVKVSCKASGYTFTSYYIHWVRQAPGQGLEWMGIINPSGGSTSYAQKFQGRVTMTRDTSTSTVYMELSSLRSEDTAVYYCARESGYHVSTAFDIWGQGTMVTVSS

SEQ ID NO: 717 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHWYALGSFDIWGQGTMVTVSS

SEQ ID NO: 718 - protein, 119 aa, synthetic construct
QVQLVQSGAEVKKPGASVKVSCKASGYTFTSYYMHWVRQAPGQGLEWMGVINPSGGSTSYAQKFQGRVTMTRDTSTSTVYMELSSLRSEDTAVYYCARGADYYAGFDYWGQGTLVTVSS

SEQ ID NO: 719 - protein, 119 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGPRLLGYFDLWGRGTLVTVSS

SEQ ID NO: 720 - protein, 120 aa, synthetic construct
EVQLVESGGGLVQPGRSLRLSCAASGFTFDDYAMHWVRQAPGKGLEWVSGITWNSGSIGYADSVKGRFTISRDNAKNSLYLQMNSLRAEDTALYYCAKGPRYSKPYFDYWGQGTLVTVSS

SEQ ID NO: 721 - protein, 120 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARQEYGDGYFDLWGRGTLVTVSS

SEQ ID NO: 722 - protein, 120 aa, synthetic construct
QVQLQESGPGLVKPSETLSLTCAVSGYSISSGYYWAWIRQPPGKGLEWIGSIYHSGSTYYNPSLKSRVTISVDTSKNQFSLKLSSVTAADTAVYYCARDLGGYEGAFDPWGQGTLVTVSS

SEQ ID NO: 723 - protein, 119 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARHDDYLSSFDPWGQGTLVTVSS

SEQ ID NO: 724 - protein, 117 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARGPSWIDVWGQGTMVTVSS

SEQ ID NO: 725 - protein, 124 aa, synthetic construct
QVQLQESGPGLVKPSETLSLTCAVSGYSISSGYYWGWIRQPPGKGLEWIGSIYHSGNTYYNPSLKSRVTISVDTSKNQFSLKLSSVTAADTAVYYCARELYAYSSPMFYGMDVWGRGTTVTVSS

SEQ ID NO: 726 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARYYSPYGMDVWGQGTTVTVSS

SEQ ID NO: 727 - protein, 121 aa, synthetic construct
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARDSGQYTGSLDVWGQGTMVTVSS

SEQ ID NO: 728 - protein, 119 aa, synthetic construct
QVQLVQSGAEVKKPGASVKVSCKASGYTFTGYYMHWVRQAPGQGLEWMGWINPNSGGTKYAQKFQGRVTMTRDTSISTAYMELSRLRSDDTAVYYCARERHSSLGYAYWGQGTLVTVSS

SEQ ID NO: 729 - protein, 122 aa, synthetic construct
QVQLVQSGAEVKKPGASVKVSCKASGYTFTSYGIHWVRQAPGQGLEWMGWISAYNGNTNYAQKLQGRVTMTTDTSTSTAYMELRSLRSDDTAVYYCARGRPSSSWGNWFDPWGQGTTVTVSS

SEQ ID NO: 730 - protein, 120 aa, synthetic construct
EVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARGSPWDGRLFDIWGQGTMVTVSS

SEQ ID NO: 731 - protein, 123 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGAGMYDGSPLGMDVWGQGTTVTVSS

SEQ ID NO: 732 - protein, 119 aa, synthetic construct
QVQLVQSGAEVKKPGASVKVSCKASGYTFTSYGIHWVRQAPGQGLEWMGWISAYNGNTNYAQKLQGRVTMTTDTSTSTAYMELRSLRSDDTAVYYCARAGTIYGRLDLWGRGTLVTVSS

SEQ ID NO: 733 - protein, 119 aa, synthetic construct
EVQLVESGGGLVQPGRSLRLSCAASGFTFGDYAMHWVRQAPGKGLEWVSGISWNSGSIGYADSVKGRFTISRDNAKNSLYLQMNSLRAEDTALYYCAKGPRRTSHLDIWGQGTMVTVSS

SEQ ID NO: 734 - protein, 120 aa, synthetic construct
EVQLVESGGGLVQPGRSLRLSCAASGFTFDDYAMHWVRQAPGKGLEWVSGISWNSGDIGYADSVKGRFTISRDNAKNSLYLQMNSLRAEDTALYYCAKGPRMTHSYFDLWGRGTLVTVSS

SEQ ID NO: 735 - protein, 119 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKAPRMYGYFDLWGRGTSVTVSS

SEQ ID NO: 736 - protein, 119 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGPRTRGYFDLWGRGTLVTVSS

SEQ ID NO: 737 - protein, 120 aa, synthetic construct
QVQLVESGGGLVQPGRSLRLSCAASGFTFDDYAMHWVRQAPGKGLEWVSGISWNSGDIGYADSVKGRFTISRDNAKNSLYLQMNSLRAEDTALYYCAKAPRTRWTYFDYWGQGTLVTVSS

SEQ ID NO: 738 - protein, 120 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARARRGALAGMDVWGQGTTVTVSS

SEQ ID NO: 739 - protein, 121 aa, synthetic construct
QVQLQESGPGLVKPSETLSLTCAVSGYSISSGYYWAWIRQPPGKGLEWIGSIYHSGSTYYNPSLKSRVTISVDTSKNQFSLKLSSVTAADTAVYYCARGGPYPWSGWFDPWGQGTLVTVSS

SEQ ID NO: 740 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDLGQYEGYFDLWGRGTLVTVSS

SEQ ID NO: 741 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARLGDGYRIWADYWGQGTLVTVSS

SEQ ID NO: 742 - protein, 127 aa, synthetic construct
QVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCARELIVGATGGLTYYYGMDVWGQGTTVTVSS

SEQ ID NO: 743 - protein, 124 aa, synthetic construct
QVQLQESGPGLVKPSETLSLTCTVSGGSISSSYWSWVRQPPGKGLEWIGYTHYSGISNYNPSLKSRVTISVDTSKNQFSLKLSSVTAADTAVYYCAREGYDILTGYEYYGMDVWGQGTTVTVSS

SEQ ID NO: 744 - protein, 122 aa, synthetic construct
QVQLQESGPGLVKPSETLSLTCTVSGGSITNYYWTWIRQPPGKGLEWIGYIYDSGYTNYNPSLKSRVTLSIDTSKNQFSLKLSSVTAADTAVYYCARGVLWFGELLPLLDYWGQGTLVTVSS

SEQ ID NO: 745 - protein, 123 aa, synthetic construct
QVQLVQSGAEVKRPGSSVKVSCKASGGTFSSSAISWVRQAPGQGLEWMGGIIPIFGTTNGAQKFQGRVTITADESTSTAYMELSSLRSEDTAVYYCAAMVRGNYFYFYGMDVWGQGTTVTVSS

SEQ ID NO: 746 - protein, 125 aa, synthetic construct
QVQLVESGGGVVQPGRSLRLSCAATEFTFSNYGMHWVRQAPGKGLEWVAVIWYDGSNKYYADSVKGRFTISRDNSKNTLYLQLNSLSAEDSAVYYCARDGRHYYGSTSYFGMDVWGQGTTVTVSS

SEQ ID NO: 747 - protein, 126 aa, synthetic construct
QVQLVQSGAEVKKPGSSVKVSCKASGGTFINSEAINWVRQAPGQGLEWMGGIIPIFDITNYAQKFQGRVTITADESMSTAYMELSSLRSEDTAVYYCAKTYYDILTYHYHYGMDVWGQGTTVTVSS

SEQ ID NO: 748 - protein, 126 aa, synthetic construct
QVQLVQSGAEVKKPGSSVKVSCKTSGGTFSSSAVSWVRQAPGQGLEWMGGITPIFGTADYAQKFQGRVTITADASTSTGYMELSSLRSEDTAVYYCAFTPRYRGSSHHYYYALGVWGQGTTVTVSS

SEQ ID NO: 749 - protein, 117 aa, synthetic construct
QVQLVQSGAEVKKPGSSVKVSCNPSGGTFSTYAISWVRQAPGQGLEWMGGINPIFGTANYAQKFQGRVTITADESTSPGYLELSSLRSEDTAVYYCARGGAVGFAYWGQGTLVTVSS

SEQ ID NO: 750 - protein, 125 aa, synthetic construct
QVQLVQSGAEVKRPGSSVKVSCKASGGTFSIYVISWVRQAPGQGLEWMGGIIPLFGTPNYAQRFQDRVTITADESTRTAYMELSSLRSEDTAVYYCARGHGPGSSHYSYYGLDVWGQGTTVTVSS

SEQ ID NO: 751 - protein, 125 aa, synthetic construct
QVQLVQSGAEVKRPGSSVKVSCKASGGTFSIYVISWVRQAPGQGLEWMGGIIPLFGTPNYAQQFQDRVTITADESTRTAYMELNSLKSEDTAVYYCARGHGPGSSHYSYYGLDVWGQGTTVTVSS

SEQ ID NO: 752 - protein, 125 aa, synthetic construct
QVQLVQSGAEVKRPGSSVKVSCKASGGTFSIYVISWVRQAPGQGLEWMGGIIPLFGTPNYAQQFQDRVTITADESTRTAYMELSSLRSEDTAVYYCARGHGPGSSHYSYYGLDVWGQGTTVTVSS

SEQ ID NO: 753 - protein, 125 aa, synthetic construct
QVQLVQSGAEVKKPGSSVKVSCKASGGTFSIYVISWVRQAPGQGLEWMGGIIPLFGTANYAQQFQDRVTITADESTRTAYMELNSLKSEDTAVYYCARGHGPGSSHYSYYGLDVWGQGTTVTVSS

SEQ ID NO: 754 - protein, 127 aa, synthetic construct
QVQLVQSGAEVKKPGSSVKVSCKASGGTFNRHAISWVRQAPGQGLEWMGGIIPLFGTANYAQEFQGRVTIAADEPTSTTYMELRSLRSEDTAVYYCASSYFYGSGSSNYYYYGLDVWGQGTTVTVSS

SEQ ID NO: 755 - protein, 127 aa, synthetic construct
QVQLVQSGAEVKKPGSSVKVSCKASGGTFNRHAISWVRQAPGQGLEWMGGIIPLFGTANYAQKFQGRVTIAADEPTSTTYMELRSLRSEDTAVYYCASSYFYGSGSSNYYYYGLDVWGQGTTVTVSS

SEQ ID NO: 756 - protein, 125 aa, synthetic construct
QVQLVQSGAEVKRPGSSVKVSCKASGGTFSIYVISWVRQAPGQGLEWMGGIIPLFGTANYAQKFQGRVTITADESTNTAYMELSSLRSEDTAVYYCARGHGPGSSHYSYYGLDVWGQGTTVTVSS

SEQ ID NO: 757 - protein, 126 aa, synthetic construct
QVQLVQSGAEVKKPGSSVKVSCKASGGTFSSSAISWVRQAPGQGLEWMGGIIPIFSTGNYAQKFQGRVTITADESTNTAYMDLSSLRSEDTAVYYCARSTRVRGVSHYYYYGLDVWGQGTTVTVSS

SEQ ID NO: 758 - protein, 127 aa, synthetic construct
QVQLVQSGAEVKKPGSSVKVSCKSSGGTFSSYAFTWVRQAPGQGLEWMGGIIPIFRTANYAQKFQGRVTITADESTSTAYMELSSLRSEDTAVYYCASSHFSGSGSSHYYYYGMHVWGQGTTVTVSS

SEQ ID NO: 759 - protein, 125 aa, synthetic construct
QVQLVQSGAEVKKPGSSVKVSCKASGGTFSRYAISWVRQAPGQGLEWMGGIIPIFGTSNYAQKFQGRVTIKADESTSTAYMELSSLRSEDTAVYYCARGGNSWTTSLYYYGMDVWGQGTTVTVSS

SEQ ID NO: 760 - protein, 127 aa, synthetic construct
QVQLVQSGAEVKKPGSSVKVSCKASGGTFNRYAFSWVRQAPGQGLEWMGGIIPIFGTPNYAQKFQGRVTITADESTSTAYMELSSLISEDTAVYYCASSHFYGSGSSHFYYYGMHVWGQGTTVTVSS

SEQ ID NO: 761 - protein, 127 aa, synthetic construct
QVQLVQSGAEVKKPGSSVKVSCKASGGTFNRYAFSWVRQAPGQGLEWMGGIIPIFGTPNYAQKFQGRVTITADESTSTAYMELSSLISEDTAVYYCASSHFYGSGSSNYYYYGLDVWGQGTTVTVSS

SEQ ID NO: 762 - protein, 126 aa, synthetic construct
QVQLVQSGAEVKKPGSSVKVSCKASGGTFSSSAISWVRQAPGQGLEWMGGIIPIFGTADSAQKFQGRVTITADESTSTAYMELNSLRSEDTAVYYCAFTPRYRGSSHHYFYALGVWGQGTTVTVSS

SEQ ID NO: 763 - protein, 125 aa, synthetic construct
QVQLVQSGAEVKKPGSSVKVSCKASGGTFSSSAISWVRQAPGQGLEWMGGIIPIFGTANYAQKFQGRVTITADESTSTAYMELSSLRSEDTAVYYCARASQSRSSNYYYYGLDVWGQGTTVTVSS

SEQ ID NO: 764 - protein, 15 aa, synthetic construct
RASESVDTFDYSFLH

SEQ ID NO: 765 - protein, 7 aa, synthetic construct
RASNLES

SEQ ID NO: 766 - protein, 15 aa, synthetic construct
RASQSVDTFDYSFLH

SEQ ID NO: 767 - protein, 9 aa, synthetic construct
QQSNQDPYT

SEQ ID NO: 768 - protein, 11 aa, synthetic construct
RASQGISSWLA

SEQ ID NO: 769 - protein, 7 aa, synthetic construct
AASSLQS

SEQ ID NO: 770 - protein, 11 aa, synthetic construct
RASQSVSSYLA

SEQ ID NO: 771 - protein, 7 aa, synthetic construct
GASSRAT

SEQ ID NO: 772 - protein, 9 aa, synthetic construct
QQYNSYPYT

SEQ ID NO: 773 - protein, 9 aa, synthetic construct
QQANSFPFT

SEQ ID NO: 774 - protein, 9 aa, synthetic construct
QQYNSYPWT

SEQ ID NO: 775 - protein, 9 aa, synthetic construct
QQYNSYPLT

SEQ ID NO: 776 - protein, 9 aa, synthetic construct
QQYNSYPIT

SEQ ID NO: 777 - protein, 9 aa, synthetic construct
QQFNSYPLT

SEQ ID NO: 778 - protein, 9 aa, synthetic construct
QQYNSYPPT

SEQ ID NO: 779 - protein, 9 aa, synthetic construct
QQYGSSPLT

SEQ ID NO: 780 - protein, 11 aa, synthetic construct
QASQDISNYLN

SEQ ID NO: 781 - protein, 12 aa, synthetic construct
RASQSVSSSYLA

SEQ ID NO: 782 - protein, 17 aa, synthetic construct
KSSQSVLYSSNNKNYLA

SEQ ID NO: 783 - protein, 11 aa, synthetic construct
RASQSVSSNLA

SEQ ID NO: 784 - protein, 16 aa, synthetic construct
RSSQSLLHSNGYNYLD

SEQ ID NO: 785 - protein, 17 aa, synthetic construct
KSSQSVLFSSNNKNYLA

SEQ ID NO: 786 - protein, 12 aa, synthetic construct
RASQSVSSSFLA

SEQ ID NO: 787 - protein, 11 aa, synthetic construct
RASQSISSWLA

SEQ ID NO: 788 - protein, 11 aa, synthetic construct
RASQSISSFLN

SEQ ID NO: 789 - protein, 11 aa, synthetic construct
RASQSIGSWLA

SEQ ID NO: 790 - protein, 11 aa, synthetic construct
RASQSISSYLN

SEQ ID NO: 791 - protein, 11 aa, synthetic construct
RASQSVGSNLA

SEQ ID NO: 792 - protein, 11 aa, synthetic construct
RASQSISRYLN

SEQ ID NO: 793 - protein, 11 aa, synthetic construct
RASQSINSWLA

SEQ ID NO: 794 - protein, 11 aa, synthetic construct
RASQDISSWLA

SEQ ID NO: 795 - protein, 11 aa, synthetic construct
RASQGIDSWLA

SEQ ID NO: 796 - protein, 16 aa, synthetic construct
RSSQSLLHRNGYNYLD

SEQ ID NO: 797 - protein, 7 aa, synthetic construct
DASNLET

SEQ ID NO: 798 - protein, 7 aa, synthetic construct
GASSRAT

SEQ ID NO: 799 - protein, 7 aa, synthetic construct
WASTRES

SEQ ID NO: 800 - protein, 7 aa, synthetic construct
GASTRAT

SEQ ID NO: 801 - protein, 7 aa, synthetic construct
LGSNRAS

SEQ ID NO: 802 - protein, 7 aa, synthetic construct
DASNRAT

SEQ ID NO: 803 - protein, 7 aa, synthetic construct
DASNLAT

SEQ ID NO: 804 - protein, 7 aa, synthetic construct
KASSLES

SEQ ID NO: 805 - protein, 7 aa, synthetic construct
AASNLQS

SEQ ID NO: 806 - protein, 7 aa, synthetic construct
DASSLES

SEQ ID NO: 807 - protein, 7 aa, synthetic construct
LGSHRAS

SEQ ID NO: 808 - protein, 7 aa, synthetic construct
DSSNRAT

SEQ ID NO: 809 - protein, 9 aa, synthetic construct
QQVYVLPFT

SEQ ID NO: 810 - protein, 9 aa, synthetic construct
QQYLGFPPT

SEQ ID NO: 811 - protein, 9 aa, synthetic construct
QQSFLTPWT

SEQ ID NO: 812 - protein, 9 aa, synthetic construct
QQFNNHPIT

SEQ ID NO: 813 - protein, 9 aa, synthetic construct
VQARQTPLT

SEQ ID NO: 814 - protein, 9 aa, synthetic construct
MQARDAPWT

SEQ ID NO: 815 - protein, 9 aa, synthetic construct
QQLASYPYT

SEQ ID NO: 816 - protein, 9 aa, synthetic construct
MQARQTPFT

SEQ ID NO: 817 - protein, 9 aa, synthetic construct
MQARQAPWT

SEQ ID NO: 818 - protein, 10 aa, synthetic construct
MQARQVPPWT

SEQ ID NO: 819 - protein, 8 aa, synthetic construct
MQARQAFT

SEQ ID NO: 820 - protein, 9 aa, synthetic construct
QQYTSWPLT

SEQ ID NO: 821 - protein, 9 aa, synthetic construct
QQLDSHPPT

SEQ ID NO: 822 - protein, 9 aa, synthetic construct
QQYDVDPLT

SEQ ID NO: 823 - protein, 9 aa, synthetic construct
QQAFISPPT

SEQ ID NO: 824 - protein, 9 aa, synthetic construct
QQADTLPIT

SEQ ID NO: 825 - protein, 9 aa, synthetic construct
QQSDIHPRT

SEQ ID NO: 826 - protein, 9 aa, synthetic construct
QQDSIYPIT

SEQ ID NO: 827 - protein, 9 aa, synthetic construct
QQANSFPLT

SEQ ID NO: 828 - protein, 10 aa, synthetic construct
QQYKSFSPFT

SEQ ID NO: 829 - protein, 8 aa, synthetic construct
QQSYSDLT

SEQ ID NO: 830 - protein, 9 aa, synthetic construct
QQYLIPPIT

SEQ ID NO: 831 - protein, 9 aa, synthetic construct
QQHQSFSPT

SEQ ID NO: 832 - protein, 9 aa, synthetic construct
QQRSVLPLT

SEQ ID NO: 833 - protein, 9 aa, synthetic construct
QQIFSTPLT

SEQ ID NO: 834 - protein, 9 aa, synthetic construct
QQSFYDPIT

SEQ ID NO: 835 - protein, 9 aa, synthetic construct
QQYLYFPLT

SEQ ID NO: 836 - protein, 9 aa, synthetic construct
QQGVNYPFT

SEQ ID NO: 837 - protein, 8 aa, synthetic construct
QQVISFPT

SEQ ID NO: 838 - protein, 10 aa, synthetic construct
QQYDDFPPIT

SEQ ID NO: 839 - protein, 9 aa, synthetic construct
QQSLDLPFT

SEQ ID NO: 840 - protein, 9 aa, synthetic construct
QQINDHPFT

SEQ ID NO: 841 - protein, 9 aa, synthetic construct
QQYGPYPYT

SEQ ID NO: 842 - protein, 9 aa, synthetic construct
QQSHSTPLT

SEQ ID NO: 843 - protein, 9 aa, synthetic construct
QQLASQPPT

SEQ ID NO: 844 - protein, 9 aa, synthetic construct
QQYAYWPLT

SEQ ID NO: 845 - protein, 9 aa, synthetic construct
QQDFSLPYT

SEQ ID NO: 846 - protein, 8 aa, synthetic construct
QQSLTHPT

SEQ ID NO: 847 - protein, 9 aa, synthetic construct
QQYDLLPYT

SEQ ID NO: 848 - protein, 10 aa, synthetic construct
QQAVIHPPYT

SEQ ID NO: 849 - protein, 10 aa, synthetic construct
QQYNVHPPRT

SEQ ID NO: 850 - protein, 9 aa, synthetic construct
MQSRNAPWT

SEQ ID NO: 851 - protein, 8 aa, synthetic construct
MQARHGFT

SEQ ID NO: 852 - protein, 9 aa, synthetic construct
MQAREVPFT

SEQ ID NO: 853 - protein, 10 aa, synthetic construct
MQARHVPPLT

SEQ ID NO: 854 - protein, 9 aa, synthetic construct
QQHDSAPYT

SEQ ID NO: 855 - protein, 9 aa, synthetic construct
MQGRQVPFT

SEQ ID NO: 856 - protein, 9 aa, synthetic construct
MQARGTPWT

SEQ ID NO: 857 - protein, 10 aa, synthetic construct
MQSRRAPPWT

SEQ ID NO: 858 - protein, 9 aa, synthetic construct
QQFQSYPFT

SEQ ID NO: 859 - protein, 10 aa, synthetic construct
QQSSADSPFT

SEQ ID NO: 860 - protein, 9 aa, synthetic construct
MQARQLPWT

SEQ ID NO: 861 - protein, 9 aa, synthetic construct
QQHDVWPIT

SEQ ID NO: 862 - protein, 8 aa, synthetic construct
MQTRHTPT

SEQ ID NO: 863 - protein, 9 aa, synthetic construct
MQDFARPPT

SEQ ID NO: 864 - protein, 9 aa, synthetic construct
QQRAVFPPT

SEQ ID NO: 865 - protein, 8 aa, synthetic construct
QQDATGIT

SEQ ID NO: 866 - protein, 9 aa, synthetic construct
QQLASFPWT

SEQ ID NO: 867 - protein, 9 aa, synthetic construct
QQLAFTPWT

SEQ ID NO: 868 - skipped

SEQ ID NO: 869 - protein, 8 aa, synthetic construct
QQDHSFIT

SEQ ID NO: 870 - protein, 8 aa, synthetic construct
QQDVSDFT

SEQ ID NO: 871 - protein, 10 aa, synthetic construct
QQLYHAPPIT

SEQ ID NO: 872 - protein, 9 aa, synthetic construct
QQYDSLPFT

SEQ ID NO: 873 - protein, 9 aa, synthetic construct
QQVYLFPWT

SEQ ID NO: 874 - protein, 9 aa, synthetic construct
QQFFLAPPT

SEQ ID NO: 875 - protein, 9 aa, synthetic construct
QQAVSLPWT

SEQ ID NO: 876 - protein, 9 aa, synthetic construct
QQFDNLPYT

SEQ ID NO: 877 - protein, 9 aa, synthetic construct
QQATAHPPT

SEQ ID NO: 878 - protein, 9 aa, synthetic construct
QQAVSHPLT

SEQ ID NO: 879 - protein, 9 aa, synthetic construct
QQATSLPLT

SEQ ID NO: 880 - protein, 8 aa, synthetic construct
MQRLQAWT

SEQ ID NO: 881 - protein, 8 aa, synthetic construct
QQYRTYPT

SEQ ID NO: 882 - protein, 9 aa, synthetic construct
QQHSLLSIT

SEQ ID NO: 883 - protein, 8 aa, synthetic construct
QHYNLWRT

SEQ ID NO: 884 - protein, 9 aa, synthetic construct
QQHSTYSWT

SEQ ID NO: 885 - protein, 9 aa, synthetic construct
QQHDVWPYT

SEQ ID NO: 886 - protein, 9 aa, synthetic construct
QQYFSTPPT

SEQ ID NO: 887 - protein, 9 aa, synthetic construct
QQYALTPYT

SEQ ID NO: 888 - protein, 9 aa, synthetic construct
QQDHDRPLT

SEQ ID NO: 889 - protein, 8 aa, synthetic construct
HQWSGYPT

SEQ ID NO: 890 - protein, 12 aa, synthetic construct
RASQSVSSSYLA

SEQ ID NO: 891 - protein, 8 aa, synthetic construct
QQYGSSPT

SEQ ID NO: 892 - protein, 11 aa, synthetic construct
RASQGISSALA

SEQ ID NO: 893 - protein, 7 aa, synthetic construct
DASSLES

SEQ ID NO: 894 - protein, 9 aa, synthetic construct
QQFNSYPYT

SEQ ID NO: 895 - protein, 10 aa, synthetic construct
DLGIRRQFAY

SEQ ID NO: 896 - protein, 111 aa, synthetic construct
DIVLTQSPDSLAVSLGERATINCRASQSVDTFDYSFLHWYQQKPGQPPKLLIYRASNLESGVPDRFSGSGSGTDFTLTISSLQAEDVAVYYCQQSNQDPYTFGQGTKLEIK

SEQ ID NO: 897 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQVYVLPFTFGGGTKVEIK

SEQ ID NO: 898 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYLGFPPTFGGGTKVEIK

SEQ ID NO: 899 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQSFLTPWTFGGGTKVEIK

SEQ ID NO: 900 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQFNNHPITFGGGTKVEIK

SEQ ID NO: 901 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCVQARQTPLTFGGGTKVEIK

SEQ ID NO: 902 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARDAPWTFGGGTKVEIK

SEQ ID NO: 903 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQLASYPYTFGGGTKVEIK

SEQ ID NO: 904 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARQTPFTFGGGTKVEIK

SEQ ID NO: 905 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARQAPWTFGGGTKVEIK

SEQ ID NO: 906 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARQVPPWTFGGGTKVEIK

SEQ ID NO: 907 - protein, 111 aa, synthetic construct
DIVMTQSPLSLPVTPGEPASISCRSSQSLLHSNGYNYLDWYLQKPGQSPQLLIFLGSNRASGVPDRFSGSGSGTDFTLKISRVEAEDVGVYYCMQARQAFTFGGGTKVEIK

SEQ ID NO: 908 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQYTSWPLTFGGGTKVEIK

SEQ ID NO: 909 - protein, 108 aa, synthetic construct
EIVLTQSPGTLSLSPGERATLSCRASQSVSSSFLAWYQQKPGQAPRLLIYGASSRATGIPDRFSGSGSGTDFTLTISRLEPEDFAVYYCQQLDSHPPTFGGGTKVEIK

SEQ ID NO: 910 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYDVDPLTFGGGTKVEIK

SEQ ID NO: 911 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAFISPPTFGGGTKVEIK

SEQ ID NO: 912 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQADTLPITFGGGTKVEIK

SEQ ID NO: 913 - protein, 107 aa, synthetic construct
DIQMTQSPSSLSASVGDRVTITCQASQDISNYLNWYQQKPGKAPKLLIYDASNLATGVPSRFSGSGSGTDFTFTISSLQPEDIATYYCQQSDIHPRTFGGGTKVEIK

SEQ ID NO: 914 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDSIYPITFGGGTKVEIK

SEQ ID NO: 915 - protein, 107 aa, synthetic construct
DIQMTQSPSSVSASVGDRVTITCRASQGISSWLAWYQQKPGKAPKLLIYAASNLQSGVPSRFSGSGSGTDFTLTISSLQPEDFATYYCQQANSFPLTFGGGTKVEIK

SEQ ID NO: 916 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYKSFSPFTFGGGTKVEIK

SEQ ID NO: 917 - protein, 106 aa, synthetic construct
DIQLTQSPSSLSASVGDRVTITCRASQSISSFLNWYQQKPGKAPKLLIYAASSLQSGVPSRFSGSGSGTDFTLTISSLQPEDFATYYCQQSYSDLTFGGGTKVEIK

SEQ ID NO: 918 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYLIPPITFGGGTKVEIK

SEQ ID NO: 919 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQHQSFSPTFGGGTKVEIK

SEQ ID NO: 920 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSVLPLTFGGGTKVEIK

SEQ ID NO: 921 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQIFSTPLTFGGGTKVEIK

SEQ ID NO: 922 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSFYDPITFGGGTKVEIK

SEQ ID NO: 923 - protein, 107 aa, synthetic construct
EIVLTQSPATLSVSPGERATLSCRASQSVGSNLAWYQQKPGQAPRLLIYGASTRATGIPARFSGSGSGTEFTLTISSLQSEDFAVYYCQQYLYFPLTFGGGTKVEIK

SEQ ID NO: 924 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQGVNYPFTFGGGTKVEIK

SEQ ID NO: 925 - protein, 106 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQVISFPTFGGGTKVEIK

SEQ ID NO: 926 - protein, 108 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDDFPPITFGGGTKVEIK

SEQ ID NO: 927 - protein, 107 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQSLDLPFTFGGGTKVEIK

SEQ ID NO: 928 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQINDHPFTFGGGTKVEIK

SEQ ID NO: 929 - protein, 107 aa, synthetic construct
DIQMTQSPSTLSASVGDRVTITCRASQSINSWLAWYQQKPGKAPKLLISDASSLESGVPSRFSGSGSGTEFTLTISSLQPDDFATYYCQQYGPYPYTFGGGTKVEIK

SEQ ID NO: 930 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQSHSTPLTFGGGTKVEIK

SEQ ID NO: 931 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLASQPPTFGGGTKVEIK

SEQ ID NO: 932 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYAYWPLTFGGGTKVEIK

SEQ ID NO: 933 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQDFSLPYTFGGGTKVEIK

SEQ ID NO: 934 - protein, 106 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQSLTHPTFGGGTKVEIK

SEQ ID NO: 935 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYDLLPYTFGGGTKVEIK

SEQ ID NO: 936 - protein, 108 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQAVIHPPYTFGGGTKVEIK

SEQ ID NO: 937 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNVHPPRTFGGGTKVEIK

SEQ ID NO: 938 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSRNAPWTFGGGTKVEIK

SEQ ID NO: 939 - protein, 111 aa, synthetic construct
DIVMTQSPLSLPVTPGEPASISCRSSQSLLHSNGYNYLDWYLQKPGQSPQVLIYLGSNRASGVPDRFSGSGSGTDFTLKISRVEAEDVGVYYCMQARHGFTFGGGTKVEIK

SEQ ID NO: 940 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQAREVPFTFGGGTKVEIK

SEQ ID NO: 941 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARHVPPLTFGGGTKVEIK

SEQ ID NO: 942 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHDSAPYTFGGGTKVEIK

SEQ ID NO: 943 - protein, 112 aa, synthetic construct
DIVMTQSPLSLPVTPGEPASISCRSSQSLLHSNGYNYLDWYLQKPGQSPQLLIYLGSHRASGVPDRFSGSGSGTDFTLKISRVEAEDVGVYYCMQGRQVPFTFGGGTKVEIK

SEQ ID NO: 944 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARGTPWTFGGGTKVEIK

SEQ ID NO: 945 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSRRAPPWTFGGGTKVEIK

SEQ ID NO: 946 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQFQSYPFTFGGGTKVEIK

SEQ ID NO: 947 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQSSADSPFTFGGGTKVEIK

SEQ ID NO: 948 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARQLPWTFGGGTKVEIK

SEQ ID NO: 949 - protein, 107 aa, synthetic construct
EIVMTQSPATLSLSPGERATLSCRASQSVSSYLAWYQQKPGQAPRLLIYDSSNRATGIPARFSGSGSGTDFTLTISSLEPEDFAVYYCQQHDVWPITFGGGTKVEIK

SEQ ID NO: 950 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQTRHTPTFGGGTKVEIK

SEQ ID NO: 951 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQDFARPPTFGGGTKVEIK

SEQ ID NO: 952 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQRAVFPPTFGGGTKVEIK

SEQ ID NO: 953 - protein, 112 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQDATGITFGGGTKVEIK

SEQ ID NO: 954 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLASFPWTFGGGTKVEIK

SEQ ID NO: 955 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQLAFTPWTFGGGTKVEIK

SEQ ID NO: 956 - protein, 112 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQDHSFITFGGGTKVEIK

SEQ ID NO: 957 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQDVSDFTFGGGTKVEIK

SEQ ID NO: 958 - protein, 108 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQLYHAPPITFGGGTKVEIK

SEQ ID NO: 959 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLEPEDVAVYYCQQYDSLPFTFGGGTKVEIK

SEQ ID NO: 960 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQVYLFPWTFGGGTKVEIK

SEQ ID NO: 961 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFFLAPPTFGGGTKVEIK

SEQ ID NO: 962 - protein, 108 aa, synthetic construct
EIVLTQSPGTLSLSPGERATLSCRASQSVSSSFLAWYQQKPGQAPRLLIYGASSRATGIPDRFSGSGSGTDFTLTISRLEPEDFAVYYCQQAVSLPWTFGGGTKVEIK

SEQ ID NO: 963 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQFDNLPYTFGGGTKVEIK

SEQ ID NO: 964 - protein, 107 aa, synthetic construct
DIQMTQSPSSVSASVGDRVTITCRASQGISSWLAWYQQKPGKAPKLLIYAASNLQSGVPSRFSGSGSGTDFTLTISSLQPEDFATYYCQQATAHPPTFGGGTKVEIK

SEQ ID NO: 965 - protein, 107 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQAVSHPLTFGGGTKVEIK

SEQ ID NO: 966 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQATSLPLTFGGGTKVEIK

SEQ ID NO: 967 - protein, 111 aa, synthetic construct
DIVMTQSPLSLPVTPGEPASISCRSSQSLLHRNGYNYLDWYLQKPGQSPQLLIYLGSNRASGVPDRFSGSGSGTDFTLKISRVEAEDVGVYYCMQRLQAWTFGGGTKVEIK

SEQ ID NO: 968 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYRTYPTFGGGTKVEIK

SEQ ID NO: 969 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQHSLLSITFGGGTKVEIK

SEQ ID NO: 970 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQHYNLWRTFGGGTKVEIK

SEQ ID NO: 971 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQHSTYSWTFGGGTKVEIK

SEQ ID NO: 972 - protein, 107 aa, synthetic construct
EIVMTQSPATLSVSPGERATLSCRASQSVGSNLAWYQQKPGQAPRLLIYGASTRATGIPARFSGSGSGTEFTLTISSLQSEDFAVYYCQQHDVWPYTFGGGTKVEIK

SEQ ID NO: 973 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYFSTPPTFGGGTKVEIK

SEQ ID NO: 974 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYALTPYTFGGGTKVEIK

SEQ ID NO: 975 - protein, 107 aa, synthetic construct
EIVMTQSPATLSVSPGERATLSCRASQSVGSNLAWYQQKPGQAPRLLIYGASTRATGIPARFSGSGSGTEFTLTISSLQSEDFAVYYCQQDHDRPLTFGGGTKVEIK

SEQ ID NO: 976 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGGGTKVEIK

SEQ ID NO: 977 - protein, 107 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 978 - protein, 108 aa, synthetic construct
EIVLTQSPGTLSLSPGERATLSCRASQSVSSSYLAWYQQKPGQAPRLLIYGASSRATGIPERFSGSGSGTDFTLTISRLEPEDFAVYYCQQYGSSPLTFGGGTKVEIK

SEQ ID NO: 979 - protein, 107 aa, synthetic construct
AIQLTQSPSSLSASVGDRVTITCRASQGISSALAWYQQKPGKAPKLLIYAASSLQSGVPSRFSGSGSGTDFTLTISSLQPEDFATYYCQQYNSYPLTFGGGTKVEIK

SEQ ID NO: 980 - protein, 107 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIK

SEQ ID NO: 981 - protein, 107 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQYNSYPITFGQGTRLEIK

SEQ ID NO: 982 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 983 - protein, 107 aa, synthetic construct
DIQMTQSPSSVSASVGDRVTITCRASQGISSWLAWYQHKPGKAPKLLIYAASSLQSGVPSRFSGSGSGTDFTLTISSLQPEDFATYYCQQANSFPFTFGGGTKVEIK

SEQ ID NO: 984 - protein, 107 aa, synthetic construct
DIQMTQSPTSLSASVGDRVTITCRASQGISSWLAWYQHKPGKAPKLLIYAASSLQSGVPSRFSGSGSGTDFTLTISSLQPEDFATYYCQQYNSYPWTFGQGTKVEIK

SEQ ID NO: 985 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 986 - protein, 107 aa, synthetic construct
DIQMTQSPTSLSASVGDRVTITCRASQGISSWLAWYQHKPGKAPKLLIYAASSLQSGVPSRFSGSGSGTDFTLTISSLQPEDFATYYCQQYNSYPYTFGQGTKLEIK

SEQ ID NO: 987 - protein, 107 aa, synthetic construct
DIQMTQSPTSLSASVGDRVTITCRASQGISSWLAWYQHKPGKAPKSLIYAASSLQSGVPSRFSGSGSGTDFTLTISSLQPEDFATYYCQQYNSYPYTFGQGTKLEIK

SEQ ID NO: 988 - protein, 107 aa, synthetic construct
DIQMTQSPTSLSASVGDRVTITCRASQGISSWLAWYQQKPGKAPKSLIYAASSLQSGVPSRFSGSGSGTDFTLTISSLQPEDFATYYCQQYNSYPLTFGGGTKVEIK

SEQ ID NO: 989 - protein, 107 aa, synthetic construct
DIQMTQSPTSLSASVGDRVTITCRASQGISSWLAWYQQKPEKAPKSLIYAASSLQSGVPSRFSGGGSGTDFTLTISSLQPEDFATYYCQQYNSYPLTFGGGTKVEIK

SEQ ID NO: 990 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPDDFATYYCQQYNSYPYTFGQGTKLEIK

SEQ ID NO: 991 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPEDFATYYCQQYNSYPITFGQGTRLEIK

SEQ ID NO: 992 - protein, 107 aa, synthetic construct
DIQMTQSPTSLSASVGDRVTITCRASQGISSWLAWYQHKPGKAPKSLIYAASSLQSGVPSRFSGSGSGTDFTLTISSLQPEDFATYYCQQYNSYPLTFGGGTKVEIK

SEQ ID NO: 993 - protein, 107 aa, synthetic construct
DIQMTQSPTSLSASVGDRVTITCRASQGISSWLAWYQQKPEKAPKSLIHAASSLQSGVPSRFSGSGSGTDFTLTISSLQPEDFATYYCQQYNSYPLTFGGGTKVEIK

SEQ ID NO: 994 - protein, 107 aa, synthetic construct
DIQMTQSPTSLSASVGDRVTITCRASQGISSWLAWYQQKPEKAPKSLIYAASSLQSGVPSRFSGSGSGTDYTLTISSLQPEDFATYYCQQYNSYPLTFGGGTKVEIK

SEQ ID NO: 995 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPEDFATYYCQQYNSYPWTFGQGTKVEIK

SEQ ID NO: 996 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPEDFATYYCQQFNSYPLTFGGGTKVEIK

SEQ ID NO: 997 - protein, 107 aa, synthetic construct
DIQMTQSPTSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIK

SEQ ID NO: 998 - protein, 107 aa, synthetic construct
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIK

SEQ ID NO: 999 - protein, 126 aa, synthetic construct
QVQLVQSGAEVKKPGASVKVSCKASGYTFPTYDINWVRQATGQGLEWMGWVNPNSGNTGYAQKFQDRVTMTRNTSISTAYMELSSLRSEDTAVYYCASDGLNMVRGVHNYYGMDVWGQGTTVTVSS

SEQ ID NO: 1000 - DNA, 1350 bp, synthetic construct
  source 1..1350: mol_type = other DNA
gaagtgcagctggttgaatccggtggggggcttgtacaaccagggggctctcttaaactttcatgtgctgcctccggttttacgtttagcacctacgcaatgcactgggttaggcaggcgtccggcaagggcctggagtgggtcggtcggattcgcacgaagtcaagcaattacgcgacgtattatgcggcatctgtcaagggaaggttcactatctctagagacgattctaagaacaccgcttatcttcagatgaactctctgaaaacagaagatacagctgtatattactgtactcgggatatgggcatacgaaggcagtttgcgtattgggggcaagggacactcgtaactgtgagcagtgctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaagcagctggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttgcagccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 1001 - DNA, 654 bp, synthetic construct
  source 1..654: mol_type = other DNA
gacattgtgctcacccaaagcccggacagccttgcggtctcccttggagaaagagcaactatcaattgtagggcgtctgaaagtgttgacacctttgattactccttcctgcactggtatcaacagaagccaggtcaaccgccaaaactcctgatctatagagcttcaaatttggagtctggtgtccccgaccgatttagcggaagcggtagcgggactgattttacgctcaccatttcctctctgcaagctgaagatgtggcagtttactactgtcaacaaagtaacgaggacccatatacatttgggcagggaactaaattggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 1002 - DNA, 1347 bp, synthetic construct
  source 1..1347: mol_type = other DNA
gaggtgcaacttgtagagtctgggggcggactcgtccagccaggaggatccctcagactcagttgtgcagcgtcaggcttcagtctctcctcctacgcaatgacgtgggttaggcaagcacccggtaaaggtctggaatggatcgggattatttacgctggggggtccccaagttacgcgagctgggctaaaggtcgatttacaataagtaaagataattccaagaacaccttgtatctgcagatgaacagcttgagggcggaggacactgcagtttattattgtgcgcggggtacgggcgatacagtctatacttattttaacatttggggccaaggaaccttggtgaccgtgtcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaagcagctggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttgcagccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 1003 - DNA, 657 bp, synthetic construct
  source 1..657: mol_type = other DNA
gacatacaactgactcaatcaccaagttttttgtcagcatcagtcggcgacagagtaacgataacttgtcaggcgagtcagaatatcggtagtgacttggcttggtatcaacaaaaaccggggaaggcaccgaagctcctcatctacaaagctgctacgctcgcatcaggcgtcccctcacgcttttccggcagcggaagcggcacagaattcacgctcaccatcagtagccttcagccagaagactttgctacttattactgccaacaatattactacggcagcgcgggtgcagatacggacacctttggaggagggaccaaagtggaaattaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 1004 - DNA, 1350 bp, synthetic construct
  source 1..1350: mol_type = other DNA
gaagtgcagctggttgaatccggtggggggcttgtacaaccagggggctctcttaaactttcatgtgctgcctccggttttacgtttagcacctacgcaatgcactgggttaggcaggcgtccggcaagggcctggagtgggtcggtcggattcgcacgaagtcaagcaattacgcgacgtattatgcggcatctgtcaagggaaggttcactatctctagagacgattctaagaacaccgcttatcttcagatgaactctctgaaaacagaagatacagctgtatattactgtactcgggatatgggcatacgaaggcagtttgcgtattgggggcaagggacactcgtaactgtgagcagtgctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaagcagctggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttgcagccccaatcgagaaaaccatttccaaggccaaaggtcaaccaagagaaccccaggtgtgtactcttccgccttctcgtgaggaaatgactaaaaatcaagtatccctttcctgtgctgttaaaggtttttatcctagcgatattgctgttgaatgggaatcgaacggtcagccggagaataattataaaacaacgccacccgtcctggatagcgacggctcattttttctggtaagcaaactgactgtagataaatcacggtggcagcagggcaatgtattcagttgctccgttatgcatgaagcgttacataatcactacacgcagaaatctcttagtctttcacccggt

SEQ ID NO: 1005 - DNA, 1347 bp, synthetic construct
  source 1..1347: mol_type = other DNA
gaggtgcaacttgtagagtctgggggcggactcgtccagccaggaggatccctcagactcagttgtgcagcgtcaggcttcagtctctcctcctacgcaatgacgtgggttaggcaagcacccggtaaaggtctggaatggatcgggattatttacgctggggggtccccaagttacgcgagctgggctaaaggtcgatttacaataagtaaagataattccaagaacaccttgtatctgcagatgaacagcttgagggcggaggacactgcagtttattattgtgcgcggggtacgggcgatacagtctatacttattttaacatttggggccaaggaaccttggtgaccgtgtcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaagcagctggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttgcagccccaatcgagaaaaccatttccaaggccaaaggtcaaccaagagaaccccaggtgtgtactcttccgccttctcgtgaggaaatgactaaaaatcaagtatccctttcctgtgctgttaaaggtttttatcctagcgatattgctgttgaatgggaatcgaacggtcagccggagaataattataaaacaacgccacccgtcctggatagcgacggctcattttttctggtaagcaaactgactgtagataaatcacggtggcagcagggcaatgtattcagttgctccgttatgcatgaagcgttacataatcactacacgcagaaatctcttagtctttcacccggt